SEQUENCE LISTING (35 sequences)

SEQ ID NO: 1 - protein, 13 aa, Artificial Sequence
  Synthetic polypeptide
DLNIAMDGLREVM

SEQ ID NO: 2 - protein, 11 aa, Artificial Sequence
  Synthetic polypeptide
DLNIAMDGLRE

SEQ ID NO: 3 - protein, 10 aa, Artificial Sequence
  Synthetic polypeptide
DLNIAMDGLR

SEQ ID NO: 4 - protein, 7 aa, Artificial Sequence
  Synthetic polypeptide
DLNIAMD

SEQ ID NO: 5 - protein, 9 aa, Artificial Sequence
  Synthetic polypeptide
AMDGLREVM

SEQ ID NO: 6 - protein, 7 aa, Artificial Sequence
  Synthetic polypeptide
DGLREVM

SEQ ID NO: 7 - protein, 28 aa, Artificial Sequence
  Synthetic polypeptide
YAHGPSVRKLSKIATLLLARNYILMLTN

SEQ ID NO: 8 - protein, 20 aa, Artificial Sequence
  Synthetic polypeptide
YAHGPSVRKLSKIATLLLAR

SEQ ID NO: 9 - protein, 20 aa, Artificial Sequence
  Synthetic polypeptide
KLSKIATLLLARNYILMLTN

SEQ ID NO: 10 - protein, 14 aa, Artificial Sequence
  Synthetic polypeptide
TLLLARNYILMLTN

SEQ ID NO: 11 - protein, 13 aa, Artificial Sequence
  Synthetic polypeptide
RKLSKIATLLLAR

SEQ ID NO: 12 - protein, 12 aa, Artificial Sequence
  Synthetic polypeptide
YAHGPSVRKLSK

SEQ ID NO: 13 - protein, 9 aa, Artificial Sequence
  Synthetic polypeptide
RNYILMLTN

SEQ ID NO: 14 - protein, 57 aa, Homo sapiens
RLKINSRERKRMHDLNIAMDGLREVMPYAHGPSVRKLSKIATLLLARNYILMLTNSL

SEQ ID NO: 15 - protein, 55 aa, Artificial Sequence
  Synthetic polypeptide
RMKANARERNRMHGLNAALDNLRKVVPCYSKTQKLSKIETLRLAKNYIWALSEIL

SEQ ID NO: 16 - protein, 57 aa, Artificial Sequence
  Synthetic polypeptide
RRVANNARERLRVRDINEAFKELGRMCQLHLNSEKPQTKLLILHQAVSVILNLEQQV

SEQ ID NO: 17 - protein, 57 aa, Artificial Sequence
  Synthetic polypeptide
RRMANNARERLRVRDINEAFKELGRMCQLHLKSEKPQTKLLILHQAVAVILSLEQQV

SEQ ID NO: 18 - protein, 57 aa, Artificial Sequence
  Synthetic polypeptide
RRMANNARERVRVRDINEAFRELGRMCQMHLKSDKAQTKLLILQQAVQVILGLEQQV

SEQ ID NO: 19 - protein, 55 aa, Artificial Sequence
  Synthetic polypeptide
RRKAATMRERRRLSKVNEAFETLKRSTSSNPNQRLPKVEILRNAIRYIEGLQALL

SEQ ID NO: 20 - protein, 55 aa, Artificial Sequence
  Synthetic polypeptide
RRKAATMRERRRLKKVNQAFETLKRCTTTNPNQRLPKVEILRNAIRYIESLQELL

SEQ ID NO: 21 - protein, 56 aa, Artificial Sequence
  Synthetic polypeptide
RRVFTNSRERWRQQNVNGAFAELRKLLPTHPPDRKLSKNEVLRLAMKYIGFLVRLL

SEQ ID NO: 22 - protein, 56 aa, Artificial Sequence
  Synthetic polypeptide
RKIFTNTRERWRQQNVNSAFAKLRKLIPTHPPDKKLSKNETLRLAMRYINFLVKVL

SEQ ID NO: 23 - protein, 55 aa, Artificial Sequence
  Synthetic polypeptide
RRRAATLREKRRLKKVNEAFEALKRSTLLNPNQRLPKVEILRSAIQYIERLQALL

SEQ ID NO: 24 - protein, 55 aa, Artificial Sequence
  Synthetic polypeptide
RRKAATLRERRRLKKINEAFEALKRRTVANPNQRLPKVEILRSAISYIERLQDLL

SEQ ID NO: 25 - protein, 56 aa, Artificial Sequence
  Synthetic polypeptide
RRMKANARERNRMHGLNAALDNLRKVVPCYSKTQKLSKIETLRLAKNYIWALSEIL

SEQ ID NO: 26 - protein, 58 aa, Artificial Sequence
  Synthetic polypeptide
LRLKINSRERKRMHDLNIAMDGLREVMPYAHGPSVRKLSKIATLLLARNYILMLTNSL

SEQ ID NO: 27 - protein, 56 aa, Artificial Sequence
  Synthetic polypeptide
RRLAANARERRRMHGLNHAFDQLRNVIPSFNNDKKLSKYETLQMAQIYINALSETP

SEQ ID NO: 28 - protein, 56 aa, Artificial Sequence
  Synthetic polypeptide
LRQAANVRERRRMQSINDAFEGLRSHIPTLPYEKRLSKVDTLRLAIGYINFLSELV

SEQ ID NO: 29 - protein, 56 aa, Artificial Sequence
  Synthetic polypeptide
CAHAGARGGARRTQSINSAFAELRECIPNVPADTKLSKIKTLRLATSYIAYLMDLL

SEQ ID NO: 30 - protein, 56 aa, Artificial Sequence
  Synthetic polypeptide
RKGSGPKKERRRTESINSAFAELRECIPNVPADTKLSKIKTLRLATSYIAYLMDVL

SEQ ID NO: 31 - protein, 56 aa, Artificial Sequence
  Synthetic polypeptide
QRNAANARERARMRVLSKAFSRLKTTLPWVPPDTKLSKLDTLRLASSYIAHLRQIL

SEQ ID NO: 32 - protein, 56 aa, Artificial Sequence
  Synthetic polypeptide
AVARRNERERNRVKLVNLGFATLREHVPNGAANKKMSKVETLRSAVEYIRALQQLL

SEQ ID NO: 33 - protein, 56 aa, Artificial Sequence
  Synthetic polypeptide
FTRKRNERERQRVKCVNEGYAQLRHHLPEEYLEKRLSKVETLRAAIKYINYLQSLL

SEQ ID NO: 34 - protein, 49 aa, Artificial Sequence
  Synthetic polypeptide
RRDRINSSIEQLKLLLEQEFARHQPNSKLEKADILEMAVSYLKHSKGER

SEQ ID NO: 35 - protein, 56 aa, Artificial Sequence
  Synthetic polypeptide
GKGPAAEEPLSLLDDMNHCYSRLRELVPGVPRGTQLSQVEILQVVLAEPAPGPPDG

(19) United States
(12) Patent Application Publication
(10) Pub. No.: US 2017/0182049 A1
(43) Pub. Date: Jun. 29, 2017

(54) NOVEL THERAPEUTICS FOR BRAIN CANCER

(71) Applicant: The Regents of the University of California, Oakland, CA (US)

(72) Inventors: Santosh Kesari, San Diego, CA (US); Milan Makale, San Diego, CA (US); Wolf Wrasidlo, La Jolla, CA (US); Rajesh Mukthavaram, La Jolla, CA (US); Igor Flint Tsigelny, San Diego, CA (US); Valentina Lea Kouznetsova, San Diego, CA (US)

(21) Appl. No.: 15/225,700

(22) Filed: Aug. 1, 2016

Related U.S. Application Data
(63) Continuation of application No. 14/549,900, filed on Nov. 21, 2014, now abandoned.
Application No. 14/549,900 is a continuation of application No. PCT/US2013/045968, filed on Jun. 14, 2013.
(60) Provisional application No. 61/660,631, filed on Jun. 15, 2012.

(51) Int. Cl. A61K 31/506; A61K 31/4545

(52) U.S. Cl. CPC A61K 31/506; A61K 31/4545

(57) ABSTRACT

Provided herein are novel compositions and methods to inhibit Olig2 activity. The Olig2 inhibitors and methods of using the same are useful, inter alia, for treating cancer. In particular the Olig2 inhibitors may be used to treat glioblastoma. Further, provided are peptide compositions capable of inhibiting Olig 2.

### CROSS-REFERENCES TO RELATED APPLICATIONS

This application is a continuation of U.S. application Ser. No. 14/549,900, filed Nov. 21, 2014, which is a continuation of PCT Application No. PCT/US2013/045968, filed Jun. 14, 2013, which claims priority to U.S. Provisional Patent Application No. 61/660,631, filed Jun. 15, 2012, all entitled “NOVEL THERAPEUTICS FOR BRAIN CANCER,” the disclosures of which are incorporated by reference herein in their entirety.

### STATEMENT AS TO RIGHTS TO INVENTIONS MADE UNDER FEDERALLY SPONSORED RESEARCH AND DEVELOPMENT

This invention was made with government support under grant numbers CA023100 and CA124804 awarded by the National Institutes of Health. The Government has certain rights in the invention.

### REFERENCE TO A SEQUENCE LISTING APPENDIX SUBMITTED AS AN ASCII TEXT FILE

The Sequence Listing written in file 88654-924304_ST.TXT, created on Jan. 28, 2015, 16,535 bytes, machine format IBM-PC, MS-Windows operating system, is hereby incorporated by reference.

### BACKGROUND OF THE INVENTION

Current brain tumor therapeutic agents, which are only able to extend median survival of patients to six months, cause significant systemic toxicity. This toxicity results in serious long term morbidity of the few patients that survive, in terms of cognition, endocrine disorders, and motor effects. Currently brain tumors are essentially incurable with a median survival of one year.

Provided herein are compositions and methods that address these and other problems in the art.

### BRIEF SUMMARY OF THE INVENTION

In one aspect, a compound having the formula

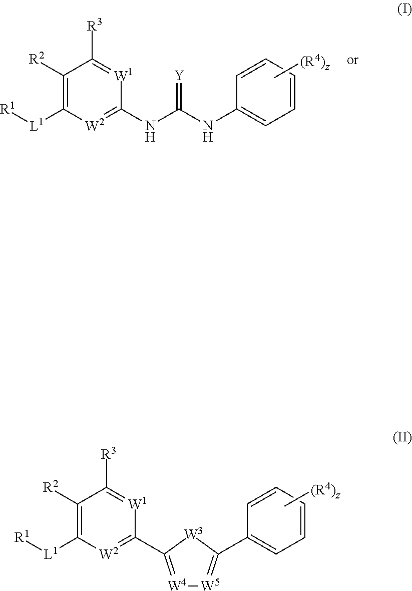

is provided. In formula (I) or (II) R¹ is independently hydrogen, halogen, —CX^{a}₃, —CN, —SO₂Cl, —SOₙ₁R⁵, —SOᵥ₁NR⁵R⁶, —NHNH₂, —ONR⁵R⁶, —NHC═(O)NHNH₂, —NHC═(O)NR⁵R⁶, —N(O)ₘ₁, —NR⁵R⁶, —NH—O—R⁵, —C(O)R⁵, —C(O)—OR⁵, —C(O)NR⁵R⁶, —OR⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R² is independently hydrogen, halogen, —CX^{b}₃, —CN, —SO₂Cl, —SOₙ₂R⁷, —SOᵥ₂NR⁷R⁸, —NHNH₂, —ONR⁷R⁸, —NHC═(O)NHNH₂, —NHC═(O)NR⁷R⁸, —N(O)ₘ₂, —NR⁷R⁸, —NH—O—R⁷, —C(O)R⁷, —C(O)—OR⁷, —C(O)NR⁷R⁸, —OR⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R³ is independently hydrogen, halogen, —CX^{c}₃, —CN, —SO₂Cl, —SOₙ₃R⁹, —SOᵥ₃NR⁹R¹⁰, —NHNH₂, —ONR⁹R¹⁰, —NHC═(O)NHNH₂, —NHC═(O)NR⁹R¹⁰, —N(O)ₘ₃, —NR⁹R¹⁰, —NH—O—R⁹, —C(O)R⁹, —C(O)—OR⁹, —C(O)NR⁹R¹⁰, —OR⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁴ is independently hydrogen, halogen, —CX^{d}₃, —CN, —SO₂Cl, —SOₙ₄R¹¹, —SOᵥ₄NR¹¹R^{12, —NHNH}₂, —ONR¹¹R¹², —NHC═(O)NHNH₂, —NHC═(O)NR¹¹R¹², —N(O)ₘ₄, —NR¹¹R¹², —NH—O—R¹¹, —C(O)R¹¹, —C(O)—OR¹¹, —C(O)NR¹¹R¹², —OR¹¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In formula (I) or (II) Y is independently O, S or NH. W¹, W², W⁴ and W⁵ are independently CR¹³ or N. W³ is O, NR¹⁴, or S. L¹ is independently a bond, —S(O)—, —S(O)₂NH—, —NHS(O)₂—, —C(O)O—, —OC(O)—, —C(O)NH—, —NH—, —NHC(O)—, —O—, —S—, —NH-L²-, —NH—R¹⁵—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. L² is independently —C(O)—, —C(O)—NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R⁵, R⁶, R⁷, R⁸, R⁹, R¹⁰, R¹¹, R¹², R¹³, R¹⁴, and R¹⁵ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. X^{a}, X^{b}, X^{c} and X^{d} are independently —F, —Cl, —Br, or —I. n₁, n₂, n₃ and n₄ are independently an integer from 0 to 4. m₁, m₂, m₃ and m₄ are independently an integer from 1 to 2. v₁, v₂, v₃ and v₄ are independently an integer from 1 to 2. z is independently an integer from 0 to 5.

In another aspect, a compound having the formula

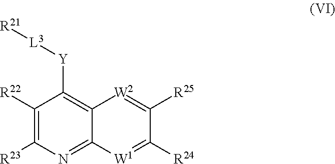

is provided. In formula (VI) R²¹ is independently hydrogen, halogen, —CX^{a}₃, —CN, —SO₂Cl, —SOₙ₁R²⁸, —SOᵥ₁NR²⁸R²⁹, —NHNH₂, —ONR²⁸R²⁹, —NHC═(O)NHNH₂, —NHC═(O)NR²⁸R²⁹, —NHC═(O)R²⁸, —N(O)ₘ₁, —NR²⁸R²⁹, —NH—O—R²⁸, —C(O)R²⁸, —C(O)—OR²⁸, —C(O)NR²⁸R²⁹, —N(R²⁸)C(O)R²⁹, —OR²⁸, —O—C(O)NR²⁸R²⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R²² is independently hydrogen, halogen, —CX^{b}₃, —CN, —SO₂Cl, —SOₙ₂R³⁰, —SOᵥ₂NR³⁰R³¹, —NHNH₂, —ONR³⁰R³¹, —NHC═(O)NHNH₂, —NHC═(O)NR³⁰R³¹, —NHC═(O)R³⁰, —N(O)ₘ₂, —NR³⁰R³¹, —NH—O—R³⁰, —C(O)R³⁰, —C(O)—OR³⁰, —C(O)NR³⁰R³¹, —N(R³⁰)C(O)R³¹, —O—C(O)NR³⁰R³¹, —OR³⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R²³ is independently hydrogen, halogen, —CX^{c}₃, —CN, —SO₂Cl, —SOₙ₃R³², —SOᵥ₃NR³²R³³, —NR³²SOᵥ₃R³³, —NHNH₂, —ONR³²R³³, —NHC═(O)NHNH₂, —NHC═(O)NR³²R³³, —NHC═(O)R³², —N(O)ₘ₃, —NR³²R³³, —NH—O—R³², —R³²NR³³NH₂, —C(O)R³², —C(O)—OR³², —C(O)NR³²R³³, —N(R³²)C(O)R³³, —O—C(O)NR³²R³³, —OR³², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R²⁴ is independently hydrogen, halogen, —CX^{d}₃, —CN, —SO₂Cl, —SOₙ₄R³⁴, —SOᵥ₄NR³⁴R³⁵, —NHNH₂, —ONR³⁴R³⁵, —NHC═(O)NHNH₂, —NHC═(O)NR³⁴R³⁵, —NHC═(O)R³⁴, —N(O)ₘ₄, —NR³⁴R³⁵, —NH—O—R³⁴, —C(O)R³⁴, —C(O)—OR³⁴, —C(O)NR³⁴R³⁵, —N(R³⁴)C(O)R³⁵, —O—C(O)NR³⁴R³⁵, —OR³⁴, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R²⁵ is independently hydrogen, halogen, —CX^{e}₃, —CN, —SO₂Cl, —SOₙ₅R³⁶, —SOᵥ₅NR³⁶R³⁷, —NHNH₂, —ONR³⁶R³⁷, —NHC═(O)NHNH₂, —NHC═(O)NR³⁶R³⁷, —NHC═(O)R³⁶, —N(O)ₘ₅, —NR³⁶R³⁷, —NH—O—R³⁶, —C(O)R³⁶, —C(O)—OR³⁶, —C(O)NR³⁶R³⁷, —N(R³⁶)C(O)R³⁷, —O—C(O)NR³⁶R³⁷, —OR³⁶, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R²⁶ is independently hydrogen, halogen, —CX^{f}₃, —CN, —SO₂Cl, —SOₙ₆R³⁸, —SOᵥ₆NR³⁸R³⁹, —NHNH₂, —ONR³⁸R³⁹, —NHC═(O)NHNH₂, —NHC═(O)NR³⁸R³⁹, —NHC═(O)R³⁸, —N(O)ₘ₆, —NR³⁸R³⁹, —NH—O—R³⁸, —C(O)R³⁸, —C(O)—OR³⁸, —C(O)NR³⁸R³⁹, —N(R³⁸)C(O)R³⁹, —O—C(O)NR³⁸R³⁹, —OR³⁸, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R²⁷ is independently hydrogen, halogen, —CX^{g}₃, —CN, —SO₂Cl, —SOₙ₇R⁴⁰, —SOᵥ₇NR⁴⁰R⁴¹, —NHNH₂, —ONR40R⁴¹, —NHC═(O)NHNH₂, —NHC═(O)NR⁴⁰_{R}⁴¹, —NHC═(O)R⁴⁰, —N(O)ₘ₇, —NR⁴⁰R⁴¹, —NH—O—R⁴⁰, —C(O)R⁴⁰, —C(O)—OR⁴⁰, —C(O)_{NR}⁴⁰R41, —N(R⁴⁰)C(O)R⁴¹, —O—C(O)NR⁴⁰R⁴¹, —OR⁴⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R²⁸ is independently hydrogen, halogen, —CX^{h}₃, —CN, —SO₂Cl, —SOₙ₈R⁴², —SOᵥ₈NR⁴²R⁴³, —NHNH₂, —ONR⁴²R⁴³, —NHC═(O)NHNH₂, —NHC═(O)NR⁴²R⁴³, —NHC═(O)R⁴², —N(O)ₘ₈, —NR⁴²R⁴³, —NH—O—R⁴², —C(O)R⁴², —C(O)—OR⁴², —C(O)NR⁴²R⁴³, —N(R⁴²)C(O)R⁴³, —O—C(O)NR⁴²R⁴³, —OR⁴², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R²⁹ is independently hydrogen, halogen, —CXⁱ₃, —CN, —SO₂Cl, —SOₙ₉R⁴⁴, —SOᵥ₉NR⁴⁴R⁴⁵, —NHNH₂, —ONR⁴⁴R⁴⁵, —NHC═(O)NHNH₂, —NHC═(O)NR⁴⁴R⁴⁵, —NHC═(O)R⁴⁴, —N(O)ₘ₉, —NR⁴⁴R⁴⁵, —NH—O—R⁴⁴, —C(O)R⁴⁴, —C(O)—OR⁴⁴, —C(O)NR⁴⁴R⁴⁵, —N(R⁴⁴)C(O)R⁴⁵, —O—C(O)NR⁴⁴R⁴⁵, —OR⁴⁴, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In formula (VI) Y is independently O or NH. W¹ is independently N or CR²⁶. W² is independently N or CR²⁷. L³ is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R³⁰, R³¹, R³², R³³, R³⁴, R³⁵ R³⁶, R³⁷, R³⁸, R³⁹, R⁴⁰, R⁴¹, R⁴², R⁴³, R⁴⁴ and R⁴⁵ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. X^{a}, X^{b}, X^{c}, X^{d}, X^{e}, X^{f}, X^{g}, X^{h} and Xⁱ are independently —F, —Cl, —Br, or —I. The symbols n₁, n₂, n₃, n₄, n₅, n₆ an, n₇, n₈ and n₉ are independently an integer from 0 to 4. The symbols m₁, m₂, m₃, m₄, m₅, m₆, m₇, m₈ and m₉ are independently an integer from 1 to 2. The symbols v₁, v₂, v₃, v₄, v₅, v₆, v₇, v₈ and v₉ are independently an integer from 1 to 2. The symbol z is independently an integer from 0 to 5.

In another aspect, a method of treating a disease in a patient in need of such treatment is provided. The method includes administering a therapeutically effective amount of a compound as provided herein (e.g., a compound of formula (I), (II), (III), (IV), (V), (VI), (VII), (VIII) or (IX)) including embodiments thereof.

In another aspect, a pharmaceutical composition including a pharmaceutically acceptable excipient and a compound as provided herein (e.g., a compound of formula (I), (II), (III), (IV), (V), (VI), (VII), (VIII) or (IX)) including embodiments thereof.

In another aspect, a method of inhibiting the activity of Olig2 in a cell is provided. The method includes contacting the cell with a compound as provided herein (e.g., a compound of formula (I), (II), (III), (IV), (V), (VI), (VII), (VIII) or (IX)) including embodiments thereof.

In another aspect, a method of treating a disease in a patient in need of such treatment is provided. The method includes administering a therapeutically effective amount of a compound of Table 1, 2, or 3.

In another aspect, a method of inhibiting the activity of Olig2 in a cell is provided. The method includes contacting the cell with a compound of Table 1, 2 or 3.

In another aspect, a pharmaceutical composition including a pharmaceutically acceptable excipient and a compound of Table 1, 2 or 3 is provided.

In another aspect, a method of identifying an inhibitor of protein dimerization is provided. The method includes constructing in silico a computer readable peptide including a steric feature and an electronic feature, wherein the steric feature and the electronic feature form part of a first protein and wherein the steric feature and the electronic feature participate in dimerization of the first protein with a second protein. A level of binding of the computer readable peptide to a compound is determined in silico. The level is compared to a control level, wherein an increase of the level compared to the control level indicates the compound is an inhibitor compound of protein dimerization.

In another aspect, a peptide, peptidomimetic, cyclic peptidomimetic, or cyclic peptide, wherein the peptide, peptidomimetic, cyclic peptidomimetic, or cyclic peptide capable of binding to Olig2 is provided.

In another aspect, a pharmaceutical composition is provided. The pharmaceutical composition includes a pharmaceutically acceptable excipient and a peptide, peptidomimetic, cyclic peptidomimetic, or cyclic peptide as provided herein.

In another aspect, a method of treating a disease in a patient in need of such treatment is provided. The method includes administering a therapeutically effective amount of a peptide, peptidomimetic, cyclic peptidomimetic, or cyclic peptide as provided herein.

In another aspect, a method of inhibiting the activity of Olig2 in a cell is provided. The method includes contacting the cell with a peptide, peptidomimetic, cyclic peptidomimetic, or cyclic peptide as provided herein.

### BRIEF DESCRIPTION OF THE DRAWINGS

FIG. 1. Certain Olig2 compounds described herein interfere with Olig2 dimerization with itself and partner proteins by blocking binding hotspots with the dimerization region. The pharmacophore for this region is shown. The pharmacophore shown in FIG. 1 was used for generating an in silico compound search of small molecules.

FIG. 2. Depicted is the Olig2 homodimer engaging DNA. The dimerization contact region of Olig2 is shown in the circle.

FIG. 3. Enlarged view of Olig2 in circle shown in FIG. 2. Shown are the amino acid residues that are most important in making direct contacts for dimerization.

FIG. 4. OLIG2 silencing ablates malignant potential of human glioblastoma. P16^{Ink4a}/P19 (ARF) null mice were bred into an OLIG1/2 double null background. Neurosphere cultures from these mice or from OLIG1/2^{+/-}, P16^{Ink4a}/P19^{-/-} littermates were transfected with the constitutively active VIII mutation of the EGF receptor and assessed for glioma formation in SCID mice. (FIG. 4A) OLIG1/2 is required for tumor development. (FIG. 4B) OLIG2 function was rescued by transfecting the OLIG1/2^{-/-} neurospheres with a retroviral vector encoding OLIG2 only. Controls (OLIG1/2^{-/-}) were transfected with a GFP retrovirus. This data validates Olig2 as an important target in GBM.

FIG. 5. PCR data for Olig2 expression for patient derived GBM cell lines (GBM4 and GBM8) which express Olig2, and the U87 cell line which expresses very little Olig2, and primary astrocytes which do not express Olig2. Both GBM4 and GBM8 cell lines are suppressed by Olig2 inhibition, while U87 and astrocytes are suppressed by much (8 and 22 fold, respectively) higher doses. The sensitivity to the inhibitor relates to Olig2 expression—see FIGS. 8 through 12 for GBM tumor responses data pertinent to this figure.

FIG. 6. Expression of P21 which is a direct genetic target of Olig2. P21 is suppressed by Olig2. The graph of FIG. 6 shows that vehicle control solution did not suppress Olig2 and the P21 levels were low, as expected. Addition of Olig2 inhibitor compound SKOG-101 suppressed Olig2 and P21 levels rose markedly. This provides evidence that the inhibitor compound SKOG-101 did in fact target Olig2.

FIG. 7. Levels of OMG. Olig2 triggers the expression of OMG, and it can be seen that when the inhibitor compound SKOG-101 was added, relative to vehicle control the levels of OMG went down markedly due to inhibition of Olig2. Again, this shows that the inhibitor compound SKOG-101 was targeting Olig2 specifically.

FIG. 8. GBM4 viability with administration of compounds SKOG-101 and SKOG-102.

FIG. 9. GBM8 viability with administration of compounds SKOG-101 and SKOG-102.

FIG. 10. U87 viability with administration of compounds SKOG-101 and SKOG-102. U87 express low amounts of Olig2, IC50 is high.

FIG. 11. Primary astrocyte viability with administration of compounds SKOG-101 and SKOG-102. Astrocytes express low amounts of Olig2, IC50 is high. Olig2 expression determined by PCR.

FIG. 12. Olig2 homodimer engaging DNA. The dimerization contact region of Olig2 is shown in the circle and enlargement. An Olig2 compound is shown.

FIGS. 13A-13C. Homology modeling and definition of the OLIG2 pharmacophore. (FIG. 13A) OLIG2-E47 heterodimer: (i) general structure—regions of OLIG2 important for interaction with E47, inset depicts topological scheme of the interface, arrow indicates the region of pharmacophore design; (ii) enlarged region of the middle interface, (iii) enlarged region of the upper interface. The lower part of the interface is saturated by negatively charged residues to complement the negative charge of the DNA. (FIG. 13B) Close-up of the interface interactions E47-OLIG2. Overall view in panel (i) of the location of key residues involved in the specific interaction between OLIG2 and E2A. The area of interaction is also indicated by the arrow in panel (ii). Panel (iii) shows the interaction zone, which includes the negative residues E18 and D15 from E2A and the positive residue K39 on OLIG2. (FIG. 13C) Scheme of the E2A interface created by TF features. The rectangle shows the region expanded in the larger drawing. Complementary combinations: Group 1 (OLIG2, NeuroD1): P2, H1, H2, P4; Group2: P1, P3, H1, H2, P4; Group3: P2, P3, H1, H2, P4. This organization leads to the definition of the main features of four pharmacophores: Pharmacophore 0 (five features): P1, 2; H1; P3; H2; P4; Pharmacophore 1 (four features): P1,2; H1; H2; P4 (OLIG2 and similar); Pharmacophore 2 (four features): P1,2; H1; P3; H2; Pharmacophore 3 (four features): H1; P3; H2; P4.

FIGS. 14A-14C. Parental and daughter pharmacophore definitions that guided conformational database searches. (FIG. 14A) Five features parental (i) and four-features daughter (ii, iii, and iv) pharmacophores. Ribbon diagram and residues presented by lines belong to the superimposed OLIG2 protein. (FIG. 14B) Venn diagram for four sets of compounds resulted from four pharmacophore-hypotheses based search in conformational database derived from the Open NCI compounds in-silico library. For example, using the gr1 daughter pharmacophore the program selected 545 compounds from which 147 were also selected using gr2, gr 3, daughter and the parental (gen) pharmacophore. (FIG. 14C) The three upper panels illustrate how Applicants' database screening identified compounds. This example engages all three subpharmacophores within the dimerization region. The three bottom panels show representative compounds from three different structure clusters that all were predicted to fit the three daughter pharmacophores.

FIGS. 15A-15B. Structural classes of potential OLIG2 inhibitors and in-vitro anti-GBM potency. (FIG. 15A) Shows representative compounds from the five final structural classes. Note how the two compounds shown for each class resemble one another. (FIG. 15B) Shows IC₅₀ curves for Ink4a/arfEGFR-VIII cells treated with the most potent compounds from each cluster.

FIGS. 16A-16D. Compounds selected by subpharmacophore modeling-driven database searches were selective for OLIG2. (FIG. 16A) The most potent OLIG2 inhibitor identified by Applicants' modeling methodology clearly inhibits human GBM4 and GBM8 cells grown as neurospheres in a dose-dependent fashion. DMSO was the vehicle control. (FIG. 16B) Light microscopy further demonstrates inhibition of neurosphere formation in GBM4 cells. GBM4 neurospheres were cultured in 96-well plates at a concentration of 2,000 cells/well in medium containing EGF, FGF and Heparin. Inhibitor compounds were added after 12 h at the concentrations indicated. (1) Vehicle (1% DMSO), (2) SKOG-149 (inactive compound), 0.1uM, (3) SKOG-149, 5 uM, (4) Vehicle (1% DMSO), (5) SKOG-102 (active compound), 0.1uM, and (6) SKOG-102, 5 uM. Cells were visualized under 4× objective after 72 h. (FIG. 16C) qPCR identifies OLIG2 expression relative to actin for patient-derived GBM lines (GBM4 and GBM8), for a serum-grown GBM cell line (U87) and for normal human astrocytes (NHA) freshly acquired from patient material. Although not indicated by the scale, more OLIG2 is expressed by U87s than by NHAs. (FIG. 16D) Data indicates that OLIG2 expression correlates with cell death induced by inhibitor compound. GBM 4 and 8 cells were markedly suppressed in vitro by Applicants' most potent OLIG2 inhibitor given alone. U87 cells, which express much less OLIG2 than GBM4/8 cells, were also suppressed, but a higher dose was needed. On the other hand, NHA, which express no OLIG2, had an IC50 of ˜20 fold greater than GBM4 cells. (All samples run in duplicate.)

FIGS. 17A-17B. OLIG2 inhibitor affects expression of direct genetic targets of OLIG2. To determine whether the OLIG2 inhibitor affects downstream targets of OLIG2, GBM4 cells were treated with 18 h with the most potent inhibitor compound (SKOG-102) or an inactive compound (SKOG-149) as control, at 3 different doses and expression of p21 and OMG were determined by qPCR. (FIG. 17A) Escalating OLIG2 inhibitor doses led to increased levels of p21. (FIG. 17B) Increasing concentrations of OLIG2 inhibitor suppressed the expression of OMG. These results strongly suggest that the inhibitor compound directly targets OLIG2.

FIG. 18. The p21 expression data were validated by a luciferase based reporter assay. The luciferase gene was transfected into 293 FT cells and its translation depended on the p21 promoter. Co-transfection with the OLIG2 gene decreased p21 expression while administration of the OLIG2 inhibitor compound significantly restored p21 expression (p═0.0089).

FIG. 19. PCR analysis of GBM stem-like cells (GBM4) derived from human primary tumors showed that OLIG2 shRNA caused a reduction in OLIG2 expression, and changed the expression of direct OLIG2 targets and linked markers (n=5 per experiment). OLIG2 shRNA allowed p21 expression levels to rise while the expression of OMG dropped, which is consistent with the well-established suppression of p21 and promotion of OMG directly triggered by OLIG2. Moreover, genetic knock-down of OLIG2 caused a significant reduction in the expression levels of oligodendrocyte markers (CNPase, MBP, and PLP1) and stem cell markers (CD133 and Nestin) on GBM stem-like cells.

FIG. 20. PCR analysis of GBM stem-like cells (GBM4) derived from human primary tumors showed that application of the most potent inhibitor compound identified in cellular screens duplicated the results acquired with OLIG2 shRNA. The expression of p21 was upregulated while OMG was downregulated, and the expression of CNPase, MBP, PLP1, CD133 and Nestin was reduced. All changes showed a clearly dose dependency and the data shown is the mean of duplicates for each test.

FIG. 21. ChIP analysis to detect Olig2 binding to the p21 promoter. OLIG2 antibody was used for chromatin immunoprecipitation and analyzed using PCR for P21 promoter expression (OLIG2 binding region of P21 promoter). Olig2 binding to P21 promoter region was detected upon induction of Olig2 expression by FGF. This binding was inhibited by the Olig2 inhibitor compound.

FIG. 22. Radiation sensitization effect of OLIG2 inhibitor compound. GBM stem-like cells (GBM4 and 8) derived from human primary tumors were incubated with 1 um of inhibitor compound, after 16 hr cells were treated with 2 gy and 10 gy radiation and cell were allowed to grow for 4 days, cell viability was assayed by alamar blue.

FIG. 23. Pretreatment with OLIG2 inhibitor compound inhibited GBM neurosphere growth in vivo. MRI tumor volume (CC) of mice harboring intracranial GBM4 neurosphere pretreated for 14 hours with OLIG2 inhibitor compound and DMSO control. Volume of tumor enhancement (A) and primary tumor volume (B) were measured using MRI after ˜4 wks.

FIGS. 24A-24B. Sequence alignment of transcription factors relevant to OLIG2. (FIG. 24A) OLIG2 and NeuroD1; (FIG. 24B). The group of transcription factors binding to E2A (E47). Sequence legend: FIG. 24A (sequences in order of appearance top to bottom): SEQ ID NOS:14-15; FIG. 24B (sequences in order of appearance top to bottom): SEQ ID NOS:16-35

### DETAILED DESCRIPTION OF THE INVENTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

An “alkenyl” by itself or as part of another substituent, means, unless otherwise stated, a straight (i.e., unbranched) or branched carbon chain having one or more double bonds.

Examples of alkenyl groups include, but are not limited to, vinyl, 2-propenyl, crotyl, 2-isopentenyl, 2-(butadienyl), 2,4-pentadienyl, and 3-(1,4-pentadienyl). An “alkynyl” by itself or as part of another substituent, means, unless otherwise stated, a straight (i.e., unbranched) or branched carbon chain having one or more triple bonds. Examples of alkenyl groups include, but are not limited to, ethynyl, 1-propynyl, 3-propynyl, and 3-butynyl.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and CH₂—O—Si(CH₃)₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, NR′NR″R′″, —ONR′R″, —NR′C═(O)NR″NR′″R″″, —CN, —NO₂, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR″R″)═NR′″, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

A heteroaryl group substituent may be a —O⁻ bonded to a ring heteroatom nitrogen.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), and silicon (Si).

A “substituent group,” as used herein, means a group selected from the following moieties:
(A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from: oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

In some embodiments, the cycloalkyl group represents a fully saturated carbon containing ring. In some embodiments, the heterocycloalkyl represents a fully saturated carbon containing ring wherein one or more of the ring carbon atoms is replaced with a heteroatom selected from O, N, P, S, and Si.

In some embodiments, substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, ONR′R″, —NR′C═(O)NR″NR′″R″″, —CN, —NO₂, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl.

In some embodiments, the substituent group as used herein, means a group selected from the following moieties:
(A) —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from: oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “ lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, and each substituted or unsubstituted heterocycloalkyl is a substituted or unsubstituted 3 to 7 membered heterocycloalkyl.

In embodiments, each substituted group described in the compounds herein is substituted with at least one substituent group. More specifically, in embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, and/or each substituted or unsubstituted heterocycloalkyl is a substituted or unsubstituted 3 to 8 membered heterocycloalkyl. In embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, and/or each substituted or unsubstituted heterocycloalkylene is a substituted or unsubstituted 3 to 8 membered heterocycloalkylene.

In embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and/or each substituted or unsubstituted heterocycloalkyl is a substituted or unsubstituted 5 to 7 membered heterocycloalkyl. In embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₅-C₇ cycloalkylene, and/or each substituted or unsubstituted heterocycloalkylene is a substituted or unsubstituted 5 to 7 membered heterocycloalkylene.

In embodiments, the compound is a chemical species set forth in the Examples section below.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)— or (S)— or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)— and (S)—, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety to the remainder of a molecule or chemical formula.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Where a moiety is substituted with an R substituent, the group may be referred to as “R-substituted.” Where a moiety is R-substituted, the moiety is substituted with at least one R substituent and each R substituent is optionally different. For example, where a moiety herein is R¹²-substituted or unsubstituted alkyl, a plurality of R¹² substituents may be attached to the alkyl moiety wherein each R¹² substituent is optionally different. Where an R-substituted moiety is substituted with a plurality R substituents, each of the R-substituents may be differentiated herein using a prime symbol (′) such as R′, R″, etc. For example, where a moiety is R¹²-substituted or unsubstituted alkyl, and the moiety is substituted with a plurality of R¹² substituents, the plurality of R¹² substituents may be differentiated as R¹²′, R¹²″, R¹²′″, etc. In embodiments, the plurality of R substituents is 3. In embodiments, the plurality of R substituents is 2.

In embodiments, a compound as described herein may include multiple instances of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹ and/or other variables. In such embodiments, each variable may optional be different and be appropriately labeled to distinguish each group for greater clarity. For example, where each R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and/or R²¹, is different, they may be referred to, for example, as R^{1.1}, R^{1.2}, R^{1.3}, R^{1.4}, R^{2.1}, R^{2.2}, R^{2.3}, R^{2.4}, R^{3.1}, R^{3.2}, R^{3.3}, R^{3.4}, R^{4.1}, R^{4.2}, R^{4.3}, R^{4.4}, R^{5.1}, R^{5.2}, R^{5.3}, R^{5.4}, R^{6.1}, R^{6.2}, R^{6.3}, R^{6.4}, R^{7.1}, R^{7.2}, R^{7.3}, R^{7.4}, R^{8.1}, R^{8.2}, R^{8.3}, R^{8.4}, R^{9.1}, R^{9.2}, R^{9.3}, R^{9.4}, R^{10.1}, R^{10.2}, R^{10.3}, R^{10.4}, R^{11.1}, R^{11.2}, R^{11.3}, R^{11.4}, R^{12.1}, R^{12.2}, R^{12.3}, R^{12.4}, R^{13.1}, R^{13.2}, R^{13.3}, R^{13.4}, R^{14.1}, R^{14.2}, R^{14.3}, R^{14.4}, R^{15.1}, R^{15.2}, R^{15.3}, R^{15.4}, R^{16.1}, R^{16.2}, R^{16.3}, R^{16.4}, R^{17.1}, R^{17.2}, R^{17.3}, R^{17.4}, R^{18.1}, R^{18.2}, R^{18.3}, R^{18.4}, R^{19.1}, R^{19.2}, R^{19.3}, R^{19.4}, R^{20.1}, R^{20.2}, R^{20.3}, R^{20.4}, R^{21.1}, R^{21.2}, R^{21.3}, and/or R^{21.4}, respectively, wherein the definition of R¹ is assumed by R^{1.1}, R^{1.2}, R^{1.3}, and/or R^{1.4}, the definition of R² is assumed by R^{2.1}, R^{2.2}, R^{2.3}, and/or R^{2.4}, the definition of R³ is assumed by R^{3.1}, R^{3.2}, R^{3.3}, and/or R^{3.4}, the definition of R⁴ is assumed by R^{4.1}, R^{4.2}, R^{4.3}, and/or R^{4.4}, the definition of R⁵ is assumed by R^{5.1}, R^{5.2}, R^{5.3}, and/or R^{5.4}, the definition of R⁶ is assumed by R^{6.1}, R^{6.2}, R^{6.3}, and/or R^{6.4}, the definition of R⁷ is assumed by R^{7.1}, R^{7.2}, R^{7.3}, and/or R^{7.4}, the definition of R⁸ is assumed by R^{8.1}, R^{8.2}, R^{8.3}, and/or R^{8.4}, the definition of R⁹ is assumed by R^{9.1}, R^{9.2}, R^{9.3}, and/or R^{9.4}, the definition of R¹⁰ is assumed by R^{10.1}, R^{10.2}, R^{10.3}, and/or R^{10.4}, the definition of R¹¹ is assumed by R^{11.1}, R^{11.2}, R^{11.3}, and/or R^{11.4}, the definition of R¹² is assumed by R^{12.1}, R^{12.2}, R^{12.3}, and/or R^{12.4}, the definition of R¹³ is assumed by R^{3.1}, R^{13.2}, R^{13.3}, and/or R^{13.4}, the definition of R¹⁴ is assumed by R^{14.1}, R^{14.2}, R^{14.3}, and/or R^{14.4}, the definition of R¹⁵ is assumed by R^{15.1}, R^{15.2}, R^{15.3}, and/or R^{15.4}, the definition of R¹⁶ is assumed by R^{16.1}, R^{16.2}, R^{16.3}, and/or R^{16.4}, the definition of R¹⁷ is assumed by R^{17.1}, R^{17.2}, R^{17.3}, and/or R^{17.4}, the definition of R¹⁸ is assumed by R^{18.1}, R^{18.2}, R^{18.3}, and/or R^{18.4}, the definition of R¹⁹ is assumed by R^{19.1}, R^{19.2}, R^{19.3}, and/or R^{19.4}, the definition of R²⁰ is assumed by R^{20.1}, R^{20.2}, R^{20.3}, and/or R^{20.4}, the definition of R²¹ is assumed by R^{21.1}, R^{21.2}, R^{21.3}, and/or R^{21.4}. The variables used within a definition of _{R}¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and/or R²¹, and/or other variables that appear at multiple instances and are different may similarly be appropriately labeled to distinguish each group for greater clarity.

Description of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The terms “peptide,” “polypeptide,” and “protein” are used interchangeably herein to refer to a polymer of amino acid residues.

The term “peptidyl” and “peptidyl moiety” means a monovalent peptide.

The term “amino acid” refers to naturally occurring and synthetic amino acids, as well as amino acid analogs. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. An oligomer comprising amino acid mimetics is a peptidomimetic. A peptidomimetic moiety is a monovalent peptidomimetic.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

A “conservative substitution” as used with respect to amino acids, refers to the substitution of an amino acid with a chemically similar amino acid. Amino acid substitutions which often preserve the structural and/or functional properties of the polypeptide in which the substitution is made are known in the art and are described, for example, by H. Neurath and R. L. Hill, 1979, in “The Proteins,” Academic Press, New York. The most commonly occurring exchanges are isoleucine/valine, tyrosine/phenylalanine, aspartic acid/glutamic acid, lysine/arginine, methionine/leucine, aspartic acid/asparagine, glutamic acid/glutamine, leucine/isoleucine, methionine/isoleucine, threonine/serine, tryptophan/phenylalanine, tyrosine/histidine, tyrosine/tryptophan, glutamine/arginine, histidine/asparagine, histidine/glutamine, lysine/asparagine, lysine/glutamine, lysine/glutamic acid, phenylalanine/leucine, phenylalanine/methionine, serine/alanine, serine/asparagine, valine/leucine, and valine/methionine. The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). In embodiments, there may be at least 1, at least 2, at least 3, at least 4, at least 5, at least 6, at least 7, at least 8, at least 9, at least 10, at least 15, at least 20, at least 25, at least 30, at least 35, or at least 40 conservative substitutions. In embodiments, there may be 1, 2, 3, 4, 5, 6, 7, 8, 9, 10, 15, 20, 25, 30, 35, or 40 conservative substitutions.

The term “amino acid substitution set” or “substitution set” refers to a group of amino acid substitutions. A substitution set can have 1, 2, 3, 4, 5, 6, 7, 8, 9, 10, 11, 12, 13, 14, 15, or more amino acid substitutions.

The term “isolated” refers to a nucleic acid, polynucleotide, polypeptide, protein, or other component that is partially or completely separated from components with which it is normally associated (other proteins, nucleic acids, cells, etc.). In embodiments, an isolated polypeptide or protein is a recombinant polypeptide or protein.

A nucleic acid (such as a polynucleotide), a polypeptide, or a cell is “recombinant” when it is artificial or engineered, or derived from or contains an artificial or engineered protein or nucleic acid (e.g. non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Identity” or “percent identity,” in the context of two or more polypeptide sequences, refers to two or more sequences or subsequences that are the same or have a specified percentage of amino acid residues that are the same (e.g., share at least about 70%, at least about 75%, at least about 80%, at least about 85%, at least about 88% identity, at least about 89%, at least about 90%, at least about 91%, at least about 92%, at least about 93%, at least about 94%, at least about 95%, at least about 96%, at least about 97%, at least about 98%, or at least about 99% identity) over a specified region to a reference sequence, when compared and aligned for maximum correspondence over a comparison window, or designated region as measured using a sequence comparison algorithms or by manual alignment and visual inspection.

Optimal alignment of sequences for comparison and determination of sequence identity can be determined by a sequence comparison algorithm or by visual inspection (see, generally, Ausubel et al., infra). When optimally aligning sequences and determining sequence identity by visual inspection, percent sequence identity is calculated as the number of residues of the test sequence that are identical to the reference sequence divided by the number of non-gap positions and multiplied by 100. When using a sequence comparison algorithm, test and reference sequences are entered into a computer, subsequence coordinates and sequence algorithm program parameters are designated. The sequence comparison algorithm then calculates the percent sequence identities for the test sequences relative to the reference sequence, based on the program parameters as known in the art, for example BLAST or BLAST 2.0. For example, comparison can be conducted, e.g., by the local homology algorithm of Smith & Waterman, 1981, Adv. Appl. Math. 2:482, by the homology alignment algorithm of Needleman & Wunsch, 1970, J. Mol. Biol. 48:443, by the search for similarity method of Pearson & Lipman, 1988, Proc. Nat'l. Acad. Sci. USA 85:2444, or by computerized implementations of these algorithms (GAP, BESTFIT, FASTA, and TFASTA in the Wisconsin Genetics Software Package, Genetics Computer Group, 575 Science Dr., Madison, Wis.). Thus alignment can be carried out for sequences that have deletions and/or additions, as well as those that have substitutions, as well as naturally occurring, e.g., polymorphic or allelic variants, and man-made variants.

The phrase “substantial sequence identity” or “substantial identity,” in the context of two polypeptide sequences, refers to a sequence that has at least 70% identity to a reference sequence. Percent identity can be any integer from 70% to 100%. Two polypeptide sequences that have 100% sequence identity are said to be “identical.” A polypeptide sequence is said to have “substantial sequence identity” to a reference sequence when the sequences have at least about 70%, at least about 75%, at least 80%, at least 85%, at least 90%, at least 91%, at least 92%, at least 93%, at least 94%, at least 95%, at least 96%, at least 97%, at least 98%, or at least 99% or greater sequence identity as determined using the methods described herein, such as BLAST using standard parameters as described above.

The term “expression” includes any step involved in the production of the polypeptide including, but not limited to, transcription, post-transcriptional modification, translation, post-translational modification, and secretion.

An amino acid or peptide is “heterologous” to another sequence with which it is operably linked if the two sequences are not associated in nature.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853). The methods above may be used to synthesize single molecular species for incorporation into a prodrug.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat cancer by decreasing the incidence of cancer and or causing remission of cancer. In embodiments, certain methods presented herein successfully treat Downs Syndrome by decreasing the incidence of Downs Syndrome or reducing one or more sympotoms or Downs Syndrome or reducing the severity of one or more symptoms of Downs Syndrome. The term “treating,” and conjugations thereof, include prevention of an injury, pathology, condition, or disease.

An “effective amount” is an amount sufficient to accomplish a stated purpose (e.g. achieve the effect for which it is administered, treat a disease, reduce enzyme activity, reduce one or more symptoms of a disease or condition, reduce kinase activity in a cell, reduce the activity of Olig2 in a cell). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme or protein (e.g. Olig2) relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

“Contacting” is used in accordance with its plain ordinary meaning and refers to the process of allowing at least two distinct species (e.g. chemical compounds including biomolecules, or cells) to become sufficiently proximal to react, interact or physically touch. It should be appreciated, however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a protein or enzyme (e.g. Olig2). In embodiments, the protein may be Olig2. In embodiments contacting includes allowing a compound described herein to interact with a protein or enzyme that is involved in transcription.

As defined herein, the term “inhibition”, “inhibit”, “inhibiting” and the like in reference to a protein-inhibitor interaction means negatively affecting (e.g. decreasing) the activity or function of the protein (e.g. decreasing gene transcription regulated by Olig2) relative to the activity or function of the protein (e.g. Olig2, transcription factor) in the absence of the inhibitor (e.g. Olig2 inhibitor or Olig2 inhibitor compound). In embodiments inhibition refers to reduction of a disease or symptoms of disease. In embodiments, inhibition refers to a reduction in the activity of a signal transduction pathway or signaling pathway (e.g. reduction of a pathway involving transcription regulation by Olig2 or transcription regulated by Olig2). Thus, inhibition includes, at least in part, partially or totally blocking stimulation, decreasing, preventing, or delaying activation, or inactivating, desensitizing, or down-regulating signal transduction or enzymatic activity or the amount of a protein (e.g. Olig2). In embodiments, inhibition refers to inhibition of Olig2.

The term “modulator” refers to a composition that increases or decreases the level of a target molecule or the function of a target molecule (e.g. a target may be a transcription factor and the function may be to increase transcription). In embodiments, an Olig2 modulator is a compound that reduces the activity of Olig2 in a cell. In embodiments, an Olig2 disease modulator is a compound that reduces the severity of one or more symptoms of a disease associated with Olig2 (e.g. cancer or Downs Syndrome).

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In embodiments, a patient is human.

“Disease” or “condition” refer to a state of being or health status of a patient or subject capable of being treated with the compounds or methods provided herein. In embodiments, the disease is a disease related to (e.g. caused by) Olig2 or aberrant Olig2 activity (e.g. brain cancer, glioblastoma multiforme, medulloblastoma, astrocytomas, brain stem gliomas, meningiomas, oligodendrogliomas, melanomas, lung cancers, breast cancer, leukemias, or Down's Syndrome). Examples of diseases, disorders, or conditions include, but are not limited to brain cancer, glioblastoma multiforme, medulloblastoma, astrocytomas, brain stem gliomas, meningiomas, oligodendrogliomas, melanomas, lung cancers, breast cancer, leukemias, Down's Syndrome, colorectal cancer, papillary thyroid cancer, hepatocellular carcinoma, Alzheimer's disease, Parkinson's disease, Huntington's Disease, frontotemporal dementia, Creutzfeldt-Jakob disease, Gerstmann-Sträussler-Scheinker syndrome, prion disease, neurodegenerative diseases, cancer, cardiovascular disease, hypertension, Syndrome X, depression, anxiety, glaucoma, human immunodeficiency virus (HIV) or acquired immunodeficiency syndrome (AIDS), neurodegeneration, Alzheimer's disease, Parkinson's disease, cognition enhancement, Cushing's Syndrome, Addison's Disease, osteoporosis, frailty, muscle frailty, inflammatory diseases, osteoarthritis, rheumatoid arthritis, asthma and rhinitis, adrenal function-related ailments, viral infection, immunodeficiency, immunomodulation, autoimmune diseases, allergies, wound healing, compulsive behavior, multi-drug resistance, addiction, psychosis, anorexia, cachexia, post-traumatic stress syndrome, post-surgical bone fracture, medical catabolism, major psychotic depression, mild cognitive impairment, psychosis, dementia, hyperglycemia, stress disorders, antipsychotic induced weight gain, delirium, cognitive impairment in depressed patients, cognitive deterioration in individuals with Down's syndrome, psychosis associated with interferon-alpha therapy, chronic pain, pain associated with gastroesophageal reflux disease, postpartum psychosis, postpartum depression, neurological disorders in premature infants, migraine headaches, stroke, aneurysm, brain aneurysm, cerebral aneurysm, brain attack, cerebrovascular accident, ischemia, thrombosis, arterial embolism, hemorrhage, transient ischemic attack, anemia, embolism, systemic hypoperfusion, venous thrombosis, arthritis, reperfusion injury, skin diseases or conditions, acne, acne vulgaris, keratosis pilaris, acute, promyelocytic leukemia, baldness, acne rosacea, harlequin ichthyosis, xeroderma pigmentosum, keratoses, neuroblastoma, fibrodysplasia ossificans progressive, eczema, rosacea, sun damage, wrinkles, or cosmetic conditions. In some instances, “disease” or “condition” refer to cancer. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

As used herein, the term “cancer” refers to all types of cancer, neoplasm or malignant tumors found in mammals, including leukemia, carcinomas and sarcomas. Exemplary cancers that may be treated with a compound or method provided herein include cancer of the thyroid, endocrine system, brain, breast, cervix, colon, head & neck, liver, kidney, lung, non-small cell lung, melanoma, mesothelioma, ovary, sarcoma, stomach, uterus or Medulloblastoma. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

A “cancer associated with aberrant Olig2 activity” (also referred to herein as “Olig2 related cancer”) is a cancer caused by aberrant Olig2 activity (e.g. a mutated Olig2 gene). Olig2 related cancers may include brain cancer, glioblastoma multiforme, medulloblastoma, astrocytomas, brain stem gliomas, meningiomas, oligodendrogliomas, melanomas, lung cancers, breast cancer, leukemias, T cell leukemias.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The term “administer (or administering) an Olig2 inhibitor” means administering a compound that inhibits the activity or level (e.g. amount) of Olig2 to a subject and, without being limited by mechanism, allowing sufficient time for the Olig2 inhibitor to reduce the activity of Olig2 or for the Olig2 inhibitor to reduce one or more symptoms of a disease (e.g. cancer).

The term “associated” or “associated with” as used herein to describe a disease (e.g. an Olig2 associated disease, a cancer associated with aberrant Olig2 activity, Olig2 associated cancer) means that the disease (e.g. cancer) is caused by, or a symptom of the disease is caused by Olig2.

The term “aberrant” as used herein refers to different from normal. When used to described enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

### II. Compounds

In one aspect, a compound having the formula

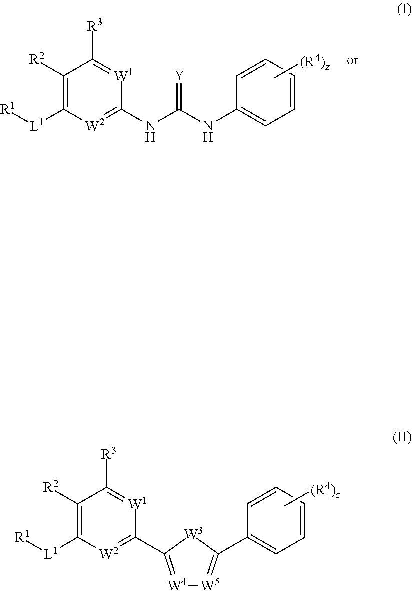

is provided. In formula (I) or (II) R¹ is hydrogen, halogen, —CX^{a}₃, —CN, —SO₂Cl, —SOₙ₁R⁵, —SOᵥ₁NR⁵R⁶, —NHNH₂, —ONR⁵R⁶, —NHC═(O)NHNH₂, —NHC═(O)NR⁵R⁶, —N(O)ₘ₁, —NR⁵R⁶, —NH—O—R⁵, —C(O)R⁵, —C(O)—OR⁵, —C(O)NR⁵R⁶, —OR⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R² is hydrogen, halogen, —CX^{b}₃, —CN, —SO₂Cl, —SOₙ₂R⁷, —SOᵥ₂NR⁷R⁸, —NHNH₂, —ONR⁷R⁸, —NHC═(O)NHNH₂, —NHC═(O)NR⁷R⁸, —N(O)ₘ₂, —NR⁷R⁸, —NH—O—R⁷, —C(O)R⁷, —C(O)—OR⁷, —C(O)NR⁷R⁸, —OR⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R³ is hydrogen, halogen, —CX^{c}₃, —CN, —SO₂Cl, —SOₙ₃R⁹, —SOᵥ₃NR⁹R¹⁰, —NHNH₂, —ONR⁹R¹⁰, —NHC═(O)NHNH₂, —NHC═(O)NR⁹R¹⁰, —N(O)ₘ₃, —NR⁹R¹⁰, —NH—O—R⁹, —C(O)R⁹, —C(O)—OR⁹, —C(O)NR⁹R¹⁰, —OR⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁴ is hydrogen, halogen, —CX^{d}₃, —CN, —SO₂Cl, —SOₙ₄R¹¹, —SOᵥ₄NR¹¹R¹², —NHNH₂, —ONR¹¹R¹², —NHC═(O)NHNH₂, —NHC═(O)NR¹¹R¹², —N(O)ₘ₄, —NR¹¹R¹², —NH—O—R¹¹, —C(O)R¹¹, —C(O)—OR¹¹, —C(O)NR¹¹R¹², —OR¹¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In formula (I) or (II) Y is O, S or NH. W¹, W², W⁴ and W⁵ are independently CR¹³ or N. W³ is O, NR¹⁴, or S. L¹ is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, —NH-L²-, —NH—R¹⁵—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. L² is —C(O)—, —C(O)—NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R⁵, R⁶, R⁷, R⁸, R⁹, R¹⁰, R¹¹, R¹², R¹³, R¹⁴ and R¹⁵ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. X^{a}, X^{b}, X^{c} and X^{d} are independently —F, —Cl, —Br, or —I. The symbols n₁, n₂, n₃ and n₄ are independently integers from 0 to 4. The symbols m₁, m₂, m₃ and m₄ are independently integers from 1 to 2. The symbols v₁, v₂, v₃ and v₄ are independently integers from 1 to 2. The symbol z is an integer from 0 to 5.

In embodiments of formula (I) or (II) R¹ is hydrogen, halogen, —CX^{a}₃, —CN, —SO₂Cl, —SOₙ₁R⁵, —SOᵥ₁NR⁵R⁶, —NHNH₂, —ONR⁵R⁶, —NHC═(O)NHNH₂, —NHC═(O)NR⁵R⁶, —N(O)ₘ₁, —NR⁵R⁶, —NH—O—R⁵, —C(O)R⁵, —C(O)—OR⁵, —C(O)NR⁵R⁶, —OR⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹ may be hydrogen, halogen, —CX^{a}₃, —CN, —SO₂Cl, —SOₙ₁R⁵, —SOᵥ₁NR⁵R⁶, —NHNH₂, —ONR⁵R⁶, —NHC═(O)NHNH₂, —NHC═(O)NR⁵R⁶, —N(O)ₘ₁, —NR⁵R⁶, —NH—O—R⁵, —C(O)R⁵, —C(O)—OR⁵, —C(O)NR⁵R⁶, —OR⁵, substituted or unsubstituted C₁-C₂₀ (e.g., C₁-C₆) alkyl, substituted or unsubstituted 2 to 20 membered (e.g., 2 to 6 membered) heteroalkyl, substituted or unsubstituted C₃-C₈ (e.g., C₅-C₇) cycloalkyl, substituted or unsubstituted 3 to 8 membered (e.g., 3 to 6 membered) heterocycloalkyl, substituted or unsubstituted C₅-C₁₀ (e.g., C₅-C₆) aryl, or substituted or unsubstituted 5 to 10 membered (e.g., 5 to 6 membered) heteroaryl.

In embodiments, R¹ is hydrogen, halogen, —CX^{a}₃, —CN, —SO₂Cl, —SOₙ₁R⁵, —SOᵥ₁NR⁵R⁶, —NHNH₂, —ONR⁵R⁶, —NHC═(O)NHNH₂, —NHC═(O)NR⁵R⁶, —N(O)ₘ₁, —NR⁵R⁶, —NH—O—R⁵, —C(O)R⁵, —C(O)—OR⁵, —C(O)NR⁵R⁶, —OR⁵, R^{1A}-substituted or unsubstituted alkyl, R^{1A}-substituted or unsubstituted heteroalkyl, R^{1A}-substituted or unsubstituted cycloalkyl, R^{1A}-substituted or unsubstituted heterocycloalkyl, R^{1A}-substituted or unsubstituted aryl, or R^{1A}-substituted or unsubstituted heteroaryl. In embodiments, R¹ is hydrogen, halogen, —CX^{a}₃, —CN, —SO₂Cl, —SOₙ₁R⁵, —SOᵥ₁NR⁵R⁶, —NHNH₂, —ONR⁵R⁶, —NHC═(O)NHNH₂, —NHC═(O)NR⁵R⁶, —N(O)ₘ₁, —NR⁵R⁶, —NH—O—R⁵, —C(O)R⁵, —C(O)—OR⁵, —C(O)NR⁵R⁶, or —OR⁵. In embodiments, R¹ is R^{1A}-substituted or unsubstituted C₁-C₂₀ (e.g., C₁-C₆) alkyl, R^{1A}-substituted or unsubstituted 2 to 20 membered (e.g., 2 to 6 membered) heteroalkyl, R^{1A}-substituted or unsubstituted C₃-C₈ (e.g., C₅-C₇) cycloalkyl, R^{1A}-substituted or unsubstituted 3 to 8 membered (e.g., 3 to 6 membered) heterocycloalkyl, R^{1A}-substituted or unsubstituted C₅-C₁₀ (e.g., C₅-C₆) aryl, or R^{1A}-substituted or unsubstituted 5 to 10 membered (e.g., 5 to 6 membered) heteroaryl.

R^{1A} may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{1B}-substituted or unsubstituted alkyl, R^{1B}-substituted or unsubstituted heteroalkyl, R^{1B}-substituted or unsubstituted cycloalkyl, R^{1B}-substituted or unsubstituted heterocycloalkyl, R^{1B}-substituted or unsubstituted aryl, or R^{1B}-substituted or unsubstituted heteroaryl. In embodiments, where R^{1A} is ═O or ═S, R¹ is not aryl or heteroaryl. In embodiments, R^{1A} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In other embodiments, R^{1A} is R^{1B}-substituted or unsubstituted alkyl, R^{1B}-substituted or unsubstituted heteroalkyl, R^{1B}-substituted or unsubstituted cycloalkyl, R^{1B}-substituted or unsubstituted heterocycloalkyl, R^{1B}-substituted or unsubstituted aryl, or R^{1B}-substituted or unsubstituted heteroaryl. R^{1A} may be R^{1B}-substituted or unsubstituted C₁-C₂₀ (e.g., C₁-C₆) alkyl, R^{1B}-substituted or unsubstituted 2 to 20 membered (e.g., 2 to 6 membered) heteroalkyl, R^{1B}-substituted or unsubstituted C₃-C₈ (e.g., C₅-C₇) cycloalkyl, R^{1B}-substituted or unsubstituted 3 to 8 membered (e.g., 3 to 6 membered) heterocycloalkyl, R^{1B}-substituted or unsubstituted C₅-C₁₀ (e.g., C₅-C₆) aryl, or R^{1B}-substituted or unsubstituted 5 to 10 membered (e.g., 5 to 6 membered) heteroaryl.

R^{1B} may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{1C}-substituted or unsubstituted alkyl, R^{1C}-substituted or unsubstituted heteroalkyl, R^{1C}-substituted or unsubstituted cycloalkyl, R^{1C}-substituted or unsubstituted heterocycloalkyl, R^{1C}-substituted or unsubstituted aryl, or R^{1C}-substituted or unsubstituted heteroaryl. In embodiments, where R^{1B} is ═O or ═S, R^{1A} is not aryl or heteroaryl. In embodiments, R^{1B} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In other embodiments, R^{1B} is R^{1C}-substituted or unsubstituted alkyl, R^{1C}-substituted or unsubstituted heteroalkyl, R^{1C}-substituted or unsubstituted cycloalkyl, R^{1C}-substituted or unsubstituted heterocycloalkyl, R^{1C}-substituted or unsubstituted aryl, or R^{1C}-substituted or unsubstituted heteroaryl. R^{1B} may be R^{1C}-substituted or unsubstituted C₁-C₂₀ (e.g., C₁-C₆) alkyl, R^{1C}-substituted or unsubstituted 2 to 20 membered (e.g., 2 to 6 membered) heteroalkyl, R^{1C}-substituted or unsubstituted C₃-C₈ (e.g., C₅-C₇) cycloalkyl, R^{1C}-substituted or unsubstituted 3 to 8 membered (e.g., 3 to 6 membered) heterocycloalkyl, R^{1C}-substituted or unsubstituted C₅-C₁₀ (e.g., C₅-C₆) aryl, or R^{1C}-substituted or unsubstituted 5 to 10 membered (e.g., 5 to 6 membered) heteroaryl.

R^{1C} may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{1D}-substituted or unsubstituted alkyl, R^{1D}-substituted or unsubstituted heteroalkyl, R^{1D}-substituted or unsubstituted cycloalkyl, R^{1D}-substituted or unsubstituted heterocycloalkyl, R^{1D}-substituted or unsubstituted aryl, or R^{1D}-substituted or unsubstituted heteroaryl. In embodiments, where R^{1C} is ═O or ═S, R^{1B} is not aryl or heteroaryl. In embodiments, R^{1C} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In other embodiments, R^{1C} is R^{1D}-substituted or unsubstituted alkyl, R^{1D}-substituted or unsubstituted heteroalkyl, R^{1D}-substituted or unsubstituted cycloalkyl, R^{1D}-substituted or unsubstituted heterocycloalkyl, R^{1D}-substituted or unsubstituted aryl, or R^{1D}-substituted or unsubstituted heteroaryl. R^{1C} may be R^{1D}-substituted or unsubstituted C₁-C₂₀ (e.g., C₁-C₆) alkyl, R^{1D}-substituted or unsubstituted 2 to 20 membered (e.g., 2 to 6 membered) heteroalkyl, R^{1D}-substituted or unsubstituted C₃-C₈ (e.g., C₅-C₇) cycloalkyl, R^{1D}-substituted or unsubstituted 3 to 8 membered (e.g., 3 to 6 membered) heterocycloalkyl, R^{1D}-substituted or unsubstituted C₅-C₁₀ (e.g., C₅-C₆) aryl, or R^{1D}-substituted or unsubstituted 5 to 10 membered (e.g., 5 to 6 membered) heteroaryl.

R^{1D} may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{1E}-substituted or unsubstituted alkyl, R^{1E}-substituted or unsubstituted heteroalkyl, R^{1E}-substituted or unsubstituted cycloalkyl, R^{1E}-substituted or unsubstituted heterocycloalkyl, R^{1E}-substituted or unsubstituted aryl, or R^{1E}-substituted or unsubstituted heteroaryl. In embodiments, where R^{1D} is ═O or ═S, R^{1C} is not aryl or heteroaryl. In embodiments, R^{1D} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In other embodiments, R^{1D} is R^{1E}-substituted or unsubstituted alkyl, R^{1E}-substituted or unsubstituted heteroalkyl, R^{1E}-substituted or unsubstituted cycloalkyl, R^{1E}-substituted or unsubstituted heterocycloalkyl, R^{1E}-substituted or unsubstituted aryl, or R^{1E}-substituted or unsubstituted heteroaryl. R^{1D} may be R^{1E}-substituted or unsubstituted C₁-C₂₀ (e.g., C₁-C₆) alkyl, R^{1E}-substituted or unsubstituted 2 to 20 membered (e.g., 2 to 6 membered) heteroalkyl, R^{1E}-substituted or unsubstituted C₃-C₈ (e.g., C₅-C₇) cycloalkyl, R^{1E}-substituted or unsubstituted 3 to 8 membered (e.g., 3 to 6 membered) heterocycloalkyl, R^{1E}-substituted or unsubstituted C₅-C₁₀ (e.g., C₅-C₆) aryl, or R^{1E}-substituted or unsubstituted 5 to 10 membered (e.g., 5 to 6 membered) heteroaryl.

R^{1E} may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. In embodiments, where R^{1E} is ═O or ═S, R^{1D} is not aryl or heteroaryl. In embodiments, R^{1E} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In other embodiments, R^{1E} is unsubstituted alkyl, unsubstituted heteroalkyl, unsubstituted cycloalkyl, unsubstituted heterocycloalkyl, unsubstituted aryl, or unsubstituted heteroaryl. R^{1E} may be unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cycloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

In embodiments, R¹ of formula (I) or (II) is independently R^{1A}-substituted or unsubstituted alkyl, R^{1A}-substituted or unsubstituted heteroalkyl, R^{1A}-substituted or unsubstituted cycloalkyl, R^{1A}-substituted or unsubstituted heterocycloalkyl, R^{1A}-substituted or unsubstituted aryl, or R^{1A}-substituted or unsubstituted heteroaryl. R^{1A} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{1B}-substituted or unsubstituted alkyl, R^{1B}-substituted or unsubstituted heteroalkyl, R^{1B}-substituted or unsubstituted cycloalkyl, R^{1B}-substituted or unsubstituted heterocycloalkyl, R^{1B}-substituted or unsubstituted aryl, or R^{1B}-substituted or unsubstituted heteroaryl. R^{1B} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{1C}-substituted or unsubstituted alkyl, R^{1C}-substituted or unsubstituted heteroalkyl, R^{1C}-substituted or unsubstituted cycloalkyl, R^{1C}-substituted or unsubstituted heterocycloalkyl, R^{1C}-substituted or unsubstituted aryl, or R^{1C}-substituted or unsubstituted heteroaryl. R^{1C} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{1D}-substituted or unsubstituted alkyl, R^{1D}-substituted or unsubstituted heteroalkyl, R^{1D}-substituted or unsubstituted cycloalkyl, R^{1D}-substituted or unsubstituted heterocycloalkyl, R^{1D}-substituted or unsubstituted aryl, or R^{1D}-substituted or unsubstituted heteroaryl. R^{1D} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{1E}-substituted or unsubstituted alkyl, R^{1E}-substituted or unsubstituted heteroalkyl, R^{1E}-substituted or unsubstituted cycloalkyl, R^{1E}-substituted or unsubstituted heterocycloalkyl, R^{1E}-substituted or unsubstituted aryl, or R^{1E}-substituted or unsubstituted heteroaryl. And R^{1E} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl.

In embodiments of formula (I) or (II), R² may be hydrogen, halogen, —CX^{b}₃, —CN, —SO₂Cl, —SOₙ₂R⁷, —SOᵥ₂NR⁷R⁸, —NHNH₂, —ONR⁷R⁸, —NHC═(O)NHNH₂, —NHC═(O)NR⁷R⁸, —N(O)ₘ₂, —NR⁷R⁸, —NH—O—R⁷, —C(O)R⁷, —C(O)—OR⁷, —C(O)NR⁷R⁸, —OR⁷, substituted or unsubstituted C₁-C₂₀ (e.g., C₁-C₆) alkyl, substituted or unsubstituted 2 to 20 membered (e.g., 2 to 6 membered) heteroalkyl, substituted or unsubstituted C₃-C₈ (e.g., C₅-C₇) cycloalkyl, substituted or unsubstituted 3 to 8 membered (e.g., 3 to 6 membered) heterocycloalkyl, substituted or unsubstituted C₅-C₁₀ (e.g., C₅-C₆) aryl, or substituted or unsubstituted 5 to 10 membered (e.g., 5 to 6 membered) heteroaryl. In embodiments of formula (I) or (II), R² may be hydrogen, halogen, —CX^{b}₃, —CN, —SO₂Cl, —SOₙ₂R⁷, —SOᵥ₂NR⁷R⁸, —NHNH₂, —ONR⁷R⁸, —NHC═(O)NHNH₂, —NHC═(O)NR⁷R⁸, —N(O)ₘ₂, —NR⁷R⁸, —NH—O—R⁷, —C(O)R⁷, —C(O)—OR⁷, —C(O)NR⁷R⁸, —OR⁷, unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cycloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

R³ may be independently hydrogen, halogen, —CX^{c}₃, —CN, —SO₂Cl, —SOₙ₃R⁹, —SOᵥ₃NR⁹R¹⁰, —NHNH₂, —ONR⁹R¹⁰, —NHC═(O)NHNH₂, —NHC═(O)NR⁹R¹⁰, —N(O)ₘ₃, —NR⁹R⁸, —NH—O—R⁹, —C(O)R⁹, —C(O)—OR⁹, —C(O)NR⁹R¹⁰, —OR⁹, substituted or unsubstituted C₁-C₂₀ (e.g., C₁-C₆) alkyl, substituted or unsubstituted 2 to 20 membered (e.g., 2 to 6 membered) heteroalkyl, substituted or unsubstituted C₃-C₈ (e.g., C₅-C₇) cycloalkyl, substituted or unsubstituted 3 to 8 membered (e.g., 3 to 6 membered) heterocycloalkyl, substituted or unsubstituted C₅-C₁₀ (e.g., C₅-C₆) aryl, or substituted or unsubstituted 5 to 10 membered (e.g., 5 to 6 membered) heteroaryl. In embodiments of formula (I) or (II) R³ is hydrogen, halogen, —CX^{c}₃, —CN, —SO₂Cl, —SOₙ₃R⁹, —SOᵥ₃NR⁹R¹⁰, —NHNH₂, —ONR⁹R¹⁰, —NHC═(O)NHNH₂, —NHC═(O)NR⁹R¹⁰, —N(O)ₘ₃, —NR⁹R⁸, —NH—O—R⁹, —C(O)R⁹, —C(O)—OR⁹, —C(O)NR⁹R¹⁰, —OR⁹, —C(O)NR⁷R⁸, unsubstituted alkyl, unsubstituted heteroalkyl, unsubstituted cycloalkyl, unsubstituted heterocycloalkyl, unsubstituted aryl, or unsubstituted heteroaryl.

In embodiments of formula (I) or (II), R⁴ may be hydrogen, halogen, —CX^{d}₃, —CN, —SO₂Cl, —SOₙ₄R⁹, —SOᵥ₄NR¹¹R¹², —NHNH₂, —ONR¹¹R¹², —NHC═(O)NHNH₂, —NHC═(O)NR¹¹R¹², —N(O)ₘ₄, —NR¹¹R¹², —NH—O—R¹¹, —C(O)R¹¹, —C(O)—OR¹¹, —C(O)NR¹¹R¹², —OR¹¹, substituted or unsubstituted C₁-C₂₀ (e.g., C₁-C₆) alkyl, substituted or unsubstituted 2 to 20 membered (e.g., 2 to 6 membered) heteroalkyl, substituted or unsubstituted C₃-C₈ (e.g., C₅-C₇) cycloalkyl, substituted or unsubstituted 3 to 8 membered (e.g., 3 to 6 membered) heterocycloalkyl, substituted or unsubstituted C₅-C₁₀ (e.g., C₅-C₆) aryl, or substituted or unsubstituted 5 to 10 membered (e.g., 5 to 6 membered) heteroaryl. In embodiments of formula (I) or (II), R⁴ may be hydrogen, halogen, —CX^{d}₃, —CN, —SO₂Cl, —SOₙ₄R⁹, —SOᵥ₄NR¹¹R¹², —NHNH₂, —ONR¹¹R¹², —NHC═(O)NHNH₂, —NHC═(O)NR¹¹R¹², —N(O)ₘ₄, —NR¹¹R¹², —NH—O—R¹¹, —C(O)R¹¹, —C(O)—OR¹¹, —C(O)NR¹¹R¹², —OR¹¹ unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cycloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

In embodiments of formula (I) or (II), L¹ may be a bond, —S(O)—, —S(O)₂NH—, —NHS(O)₂—, —C(O)O—, —OC(O)—, —C(O)—, —C(O)NH—, —NH—, —NHC(O)—, —O—, —S—, —NH-L²-, —NH—R¹⁵—, substituted or unsubstituted C₁-C₂₀ (e.g., C₁-C₆) alkylene, substituted or unsubstituted 2 to 20 membered (e.g., 2 to 6 membered) heteroalkylene, substituted or unsubstituted C₃-C₈ (e.g., C₅-C₇) cycloalkylene, substituted or unsubstituted 3 to 8 membered (e.g., 3 to 6 membered) heterocycloalkylene, substituted or unsubstituted C₅-C₁₀ (e.g., C₅-C₆) arylene, or substituted or unsubstituted 5 to 10 membered (e.g., 5 to 6 membered) heteroarylene. In embodiments of formula (I) or (II), L¹ may be a bond, —S(O)—, —S(O)₂NH—, —NHS(O)₂—, —C(O)O—, —OC(O)—, —C(O)—, —C(O)NH—, —NH—, —NHC(O)—, —O—, —S—, —NH-L²-, —NH—R¹⁵—, unsubstituted C₁-C₂₀ (e.g., C₁-C₆) alkylene, unsubstituted 2 to 20 membered (e.g., 2 to 6 membered) heteroalkylene, unsubstituted C₃-C₈ (e.g., C₅-C₇) cycloalkylene, unsubstituted 3 to 8 membered (e.g., 3 to 6 membered) heterocycloalkylene, unsubstituted C₅-C₁₀ (e.g., C₅-C₆) arylene, or unsubstituted 5 to 10 membered (e.g., 5 to 6 membered) heteroarylene.

In embodiments of formula (I) or (II), L² may be independently —C(O)—, —C(O)—NH—, substituted or unsubstituted C₁-C₂₀ (e.g., C₁-C₆) alkylene, substituted or unsubstituted 2 to 20 membered (e.g., 2 to 6 membered) heteroalkylene, substituted or unsubstituted C₃-C₈ (e.g., C₅-C₇) cycloalkylene, substituted or unsubstituted 3 to 8 membered (e.g., 3 to 6 membered) heterocycloalkylene, substituted or unsubstituted C₅-C₁₀ (e.g., C₅-C₆) arylene, or substituted or unsubstituted 5 to 10 membered (e.g., 5 to 6 membered) heteroarylene.

In embodiments, L² is independently —C(O)—, —C(O)—NH—, R¹⁶-substituted or unsubstituted alkylene, R¹⁶-substituted or unsubstituted heteroalkylene, R¹⁶-substituted or unsubstituted cycloalkylene, R¹⁶-substituted or unsubstituted heterocycloalkylene, R¹⁶-substituted or unsubstituted arylene, or R¹⁶-substituted or unsubstituted heteroarylene. In embodiments, L² is —C(O)— or —C(O)—NH—. In embodiments, L² is independently R¹⁶-substituted or unsubstituted alkylene, R¹⁶-substituted or unsubstituted heteroalkylene, R¹⁶-substituted or unsubstituted cycloalkylene, R¹⁶-substituted or unsubstituted heterocycloalkylene, R¹⁶-substituted or unsubstituted arylene, or R¹⁶-substituted or unsubstituted heteroarylene. L² may be independently R¹⁶-substituted or unsubstituted C₁-C₂₀ (e.g., C₁-C₆) alkylene, R¹⁶-substituted or unsubstituted 2 to 20 membered (e.g., 2 to 6 membered) heteroalkylene, R¹⁶-substituted or unsubstituted C₃-C₈ (e.g., C₅-C₇) cycloalkylene, R¹⁶-substituted or unsubstituted 3 to 8 membered (e.g., 3 to 6 membered) heterocycloalkylene, R¹⁶-substituted or unsubstituted C₅-C₁₀ (e.g., C₅-C₆) arylene, or R¹⁶-substituted or unsubstituted 5 to 10 membered (e.g., 5 to 6 membered) heteroarylene.

R¹⁶ may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R¹⁷-substituted or unsubstituted alkyl, R¹⁷-substituted or unsubstituted heteroalkyl, R¹⁷-substituted or unsubstituted cycloalkyl, R¹⁷-substituted or unsubstituted heterocycloalkyl, R¹⁷-substituted or unsubstituted aryl, or R¹⁷-substituted or unsubstituted heteroaryl. In embodiments, where R¹⁶ is ═O or ═S, L² is not arylene or heteroarylene. In embodiments, R¹⁶ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂ or —NHC═(O)NHNH₂. In embodiments, R¹⁶ is independently R¹⁷-substituted or unsubstituted alkyl, R¹⁷-substituted or unsubstituted heteroalkyl, R¹⁷-substituted or unsubstituted cycloalkyl, R¹⁷-substituted or unsubstituted heterocycloalkyl, R¹⁷-substituted or unsubstituted aryl, or R¹⁷-substituted or unsubstituted heteroaryl. R¹⁶ may be independently R¹⁷-substituted or unsubstituted C₁-C₂₀ (e.g., C₁-C₆) alkyl, R¹⁷-substituted or unsubstituted 2 to 20 membered (e.g., 2 to 6 membered) heteroalkyl, substituted or unsubstituted C₃-C₈ (e.g., C₅-C₇) cycloalkyl, R¹⁷-substituted or unsubstituted 3 to 8 membered (e.g., 3 to 6 membered) heterocycloalkyl, R¹⁷-substituted or unsubstituted C₅-C₁₀ (e.g., C₅-C₆) aryl, or R¹⁷-substituted or unsubstituted 5 to 10 membered (e.g., 5 to 6 membered) heteroaryl.

R¹⁷ may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R¹⁸-substituted or unsubstituted alkyl, R¹⁸-substituted or unsubstituted heteroalkyl, R¹⁸-substituted or unsubstituted cycloalkyl, R¹⁸-substituted or unsubstituted heterocycloalkyl, R¹⁸-substituted or unsubstituted aryl, or R¹⁸-substituted or unsubstituted heteroaryl. In embodiments, where R¹⁷ is ═O or ═S, R¹⁶ is not aryl or heteroaryl. In embodiments, R¹⁷ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂ or —NHC═(O)NHNH₂. In embodiments, R¹⁷ is independently R¹⁸-substituted or unsubstituted alkyl, R¹⁸-substituted or unsubstituted heteroalkyl, R¹⁸-substituted or unsubstituted cycloalkyl, R¹⁸-substituted or unsubstituted heterocycloalkyl, R¹⁸-substituted or unsubstituted aryl, or R¹⁸-substituted or unsubstituted heteroaryl. R¹⁷ may be independently R¹⁸-substituted or unsubstituted C₁-C₂₀ (e.g., C₁-C₆) alkyl, R¹⁸-substituted or unsubstituted 2 to 20 membered (e.g., 2 to 6 membered) heteroalkyl, R¹⁸-substituted or unsubstituted C₃-C₈ (e.g., C₅-C₇) cycloalkyl, R¹⁸-substituted or unsubstituted 3 to 8 membered (e.g., 3 to 6 membered) heterocycloalkyl, R¹⁸-substituted or unsubstituted C₅-C₁₀ (e.g., C₅-C₆) aryl, or R¹⁸-substituted or unsubstituted 5 to 10 membered (e.g., 5 to 6 membered) heteroaryl.

R¹⁸ may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R¹⁹-substituted or unsubstituted alkyl, R¹⁹-substituted or unsubstituted heteroalkyl, R¹⁹-substituted or unsubstituted cycloalkyl, R¹⁹-substituted or unsubstituted heterocycloalkyl, R¹⁹-substituted or unsubstituted aryl, or R¹⁹-substituted or unsubstituted heteroaryl. In embodiments, where R¹⁸ is ═O or ═S, R¹⁷ is not aryl or heteroaryl. In embodiments, R¹⁸ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂ or —NHC═(O)NHNH₂. In embodiments, R¹⁸ is independently R¹⁹-substituted or unsubstituted alkyl, R¹⁹-substituted or unsubstituted heteroalkyl, R¹⁹-substituted or unsubstituted cycloalkyl, R¹⁹-substituted or unsubstituted heterocycloalkyl, R¹⁹-substituted or unsubstituted aryl, or R¹⁹-substituted or unsubstituted heteroaryl. R¹⁸ may be independently R¹⁹-substituted or unsubstituted C₁-C₂₀ (e.g., C₁-C₆) alkyl, R¹⁹-substituted or unsubstituted 2 to 20 membered (e.g., 2 to 6 membered) heteroalkyl, R¹⁹-substituted or unsubstituted C₃-C₈ (e.g., C₅-C₇) cycloalkyl, R¹⁹-substituted or unsubstituted 3 to 8 membered (e.g., 3 to 6 membered) heterocycloalkyl, R¹⁹-substituted or unsubstituted C₅-C₁₀ (e.g., C₅-C₆) aryl, or R¹⁹-substituted or unsubstituted 5 to 10 membered (e.g., 5 to 6 membered) heteroaryl.

R¹⁹ may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R²⁰-substituted or unsubstituted alkyl, R²⁰-substituted or unsubstituted heteroalkyl, R²⁰-substituted or unsubstituted cycloalkyl, R²⁰-substituted or unsubstituted heterocycloalkyl, R²⁰-substituted or unsubstituted aryl, or R²⁰-substituted or unsubstituted heteroaryl. In embodiments, where R¹⁹ is ═O or ═S, R¹⁸ is not aryl or heteroaryl. In embodiments, R¹⁹ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂ or —NHC—(O)NHNH₂. In embodiments, R¹⁹ is independently R²⁰-substituted or unsubstituted alkyl, R²⁰-substituted or unsubstituted heteroalkyl, R²⁰-substituted or unsubstituted cycloalkyl, R²⁰-substituted or unsubstituted heterocycloalkyl, R²⁰-substituted or unsubstituted aryl, or R²⁰-substituted or unsubstituted heteroaryl. R¹⁹ may be independently R²⁰-substituted or unsubstituted C₁-C₂₀ (e.g., C₁-C₆) alkyl, R²⁰-substituted or unsubstituted 2 to 20 membered (e.g., 2 to 6 membered) heteroalkyl, R²⁰-substituted or unsubstituted C₃-C₈ (e.g., C₅-C₇) cycloalkyl, R²⁰-substituted or unsubstituted 3 to 8 membered (e.g., 3 to 6 membered) heterocycloalkyl, R²⁰-substituted or unsubstituted C₅-C₁₀ (e.g., C₅-C₆) aryl, or R²⁰-substituted or unsubstituted 5 to 10 membered (e.g., 5 to 6 membered) heteroaryl.

R²⁰ may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. In embodiments, where R²⁰ is ═O or ═S, R¹⁹ is not aryl or heteroaryl. In embodiments, R²⁰ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂ or —NHC═(O)NHNH₂. In embodiments, R²⁰ is independently unsubstituted alkyl, unsubstituted heteroalkyl, unsubstituted cycloalkyl, unsubstituted heterocycloalkyl, unsubstituted aryl, or unsubstituted heteroaryl. R²⁰ may be independently unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cycloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

In embodiments, L² is R¹⁶-substituted or unsubstituted alkylene, R¹⁶-substituted or unsubstituted heteroalkylene, R¹⁶-substituted or unsubstituted cycloalkylene, R¹⁶-substituted or unsubstituted heterocycloalkylene, R¹⁶-substituted or unsubstituted arylene, or R¹⁶-substituted or unsubstituted heteroarylene. R¹⁶ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R¹⁷-substituted or unsubstituted alkyl, R¹⁷-substituted or unsubstituted heteroalkyl, R¹⁷-substituted or unsubstituted cycloalkyl, R¹⁷-substituted or unsubstituted heterocycloalkyl, R¹⁷-substituted or unsubstituted aryl, or R¹⁷-substituted or unsubstituted heteroaryl. R¹⁷ is independently hydrogen, halogen, ═O, ═S, —CF₃, —CN, —CCl₃, —COOH, —CH₂COOH, —CONH₂, —OH, —SH, —SO₂Cl, —SO₂NH₂, —NO₂, —NH₂, —NHNH₂, —ONH₂, —NHC═(O)NHNH₂, R¹⁸-substituted or unsubstituted alkyl, R¹⁸-substituted or unsubstituted heteroalkyl, R¹⁸-substituted or unsubstituted cycloalkyl, R¹⁸-substituted or unsubstituted heterocycloalkyl, R¹⁸-substituted or unsubstituted aryl, or R¹⁸-substituted or unsubstituted heteroaryl. R¹⁸ is independently hydrogen, halogen, ═O, ═S, —CF₃, —CN, —CCl₃, —COOH, —CH₂COOH, —CONH₂, —OH, —SH, —SO₂Cl, —SO₂NH₂, —NO₂, —NH₂, —NHNH₂, —ONH₂, —NHC═(O)NHNH₂, R¹⁹-substituted or unsubstituted alkyl, R¹⁹-substituted or unsubstituted heteroalkyl, R¹⁹-substituted or unsubstituted cycloalkyl, R¹⁹-substituted or unsubstituted heterocycloalkyl, R¹⁹-substituted or unsubstituted aryl, or R¹⁹-substituted or unsubstituted heteroaryl. R¹⁹ is independently hydrogen, halogen, ═O, ═S, —CF₃, —CN, —CCl₃, —COOH, —CH₂COOH, —CONH₂, —OH, —SH, —SO₂Cl, —SO₂NH₂, —NO₂, —NH₂, —NHNH₂, —ONH₂, —NHC═(O)NHNH₂, R^{N}-substituted or unsubstituted alkyl, R²⁰-substituted or unsubstituted heteroalkyl, R²⁰-substituted or unsubstituted cycloalkyl, R²⁰-substituted or unsubstituted heterocycloalkyl, R²⁰-substituted or unsubstituted aryl, or R²⁰-substituted or unsubstituted heteroaryl. And R²⁰ is independently hydrogen, halogen, ═O, ═S, —CF₃, —CN, —CCl₃, —COOH, —CH₂COOH, —CONH₂, —OH, —SH, —SO₂Cl, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl.

In embodiments of formula (I) or (II) R⁵, R⁶, R⁷, R⁸, R⁹, R¹⁰, R¹¹, R¹², R¹³, R¹⁴ and R¹⁵ are independently hydrogen, halogen, ═O, ═S, —CF₃, —CN, —CCl₃, —COOH, —CH₂COOH, —CONH₂, —OH, —SH, —SO₂Cl, —SO₃H, —SO₂NH₂, —NO₂, —NH₂, —NHNH₂, —ONH₂ or —NHC═(O)NHNH₂. In embodiments, R⁵, R⁶, R⁷, R⁸, R⁹, R¹⁰, R¹¹, R¹², R¹³, R¹⁴ and R¹⁵ are independently substituted or unsubstituted C₁-C₂₀ (e.g., C₁-C₆) alkyl, substituted or unsubstituted 2 to 20 membered (e.g., 2 to 6 membered) heteroalkyl, substituted or unsubstituted C₃-C₈ (e.g., C₅-C₇) cycloalkyl, substituted or unsubstituted 3 to 8 membered (e.g., 3 to 6 membered) heterocycloalkyl, substituted or unsubstituted C₅-C₁₀ (e.g., C₅-C₆) aryl, or substituted or unsubstituted 5 to 10 membered (e.g., 5 to 6 membered) heteroaryl.

In embodiments, R⁵, R⁶, R⁷, R⁸, R⁹, R¹⁰, R¹¹, R¹², R¹³, R¹⁴ and R¹⁵ are independently hydrogen, halogen, ═O, ═S, —CF₃, —CN, —CCl₃, —COOH, —CH₂COOH, —CONH₂, —OH, —SH, —SO₂Cl, —SO₂NH₂, —NO₂, —NH₂, —NHNH₂, —ONH₂, —NHC═(O)NHNH₂, R⁵¹-substituted or unsubstituted alkyl, R⁵¹-substituted or unsubstituted heteroalkyl, R⁵¹-substituted or unsubstituted cycloalkyl, R⁵¹-substituted or unsubstituted heterocycloalkyl, R⁵¹-substituted or unsubstituted aryl, or R⁵¹-substituted or unsubstituted heteroaryl. In embodiments, R⁵, R⁶, R⁷, R⁸, R⁹, R¹⁰, R¹¹, R¹², R¹³, R¹⁴, and R¹⁵ are independently hydrogen, halogen, ═O, ═S, —CF₃, —CN, —CCl₃, —COOH, —CH₂COOH, —CONH₂, —OH, —SH, —SO₂Cl, —SO₂NH₂, —NO₂, —NH₂, —NHNH₂, —ONH₂ or —NHC═(O)NHNH₂. In embodiments, R⁵, R⁶, R⁷, R⁸, R⁹, R¹⁰, R¹¹, R¹², R¹³, R¹⁴ and R15 are independently R⁵¹-substituted or unsubstituted alkyl, R⁵¹-substituted or unsubstituted heteroalkyl, R⁵¹-substituted or unsubstituted cycloalkyl, R⁵¹-substituted or unsubstituted heterocycloalkyl, R⁵¹-substituted or unsubstituted aryl, or R⁵¹-substituted or unsubstituted heteroaryl. R⁵, R⁶, R⁷, R⁸, R⁹, R¹⁰, R¹¹, R¹², R¹³, R¹⁴ and R¹⁵ may be independently R⁵¹-substituted or unsubstituted C₁-C₂₀ (e.g., C₁-C₆) alkyl, R⁵¹-substituted or unsubstituted 2 to 20 membered (e.g., 2 to 6 membered) heteroalkyl, R⁵¹-substituted or unsubstituted C₃-C₈ (e.g., C₅-C₇) cycloalkyl, R⁵¹-substituted or unsubstituted 3 to 8 membered (e.g., 3 to 6 membered) heterocycloalkyl, R⁵¹-substituted or unsubstituted C₅-C₁₀ (e.g., C₅-C₆) aryl, or R⁵¹-substituted or unsubstituted 5 to 10 membered (e.g., 5 to 6 membered) heteroaryl.

R⁵¹ as provided herein may be independently hydrogen, halogen, ═O, ═S, —CF₃, —CN, —CCl₃, —COOH, —CH₂COOH, —CONH₂, —OH, —SH, —SO₂Cl, —SO₂NH₂, —NO₂, —NH₂, —NHNH₂, —ONH₂, —NHC═(O)NHNH₂, R⁵²-substituted or unsubstituted alkyl, R⁵²-substituted or unsubstituted heteroalkyl, R⁵²-substituted or unsubstituted cycloalkyl, R⁵²-substituted or unsubstituted heterocycloalkyl, R⁵²-substituted or unsubstituted aryl, or R⁵²-substituted or unsubstituted heteroaryl. In embodiments, R⁵¹is independently hydrogen, halogen, ═O, ═S, —CF₃, —CN, —CCl₃, —COOH, —CH₂COOH, —CONH₂, —OH, —SH, —SO₂Cl, —SO₂NH₂, —NO₂, —NH₂, —NHNH₂, —ONH₂ or —NHC═(O)NHNH₂. In embodiments, where R⁵¹ is ═O or ═S, R⁵, R⁶, R⁷, R⁸, R⁹, R¹⁰, R¹¹, R¹², R¹³, R¹⁴ and R¹⁵ is not aryl or heteroaryl. In embodiments, R⁵¹ is independently R⁵²-substituted or unsubstituted alkyl, R⁵²-substituted or unsubstituted heteroalkyl, R⁵²-substituted or unsubstituted cycloalkyl, R⁵²-substituted or unsubstituted heterocycloalkyl, R⁵²-substituted or unsubstituted aryl, or R⁵²-substituted or unsubstituted heteroaryl. R⁵¹ may be independently R⁵²-substituted or unsubstituted C₁-C₂₀ (e.g., C₁-C₆) alkyl, R⁵²-substituted or unsubstituted 2 to 20 membered (e.g., 2 to 6 membered) heteroalkyl, R⁵²-substituted or unsubstituted C₃-C₈ (e.g., C₅-C₇) cycloalkyl, R⁵²-substituted or unsubstituted 3 to 8 membered (e.g., 3 to 6 membered) heterocycloalkyl, R⁵²-substituted or unsubstituted C₅-C₁₀ (e.g., C₅-C₆) aryl, or R⁵²-substituted or unsubstituted 5 to 10 membered (e.g., 5 to 6 membered) heteroaryl.

R⁵² as provided herein may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R⁵³-substituted or unsubstituted alkyl, R⁵³-substituted or unsubstituted heteroalkyl, R⁵³-substituted or unsubstituted cycloalkyl, R⁵³-substituted or unsubstituted heterocycloalkyl, R⁵³-substituted or unsubstituted aryl, or R⁵³-substituted or unsubstituted heteroaryl. In embodiments, R⁵²is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂ or —NHC═(O)NHNH₂. In embodiments, where R⁵² is ═O or ═S, R⁵¹ is not aryl or heteroaryl. In embodiments, R⁵² is independently R⁵³-substituted or unsubstituted alkyl, R⁵³-substituted or unsubstituted heteroalkyl, R⁵³-substituted or unsubstituted cycloalkyl, R⁵³-substituted or unsubstituted heterocycloalkyl, R⁵³-substituted or unsubstituted aryl, or R⁵³-substituted or unsubstituted heteroaryl. R⁵² may be independently R⁵³-substituted or unsubstituted C₁-C₂₀ (e.g., C₁-C₆) alkyl, R⁵³-substituted or unsubstituted 2 to 20 membered (e.g., 2 to 6 membered) heteroalkyl, R⁵³-substituted or unsubstituted C₃-C₈ (e.g., C₅-C₇) cycloalkyl, R⁵³-substituted or unsubstituted 3 to 8 membered (e.g., 3 to 6 membered) heterocycloalkyl, R⁵³-substituted or unsubstituted C₅-C₁₀ (e.g., C₅-C₆) aryl, or R⁵³-substituted or unsubstituted 5 to 10 membered (e.g., 5 to 6 membered) heteroaryl.

R⁵³ may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R⁵⁴-substituted or unsubstituted alkyl, R⁵⁴-substituted or unsubstituted heteroalkyl, R⁵⁴-substituted or unsubstituted cycloalkyl, R⁵⁴-substituted or unsubstituted heterocycloalkyl, R⁵⁴-substituted or unsubstituted aryl, or R⁵⁴-substituted or unsubstituted heteroaryl. In embodiments, R⁵³ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂ or —NHC═(O)NHNH₂. In embodiments, where R⁵³ is ═O or ═S, R⁵² is not aryl or heteroaryl. In embodiments, R⁵³ is independently R⁵⁴-substituted or unsubstituted alkyl, R⁵⁴-substituted or unsubstituted heteroalkyl, R⁵⁴-substituted or unsubstituted cycloalkyl, R⁵⁴-substituted or unsubstituted heterocycloalkyl, R⁵⁴-substituted or unsubstituted aryl, or R⁵⁴-substituted or unsubstituted heteroaryl. R⁵³ may be independently R⁵⁴-substituted or unsubstituted C₁-C₂₀ (e.g., C₁-C₆) alkyl, R⁵⁴-substituted or unsubstituted 2 to 20 membered (e.g., 2 to 6 membered) heteroalkyl, R⁵⁴-substituted or unsubstituted C₃-C₈ (e.g., C₅-C₇) cycloalkyl, R⁵⁴-substituted or unsubstituted 3 to 8 membered (e.g., 3 to 6 membered) heterocycloalkyl, R⁵⁴-substituted or unsubstituted C₅-C₁₀ (e.g., C₅-C₆) aryl, or R⁵⁴-substituted or unsubstituted 5 to 10 membered (e.g., 5 to 6 membered) heteroaryl.

R⁵⁴ may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl or unsubstituted heteroaryl. In embodiments, R⁵⁴ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂ or —NHC═(O)NHNH₂. In embodiments, where R⁵⁴ is ═O or ═S, R⁵³ is not aryl or heteroaryl. In embodiments, R⁵⁴ is independently unsubstituted alkyl, unsubstituted heteroalkyl, unsubstituted cycloalkyl, unsubstituted heterocycloalkyl, unsubstituted aryl or unsubstituted heteroaryl. R⁵⁴ may be independently unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cycloalkyl, unsubstituted 3 to 8 membered (e.g., 3 to 6 membered) heterocycloalkyl, unsubstituted C₅-C₁₀ (e.g., C₅-C₆) aryl or unsubstituted 5 to 10 membered (e.g., 5 to 6 membered) heteroaryl.

In embodiments, the compound of formula (I) or (II) including embodiments thereof may include multiple instances of R⁵¹, R⁵², R⁵³, and/or R⁵⁴ (e.g., R¹ may be —ONR⁵R⁶ and R⁵ and R⁶ may be independently R⁵¹-substituted). In such embodiments, each variable may optional be different and be appropriately labeled to distinguish each group for greater clarity. For example, where each R⁵¹, R⁵², R⁵³, and/or R⁵⁴ is different, they may be referred to, for example, as R^{51.1}, R^{51.2}, R^{51.3}, R^{51.4}, R^{52.1}, R^{52.2}, R^{52.3}, R^{52.4}, R^{53.1}, R^{53.2}, R^{53.3}, R^{53.4}, R^{54.1}, R^{54.2}, R^{54.3}, and/or R^{54.4}, respectively, wherein the definition of R⁵¹ is assumed by R^{51.1}, R^{51.2}, R^{51.3}, and/or R^{51.4}, the definition of R⁵² is assumed by R^{52.1}, R^{52.2}, R^{52.3}, and/or R^{52.4}, the definition of R⁵³ is assumed by R^{53.1}, R^{53.2}, R^{53.3}, and/or R^{53.4}, the definition of R⁵⁴ is assumed by R^{54.1}, R^{54.2}, R^{54.3}, and/or R^{54.4}. The variables used within a definition of R⁵¹, R⁵², R⁵³, and/or R⁵⁴ and/or other variables that appear at multiple instances and are different may similarly be appropriately labeled to distinguish each group for greater clarity.

In embodiments, the compounds as provided herein do not include compounds having the structure:

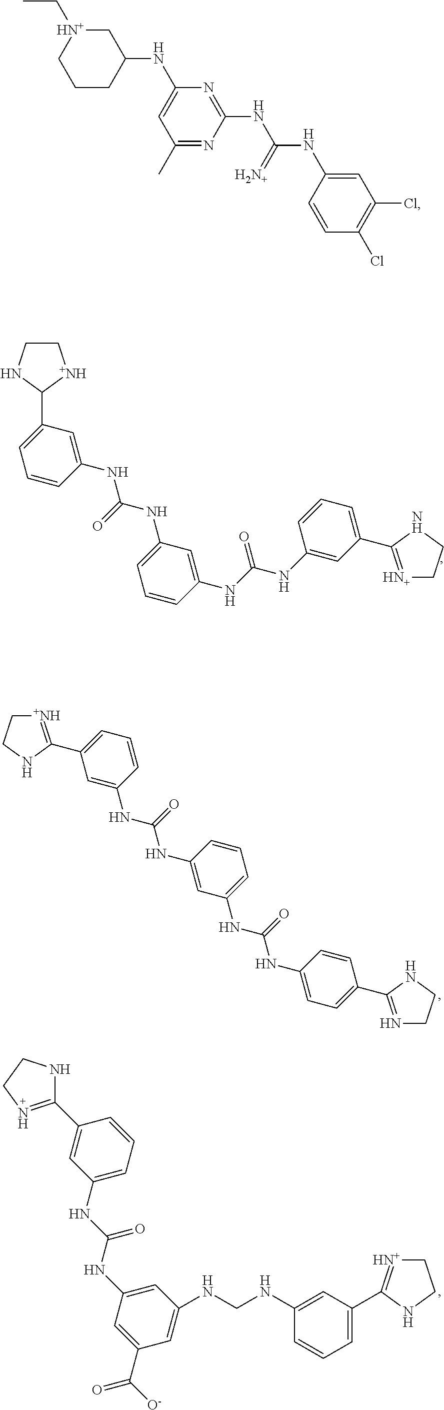 [SMILES: CC[NH+]1CCCC(Nc2cc(C)nc(NC(=[NH2+])Nc3ccc(Cl)c(Cl)c3)n2)C1 ; O=C(Nc1cccc(NC(=O)Nc2cccc(C3NCC[NH2+]3)c2)c1)Nc1cccc(C2=[NH+]CCN2)c1 ; O=C(Nc1ccc(C2=NCCN2)cc1)Nc1cccc(NC(=O)Nc2cccc(C3=[NH+]CCN3)c2)c1 ; O=C(Nc1cc(NCNc2cccc(C3=[NH+]CCN3)c2)cc(C(=O)[O-])c1)Nc1cccc(C2=NCCN2)c1]

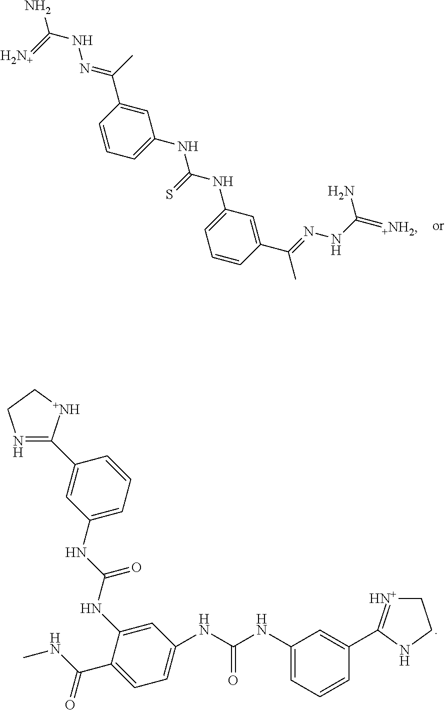

In embodiments, the compositions disclosed herein (e.g. of Formula (I) or (II)) do not include a compound having the formula:

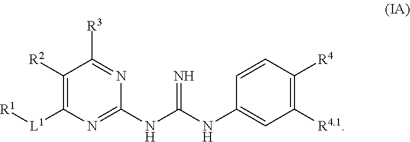

In embodiments, the composition does not include a compound of formula (IA), wherein R¹, R², R³, R⁴, R^{4.1} and L¹ are as defined herein (including embodiments thereof). In embodiments, compounds are not included wherein L¹ is a bond, R¹ is methyl, R² is hydrogen, R³ is —NR⁹R¹⁰, wherein R⁹ is hydrogen and R¹⁰ is

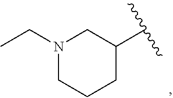 [SMILES: *C1CCCN(CC)C1]

and R⁴ and R^{4.1} are both —Cl. In embodiments, the composition does not include a compound of formula (IA), wherein L¹ is a bond, R¹ is methyl, R² is hydrogen, R³ is —NR⁹R¹⁰, wherein R⁹ is hydrogen and R¹⁰ is

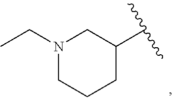 [SMILES: *C1CCCN(CC)C1]

and R⁴ and R^{4.1} are both halogen. In embodiments, the composition does not include a compound of formula (IA), wherein L¹ is a bond, R¹ is methyl, R² is hydrogen, R³ is —NR⁹R¹⁰, wherein R⁹ is hydrogen and R¹⁰ is R⁵¹-substituted 6 membered heterocycloalkyl, wherein R⁵¹ is propyl, and R⁴ and R^{4.1} are both halogen. In embodiments, the composition does not include a compound of formula (IA), wherein L¹ is a bond, R¹ is methyl, R² is hydrogen, R³ is —NR⁹R¹⁰, wherein R⁹ is hydrogen and R¹⁰ is R⁵¹-substituted or unsubstituted 6 to 10 membered heterocycloalkyl, wherein R⁵¹ is propyl, and R⁴ and R^{4.1} are both halogen. In embodiments, the composition does not include a compound of formula (IA), wherein L¹ is a bond, R¹ is methyl, R² is hydrogen, R³ is —NR⁹R¹⁰, wherein R⁹ is hydrogen and R¹⁰ is R⁵¹-substituted or unsubstituted heterocycloalkyl, wherein R⁵¹ is propyl, and R⁴ and R^{4.1} are both halogen.

In embodiments, the composition does not include a compound of formula (IA), wherein L¹ is a bond, R¹ is methyl, R² is hydrogen, R³ is —NR⁹R¹⁰, wherein R⁹ is hydrogen and R¹⁰ is R⁵¹-substituted or unsubstituted heterocycloalkyl, wherein R⁵¹ is unsubstituted C₁-C₃ alkyl, and R⁴ and R^{4.1} are both halogen. In embodiments, the composition does not include a compound of formula (IA), wherein L¹ is a bond, R¹ is methyl, R² is hydrogen, R³ is —NR⁹R¹⁰, wherein R⁹ is hydrogen and R¹⁰ is R⁵¹-substituted or unsubstituted heterocycloalkyl, wherein R⁵¹ is substituted or unsubstituted C₁-C₃ alkyl, and R⁴ and R^{4.1} are both halogen. In embodiments, the composition does not include a compound of formula (IA), wherein L¹ is a bond, R¹ is methyl, R² is hydrogen, R³ is —NR⁹R¹⁰, wherein R⁹ is hydrogen and R^{th} is R⁵¹-substituted or unsubstituted heterocycloalkyl, wherein R⁵¹ is unsubstituted C₁-C₅ alkyl, and R⁴ and R^{4.1} are both halogen. In embodiments, the composition does not include a compound of formula (IA), wherein L¹ is a bond, R¹ is methyl, R² is hydrogen, R³ is —NR⁹R¹⁰, wherein R⁹ is hydrogen and R¹⁰ is R⁵¹-substituted or unsubstituted heterocycloalkyl, wherein R⁵¹ is substituted or unsubstituted C₁-C₅ alkyl, and R⁴ and R^{4.1} are both halogen.

In embodiments, the composition does not include a compound of formula (IA), wherein L¹ is a bond, R¹ is methyl, R² is hydrogen, R³ is —NR⁹R¹⁰, wherein R⁹ is hydrogen and R¹⁰ is R⁵¹-substituted or unsubstituted heterocycloalkyl, wherein R⁵¹ is unsubstituted C₁-C₇ alkyl, and R⁴ and R^{4.1} are both halogen. In embodiments, the composition does not include a compound of formula (IA), wherein L¹ is a bond, R¹ is methyl, R² is hydrogen, R³ is —NR⁹R¹⁰, wherein R⁹ is hydrogen and R¹⁰ is R⁵¹-substituted or unsubstituted heterocycloalkyl, wherein R⁵¹ is substituted or unsubstituted C₁-C₇ alkyl, and R⁴ and R^{4.1} are both halogen. In embodiments, the composition does not include a compound of formula (IA), wherein L¹ is a bond, R¹ is unsubstituted C₁-C₃ alkyl, R² is hydrogen, R³ is —NR⁹R¹⁰, wherein R⁹ is hydrogen and R¹⁰ is R⁵¹-substituted or unsubstituted heterocycloalkyl, wherein R⁵¹ is substituted or unsubstituted C₁-C₇ alkyl, and R⁴ and R^{4.1} are both halogen. In embodiments, the composition does not include a compound of formula (IA), wherein L¹ is a bond, R¹ is substituted or unsubstituted C₁-C₃ alkyl, R² is hydrogen, R³ is —NR⁹R¹⁰, wherein R⁹ is hydrogen and R¹⁰ is R⁵¹-substituted or unsubstituted heterocycloalkyl, wherein R⁵¹ is substituted or unsubstituted C₁-C₇ alkyl, and R⁴ and R^{4.1} are both halogen. In embodiments, the composition does not include a compound of formula (IA), wherein L¹ is a bond, R¹ is unsubstituted C₁-C₅ alkyl, R² is hydrogen, R³ is —NR⁹R¹⁰, wherein R⁹ is hydrogen and R¹⁰ is R⁵¹-substituted or unsubstituted heterocycloalkyl, wherein R⁵¹ is substituted or unsubstituted C₁-C₇ alkyl, and R⁴ and R^{4.1} are both halogen. In embodiments, the composition does not include a compound of formula (IA), wherein L¹ is a bond, R¹ is substituted or unsubstituted C₁-C₅ alkyl, R² is hydrogen, R³ is —NR⁹R¹⁰, wherein R⁹ is hydrogen and R¹⁰ is R⁵¹-substituted or unsubstituted heterocycloalkyl, wherein R⁵¹ is substituted or unsubstituted C₁-C₇ alkyl, and R⁴ and R^{4.1} are both halogen.

In embodiments, the composition does not include a compound of formula (IA), wherein L¹ is a bond, R¹ is unsubstituted C₁-C₇ alkyl, R² is hydrogen, R³ is —NR⁹R¹⁰, wherein R⁹ is hydrogen and R¹⁰ is R⁵¹-substituted or unsubstituted heterocycloalkyl, wherein R⁵¹ is substituted or unsubstituted C₁-C₇ alkyl, and R⁴ and R^{4.1} are both halogen. In embodiments, the composition does not include a compound of formula (IA), wherein L¹ is a bond, R¹ is substituted or unsubstituted C₁-C₇ alkyl, R² is hydrogen, R³ is —NR⁹R¹⁰, wherein R⁹ is hydrogen and R¹⁰ is R⁵¹-substituted or unsubstituted heterocycloalkyl, wherein R⁵¹ is substituted or unsubstituted C₁-C₇ alkyl, and R⁴ and R^{4.1} are both halogen.

In embodiments, the composition does not include a compound of formula (IA), wherein L¹ is a bond, R¹ is substituted or unsubstituted C₁-C₇ alkyl, R² is hydrogen or unsubstituted C₁-C₃ alkyl, R³ is —NR⁹R¹⁰, wherein R⁹ is hydrogen and R¹⁰ is R⁵¹-substituted or unsubstituted heterocycloalkyl, wherein R⁵¹ is substituted or unsubstituted C₁-C₇ alkyl, and R⁴ and R^{4.1} are both halogen. In embodiments, the composition does not include a compound of formula (IA), wherein L¹ is a bond, R¹ is substituted or unsubstituted C₁-C₇ alkyl, R² is hydrogen or substituted or unsubstituted C₁-C₃ alkyl, R³ is —NR⁹R¹⁰, wherein R⁹ is hydrogen and R¹⁰ is R⁵¹-substituted or unsubstituted heterocycloalkyl, wherein R⁵¹ is substituted or unsubstituted C₁-C₇ alkyl, and R⁴ and R^{4.1} are both halogen. In embodiments, the composition does not include a compound of formula (IA), wherein L¹ is a bond, R¹ is substituted or unsubstituted C₁-C₇ alkyl, R² is hydrogen or unsubstituted C₁-C₅ alkyl, R³ is —NR⁹R¹⁰, wherein R⁹ is hydrogen and R¹⁰ is R⁵¹-substituted or unsubstituted heterocycloalkyl, wherein R⁵¹ is substituted or unsubstituted C₁-C₇ alkyl, and R⁴ and R^{4.1} are both halogen. In embodiments, the composition does not include a compound of formula (IA), wherein L¹ is a bond, R¹ is substituted or unsubstituted C₁-C₇ alkyl, R² is hydrogen or substituted or unsubstituted C₁-C₅ alkyl, R³ is —NR⁹R¹⁰, wherein R⁹ is hydrogen and R¹⁰ is R⁵¹-substituted or unsubstituted heterocycloalkyl, wherein R⁵¹ is substituted or unsubstituted C₁-C₇ alkyl, and R⁴ and R^{4.1} are both halogen.

In embodiments, the composition does not include a compound of formula (IA), wherein L¹ is unsubstituted C₁-C₃ alkylene, R¹ is substituted or unsubstituted C₁-C₇ alkyl, R² is hydrogen or substituted or unsubstituted C₁-C₅ alkyl, R³ is —NR⁹R¹⁰, wherein R⁹ is hydrogen and R¹⁰ is R⁵¹-substituted or unsubstituted heterocycloalkyl, wherein R⁵¹ is substituted or unsubstituted C₁-C₇ alkyl, and R⁴ and R^{4.1} are both halogen. In embodiments, the composition does not include a compound of formula (IA), wherein L¹ is substituted or unsubstituted C₁-C₃ alkylene, R¹ is substituted or unsubstituted C₁-C₇ alkyl, R² is hydrogen or substituted or unsubstituted C₁-C₅ alkyl, R³ is —NR⁹R¹⁰, wherein R⁹ is hydrogen and R¹⁰ is R⁵¹-substituted or unsubstituted heterocycloalkyl, wherein R⁵¹ is substituted or unsubstituted C₁-C₇ alkyl, and R⁴ and R^{4.1} are both halogen. In embodiments, the composition does not include a compound of formula (IA), wherein L¹ is unsubstituted C₁-C₅ alkylene, R¹ is substituted or unsubstituted C₁-C₇ alkyl, R² is hydrogen or substituted or unsubstituted C₁-C₅ alkyl, R³ is —NR⁹R¹⁰, wherein R⁹ is hydrogen and R¹⁰ is R⁵¹-substituted or unsubstituted heterocycloalkyl, wherein R⁵¹ is substituted or unsubstituted C₁-C₇ alkyl, and R⁴ and R^{4.1} are both halogen.

In embodiments, the composition does not include a compound of formula (IA), wherein L¹ is substituted or unsubstituted C₁-C₅ alkylene, R¹ is substituted or unsubstituted C₁-C₇ alkyl, R² is hydrogen or substituted or unsubstituted C₁-C₅ alkyl, R³ is —NR⁹R¹⁰, wherein R⁹ is hydrogen and R¹⁰ is R⁵¹-substituted or unsubstituted heterocycloalkyl, wherein R⁵¹ is substituted or unsubstituted C₁-C₇ alkyl, and R⁴ and R^{4.1} are both halogen.

In embodiments, if L¹ is a bond, R¹ is methyl, R² is hydrogen, or R⁴ and R^{4.1} are simultaneously —Cl, then R³ is not —NR⁹R¹⁰, wherein R⁹ is hydrogen and R¹⁰ is

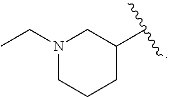 [SMILES: *C1CCCN(CC)C1]

In embodiments, the compositions disclosed herein (e.g. of Formula (I) or (II)) do not include a compound having the formula:

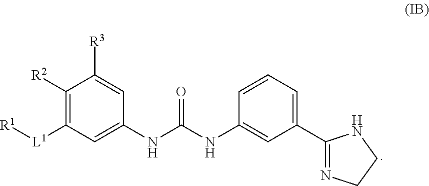

In embodiments, the composition does not include a compound of formula (IB), wherein R¹, R², R³ and L¹ are as defined herein (including embodiments thereof). In embodiments, the composition does not include a compound of formula (IB), wherein L¹ is a bond, R¹ is hydrogen, R² is hydrogen, R³ is —NHC═(O)R⁹R¹⁰, wherein R⁹ is hydrogen and R¹⁰ is

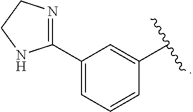 [SMILES: *c1cccc(C2=NCCN2)c1]

In embodiments, the composition does not include a compound of formula (IB), wherein L¹ is a bond, R¹ is hydrogen, R² is hydrogen, R³ is —NHC═(O)R⁹R¹⁰, wherein R⁹ is hydrogen and R¹⁰ is R⁵¹-substituted phenyl, wherein R⁵¹ is unsubstituted dihydro imidazolyl. In embodiments, the composition does not include a compound of formula (IB), wherein L¹ is a bond, R¹ is hydrogen, R² is hydrogen, R³ is —NHC═(O)R⁹R¹⁰, wherein R⁹ is hydrogen and R¹⁰ is R⁵¹-substituted phenyl, wherein R⁵¹ is substituted or unsubstituted dihydro imidazolyl. In embodiments, the composition does not include a compound of formula (IB), wherein L¹ is a bond, R¹ is hydrogen, R² is hydrogen, R³ is —NHC═(O)R⁹R¹⁰, wherein R⁹ is hydrogen and R¹⁰ is R⁵¹-substituted phenyl, wherein R⁵¹ is unsubstituted 5 membered heteroaryl. In embodiments, the composition does not include a compound of formula (IB), wherein L¹ is a bond, R¹ is hydrogen, R² is hydrogen, R³ is —NHC═(O)R⁹R¹⁰, wherein R⁹ is hydrogen and R¹⁰ is R⁵¹-substituted phenyl, wherein R⁵¹ is substituted or unsubstituted 5 membered heteroaryl. In embodiments, the composition does not include a compound of formula (IB), wherein L¹ is a bond, R¹ is hydrogen, R² is hydrogen, R³ is —NHC═(O)R⁹R¹⁰, wherein R⁹ is hydrogen and R¹⁰ is R⁵¹-substituted aryl, wherein R⁵¹ is substituted or unsubstituted 5 membered heteroaryl.

In embodiments, the composition does not include a compound of formula (IB), wherein L¹ is a bond, R¹ is hydrogen, R² is hydrogen, R³ is —NHC═(O)R⁹R¹⁰, wherein R⁹ is hydrogen and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond, R¹ is hydrogen, R² is hydrogen, R³ is —NHC═(O)R⁹R¹⁰, wherein R⁹ is hydrogen or C₁-C₃ unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond, R¹ is hydrogen, R² is hydrogen, R³ is —NHC═(O)R⁹R¹⁰, wherein R⁹ is hydrogen or C₁-C₃ substituted or unsubstituted alkyl and R¹⁰ is R⁵¹-substituted or unsubstituted aryl, wherein R⁵¹ is substituted or unsubstituted 5 membered heteroaryl.

In embodiments, the composition does not include a compound of formula (IB), wherein L¹ is a bond, R¹ is hydrogen, R² is hydrogen, R³ is —NHC═(O)R⁹R¹⁰, wherein R⁹ is hydrogen or C₁-C₅ unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond, R¹ is hydrogen, R² is hydrogen, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond, R¹ is hydrogen, R² is hydrogen or unsubstituted C₁-C₃ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl.

In embodiments, the composition does not include a compound of formula (IB), wherein L¹ is a bond, R¹ is hydrogen, R² is hydrogen or substituted or unsubstituted C₁-C₃ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond, R¹ is hydrogen, R² is hydrogen or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond, R¹ is hydrogen, R² is hydrogen or substituted or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl.

In embodiments, the composition does not include a compound of formula (IB), wherein L¹ is a bond, R¹ is hydrogen or unsubstituted C₁-C₃ alkyl, R² is hydrogen or substituted or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond, R¹ is hydrogen or substituted or unsubstituted C₁-C₃ alkyl, R² is hydrogen or substituted or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl.

In embodiments, the composition does not include a compound of formula (IB), wherein L¹ is a bond, R¹ is hydrogen or unsubstituted C₁-C₅ alkyl, R² is hydrogen or substituted or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond, R¹ is hydrogen or substituted or unsubstituted C₁-C₅ alkyl, R² is hydrogen or substituted or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond or unsubstituted C₁-C₃ alkylene, R¹ is hydrogen or substituted or unsubstituted C₁-C₅ alkyl, R² is hydrogen or substituted or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond or substituted or unsubstituted C₁-C₃ alkylene, R¹ is hydrogen or substituted or unsubstituted C₁-C₅ alkyl, R² is hydrogen or substituted or unsubstituted C₁-0₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond or unsubstituted C₁-C₅ alkylene, R¹ is hydrogen or substituted or unsubstituted C₁-C₅ alkyl, R² is hydrogen or substituted or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond or substituted or unsubstituted C₁-C₅ alkylene, R¹ is hydrogen or substituted or unsubstituted C₁-C₅ alkyl, R² is hydrogen or substituted or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl.

In embodiments, if L¹ is a bond, R¹ is hydrogen, or R² is hydrogen, then R³ is not —NHC═(O)R⁹R¹⁰, wherein R⁹ is hydrogen and R¹⁰ is

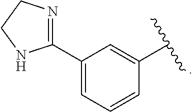 [SMILES: *c1cccc(C2=NCCN2)c1]

In embodiments, the composition does not include a compound of formula (IB), wherein L¹ is a bond, R¹ is —C(O)—OR⁵, wherein R⁵ is hydrogen, R² is hydrogen, R³ is —NHC═(O)R⁹R¹⁰, wherein R⁹ is hydrogen and R¹⁰ is R⁵¹-substituted phenyl, wherein R⁵¹ is unsubstituted dihydro imidazolyl. In embodiments, the composition does not include a compound of formula (IB), wherein L¹ is a bond, R¹ is —C(O)—OR⁵, wherein R⁵ is hydrogen, R² is hydrogen, R³ is —NHC═(O)R⁹R¹⁰, wherein R⁹ is hydrogen and R¹⁰ is R⁵¹-substituted phenyl, wherein R⁵¹ is substituted or unsubstituted dihydro imidazolyl. In embodiments, the composition does not include a compound of formula (IB), wherein L¹ is a bond, R¹ is —C(O)—OR⁵, wherein R⁵ is hydrogen, R² is hydrogen, R³ is —NHC═(O)R⁹R¹⁰, wherein R⁹ is hydrogen and R¹⁰ is R⁵¹-substituted phenyl, wherein R⁵¹ is unsubstituted 5 membered heteroaryl. In embodiments, the composition does not include a compound of formula (IB), wherein L¹ is a bond, R¹ is —C(O)—OR⁵, wherein R⁵ is hydrogen, R² is hydrogen, R³ is —NHC═(O)R⁹R¹⁰, wherein R⁹ is hydrogen and R¹⁰ is R⁵¹-substituted phenyl, wherein R⁵¹ is substituted or unsubstituted 5 membered heteroaryl. In embodiments, the composition does not include a compound of formula (IB), wherein L¹ is a bond, R¹ is —C(O)—OR⁵, wherein R⁵ is hydrogen, R² is hydrogen, R³ is —NHC═(O)R⁹R¹⁰, wherein R⁹ is hydrogen and R¹⁰ is R⁵¹-substituted aryl, wherein R⁵¹ is substituted or unsubstituted 5 membered heteroaryl. In embodiments, the composition does not include a compound of formula (IB), wherein L¹ is a bond, R¹ is —C(O)—OR⁵, wherein R⁵ is hydrogen, R² is hydrogen, R³ is —NHC═(O)R⁹R¹⁰, wherein R⁹ is hydrogen and R¹⁰ is R⁵¹-substituted or unsubstituted aryl, wherein R⁵¹ is substituted or unsubstituted 5 membered heteroaryl.

In embodiments, the composition does not include a compound of formula (IB), wherein L¹ is a bond, R¹ is C(O)—OR⁵, wherein R⁵ is hydrogen, R² is hydrogen, R³ is —NHC═(O)R⁹R¹⁰, wherein R⁹ is hydrogen or C₁-C₃ unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond, R¹ is —C(O)—OR⁵, wherein R⁵ is hydrogen, R² is hydrogen, R³ is —NHC═(O)R⁹R¹⁰, wherein R⁹ is hydrogen or C₁-C₃ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond, R¹ is —C(O)—OR⁵, wherein R⁵ is hydrogen, R² is hydrogen, R³ is —NHC═(O)R⁹R¹⁰, wherein R⁹ is hydrogen or C₁-C₅ unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond, R¹ is —C(O)—OR⁵, wherein R⁵ is hydrogen, R² is hydrogen, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl.

In embodiments, the composition does not include a compound of formula (IB), wherein L¹ is a bond, R¹ is —C(O)—OR⁵, wherein R⁵ is hydrogen, R² is hydrogen or unsubstituted C₁-C₃ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond, R¹ is —C(O)—OR⁵, wherein R⁵ is hydrogen, R² is hydrogen or substituted or unsubstituted C₁-C₃ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond, R¹ is —C(O)—OR⁵, wherein R⁵ is hydrogen, R² is hydrogen or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond, R¹ is —C(O)—OR⁵, wherein R⁵ is hydrogen, R² is hydrogen or substituted or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl.

In embodiments, the composition does not include a compound of formula (IB), wherein L¹ is a bond, R¹ is —C(O)—OR⁵, wherein R⁵ is hydrogen or unsubstituted C₁-C₃ alkyl, R² is hydrogen or substituted or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond, R¹ is —C(O)—OR⁵, wherein R⁵ is hydrogen or substituted or unsubstituted C₁-C₃ alkyl, R² is hydrogen or substituted or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond, R¹ is —C(O)—OR⁵, wherein R⁵ is hydrogen or unsubstituted C₁-C₅ alkyl, R² is hydrogen or substituted or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl.

In embodiments, the composition does not include a compound of formula (IB), wherein L¹ is a bond, R¹ is —C(O)—OR⁵, wherein R⁵ is hydrogen or substituted or unsubstituted C₁-C₅ alkyl, R² is hydrogen or substituted or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond or unsubstituted C₁-C₃ alkylene, R¹ is —C(O)—OR⁵, wherein R⁵ is hydrogen or substituted or unsubstituted C₁-C₅ alkyl, R² is hydrogen or substituted or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond or substituted or unsubstituted C₁-C₃ alkylene, R¹ is —C(O)—OR⁵, wherein R⁵ is hydrogen or substituted or unsubstituted C₁-C₅ alkyl, R² is hydrogen or substituted or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond or unsubstituted C₁-C₅ alkylene, R¹ is —C(O)—OR⁵, wherein R⁵ is hydrogen or substituted or unsubstituted C₁-C₅ alkyl, R² is hydrogen or substituted or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl.

In embodiments, the composition does not include a compound of formula (IB), wherein L¹ is a bond or substituted or unsubstituted C₁-C₅ alkylene, R¹ is —C(O)—OR⁵, wherein R⁵ is hydrogen or substituted or unsubstituted C₁-C₅ alkyl, R² is hydrogen or substituted or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl.

In embodiments, if L¹ is a bond, R¹ is —C(O)OH, or R² is hydrogen, then R³ is not —NHC═(O)R⁹R¹⁰, wherein R⁹ is hydrogen and R¹⁰ is

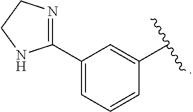 [SMILES: *c1cccc(C2=NCCN2)c1]

In embodiments, the composition does not include a compound of formula (IB), wherein L¹ is a bond, R¹ is hydrogen, R² is —C(O)NR⁷R⁸, wherein R⁷ is hydrogen and R⁸ is methyl, R³ is —NHC═(O)R⁹R¹⁰, wherein R⁹ is hydrogen and R¹⁰ is R⁵¹-substituted phenyl, wherein R⁵¹ is unsubstituted dihydro imidazolyl. In embodiments, the composition does not include a compound of formula (IB), wherein L¹ is a bond, R¹ is hydrogen, R² is —C(O)NR⁷R⁸, wherein R⁷ is hydrogen and R⁸ is methyl, R³ is —NHC═(O)R⁹R¹⁰, wherein R⁹ is hydrogen and R¹⁰ is R⁵¹-substituted phenyl, wherein R⁵¹ is substituted or unsubstituted dihydro imidazolyl. In embodiments, the composition does not include a compound of formula (IB), wherein L¹ is a bond, R¹ is hydrogen, R² is —C(O)NR⁷R⁸, wherein R⁷ is hydrogen and R⁸ is methyl, R³ is —NHC═(O)R⁹R¹⁰, wherein R⁹ is hydrogen and R¹⁰ is R⁵¹-substituted phenyl, wherein R⁵¹ is unsubstituted 5 membered heteroaryl. In embodiments, the composition does not include a compound of formula (IB), wherein L¹ is a bond, R¹ is hydrogen, R² is —C(O)NR⁷R⁸, wherein R⁷ is hydrogen and R⁸ is methyl, R³ is —NHC═(O)R⁹R¹⁰, wherein R⁹ is hydrogen and R¹⁰ is R⁵¹-substituted phenyl, wherein R⁵¹ is substituted or unsubstituted 5 membered heteroaryl. In embodiments, the composition does not include a compound of formula (IB), wherein L¹ is a bond, R¹ is hydrogen, R² is —C(O)NR⁷R⁸, wherein R⁷ is hydrogen and R⁸ is methyl, R³ is —NHC═(O)R⁹R¹⁰, wherein R⁹ is hydrogen and R¹⁰ is R⁵¹-substituted aryl, wherein R⁵¹ is substituted or unsubstituted 5 membered heteroaryl.

In embodiments, the composition does not include a compound of formula (IB), wherein L¹ is a bond, R¹ is hydrogen, R² is —C(O)NR⁷R⁸, wherein R⁷ is hydrogen and R⁸ is methyl, R³ is —NHC═(O)R⁹R¹⁰, wherein R⁹ is hydrogen and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond, R¹ is hydrogen, R² is —C(O)NR⁷R⁸, wherein R⁷ is hydrogen and R⁸ is methyl, R³ is —NHC═(O)R⁹R¹⁰, wherein R⁹ is hydrogen or C₁-C₃ unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond, R¹ is hydrogen, R² is —C(O)NR⁷R⁸, wherein R⁷ is hydrogen and R⁸ is methyl, R³ is —NHC═(O)R⁹R¹⁰, wherein R⁹ is hydrogen or C₁-C₃ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond, R¹ is hydrogen, R² is —C(O)NR⁷R⁸, wherein R⁷ is hydrogen and R⁸ is methyl, R³ is —NHC═(O)R⁹R¹⁰, wherein R⁹ is hydrogen or C₁-C₅ unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond, R¹ is hydrogen, R² is —C(O)NR⁷R⁸, wherein R⁷ is hydrogen and R⁸ is methyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl.

In embodiments, the composition does not include a compound of formula (IB), wherein L¹ is a bond, R¹ is hydrogen, R² is —C(O)NR⁷R⁸, wherein R⁷ is hydrogen and R⁸ is unsubstituted C₁-C₃ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond, R¹ is hydrogen, R² is —C(O)NR⁷R⁸, wherein R⁷ is hydrogen and R⁸ is substituted or unsubstituted C₁-C₃ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond, R¹ is hydrogen, R² is —C(O)NR⁷R⁸, wherein R⁷ is hydrogen and R⁸ is unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond, R¹ is hydrogen, R² is —C(O)NR⁷R⁸, wherein R⁷ is hydrogen and R⁸ is substituted or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl.

In embodiments, the composition does not include a compound of formula (IB), wherein L¹ is a bond, R¹ is hydrogen or unsubstituted C₁-C₃ alkyl, R² is —C(O)NR⁷R⁸, wherein R⁷ is hydrogen and R⁸ is substituted or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond, R¹ is hydrogen or substituted or unsubstituted C₁-C₃ alkyl, R² is —C(O)NR⁷R⁸, wherein R⁷ is hydrogen and R⁸ is substituted or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond, R¹ is hydrogen or unsubstituted C₁-C₅ alkyl, R² is —C(O)NR⁷R⁸, wherein R⁷ is hydrogen and R⁸ is substituted or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond, R¹ is hydrogen or substituted or unsubstituted C₁-C₅ alkyl, R² is —C(O)NR⁷R⁸, wherein R⁷ is hydrogen and R⁸ is substituted or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl.

In embodiments, the composition does not include a compound of formula (IB), wherein L¹ is a bond or unsubstituted C₁-C₃ alkylene, R¹ is hydrogen or substituted or unsubstituted C₁-C₅ alkyl, R² is —C(O)NR⁷R⁸, wherein R⁷ is hydrogen and R⁸ is substituted or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (TB), wherein L¹ is a bond or substituted or unsubstituted C₁-C₃ alkylene, R¹ is hydrogen or substituted or unsubstituted C₁-C₅ alkyl, R² is —C(O)NR⁷R⁸, wherein R⁷ is hydrogen and R⁸ is substituted or unsubstituted C₁-C₅ alkyl, R³ is —NHC═(O)R⁹R¹⁶, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond or unsubstituted C₁-C₅ alkylene, R¹ is hydrogen or substituted or unsubstituted C₁-C₅ alkyl, R² is —C(O)NR⁷R⁸, wherein R⁷ is hydrogen and R⁸ is substituted or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl. In embodiments, the composition does not include a compound of formula (IB), wherein L¹ is a bond or substituted or unsubstituted C₁-C₅ alkylene, R¹ is hydrogen or substituted or unsubstituted C₁-C₅ alkyl, R² is —C(O)NR⁷R⁸, wherein R⁷ is hydrogen and R⁸ is substituted or unsubstituted C₁-C₅ alkyl, R³ is —NHC═(O)R⁹R¹⁰, wherein R⁹ is hydrogen or C₁-C₅ substituted or unsubstituted alkyl and R¹⁰ is R⁵¹-substituted or unsubstituted aryl, wherein R⁵¹ is substituted or unsubstituted 5 membered heteroaryl.

In embodiments, if L¹ is a bond, R¹ is hydrogen, R² is —C(O)NR⁷R⁸, wherein R⁷ is hydrogen and R⁸ is methyl, then R³ is not —NHC═(O)R⁹R¹⁰, wherein R⁹ is hydrogen and R¹⁰ is

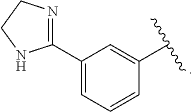 [SMILES: *c1cccc(C2=NCCN2)c1]

In embodiments, the compositions disclosed herein (e.g. of Formula (I) or (II)) do not include a compound having the formula:

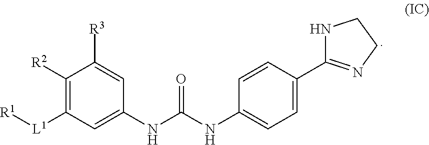

In formula (IC), R¹, R², R³ and L¹ are as defined herein (including embodiments thereof). In embodiments, L¹ is a bond, R¹ is —NHC═(O)R⁵R⁶, wherein R⁵ is hydrogen and R⁶ is

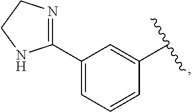 [SMILES: *c1cccc(C2=NCCN2)c1]

R² is hydrogen, and R³ is hydrogen. In embodiments, the composition does not include a compound of formula (IC), wherein L¹ is a bond, R¹ is —NHC═(O)R⁵R⁶, wherein R⁵ is hydrogen and R⁶ is

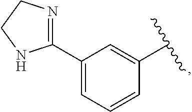 [SMILES: *c1cccc(C2=NCCN2)c1]

R² is hydrogen or unsubstituted C₁-C₃ alkyl, and R³ is hydrogen or unsubstituted C₁-C₃ alkyl. In embodiments, the composition does not include a compound of formula (IC), wherein L¹ is a bond, R¹ is —NHC═(O)R⁵R⁶, wherein R⁵ is hydrogen and R⁶ is

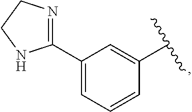 [SMILES: *c1cccc(C2=NCCN2)c1]

R² is hydrogen or substituted or unsubstituted C₁-C₃ alkyl, and R³ is hydrogen or substituted or unsubstituted C₁-C₃ alkyl. In embodiments, the composition does not include a compound of formula (IC), wherein L¹ is a bond, R¹ is —NHC═(O)R⁵R⁶, wherein R⁵ is hydrogen and R⁶ is

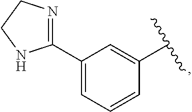 [SMILES: *c1cccc(C2=NCCN2)c1]

R² is hydrogen or unsubstituted C₁-C₅ alkyl, and R³ is hydrogen or unsubstituted C₁-C₅ alkyl. In embodiments, the composition does not include a compound of formula (IC), wherein L¹ is a bond, R¹ is —NHC═(O)R⁵R⁶, wherein R⁵ is hydrogen and R⁶ is

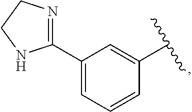 [SMILES: *c1cccc(C2=NCCN2)c1]

R² is hydrogen or substituted or unsubstituted C₁-C₅ alkyl, and R³ is hydrogen or substituted or unsubstituted C₁-C₅ alkyl. In embodiments, the composition does not include a compound of formula (IC), wherein L¹ is a bond, R¹ is —NHC═(O)R⁵R⁶, wherein R⁵ is hydrogen and R⁶ is R⁵¹-substituted phenyl, wherein R⁵¹ is unsubstituted dihydro imidazolyl, R² is hydrogen or substituted or unsubstituted C₁-C₅ alkyl, and R³ is hydrogen or substituted or unsubstituted C₁-C₅ alkyl.

In embodiments, the composition does not include a compound of formula (IC), wherein L¹ is a bond, R¹ is —NHC═(O)R⁵R⁶, wherein R⁵ is hydrogen and R⁶ is R⁵¹-substituted phenyl, wherein R⁵¹ is substituted or unsubstituted dihydro imidazolyl, R² is hydrogen or substituted or unsubstituted C₁-C₅ alkyl, and R³ is hydrogen or substituted or unsubstituted C₁-C₅ alkyl. In embodiments, the composition does not include a compound of formula (IC), wherein L¹ is a bond, R¹ is —NHC═(O)R⁵R⁶, wherein R⁵ is hydrogen and R⁶ is R⁵¹-substituted phenyl, wherein R⁵¹ is unsubstituted 5 membered heteroaryl, R² is hydrogen or substituted or unsubstituted C₁-C₅ alkyl, and R³ is hydrogen or substituted or unsubstituted C₁-C₅ alkyl. In embodiments, the composition does not include a compound of formula (IC), wherein L¹ is a bond, R¹ is —NHC═(O)R⁵R⁶, wherein R⁵ is hydrogen and R⁶ is R⁵¹-substituted phenyl, wherein R⁵¹ is substituted or unsubstituted 5 membered heteroaryl, R² is hydrogen or substituted or unsubstituted C₁-C₅ alkyl, and R³ is hydrogen or substituted or unsubstituted C₁-C₅ alkyl. In embodiments, the composition does not include a compound of formula (IC), wherein L¹ is a bond, R¹ is —NHC═(O)R⁵R⁶, wherein R⁵ is hydrogen and R⁶ is R⁵¹-substituted aryl, wherein R⁵¹ is substituted or unsubstituted 5 membered heteroaryl, R² is hydrogen or substituted or unsubstituted C₁-C₅ alkyl, and R³ is hydrogen or substituted or unsubstituted C₁-C₅ alkyl.

In embodiments, the composition does not include a compound of formula (IC), wherein L¹ is a bond, R¹ is —NHC═(O)R⁵R⁶, wherein R⁵ is hydrogen and R⁶ is R⁵¹-substituted or unsubstituted aryl, wherein R⁵¹ is substituted or unsubstituted 5 membered heteroaryl, R² is hydrogen or substituted or unsubstituted C₁-C₅ alkyl, and R³ is hydrogen or substituted or unsubstituted C₁-C₅ alkyl. In embodiments, the composition does not include a compound of formula (IC), wherein L¹ is a bond or unsubstituted C₁-C₃ alkylene, R¹ is —NHC═(O)R⁵R⁶, wherein R⁵ is hydrogen and R⁶ is R⁵¹-substituted or unsubstituted aryl, wherein R⁵¹ is substituted or unsubstituted 5 membered heteroaryl, R² is hydrogen or substituted or unsubstituted C₁-C₅ alkyl, and R³ is hydrogen or substituted or unsubstituted C₁-C₅ alkyl. In embodiments, the composition does not include a compound of formula (IC), wherein L¹ is a bond or substituted or unsubstituted C₁-C₃ alkylene, R¹ is —NHC═(O)R⁵R⁶, wherein R⁵ is hydrogen and R⁶ is R⁵¹-substituted or unsubstituted aryl, wherein R⁵¹ is substituted or unsubstituted 5 membered heteroaryl, R² is hydrogen or substituted or unsubstituted C₁-C₅ alkyl, and R³ is hydrogen or substituted or unsubstituted C₁-C₅ alkyl.

In embodiments, the composition does not include a compound of formula (IC), wherein L¹ is a bond or unsubstituted C₁-C₅ alkylene, R¹ is —NHC═(O)R⁵R⁶, wherein R⁵ is hydrogen and R⁶ is R⁵¹-substituted or unsubstituted aryl, wherein R⁵¹ is substituted or unsubstituted 5 membered heteroaryl, R² is hydrogen or substituted or unsubstituted C₁-C₅ alkyl, and R³ is hydrogen or substituted or unsubstituted C₁-C₅ alkyl. In embodiments, the composition does not include a compound of formula (IC), wherein L¹ is a bond or substituted or unsubstituted C₁-C₅ alkylene, R¹ is —NHC═(O)R⁵R⁶, wherein R⁵ is hydrogen and R⁶ is R⁵¹-substituted or unsubstituted aryl, wherein R⁵¹ is substituted or unsubstituted 5 membered heteroaryl, R² is hydrogen or substituted or unsubstituted C₁-C₅ alkyl, and R³ is hydrogen or substituted or unsubstituted C₁-C₅ alkyl.

In embodiments, if L¹ is a bond, R² is hydrogen, or R³ is hydrogen, then R¹ is not —NHC═(O)R⁵R⁶, wherein R⁵ is hydrogen and R⁶ is

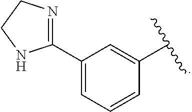 [SMILES: *c1cccc(C2=NCCN2)c1]

In embodiments, the compositions disclosed herein (e.g. of Formula (I) or (II)) do not include a compound having the formula:

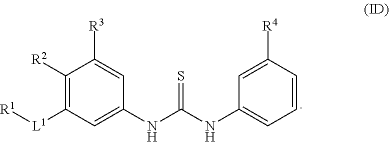

In formula (ID), R¹, R², R³ and L¹ are as defined herein (including embodiments thereof). In embodiments, L¹ is a bond, R¹ is hydrogen, R² is hydrogen, and R³ and R⁴ are both not

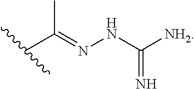 [SMILES: */C(C)=N/NC(=N)N]

In other words, where L¹ is a bond, R¹ is hydrogen, R² is hydrogen, and R³ and R⁴ are both not

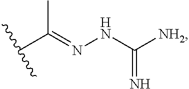 [SMILES: */C(C)=N/NC(=N)N]

R³ and are R⁴ are not simultaneously

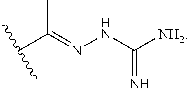 [SMILES: */C(C)=N/NC(=N)N]

In embodiments, the composition does not include a compound of formula (ID), wherein L¹ is a bond, R¹ is hydrogen, R² is hydrogen, and R³ and R⁴ are both substituted 5 membered heteroalkyl. In other words, where L¹ is a bond, R¹ is hydrogen, R² is hydrogen, and R³ and R⁴ are not simultaneously substituted 5 membered heteroalkyl. In embodiments, the composition does not include a compound of formula (ID), wherein L¹ is a bond, R¹ is hydrogen, R² is hydrogen, and R³ and R⁴ are both substituted or unsubstituted 5 membered heteroalkyl. In embodiments, the composition does not include a compound of formula (ID), wherein L¹ is a bond, R¹ is hydrogen, R² is hydrogen, and R³ and R⁴ are both substituted or unsubstituted heteroalkyl. In other words, where L¹ is a bond, R¹ is hydrogen, R² is hydrogen, and R³ and R⁴ are not simultaneously substituted heteroalkyl or not simultaneously unsubstituted heteroalkyl.

In embodiments, the composition does not include a compound of formula (ID), wherein L¹ is a bond, R¹ is hydrogen or unsubstituted C₁-C₃ alkyl, R² is hydrogen or unsubstituted C₁-C₃ alkyl, and R³ and R⁴ are both substituted or unsubstituted heteroalkyl. In embodiments, the composition does not include a compound of formula (ID), wherein L¹ is a bond, R¹ is hydrogen or substituted or unsubstituted C₁-C₃ alkyl, R² is hydrogen or substituted or unsubstituted C₁-C₃ alkyl, and R³ and R⁴ are both substituted or unsubstituted heteroalkyl. In embodiments, the composition does not include a compound of formula (ID), wherein L¹ is a bond, R¹ is hydrogen or unsubstituted C₁-C₅ alkyl, R² is hydrogen or unsubstituted C₁-C₅ alkyl, and R³ and R⁴ are both substituted or unsubstituted heteroalkyl. In embodiments, the composition does not include a compound of formula (ID), wherein L¹ is a bond, R¹ is hydrogen or substituted or unsubstituted C₁-C₅ alkyl, R² is hydrogen or substituted or unsubstituted C₁-C₅ alkyl, and R³ and R⁴ are both substituted or unsubstituted heteroalkyl.

In embodiments, if R³ and R⁴ are both

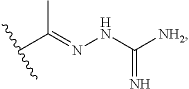 [SMILES: */C(C)=N/NC(=N)N]

L¹ is a not bond, R¹ is not hydrogen, or R² is not hydrogen. In other words, if R³ and R⁴ are simultaneously

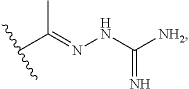 [SMILES: */C(C)=N/NC(=N)N]

L¹ is a not bond, R¹ is not hydrogen, or R² is not hydrogen.

In embodiments of formula (I) or (II), R¹ is substituted (e.g., R^{1A}-substituted) or unsubstituted C₁-C₂₀ (e.g., C₁-C₆) alkyl or substituted (e.g., R^{1A}-substituted) or unsubstituted 2 to 20 membered (e.g., 2 to 6 membered) heterocycloalkyl. In embodiments, R¹ is substituted or unsubstituted C₁-C₂₀ (e.g., C₁-C₁₅) alkyl or substituted or unsubstituted 3 to 8 membered (e.g., 6 membered) heterocycloalkyl. In embodiments, R¹ is substituted or unsubstituted C₁-C₂₀ (e.g., C₁-C₁₅) alkyl. In embodiments, R¹ is substituted or unsubstituted C₁-C₁₀ (e.g., C₁-C₆) alkyl. In embodiments, R¹ is substituted or unsubstituted C₁-C₅ (e.g., methyl, ethyl) alkyl. In embodiments, R¹ is unsubstituted ethyl.

In embodiments, R¹ is substituted (e.g., R^{1A}-substituted) or unsubstituted 3 to 8 membered (e.g., 6 membered) heterocycloalkyl. In embodiments, R¹ is substituted (e.g., R^{1A}-substituted) or unsubstituted 5 or 6 membered heterocycloalkyl. In embodiments, R¹ is unsubstituted 6 membered heterocycloalkyl. In embodiments, R¹ is substituted (e.g., R^{1A}-substituted) 6 membered heterocycloalkyl. In embodiments, R¹ is substituted (e.g., R^{1A}-substituted) or unsubstituted piperidinyl. In embodiments, R¹ is unsubstituted piperidinyl. In embodiments, R¹ is substituted (e.g., R^{1A}-substituted) piperidinyl. In embodiments, R¹ is substituted (e.g., R^{1A}-substituted) or unsubstituted 5 membered heterocycloalkyl. In embodiments, R¹ is unsubstituted 5 membered heterocycloalkyl. In embodiments, R¹ is substituted (e.g., R^{1A}-substituted) 5 membered heterocycloalkyl. In embodiments, R¹ is substituted (e.g., R^{1A}-substituted) or unsubstituted pyrrolidinyl. In embodiments, R¹ is unsubstituted pyrrolidinyl. In embodiments, R¹ is substituted (e.g., R^{1A}-substituted) pyrrolidinyl.

R¹ may be R^{1A}-substituted or unsubstituted 5 or 6 membered heterocycloalkyl. Thus, in embodiments, R¹ is R^{1A}-substituted or unsubstituted 5 or 6 membered heterocycloalkyl. In embodiments, R¹ is R^{1A}-substituted 5 or 6 membered heterocycloalkyl. In embodiments, R¹ is R^{1A}-substituted or unsubstituted 5 membered heterocycloalkyl. In embodiments, R¹ is R^{1A}-substituted 5 membered heterocycloalkyl. In embodiments, R¹ is

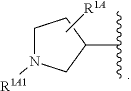

In embodiments, the compound of formula (I) or (II) including embodiments thereof may include multiple instances of R^{1A} (e.g., R¹ may be substituted with more than one R^{1A} substituent and each R^{1A} substituent may be independently different). In such embodiments, each R^{1A} substituent may optionally be different and be appropriately labeled to distinguish each R^{1A} substituent for greater clarity. For example, where R¹ is substituted with more than one R^{1A} substituent each of which is different, they may be referred to, for example, as R^{1A}, R^{1A1}, R^{1A2} and/or R^{1A3}, respectively, wherein the definition of R^{1A} is assumed by R^{1A1}, R^{1A2}, and/or R^{1A3}, respectively. The variables used within a definition of R^{1A} and/or other variables that appear at multiple instances and are different may similarly be appropriately labeled to distinguish each group for greater clarity. Thus, in embodiments, where R¹ is

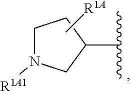

R¹ is substituted with more than one substituent (R^{1A} and R^{1A1}) each of which is different.

In the compositions provided herein including embodiments thereof, R^{1A} may be R^{1B}-substituted or unsubstituted C₁-C₂₀ (e.g., C₁-C₆) alkyl, R^{1B}-substituted or unsubstituted 2 to 20 membered (e.g., 2 to 6 membered) heteroalkyl, R^{1B}-substituted or unsubstituted C₃-C₈ (e.g., C₅-C₇) cycloalkyl, R^{1B}-substituted or unsubstituted 3 to 8 membered (e.g., 3 to 6 membered) heterocycloalkyl, R^{1B}-substituted or unsubstituted C₅-C₁₀ (e.g., C₅-C₆) aryl, or R^{1B}-substituted or unsubstituted 5 to 10 membered (e.g., 5 to 6 membered) heteroaryl. In embodiments, R^{1A1} is substituted (e.g., R^{1B1}-substituted) or unsubstituted alkyl (e.g., C₁-C₈ alkyl). In embodiments, R^{1A1} is substituted (e.g., R^{1B1}-substituted) or unsubstituted C₁-C₅ alkyl. In embodiments, R^{1A1} is substituted (e.g., R^{1B1}-substituted) or unsubstituted C₁-C₃ alkyl. In embodiments, R^{1A1} is unsubstituted C₁-C₃ alkyl. In embodiments, R^{1A1} is unsubstituted methyl or ethyl. In embodiments, R^{1A1} is unsubstituted methyl. In related embodiments, R^{1A} is hydrogen.

In embodiments, R^{1A} is R^{1B}-substituted (e.g., R^{1C}-substituted) or unsubstituted C₁-C₅ alkyl, R^{1B} is R^{1C}-substituted or unsubstituted 5 to 10 membered heteroaryl, and R^{1C} is substituted (e.g., R^{1D}-substituted) or unsubstituted 1 to 5 membered heteroalkyl. In embodiments, R^{1A} is R^{1B}-substituted or unsubstituted unsaturated C₁-C₅ alkyl. In embodiments, R^{1A} is unsubstituted unsaturated C₁-C₅ alkyl. In embodiments, R^{1A} is R^{1B}-substituted unsaturated C₁-C₅ alkyl. In embodiments, R^{1A} is R^{1B}-substituted or unsubstituted unsaturated C₁-C₃ alkyl. In embodiments, R^{1A} is unsubstituted unsaturated C₁-C₃ alkyl. In embodiments, R^{1A} is R^{1B}-substituted unsaturated C₁-C₃ alkyl. In embodiments, R^{1A} is R^{1B}-substituted or unsubstituted ethenyl. In embodiments, R^{1A} is unsubstituted ethenyl. In embodiments, R^{1A} is R^{1B}-substituted ethenyl. In relate embodiments, R^{1B} is R^{1C}-substituted 9 membered heteroaryl. In related embodiments, R^{1B} is R^{1C}-substituted benzofuranyl. In embodiments, R^{1C} is —C(NH)NH₂.

In embodiments, R^{1A} is R^{1B}-substituted or unsubstituted 3 to 10 membered (e.g., 4 or 6 membered) heteroalkyl. In embodiments, R^{1A} is R^{1B}-substituted or unsubstituted 3 membered heteroalkyl. In embodiments, R^{1A} is R^{1B}-substituted or unsubstituted 4 membered heteroalkyl. In embodiments, R^{1A} is R^{1B}-substituted or unsubstituted 5 membered heteroalkyl. In embodiments, R^{1A} is R^{1B}-substituted or unsubstituted 6 membered heteroalkyl. In embodiments, R^{1A} is R^{1B}-(substituted or unsubstituted 7 membered heteroalkyl. In embodiments, R^{1A} is R^{1B}-substituted or unsubstituted 8 membered heteroalkyl. In embodiments, R^{1A} is R^{1B}-substituted or unsubstituted 9 membered heteroalkyl. In embodiments, R^{1A} is R^{1B}-substituted or unsubstituted 10 membered heteroalkyl. In embodiments, R^{1B} is unsubstituted (e.g. C₁-C₅) alkyl, ═O or ═S. In embodiments, R^{1B} is unsubstituted C₁-C₃ alkyl, ═O or ═S. In embodiments, R^{1B} is methyl, ═O or ═S. In embodiments, R^{1A} is R^{1B}-substituted 6 membered heteroalkyl and R^{1B} is independently unsubstituted methyl, ═O or ═S. In embodiments, R^{1A} is

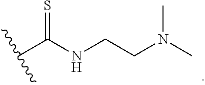 [SMILES: *C(=S)NCCN(C)C]

In embodiments, R¹ is R^{1A}-substituted or unsubstituted 6 membered heterocycloalkyl. In embodiments, R¹ is

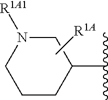

and R^{1A1} is hydrogen or substituted (e.g., R^{1B1}-substituted) or unsubstituted C₁-C₅ alkyl. In embodiments, R^{1A1} is hydrogen or substituted (e.g., R^{1B1}-substituted) or unsubstituted C₁-C₅ alkyl. In embodiments, R^{1A1} is hydrogen or substituted (e.g., R^{1B1}-substituted) or unsubstituted C₁-C₃ alkyl. In embodiments, R^{1A1} is hydrogen or unsubstituted C₁-C₃ alkyl. In embodiments, R^{1A1} is unsubstituted methyl or ethyl. In embodiments, R^{1A1} is unsubstituted methyl. In embodiments, R^{1A1} is unsubstituted ethyl. In embodiments, R^{1A1} and R^{1A} are independently hydrogen.

In embodiments, R^{1A} is hydrogen, halogen, —NO₂ or substituted (e.g., R^{1B}-substituted) or unsubstituted heteroalkyl (e.g., 3 to 10 membered heteroalkyl). In embodiments, R^{1A} is R^{1B}-substituted or unsubstituted 3 to 10 membered (e.g., 4 or 6 membered) heteroalkyl. In embodiments, R^{1A} is R^{1B}-substituted or unsubstituted 3 membered heteroalkyl. In embodiments, R^{1A} is R^{1B}-substituted or unsubstituted 4 membered heteroalkyl. In embodiments, R^{1A} is R^{1B}-substituted or unsubstituted 5 membered heteroalkyl. In embodiments, R^{1A} is R^{1B}-substituted or unsubstituted 6 membered heteroalkyl. In embodiments, R^{1A} is R^{1B}-substituted or unsubstituted 7 membered heteroalkyl. In embodiments, R^{1A} is R^{1B}-substituted or unsubstituted 8 membered heteroalkyl. In embodiments, R^{1A} is R^{1B}-substituted or unsubstituted 9 membered heteroalkyl. In embodiments, R^{1A} is R^{1B}-substituted or unsubstituted 10 membered heteroalkyl. In embodiments, R^{1B} is unsubstituted (e.g. C₁-C₅) alkyl, ═O or ═S. In embodiments, R^{1B} is unsubstituted C₁-C₃ alkyl, ═O or ═S. In embodiments, R^{1B} is methyl, ═O or ═S. In embodiments, R^{1A} is R^{1B}-substituted 6 membered heteroalkyl and R^{1B} is independently unsubstituted methyl, ═O or ═S. In embodiments, R^{1A} is

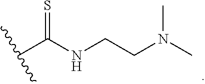 [SMILES: *C(=S)NCCN(C)C]

In the compositions provided herein including embodiments thereof, R² may be independently hydrogen, halogen, —CX^{b}₃, —CN, —SO₂Cl, —SOₙ₂R⁷, —SOᵥ₂NR⁷R⁸, —NHNH₂, —ONR⁷R⁸, —NHC═(O)NHNH₂, —NHC═(O)NR⁷R⁸, —N(O)ₘ₂, —NR⁷R⁸, —NH—O—R⁷, —C(O)R⁷, —C(O)—OR⁷, —C(O)NR⁷R⁸, —OR⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R² is hydrogen, substituted or unsubstituted (e.g. C₁-C₅) alkyl or OR⁷. In embodiments, R² is hydrogen, unsubstituted C₁-C₅ alkyl or OR⁷. In embodiments, R² is hydrogen, unsubstituted C₁-C₃ alkyl or OR⁷. In embodiments, R² is hydrogen or —OR⁷. In embodiments, R⁷ is substituted (e.g. R⁵¹-substituted) or unsubstituted C₁-C₅ alkyl. In embodiments, R⁷ is substituted (e.g. R⁵¹-substituted) or unsubstituted C₁-C₃ alkyl. In embodiments, R⁷ is unsubstituted C₁-C₃ alkyl. In embodiments, R⁷ is methyl.

In the compositions provided herein including embodiments thereof, R³ may be independently hydrogen, halogen, —CX^{c}₃, —CN, —SO₂Cl, —SOₙ₃R⁹, —SOᵥ₃NR⁹R¹⁰, —NHNH₂, —ONR⁹R¹⁰, —NHC═(O)NHNH₂, —NHC═(O)NR⁹R¹⁰, —N(O)ₘ₃, —NR⁹R⁸, —NH—O—R⁹, —C(O)R⁹, —C(O)—OR⁹, —C(O)NR⁹R¹⁰, —OR⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R³ is substituted or unsubstituted (e.g., C₁-C₈) alkyl, —NR⁹R¹⁰ or —NH—OR⁹. In embodiments, R³ is substituted or unsubstituted C₁-C₅ alkyl, —NR⁹R¹⁰ or —NH—OR⁹. In embodiments, R³ is substituted or unsubstituted C₁-C₃ alkyl, —NR⁹R¹⁰ or —NH—OR⁹. In embodiments, R³ is unsubstituted C₁-C₃ alkyl, —NR⁹R¹⁰ or —NH—OR⁹. In embodiments, R³ is hydrogen, methyl, —NR⁹R¹⁰ or —NH—OR⁹. In embodiments, R³ is hydrogen. In embodiments, R³ is methyl. In embodiments, R³ is —NR⁹R¹⁰ . In embodiments, R³ is —NH—OR⁹.

Where R³ is —NR⁹R¹⁰ or —NH—OR⁹, R⁹ and R¹⁰ may be independently hydrogen or substituted or unsubstituted C₁-C₅ alkyl. Thus, in embodiments, R⁹ and R¹⁰ are independently hydrogen or substituted or unsubstituted C₁-C₅ alkyl. In embodiments, R⁹ and R¹⁰ are independently hydrogen or substituted or unsubstituted C₁-C₃ alkyl. In embodiments, R⁹ and R¹⁰ are independently hydrogen or unsubstituted C₁-C₃ alkyl. In embodiments, R⁹ and R¹⁰ are independently hydrogen, methyl or ethyl. In embodiments, R³ is NR⁹R¹⁰ and R⁹ and R¹⁰ are independently hydrogen. In embodiments, R³ is —NR⁹K¹⁰ and R⁹ and R¹⁰ are independently methyl.

In embodiments, R³ is —NH—OR⁹. In embodiments, R⁹ is hydrogen or substituted or unsubstituted (e.g., C₁-C₈) alkyl. In embodiments, R⁹ is hydrogen or substituted or unsubstituted C₁-C₅ alkyl. In embodiments, R⁹ is hydrogen or substituted or unsubstituted C₁-C₃ alkyl. In embodiments, R⁹ is hydrogen or unsubstituted C₁-C₃ alkyl. In embodiments, R⁹ is methyl.

In the compositions provided herein including embodiments thereof, R⁴ may be independently hydrogen, halogen, —CX^{d}₃, —CN, —SO₂Cl, —SOᵥ₄NR¹¹R¹², —NHNH₂, —ONR¹¹R¹², —NHC═(O)NHNH₂, —NHC═(O)NR¹¹R¹², —N(O)ₘ₄, —NR¹¹R¹², —NH—O—R¹¹, —C(O)R¹¹, —C(O)—OR¹¹, —C(O)NR¹¹R¹², —OR¹¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁴ is independently hydrogen, halogen, —CX^{d}₃, —SOᵥ₄NR¹¹R¹², —OR¹¹, or substituted or unsubstituted (e.g. C₁-C₅) alkyl. In embodiments, R⁴ is independently hydrogen, halogen, —CX^{d}₃, —SOᵥ₄NR¹¹R¹², —OR¹¹, or unsubstituted C₁-C₅ alkyl. In embodiments, R⁴ is independently hydrogen, halogen, —CX^{d}₃, —SOᵥ₄NR¹¹R¹², —OR¹¹, or unsubstituted C₁-C₃ alkyl. In embodiments, R⁴ is independently hydrogen, halogen, —CX^{d}₃, —SOᵥ₄NR¹¹R¹², —OR¹¹, or methyl. In embodiments, R⁴ is methyl and z is 2.

In the compositions provided herein including embodiments thereof, R⁴ may be independently hydrogen, halogen, —CX^{d}₃, —SOᵥ₄NR¹¹R¹², —OR¹¹, or substituted or unsubstituted (e.g. C₁-C₅) alkyl. In embodiments, X^{d} is —F, v₄ is 2, R¹¹ and R¹² are independently hydrogen or substituted or unsubstituted (e.g. C₁-C₅) alkyl. In embodiments, R⁴ is independently hydrogen, —Cl, —F, —CF₃, —SO₂NH₂ or methyl. In embodiments, R⁴ is independently —Cl or —CF₃. In embodiments, R⁴ is independently —Cl or —CF₃ and z is 2. In embodiments, R⁴ is independently hydrogen or —Cl. In embodiments, R⁴ is independently —Cl or hydrogen and z is 2. In embodiments, R⁴ is independently hydrogen or —F. In embodiments, R⁴ is independently hydrogen or —CF₃. In embodiments, R¹¹ and R¹² are independently hydrogen and z is 1. In embodiments, R⁴ is independently hydrogen or —SO₂NH₂. In embodiments, R⁴ is independently —SO₂NH₂ or hydrogen and z is 2. In embodiments, R⁴ is independently hydrogen or methyl. In related embodiments, z is 2. In embodiments, R⁴ is —OR¹¹ and z is 1. In embodiments, R¹¹ is —CF₃.

In the compositions provided herein including embodiments thereof, W¹, W², W⁴ and W⁵ may be independently CR¹³ or N and R¹³ may be hydrogen. Thus, in embodiments, W¹, W², W⁴, and W⁵ are independently N or CH.

In the compositions provided herein including embodiments thereof, W³ may O, NR¹⁴, or S. In embodiments, W³ is independently O, NH or S. In embodiments, W³ is O. In embodiments, W³ is NH. In embodiments, W³ is S.

In the compositions provided herein including embodiments thereof, L¹ may be independently a bond, —S(O)—, —S(O)₂NH—, —NHS(O)₂—, —C(O)O—, —OC(O)—, —C(O)—, —C(O)NH—, —NH—, —NHC(O)—, —O—, —S—, —NH-L²-, —NH—R¹⁵—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L¹ is substituted or unsubstituted C₁-C₅ alkylene, —NH— or —NH-L²-. In embodiments, L¹ is unsubstituted C₁-C₅ alkylene, —NH— or —NH-L²-. In embodiments, L¹ is unsubstituted C₁-C₃ alkylene, —NH— or —NH-L²-. In embodiments, L¹is unsubstituted methylene.

In one embodiment, L² may be independently —C(O)—, —C(O)—NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L² is —C(O)—, —C(O)—NH—, substituted (e.g., R¹⁶-substituted) or unsubstituted C₁-C₂₀ (e.g., C₁-C₆) alkylene, substituted (e.g., R¹⁶-substituted) or unsubstituted 2 to 20 membered (e.g., 2 to 6 membered) heteroalkylene, substituted (e.g., R¹⁶-substituted) or unsubstituted C₃-C₈ (e.g., C₅-C₇) cycloalkylene, substituted (e.g., R¹⁶-substituted) or unsubstituted 3 to 8 membered (e.g., 3 to 6 membered) heterocycloalkylene, substituted (e.g., R¹⁶-substituted) or unsubstituted C₅-C₁₀ (e.g., C₅-C₆) arylene, or substituted (e.g., R¹⁶-substituted) or unsubstituted 5 to 10 membered (e.g., 5 to 6 membered) heteroarylene.

In embodioments, L² is substituted (e.g., R¹⁶-substituted) or unsubstituted C₁-C₅ alkylene. In embodioments, L² is unsubstituted C₁-C₅ alkylene. In embodioments, L² is unsubstituted C₁-C₃ alkylene. In embodiments, L² is unsubstituted methylene.

In embodiments, L² is R¹⁶-substituted or unsubstituted C₁-C₅ alkylene. In embodiments, L² is R¹⁶-substituted or unsubstituted C₁-C₃ alkylene. In embodiments, L² is R¹⁶-substituted C₁-C₃ alkylene. In embodiments, L² is R¹⁶-substituted methylene.

In the compositions provided herein including embodiments thereof, R¹⁶ may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R¹⁷-substituted or unsubstituted C₁-C₂₀ (e.g., C₁-C₆) alkyl, R¹⁷-substituted or unsubstituted 2 to 20 membered (e.g., 2 to 6 membered) heteroalkyl, substituted or unsubstituted C₃-C₈ (e.g., C₅-C₇) cycloalkyl, R¹⁷-substituted or unsubstituted 3 to 8 membered (e.g., 3 to 6 membered) heterocycloalkyl, R¹⁷-substituted or unsubstituted C₅-C₁₀ (e.g., C₅-C₆) aryl, or R¹⁷-substituted or unsubstituted 5 to 10 membered (e.g., 5 to 6 membered) heteroaryl. In embodiments, R^{16 is} R¹⁷⁻substituted or unsubstituted C₁-C₅ alkyl, R¹⁷ is R¹⁸-substituted or unsubstituted 5 to 10 membered heteroaryl, and R¹⁸ is substituted or unsubstituted 1 to 5 membered heteroalkyl. In embodiments, R¹⁶ is R¹⁷-substituted or unsubstituted saturated C₁-C₅ alkyl. In embodiments, R¹⁶ is R¹⁷-substituted or unsubstituted ethenyl. In embodiments, R¹⁷ is R¹⁸-substituted 9 membered heteroaryl. In embodiments, R¹⁷ is R¹⁸-substituted benzofuranyl. In embodiments, R¹⁸ is —C(NH)NH₂. In related embodiments, R¹ is substituted or unsubstituted 5 membered heterocycloalkyl. In related embodiments, R¹ is pyrrolidinyl.

In embodiments, the compound provided herein including embodiments thereof has the formula:

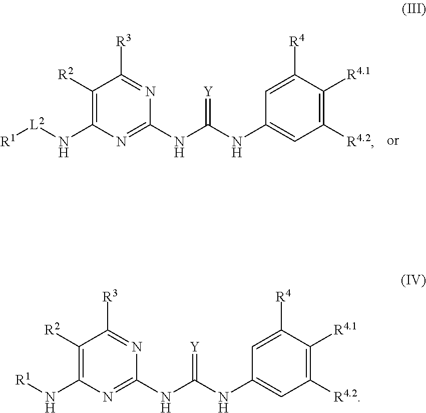

In formula (III) or (IV) R¹, R², R³, L², Y, W¹, W², W³, and W⁴ are as defined herein (including embodiments thereof) and R⁴, R^{4.1} and R^{4.2} are independently hydrogen, halogen, —CX^{d}₃, —SOᵥ₄NR¹¹R¹², —OR¹¹, or substituted or unsubstituted alkyl. In related embodiments, R¹ is substituted or unsubstituted piperidinyl or substituted or unsubstituted pyrrolidinyl.

In embodiments, the compound provided herein including embodiments thereof has the formula:

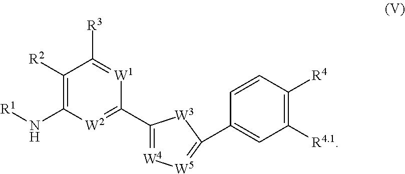

In formula (V) R¹, R², R³, L², Y, W¹, W², W³, and W⁴ are as defined herein (including embodiments thereof) and R⁴ and R^{4.1} are independently halogen or —CF₃. In related embodiments, R¹ is substituted or unsubstituted piperidinyl.

In some embodiments, a compound of formula (I), (II), (III), (IV) or (V) is one or more compounds set forth in Table 1 below.

In another aspect, a compound having the formula

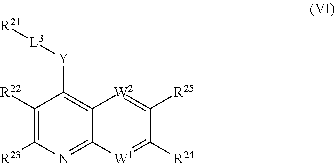

is provided. In formula (VI) R²¹ is hydrogen, halogen, —CX^{a}₃, —CN, —SO₂Cl, —SOₙ₁R²⁸, —SOᵥ₁NR²⁸R²⁹, —NHNH₂, —ONR²⁸R²⁹, —NHC═(O)NHNH₂, —NHC═(O)NR²⁸R²⁹, —NHC═(O)R²⁸, —N(O)ₘ₁, —NR²⁸R²⁹, —NH—O—R²⁸, —C(O)R²⁸, —C(O)—OR²⁸, —C(O)NR²⁸R²⁹, —N(R²⁸)C(O)R²⁹, —OR²⁸, —O—C(O)NR²⁸R²⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R²² is hydrogen, halogen, —CX^{b}₃, —CN, —SO₂Cl, —SOₙ₂R³⁰, —SOᵥ₂NR³⁰R³¹, —NHNH₂, —ONR³⁰R³¹, —NHC═(O)NHNH₂, —NHC═(O)NR³⁰R³¹, —NHC═(O)R³⁰, —N(O)ₘ₂, —NR³⁰R³¹, —NH—O—R³⁰, —C(O)R³⁰, —C(O)—OR³⁰, —C(O)NR³⁰R³¹, —N(R³⁰)C(O)R³¹, —O—C(O)NR³⁰R³¹, —OR³⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R²³ is hydrogen, halogen, —CX^{c}₃, —CN, —SO₂Cl, —SOₙ₃R³², —SOᵥ₃NR³²R³³, —NR³²SOᵥ₃R³³, —NHNH₂, —ONR³²R³³, —NHC═(O)NHNH₂, —NHC═(O)NR³²R³³, —NHC═(O)R³², —N(O)ₘ₃, —NR³²R³³, —NH—O—R³², —R³²NR³³NH₂, —C(O)R³², —C(O)—OR³², —C(O)NR³²R³³, —N(R³²)C(O)R³³, —O—C(O)NR³²R³³, —OR³², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R²⁴ is hydrogen, halogen, —CX^{d}₃, —CN, —SO₂Cl, —SOₙ₄R³⁴, —SOᵥ₄NR³⁴R³⁵, —NHNH₂, —ONR³⁴R³⁵, —NHC═(O)NHNH₂, —NHC═(O)NR³⁴R³⁵, —NHC═(O)R³⁴, —N(O)ₘ₄, —NR³⁴R³⁵, —NH—O—R³⁴, —C(O)R³⁴, —C(O)—OR³⁴, —C(O)NR³⁴R³⁵, —N(R³⁴)C(O)R³⁵, —O—C(O)NR³⁴R³⁵, —OR³⁴, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R²⁵ is hydrogen, halogen, —CX^{e}₃, —CN, —SO₂Cl, —SOₙ₅R³⁶, —SOᵥ₅NR³⁶R³⁷, —NHNH₂, —ONR³⁶R³⁷, —NHC═(O)NHNH₂, —NHC═(O)NR³⁶R³⁷, —NHC═(O)R³⁶, —N(O)ₘ₅, —NR³⁶R³⁷, —NH—O—R³⁶, —C(O)R³⁶, —C(O)—OR³⁶, —C(O)NR³⁶R³⁷, —N(R³⁶)C(O)R³⁷, —O—C(O)NR³⁶R³⁷, —OR³⁶, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R²⁶ is independently hydrogen, halogen, —CX^{f}₃, —CN, —SO₂Cl, —SOₘ₆R³⁸, —SOᵥ₆NR³⁸R³⁹, —NHNH₂, —ONR³⁸R³⁹, —NHC═(O)NHNH₂, —NHC═(O)NR³⁸R³⁹, —NHC═(O)R³⁸, —N(O)ₘ₆, —NR³⁸R³⁹, —NH—O—R³⁸, —C(O)R³⁸, —C(O)—OR³⁸, —C(O)NR³⁸R³⁹, —N(R³⁸)C(O)R³⁹, —O—C(O)NR³⁸R³⁹, —OR³⁸, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R²⁷ is hydrogen, halogen, —CX^{g}₃, —CN, —SO₂Cl, —SOₙ₇R⁴⁰, —SOᵥ₇NR⁴⁰R⁴¹, —NHNH₂, —ONR⁴⁰R⁴¹, —NHC═(O)NHNH₂, —NHC═(O)NR⁴⁰R⁴¹, —NHC═(O)R⁴⁰, —N(O)ₘ₇, —NR⁴⁰R⁴¹, —NH—O—R⁴⁰, —C(O)R⁴⁰, —C(O)—OR⁴⁰, —C(O)NR⁴⁰R⁴¹, —N(R⁴⁰)C(O)R⁴¹, —O—C(O)NR⁴⁰R⁴¹, —OR⁴⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R²⁸ is hydrogen, halogen, —CX^{h}₃, —CN, —SO₂Cl, —SOₙ₈R⁴², —SOᵥ₈NR⁴²R⁴³, —NHNH₂, —ONR⁴²R⁴³, —NHC═(O)NHNH₂, —NHC═(O)NR⁴²R⁴³, —NHC═(O)R⁴², —N(O)ₘ₈, —NR⁴²R⁴³, —NH—O—R⁴², —C(O)R⁴², —C(O)—OR⁴², —C(O)NR⁴²R⁴³, —N(R⁴²)C(O)R⁴³, —O—C(O)NR⁴²R⁴³, —OR⁴², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R²⁹ is hydrogen, halogen, —CXⁱ₃, —CN, —SO₂Cl, —SOₙ₉R⁴⁴, —SOᵥ₉NR⁴⁴R⁴⁵, —NHNH₂, —ONR⁴⁴R⁴⁵, —NHC═(O)NHNH₂, —NHC═(O)NR⁴⁴R⁴⁵, —NHC═(O)R⁴⁴, —N(O)ₘ₉,₉, —NR⁴⁴R⁴⁵, —NH—O—R⁴⁴, —C(O)R⁴⁴, —C(O)—OR⁴⁴, —C(O)NR⁴⁴R⁴⁵, —N(R⁴⁴)C(O)R⁴⁵, —O—C(O)NR⁴⁴R⁴⁵, —OR⁴⁴, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In formula (VI) Y is O or NH. W¹ is N or CR²⁶. W² is independently N or CR²⁷. L¹ is a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R³⁰, R³¹, R³², R³³, R³⁴, R³⁵ R³⁶, R³⁷, R³⁸, R³⁹, R⁴⁰, R⁴¹, R⁴², R⁴³, R⁴⁴ and R⁴⁵ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. X^{a}, X^{b}, X^{c}, X^{d}, X^{e}, X^{f}, X^{g}, X^{h} and Xⁱ are independently —F, —Cl, —Br, or —I. The symbols n₁, n₂, n₃, n₄, n₅, n₆ an, n₇, n₈ and n₉ are independently integers from 0 to 4. The symbols m₁, m₂, m₃, m₄, m₅, m₆, m₇, m₈ and m₉ are independently integers from 1 to 2. The symbols v₁, v₂, v₃, v₄, v₅, v₆, v₇, v₈ and v₉ are independently integers from 1 to 2. The symbol z is independently an integer from 0 to 5.

In formula (VI) R²¹ may be independently hydrogen, halogen, —CX^{a}₃, —CN, —SO₂Cl, —SOₙ₁R²⁸, —SOᵥ₁NR²⁸R²⁹, —NHNH₂, —ONR²⁸R²⁹, —NHC═(O)NHNH₂, —NHC═(O)NR²⁸R²⁹, —NHC═(O)R²⁸, —N(O)ₘ₁, —NR²⁸R²⁹, —NH—O—R²⁸, —C(O)R²⁸, —C(O)—OR²⁸, —C(O)NR²⁸R²⁹, —N(R²⁸)C(O)R²⁹, —OR²⁸, —O—C(O)NR²⁸R²⁹, substituted or unsubstituted C₁-C₂₀ (e.g., C₁-C₆) alkyl, substituted or unsubstituted 2 to 20 membered (e.g., 2 to 6 membered) heteroalkyl, substituted or unsubstituted C₃-C₈ (e.g., C₅-C₇) cycloalkyl, substituted or unsubstituted 3 to 8 membered (e.g., 3 to 6 membered) heterocycloalkyl, substituted or unsubstituted C₅-C₁₀ (e.g., C₅-C₆) aryl, or substituted or unsubstituted 5 to 10 membered (e.g., 5 to 6 membered) heteroaryl.

In embodiments, R²¹ is independently hydrogen, halogen, —CX^{a}₃, —CN, —SO₂Cl, —SOₙ₁R²⁸, —SOᵥ₁NR²⁸R²⁹, —NHNH₂, —ONR²⁸R²⁹, —NHC═(O)NHNH₂, —NHC═(O)NR²⁸R²⁹, —NHC═(O)R²⁸, —N(O)ₘ₁, —NR²⁸R²⁹, —NH—O—R²⁸, —C(O)R²⁸, —C(O)—OR²⁸, —C(O)NR²⁸R²⁹, —N(R²⁸)C(O)R²⁹, —OR²⁸, —O—C(O)NR²⁸R²⁹, R^{21A}-substituted or unsubstituted alkyl, R^{21A}-substituted or unsubstituted heteroalkyl, R^{21A}-substituted or unsubstituted cycloalkyl, R^{21A}-substituted or unsubstituted heterocycloalkyl, R^{21A}-substituted or unsubstituted aryl, or R^{21A}-substituted or unsubstituted heteroaryl. In embodiments, R²¹ is independently hydrogen, halogen, —CX^{a}₃, —CN, —SO₂Cl, —SOₙ₁R²⁸, —SOᵥ₁NR²⁸R²⁹, —NHNH₂, —ONR²⁸R²⁹, —NHC═(O)NHNH₂, —NHC═(O)NR²⁸R²⁹, —NHC═(O)R²⁸, —N(O)ₘ₁, —NR²⁸R²⁹, —NH—O—R²⁸, —C(O)R²⁸, —C(O)—OR²⁸, —C(O)NR²⁸R²⁹, —N(R²⁸)C(O)R²⁹, —OR²⁸, or —O—C(O)NR²⁸R²⁹. In embodiments, R²¹ is R^{21A}-substituted or unsubstituted C₁-C₂₀ (e.g., C₁-C₆) alkyl, R^{21A}-substituted or unsubstituted 2 to 20 membered (e.g., 2 to 6 membered) heteroalkyl, R^{21A}-substituted or unsubstituted C₃-C₈ (e.g., C₅-C₇) cycloalkyl, R^{21A}-substituted or unsubstituted 3 to 8 membered (e.g., 3 to 6 membered) heterocycloalkyl, R^{21A}-substituted or unsubstituted C₅-C₁₀ (e.g., C₅-C₆) aryl, or R^{21A}-substituted or unsubstituted 5 to 10 membered (e.g., 5 to 6 membered) heteroaryl.

R^{21A} may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{21B}-substituted or unsubstituted alkyl, R^{21B}-substituted or unsubstituted heteroalkyl, R^{21B}-substituted or unsubstituted cycloalkyl, R^{21B}-substituted or unsubstituted heterocycloalkyl, R^{21B}-substituted or unsubstituted aryl, or R^{21B}-substituted or unsubstituted heteroaryl. In embodiments, where R^{21A} is ═O or ═S, R²¹ is not aryl or heteroaryl. In embodiments, R^{21A} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂. In other embodiments, R^{21A is} R^{21B}-substituted or unsubstituted alkyl, R^{21B}-substituted or unsubstituted heteroalkyl, R^{21B}-substituted or unsubstituted cycloalkyl, R^{21B}-substituted or unsubstituted heterocycloalkyl, R^{21B}-substituted or unsubstituted aryl, or R^{21B}-substituted or unsubstituted heteroaryl. R^{21A} may be R^{21B}-substituted or unsubstituted C₁-C₂₀ (e.g., C₁-C₆) alkyl, R^{21B}-substituted or unsubstituted 2 to 20 membered (e.g., 2 to 6 membered) heteroalkyl, R^{21B}-substituted or unsubstituted C₃-C₈ (e.g., C₅-C₇) cycloalkyl, R^{21B}-substituted or unsubstituted 3 to 8 membered (e.g., 3 to 6 membered) heterocycloalkyl, R^{21B}-substituted or unsubstituted C₅-C₁₀ (e.g., C₅-C₆) aryl, or R^{21B}-substituted or unsubstituted 5 to 10 membered (e.g., 5 to 6 membered) heteroaryl.

R^{21B} may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{21C}-substituted or unsubstituted alkyl, R^{21C}-substituted or unsubstituted heteroalkyl, R^{21C}-substituted or unsubstituted cycloalkyl, R^{21C}-substituted or unsubstituted heterocycloalkyl, R^{21C}-substituted or unsubstituted aryl, or R^{21C}-substituted or unsubstituted heteroaryl. In embodiments, where R^{21B} is ═O or ═S, R^{21A} is not aryl or heteroaryl. In embodiments, R^{21B} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂. In other embodiments, R^{21B} is R^{21C}-substituted or unsubstituted alkyl, R^{21C}-substituted or unsubstituted heteroalkyl, R^{21C}-substituted or unsubstituted cycloalkyl, R^{21C}-substituted or unsubstituted heterocycloalkyl, R^{21C}-substituted or unsubstituted aryl, or R^{21C}-substituted or unsubstituted heteroaryl. R^{21B} may be R^{21C}-substituted or unsubstituted C₁-C₂₀ (e.g., C₁-C₆) alkyl, R^{21C}-substituted or unsubstituted 2 to 20 membered (e.g., 2 to 6 membered) heteroalkyl, R^{21C}-substituted or unsubstituted C₃-C₈ (e.g., C₅-C₇) cycloalkyl, R^{21C}-substituted or unsubstituted 3 to 8 membered (e.g., 3 to 6 membered) heterocycloalkyl, R^{21C}-substituted or unsubstituted C₅-C₁₀ (e.g., C₅-C₆) aryl, or R^{21C}-substituted or unsubstituted 5 to 10 membered (e.g., 5 to 6 membered) heteroaryl.

R^{21C} may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{21D}-substituted or unsubstituted alkyl, R^{21D}-substituted or unsubstituted heteroalkyl, R^{21D}-substituted or unsubstituted cycloalkyl, R^{21D}-substituted or unsubstituted heterocycloalkyl, R^{21D}-substituted or unsubstituted aryl, or R^{21D}-substituted or unsubstituted heteroaryl. In embodiments, where R^{21C} is ═O or ═S, R^{21B} is not aryl or heteroaryl. In embodiments, R^{21C} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂. In other embodiments, R^{21C} is R^{21D}-substituted or unsubstituted alkyl, R^{21D}-substituted or unsubstituted heteroalkyl, R^{21D}-substituted or unsubstituted cycloalkyl, R^{21D}-substituted or unsubstituted heterocycloalkyl, R^{21D}-substituted or unsubstituted aryl, or R^{21D}-substituted or unsubstituted heteroaryl. R^{21C} may be R^{21D}-substituted or unsubstituted C₁-C₂₀ (e.g., C₁-C₆) alkyl, R^{21D}-substituted or unsubstituted 2 to 20 membered (e.g., 2 to 6 membered) heteroalkyl, R^{21D}-substituted or unsubstituted C₃-C₈ (e.g., C₅-C₇) cycloalkyl, R^{21D}-substituted or unsubstituted 3 to 8 membered (e.g., 3 to 6 membered) heterocycloalkyl, R^{21D}-substituted or unsubstituted C₅-C₁₀ (e.g., C₅-C₆) aryl, or R^{21D}-substituted or unsubstituted 5 to 10 membered (e.g., 5 to 6 membered) heteroaryl.

R^{21D} may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{21E}-substituted or unsubstituted alkyl, R^{21E}-substituted or unsubstituted heteroalkyl, R^{21E}-substituted or unsubstituted cycloalkyl, R^{21E}-substituted or unsubstituted heterocycloalkyl, R^{21E}-substituted or unsubstituted aryl, or R^{21E}-substituted or unsubstituted heteroaryl. In embodiments, where R^{21D} is ═O or ═S, R^{21C} is not aryl or heteroaryl. In embodiments, R^{21D} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂. In other embodiments, R^{21D} is R^{21E}-substituted or unsubstituted alkyl, R^{21E}-substituted or unsubstituted heteroalkyl, R^{21E}-substituted or unsubstituted cycloalkyl, R^{21E}-substituted or unsubstituted heterocycloalkyl, R^{21E}-substituted or unsubstituted aryl, or R^{21E}-substituted or unsubstituted heteroaryl. R^{21D} may be R^{21E}-substituted or unsubstituted C₁-C₂₀ (e.g., C₁-C₆) alkyl, R^{21E}-substituted or unsubstituted 2 to 20 membered (e.g., 2 to 6 membered) heteroalkyl, R^{21E}-substituted or unsubstituted C₃-C₈ (e.g., C₅-C₇) cycloalkyl, R^{21E}-substituted or unsubstituted 3 to 8 membered (e.g., 3 to 6 membered) heterocycloalkyl, R^{21E}-substituted or unsubstituted C₅-C₁₀ (e.g., C₅-C₆) aryl, or R^{21E}-substituted or unsubstituted 5 to 10 membered (e.g., 5 to 6 membered) heteroaryl.

R^{21E} may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. In embodiments, where R^{21E} is ═O or ═S, R^{21D} is not aryl or heteroaryl. In embodiments, R^{21E} is independently hydrogen, halogen, ═O, ═S, —CF₃, —CN, —CCl₃, —COOH, —CH₂COOH, —CONH₂, —OH, —SH, —SO₂Cl, —SO₃H, —SO₄, —SO₂NH₂, —NO₂, —NH₂, —NHNH₂, —ONH₂, —NHC═(O)NHNH₂. In other embodiments, R^{21E} is unsubstituted alkyl, unsubstituted heteroalkyl, unsubstituted cycloalkyl, unsubstituted heterocycloalkyl, unsubstituted aryl, or unsubstituted heteroaryl. R^{21E} may be unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cycloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

In embodiments, R²¹ of formula (VI) is independently R^{21A}-substituted or unsubstituted alkyl, R^{21A}-substituted or unsubstituted heteroalkyl, R^{21A}-substituted or unsubstituted cycloalkyl, R^{21A}-substituted or unsubstituted heterocycloalkyl, R^{21A}-substituted or unsubstituted aryl, or R^{21A}-substituted or unsubstituted heteroaryl. R^{21A} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{21B}-substituted or unsubstituted alkyl, R^{21B}-substituted or unsubstituted heteroalkyl, R^{21B}-substituted or unsubstituted cycloalkyl, R^{21B}-substituted or unsubstituted heterocycloalkyl, R^{21B}-substituted or unsubstituted aryl, or R^{21B}-substituted or unsubstituted heteroaryl. R^{21B} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{21C}-substituted or unsubstituted alkyl, R^{21C}-substituted or unsubstituted heteroalkyl, R^{21C}-substituted or unsubstituted cycloalkyl, R^{21C}-substituted or unsubstituted heterocycloalkyl, R^{21C}-substituted or unsubstituted aryl, or R^{21C}-substituted or unsubstituted heteroaryl. R^{21C} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{21D}-substituted or unsubstituted alkyl, R^{21D}-substituted or unsubstituted heteroalkyl, R^{21D}-substituted or unsubstituted cycloalkyl, R^{21D}-substituted or unsubstituted heterocycloalkyl, R^{21D}-substituted or unsubstituted aryl, or R^{21D}-substituted or unsubstituted heteroaryl. R^{21D} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{21E}-substituted or unsubstituted alkyl, R^{21E}-substituted or unsubstituted heteroalkyl, R^{21E}-substituted or unsubstituted cycloalkyl, R^{21E}-substituted or unsubstituted heterocycloalkyl, R^{21E}-substituted or unsubstituted aryl, or R^{21E}-substituted or unsubstituted heteroaryl. And R^{21E} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl.

In formula (VI) R²² may be independently hydrogen, halogen, —CX^{b}₃, —CN, —SO₂Cl, —SOₙ₂R³⁰, —SOᵥ₂NR³⁰R³¹, —NHNH₂, —ONR³⁰R³¹, —NHC═(O)NHNH₂, —NHC═(O)NR³⁰R³¹, —NHC═(O)R³⁰, —N(O)ₘ₂, —NR³⁰R³¹, —NH—O—R³⁰, —C(O)R³⁰, —C(O)—OR³⁰, —C(O)NR³⁰R³¹, —N(R³⁰)C(O)R³¹, —O—C(O)NR³⁰R³¹, —OR³⁰, substituted or unsubstituted C₁-C₂₀ (e.g., C₁-C₆) alkyl, substituted or unsubstituted 2 to 20 membered (e.g., 2 to 6 membered) heteroalkyl, substituted or unsubstituted C₃-C₈ (e.g., C₅-C₇) cycloalkyl, substituted or unsubstituted 3 to 8 membered (e.g., 3 to 6 membered) heterocycloalkyl, substituted or unsubstituted C₅-C₁₀ (e.g., C₅-C₆) aryl, or substituted or unsubstituted 5 to 10 membered (e.g., 5 to 6 membered) heteroaryl. In formula (VI) R²² may be independently hydrogen, halogen, —CX^{b}₃, —CN, —SOₙ₂Cl, —SOₙ₂R³⁰, —SOᵥ₂NR³⁰R³¹, —NHNH₂, —ONR³⁰R³¹, —NHC═(O)NHNH₂, —NHC═(O)NR³⁰R³¹, —NHC═(O)R³⁰, —N(O)ₘ₂, —NR³⁰R³¹, —NH—O—R³⁰, —C(O)R³⁰, —C(O)—OR³⁰, —C(O)NR³⁰R³¹, —N(R³⁰)C(O)R³¹, —O—C(O)NR³⁰R³¹, —OR³⁰, unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cycloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

In formula (VI) R²³ may be independently hydrogen, halogen, —CX^{c}₃, —CN, —SO₂Cl, —SOₙ₃R³², —SOᵥ₃NR³²R³³, —NR³²SOᵥ₃R³³, —NHNH₂, —ONR³²R³³, —NHC═(O)NHNH₂, —NHC═(O)NR³²R³³, —NHC═(O)R³², —N(O)ₘ₃, —NR³²R³³, —NH—O—R³², —R³²NR³³NH₂, —C(O)R³², —C(O)—OR³², —C(O)NR³²R³³, —N(R³²)C(O)R³³, —O—C(O)NR³²R³³, —OR³², substituted or unsubstituted C₁-C₂₀ (e.g., C₁-C₆) alkyl, substituted or unsubstituted 2 to 20 membered (e.g., 2 to 6 membered) heteroalkyl, substituted or unsubstituted C₃-C₈ (e.g., C₅-C₇) cycloalkyl, substituted or unsubstituted 3 to 8 membered (e.g., 3 to 6 membered) heterocycloalkyl, substituted or unsubstituted C₅-C₁₀ (e.g., C₅-C₆) aryl, or substituted or unsubstituted 5 to 10 membered (e.g., 5 to 6 membered) heteroaryl. In formula (VI) R²³ may be independently hydrogen, halogen, —CX^{c}₃, —CN, —SO₂Cl, —SOₙ₃R³², —SOᵥ₃NR³²R³³, —NR³²SOᵥ₃R³³, —NHNH₂, —ONR³²R³³, —NHC═(O)NHNH₂, —NHC═(O)NR³²R³³ —NHC═(O)R³², —N(O)ₘ₃, —NR³²R³³, —NH—O—R³², —R³²NR³³NH₂, —C(O)R³², —C(O)—OR³², —C(O)NR³²R³³, —N(R³²)C(O)R³³, —O—C(O)NR³²R³³, —OR³², unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cycloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

In formula (VI) R²⁴ may be independently hydrogen, halogen, —CX^{d}₃, —CN, —SO₂Cl, —SOₙ₄R³⁴, —SOᵥ₄NR³⁴R³⁵, —NHNH₂, —ONR³⁴R³⁵, —NHC═(O)NHNH₂, —NHC═(O)NR³⁴R³⁵, —NHC═(O)R³⁴, —N(O)ₘ₄, —NR³⁴R³⁵, —NH—O—R³⁴, —C(O)R³⁴, —C(O)—OR³⁴, —C(O)NR³⁴R³⁵, —N(R³⁴)C(O)R³⁵, —O—C(O)NR³⁴R³⁵, —OR³⁴, substituted or unsubstituted C₁-C₂₀ (e.g., C₁-C₆) alkyl, substituted or unsubstituted 2 to 20 membered (e.g., 2 to 6 membered) heteroalkyl, substituted or unsubstituted C₃-C₈ (e.g., C₅-C₇) cycloalkyl, substituted or unsubstituted 3 to 8 membered (e.g., 3 to 6 membered) heterocycloalkyl, substituted or unsubstituted C₅-C₁₀ (e.g., C₅-C₆) aryl, or substituted or unsubstituted 5 to 10 membered (e.g., 5 to 6 membered) heteroaryl.

In embodiments, R²⁴ is independently hydrogen, halogen, —CX^{d}₃, —CN, —SO₂Cl, —SOₙ₄R³⁴, —SOᵥ₄NR³⁴R³⁵, —NHNH₂, —ONR³⁴R³⁵, —NHC═(O)NHNH₂, —NHC═(O)NR³⁴R³⁵, —NHC═(O)R³⁴, —N(O)ₘ₄, —NR³⁴R³⁵, —NH—O—R³⁴, —C(O)R³⁴, —C(O)—OR³⁴, —C(O)NR³⁴R³⁵, —N(R³⁴)C(O)R³⁵, —O—C(O)NR³⁴R³⁵, —OR³⁴, R^{24A}-substituted or unsubstituted alkyl, R^{24A}-substituted or unsubstituted heteroalkyl, R^{24A}-substituted or unsubstituted cycloalkyl, R^{24A}-substituted or unsubstituted heterocycloalkyl, R^{24A}-substituted or unsubstituted aryl, or R^{24A}-substituted or unsubstituted heteroaryl. In embodiments, R²⁴ is independently hydrogen, halogen, —CX^{d}₃, —CN, —SO₂Cl, —SOₙ₄R³⁴, —SOᵥ₄NR³⁴R³⁵, —NHNH₂, —ONR³⁴R³⁵, —NHC═(O)NHNH₂, —NHC═(O)NR³⁴R³⁵, —NHC═(O)R³⁴, —N(O)ₘ₄, —NR³⁴R³⁵, —NH—O—R³⁴, —C(O)R³⁴, —C(O)—OR³⁴, —C(O)NR³⁴R³⁵, —N(R³⁴)C(O)R³⁵, —O—C(O)NR³⁴R³⁵, —OR³⁴. In embodiments, R²⁴ is R^{24A}-substituted or unsubstituted C₁-C₂₀ (e.g., C₁-C₆) alkyl, R^{24A}-substituted or unsubstituted 2 to 20 membered (e.g., 2 to 6 membered) heteroalkyl, R^{24A}-substituted or unsubstituted C₃-C₈ (e.g., C₅-C₇) cycloalkyl, R^{24A}-substituted or unsubstituted 3 to 8 membered (e.g., 3 to 6 membered) heterocycloalkyl, R^{24A}-substituted or unsubstituted C₅-C₁₀ (e.g., C₅-C₆) aryl, or R^{24A}-substituted or unsubstituted 5 to 10 membered (e.g., 5 to 6 membered) heteroaryl.

R^{24A} may be independently hydrogen, halogen, ═O, ═S, —CF₃, —CN, —CCl₃, —COOH, —CH₂COOH, —CONH₂, —OH, —SH, —SO₂Cl, —SO₄H, —SO₂NH₂, —NO₂, —NH₂, —NHNH₂, —ONH₂, —NHC═(O)NHNH₂, R^{24B}-substituted or unsubstituted alkyl, R^{24B}-substituted or unsubstituted heteroalkyl, R^{24B}-substituted or unsubstituted cycloalkyl, R^{24B}-substituted or unsubstituted heterocycloalkyl, R^{24B}-substituted or unsubstituted aryl, or R^{24B}-substituted or unsubstituted heteroaryl. In embodiments, where R^{24A} is ═O or ═S, R²⁴ is not aryl or heteroaryl. In embodiments, R^{24A} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂. In other embodiments, R^{24A is} R^{24B-}substituted or unsubstituted alkyl, R^{24B}-substituted or unsubstituted heteroalkyl, R^{24B}-substituted or unsubstituted cycloalkyl, R^{24B}-substituted or unsubstituted heterocycloalkyl, R^{24B}-substituted or unsubstituted aryl, or R^{24B}-substituted or unsubstituted heteroaryl. R^{24A may} be R^{24B}-substituted or unsubstituted C₁-C₂₀ (e.g., C₁-C₆) alkyl, R^{24B}-substituted or unsubstituted 2 to 20 membered (e.g., 2 to 6 membered) heteroalkyl, R^{24B}-substituted or unsubstituted C₃-C₈ (e.g., C₅-C₇) cycloalkyl, R^{24B}-substituted or unsubstituted 3 to 8 membered (e.g., 3 to 6 membered) heterocycloalkyl, R^{24B}-substituted or unsubstituted C₅-C₁₀ (e.g., C₅-C₆) aryl, or R^{24B}-substituted or unsubstituted 5 to 10 membered (e.g., 5 to 6 membered) heteroaryl.

R^{24B} may be independently hydrogen, halogen, ═O, ═S, —CF₃, —CN, —CCl₃, —COOH, —CH₂COOH, —OH, —SH, —SO₂Cl, —SO₃H, —SO₄H, —SO₂NH₂, —NO₂, —NH₂, —NHNH₂, —ONH₂, —NHC═(O)NHNH₂, R^{24C}-substituted or unsubstituted alkyl, R^{24C}-substituted or unsubstituted heteroalkyl, R^{24C}-substituted or unsubstituted cycloalkyl, R^{24C}-substituted or unsubstituted heterocycloalkyl, R^{24C}-substituted or unsubstituted aryl, or R^{24C}-substituted or unsubstituted heteroaryl. In embodiments, where R^{24B} is ═O or ═S, R^{24A} is not aryl or heteroaryl. In embodiments, R^{24B} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂. In other embodiments, R^{24B} is R^{24C}-substituted or unsubstituted alkyl, R^{24C}-substituted or unsubstituted heteroalkyl, R^{24C}-substituted or unsubstituted cycloalkyl, R^{24C}-substituted or unsubstituted heterocycloalkyl, R^{24C}-substituted or unsubstituted aryl, or R^{24C}-substituted or unsubstituted heteroaryl. R^{24B} may be R^{24C}-substituted or unsubstituted C₁-C₂₀ (e.g., C₁-C₆) alkyl, R^{24C}-substituted or unsubstituted 2 to 20 membered (e.g., 2 to 6 membered) heteroalkyl, R^{24C}-substituted or unsubstituted C₃-C₈ (e.g., C₅-C₇) cycloalkyl, R^{24C}-substituted or unsubstituted 3 to 8 membered (e.g., 3 to 6 membered) heterocycloalkyl, R^{24C}-substituted or unsubstituted C₅-C₁₀ (e.g., C₅-C₆) aryl, or R^{24C}-substituted or unsubstituted 5 to 10 membered (e.g., 5 to 6 membered) heteroaryl.

R^{24C} may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{24D}-substituted or unsubstituted alkyl, R^{24D}-substituted or unsubstituted heteroalkyl, R^{24D}-substituted or unsubstituted cycloalkyl, R^{24D}-substituted or unsubstituted heterocycloalkyl, R^{24D}-substituted or unsubstituted aryl, or R^{24D}-substituted or unsubstituted heteroaryl. In embodiments, where R^{24C} is ═O or ═S, R^{24B} is not aryl or heteroaryl. In embodiments, R^{24C} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂. In other embodiments, R^{24C} is R^{24D}-substituted or unsubstituted alkyl, R^{24D}-substituted or unsubstituted heteroalkyl, R^{24D}-substituted or unsubstituted cycloalkyl, R^{24D}-substituted or unsubstituted heterocycloalkyl, R^{24D}-substituted or unsubstituted aryl, or R^{24D}-substituted or unsubstituted heteroaryl. R^{24C} may be R^{24D}-substituted or unsubstituted C₁-C₂₀ (e.g., C₁-C₆) alkyl, R^{24D}-substituted or unsubstituted 2 to 20 membered (e.g., 2 to 6 membered) heteroalkyl, R^{24D}-substituted or unsubstituted C₃-C₈ (e.g., C₅-C₇) cycloalkyl, R^{24D}-substituted or unsubstituted 3 to 8 membered (e.g., 3 to 6 membered) heterocycloalkyl, R^{24D}-substituted or unsubstituted C₅-C₁₀ (e.g., C₅-C₆) aryl, or R^{24D}-substituted or unsubstituted 5 to 10 membered (e.g., 5 to 6 membered) heteroaryl.

R^{24D} may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. In embodiments, where R^{24D} is ═O or ═S, R^{24C} is not aryl or heteroaryl. In embodiments, R^{24D} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂. In other embodiments, R^{24D} is unsubstituted alkyl, unsubstituted heteroalkyl, unsubstituted cycloalkyl, unsubstituted heterocycloalkyl, unsubstituted aryl, or unsubstituted heteroaryl. R^{24D} may be unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cycloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

In formula (VI) R²⁵ may be independently hydrogen, halogen, —CX^{e}₃, —CN, —SO₂Cl, —SOₙ₅R³⁶, —SOᵥ₅NR³⁶R³⁷, —NHNH₂, —ONR³⁶R³⁷, —NHC═(O)NHNH₂, —NHC═(O)NR³⁶R³⁷, —NHC═(O)R³⁶, —N(O)ₘ₅, —NR³⁶R³⁷, —NH—O—R³⁶, —C(O)R³⁶, —C(O)—OR³⁶, —C(O)NR³⁶R³⁷, —N(R³⁶)C(O)R³⁷, —O—C(O)NR³⁶R³⁷, —OR³⁶, substituted or unsubstituted C₁-C₂₀ (e.g., C₁-C₆) alkyl, substituted or unsubstituted 2 to 20 membered (e.g., 2 to 6 membered) heteroalkyl, substituted or unsubstituted C₃-C₈ (e.g., C₅-C₇) cycloalkyl, substituted or unsubstituted 3 to 8 membered (e.g., 3 to 6 membered) heterocycloalkyl, substituted or unsubstituted C₅-C₁₀ (e.g., C₅-C₆) aryl, or substituted or unsubstituted 5 to 10 membered (e.g., 5 to 6 membered) heteroaryl. In formula (VI) R²⁵ may be independently hydrogen, halogen, —CX^{e}₃, —CN, —SO₂Cl, —SOₙ₅R³⁶, —SOᵥ₅NR³⁶R³⁷, —NHNH₂, —ONR³⁶R³⁷, —NHC═(O)NHNH₂, —NHC═(O)NR³⁶R³⁷, —NHC═(O)R³⁶, —N(O)ₘ₅, —NR³⁶R³⁷, —NH—O—R³⁶, —C(O)R³⁶, —C(O)—OR³⁶, —C(O)NR³⁶R³⁷, —N(R³⁶)C(O)R³⁷, —O—C(O)NR³⁶R³⁷, —OR³⁶, unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cycloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

In formula (VI) R²⁶ may be independently hydrogen, halogen, —CX^{f}₃, —CN, —SO₂Cl, —SOₙ₆R³⁸, —SOᵥ₆NR³⁸R³⁹, —NHNH₂, —ONR³⁸R³⁹, —NHC═(O)NHNH₂, —NHC═(O)NR³⁸R³⁹, —NHC═(O)R³⁸, —N(O)ₘ₆, —NR³⁸R³⁹, —NH—O—R³⁸, —C(O)R³⁸, —C(O)—OR³⁸, —C(O)NR³⁸R³⁹, —N(R³⁸)C(O)R³⁹, —O—C(O)NR³⁸R³⁹, —OR³⁸, substituted or unsubstituted C₁-C₂₀ (e.g., C₁-C₆) alkyl, substituted or unsubstituted 2 to 20 membered (e.g., 2 to 6 membered) heteroalkyl, substituted or unsubstituted C₃-C₈ (e.g., C₅-C₇) cycloalkyl, substituted or unsubstituted 3 to 8 membered (e.g., 3 to 6 membered) heterocycloalkyl, substituted or unsubstituted C₅-C₁₀ (e.g., C₅-C₆) aryl, or substituted or unsubstituted 5 to 10 membered (e.g., 5 to 6 membered) heteroaryl. In formula (VI) R²⁶ may be independently hydrogen, halogen, —CX^{f}₃, —CN, —SO₂Cl, —SOₙ₆R³⁸, —SOᵥ₆NR³⁸R³⁹, —NHNH₂, —ONR³⁸R³⁹, —NHC═(O)NHNH₂, —NHC═(O)NR³⁸R³⁹, —NHC═(O)R³⁸, —N(O)ₘ₆, —NR³⁸R³⁹, —NH—O—R³⁸, —C(O)R³⁸, —C(O)—OR³⁸, —C(O)NR³⁸R³⁹, —N(R³⁸)C(O)R³⁹, —O—C(O)NR³⁸R³⁹, —OR³⁸, unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cycloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

In formula (VI) R²⁷ may be independently hydrogen, halogen, —CX^{g}₃, —CN, —SO₂Cl, —SOₙ₇R⁴⁰, —SOᵥ₇NR⁴⁰R⁴¹, —NHNH₂, —ONR⁴⁰R⁴¹, —NHC═(O)NHNH₂, —NHC═(O)NR⁴⁰R⁴¹, —NHC═(O)R⁴⁰, —N(O)ₘ₇, —NR⁴⁰R⁴¹, —NH—O—R⁴⁰, —C(O)R⁴⁰, —C(O)—OR⁴⁰, —C(O)NR⁴⁰R⁴¹, —N(R⁴⁰)C(O)R⁴¹, —O—C(O)NR⁴⁰R⁴¹, —OR⁴⁰, substituted or unsubstituted C₁-C₂₀ (e.g., C₁-C₆) alkyl, substituted or unsubstituted 2 to 20 membered (e.g., 2 to 6 membered) heteroalkyl, substituted or unsubstituted C₃-C₈ (e.g., C₅-C₇) cycloalkyl, substituted or unsubstituted 3 to 8 membered (e.g., 3 to 6 membered) heterocycloalkyl, substituted or unsubstituted C₅-C₁₀ (e.g., C₅-C₆) aryl, or substituted or unsubstituted 5 to 10 membered (e.g., 5 to 6 membered) heteroaryl. In formula (VI) R²⁷ may be independently hydrogen, halogen, —CX^{g}₃, —CN, —SO₂Cl, —SOₙ₇R⁴⁰, —SOᵥ₇NR⁴⁰R⁴¹, —NHNH₂, —ONR⁴⁰R⁴¹, —NHC═(O)NHNH₂, —NHC═(O)NR⁴⁰R⁴¹, —NHC═(O)R⁴⁰, —N(O)ₘ₇, —NR⁴⁰R⁴¹, —NH—O—R⁴⁰, —C(O)R⁴⁰, —C(O)—OR⁴⁰, C(O)NR⁴⁰R⁴¹, —N(R⁴⁰C(O)R⁴¹, —O—C(O)NR⁴⁰R⁴¹, —OR⁴⁰, unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cycloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

In formula (VI) R²⁸ may be independently hydrogen, halogen, —CX^{h}₃, —CN, —SO₂Cl, —SOₙ₈R⁴², —SOᵥ₈NR⁴²R⁴³, —NHNH₂, —ONR⁴²R⁴³, —NHC═(O)NHNH₂, —NHC═(O)NR⁴²R⁴³, —NHC═(O)R⁴², —N(O)ₘ₈, —NR⁴²R⁴³, —NH—O—R⁴², —C(O)R⁴², —C(O)—OR⁴², —C(O)NR⁴²R⁴³, —N(R⁴²)C(O)R⁴³, —O—C(O)NR⁴²R⁴³, —OR⁴², substituted or unsubstituted C₁-C₂₀ (e.g., C₁-C₆) alkyl, substituted or unsubstituted 2 to 20 membered (e.g., 2 to 6 membered) heteroalkyl, substituted or unsubstituted C₃-C₈ (e.g., C₅-C₇) cycloalkyl, substituted or unsubstituted 3 to 8 membered (e.g., 3 to 6 membered) heterocycloalkyl, substituted or unsubstituted C₅-C₁₀ (e.g., C₅-C₆) aryl, or substituted or unsubstituted 5 to 10 membered (e.g., 5 to 6 membered) heteroaryl.

In embodiments, R²⁸ is independently hydrogen, halogen, —CX^{h}₃, —CN, —SO₂Cl, —SOₙ₈R⁴², —SOᵥ₈NR⁴²R⁴³, —NHNH₂, —ONR⁴²R⁴³, —NHC═(O)NHNH₂, —NHC═(O)NR⁴²R⁴³, —NHC═(O)R^{42, —N(O)}ₘ₈, —NR⁴²R⁴³, —NH—O—R⁴², —C(O)R⁴², —C(O)—OR⁴², —C(O)NR⁴²R⁴³, —N(R⁴²)C(O)R⁴³, —O—C(O)NR⁴²R⁴³, —OR⁴², R^{28A}-substituted or unsubstituted alkyl, R^{28A}-substituted or unsubstituted heteroalkyl, R^{28A}-substituted or unsubstituted cycloalkyl, R^{28A}-substituted or unsubstituted heterocycloalkyl, R^{28A}-substituted or unsubstituted aryl, or R^{28A}-substituted or unsubstituted heteroaryl. In embodiments, R²⁸ is independently hydrogen, halogen, —CX^{h}₃, —CN, —SO₂Cl, —SOₙ₈R⁴², —SOᵥ₈NR⁴²R⁴³, —NHNH₂, —ONR⁴²R⁴³, —NHC═(O)NHNH₂, —NHC═(O)NR⁴²R⁴³, —NHC═(O)R⁴², —N(O)ₘ₈, —NR⁴²R⁴³, —NH—O—R⁴², —C(O)R⁴², —C(O)—OR⁴², —C(O)NR⁴²R⁴³, —N(R⁴²)C(O)R⁴³, —O—C(O)NR⁴²R⁴³, —OR⁴². In embodiments, R^{28 is} R^{28A}-substituted or unsubstituted C₁-C₂₀ (e.g., C₁-C₆) alkyl, R^{28A}-substituted or unsubstituted 2 to 20 membered (e.g., 2 to 6 membered) heteroalkyl, R^{28A}-substituted or unsubstituted C₃-C₈ (e.g., C₅-C₇) cycloalkyl, R^{28A}-substituted or unsubstituted 3 to 8 membered (e.g., 3 to 6 membered) heterocycloalkyl, R^{28A}-substituted or unsubstituted C₅-C₁₀ (e.g., C₅-C₆) aryl, or R^{28A}-substituted or unsubstituted 5 to 10 membered (e.g., 5 to 6 membered) heteroaryl.

R^{28A} may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{28B}-substituted or unsubstituted alkyl, R^{28B}-substituted or unsubstituted heteroalkyl, R^{28B}-substituted or unsubstituted cycloalkyl, R^{28B}-substituted or unsubstituted heterocycloalkyl, R^{28B}-substituted or unsubstituted aryl, or R^{28B}-substituted or unsubstituted heteroaryl. In embodiments, where R^{28A} is ═O or ═S, R²⁸ is not aryl or heteroaryl. In embodiments, R^{28A} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂. In other embodiments, R^{28A} is R^{28B}-substituted or unsubstituted alkyl, R^{28B}-substituted or unsubstituted heteroalkyl, R^{28B}-substituted or unsubstituted cycloalkyl, R^{28B}-substituted or unsubstituted heterocycloalkyl, R^{28B}-substituted or unsubstituted aryl, or R^{28B}-substituted or unsubstituted heteroaryl. R^{28A} may be R^{28B}-substituted or unsubstituted C₁-C₂₀ (e.g., C₁-C₆) alkyl, R^{28B}-substituted or unsubstituted 2 to 20 membered (e.g., 2 to 6 membered) heteroalkyl, R^{28B}-substituted or unsubstituted C₃-C₈ (e.g., C₅-C₇) cycloalkyl, R^{28B}-substituted or unsubstituted 3 to 8 membered (e.g., 3 to 6 membered) heterocycloalkyl, R^{28B}-substituted or unsubstituted C₅-C₁₀ (e.g., C₅-C₆) aryl, or R^{28B}-substituted or unsubstituted 5 to 10 membered (e.g., 5 to 6 membered) heteroaryl.

R^{28B} may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{28C}-substituted or unsubstituted alkyl, R^{28C}-substituted or unsubstituted heteroalkyl, R^{28C}-substituted or unsubstituted cycloalkyl, R^{28C}-substituted or unsubstituted heterocycloalkyl, R^{28C}-substituted or unsubstituted aryl, or R^{28C}-substituted or unsubstituted heteroaryl. In embodiments, where R^{28B} is ═O or ═S, R^{28A} is not aryl or heteroaryl. In embodiments, R^{28B} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂. In other embodiments, R^{28B} is R^{28C}-substituted or unsubstituted alkyl, R^{28C}-substituted or unsubstituted heteroalkyl, R^{28C}-substituted or unsubstituted cycloalkyl, R^{28C}-substituted or unsubstituted heterocycloalkyl, R^{28C}-substituted or unsubstituted aryl, or R^{28C}-substituted or unsubstituted heteroaryl. R^{28B} may be R^{28C}-substituted or unsubstituted C₁-C₂₀ (e.g., C₁-C₆) alkyl, R^{28C}-substituted or unsubstituted 2 to 20 membered (e.g., 2 to 6 membered) heteroalkyl, R^{28C}-substituted or unsubstituted C₃-C₈ (e.g., C₅-C₇) cycloalkyl, R^{28C}-substituted or unsubstituted 3 to 8 membered (e.g., 3 to 6 membered) heterocycloalkyl, R^{28C}-substituted or unsubstituted C₅-C₁₀ (e.g., C₅-C₆) aryl, or R^{28C}-substituted or unsubstituted 5 to 10 membered (e.g., 5 to 6 membered) heteroaryl.

R^{28C} may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{28D}-substituted or unsubstituted alkyl, R^{28D} -substituted or unsubstituted heteroalkyl, R^{28D}-substituted or unsubstituted cycloalkyl, R^{28D}-substituted or unsubstituted heterocycloalkyl, R^{28D}-substituted or unsubstituted aryl, or R^{28D}-substituted or unsubstituted heteroaryl. In embodiments, where R^{28C} is ═O or ═S, R^{28B} is not aryl or heteroaryl. In embodiments, R^{28C} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂. In other embodiments, R^{28C} is R^{28D}-substituted or unsubstituted alkyl, R^{28D}-substituted or unsubstituted heteroalkyl, R^{28D}-substituted or unsubstituted cycloalkyl, R^{28D}-substituted or unsubstituted heterocycloalkyl, R^{28D}-substituted or unsubstituted aryl, or R^{28D}-substituted or unsubstituted heteroaryl. R^{28C} may be R^{28D}-substituted or unsubstituted C₁-C₂₀ (e.g., C₁-C₆) alkyl, R^{28D}-substituted or unsubstituted 2 to 20 membered (e.g., 2 to 6 membered) heteroalkyl, R^{28D}-substituted or unsubstituted C₃-C₈ (e.g., C₅-C₇) cycloalkyl, R^{28D}-substituted or unsubstituted 3 to 8 membered (e.g., 3 to 6 membered) heterocycloalkyl, R^{28D}-substituted or unsubstituted C₅-C₁₀ (e.g., C₅-C₆) aryl, or R^{28D}-substituted or unsubstituted 5 to 10 membered (e.g., 5 to 6 membered) heteroaryl.

R^{28D} may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. In embodiments, where R^{28D} is ═O or ═S, R^{28C} is not aryl or heteroaryl. In embodiments, R^{28D} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂. In other embodiments, R^{28D} is unsubstituted alkyl, unsubstituted heteroalkyl, unsubstituted cycloalkyl, unsubstituted heterocycloalkyl, unsubstituted aryl, or unsubstituted heteroaryl. R^{28D} may be unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cycloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

R²⁹ may be independently hydrogen, halogen, —CXⁱ₃, —CN, —SO₂Cl, —SOₙ₉R⁴⁴, —SOᵥ₉NR⁴⁴R⁴⁵, —NHNH₂, —ONR⁴⁴R⁴⁵, —NHC═(O)NHNH₂, —NHC═(O)NR⁴⁴R⁴⁵, —NHC═(O)R⁴⁴, —N(O)ₘ₉, —NR⁴⁴R⁴⁵, —NH—O—R⁴⁴, —C(O)R⁴⁴, —C(O)—OR⁴⁴, —C(O)NR⁴⁴R⁴⁵, —N(R⁴⁴)C(O)R⁴⁵, —O—C(O)NR⁴⁴R⁴⁵, —OR⁴⁴, substituted or unsubstituted C₁-C₂₀ (e.g., C₁-C₆) alkyl, substituted or unsubstituted 2 to 20 membered (e.g., 2 to 6 membered) heteroalkyl, substituted or unsubstituted C₃-C₈ (e.g., C₅-C₇) cycloalkyl, substituted or unsubstituted 3 to 8 membered (e.g., 3 to 6 membered) heterocycloalkyl, substituted or unsubstituted C₅-C₁₀ (e.g., C₅-C₆) aryl, or substituted or unsubstituted 5 to 10 membered (e.g., 5 to 6 membered) heteroaryl.

In embodiments, R²⁹ is independently hydrogen, halogen, —CXⁱ₃, —CN, —SO₂Cl, —SOₙ₉R⁴⁴, —SOᵥ₉NR⁴⁴R⁴⁵, —NHNH₂, —ONR⁴⁴R⁴⁵, —NHC═(O)NHNH₂, —NHC═(O)NR⁴⁴R⁴⁵, —NHC═(O)R⁴⁴, —N(O)ₘ₉, —NR⁴⁴R⁴⁵, —NH—O—R⁴⁴, —C(O)R⁴⁴, —C(O)—OR⁴⁴, —C(O)NR⁴⁴R⁴⁵, —N(R⁴⁴)C(O)R⁴⁵, —O—C(O)NR⁴⁴R⁴⁵, —OR⁴⁴, R^{29A}-substituted or unsubstituted alkyl, R^{29A}-substituted or unsubstituted heteroalkyl, R^{29A}-substituted or unsubstituted cycloalkyl, R^{29A}-substituted or unsubstituted heterocycloalkyl, R^{29A}-substituted or unsubstituted aryl, or R^{29A}-substituted or unsubstituted heteroaryl. In embodiments, R²⁹ is independently hydrogen, halogen, —CXⁱ₃, —CN, —SO₂Cl, —SOₙ₉R⁴⁴, —SOᵥ₉NR⁴⁴R45, —NHNH₂, —ONR⁴⁴_{R}⁴⁵, —NHC═(O)NHNH₂, —NHC═(O)NR⁴⁴R⁴⁵, —NHC═(O)R⁴⁴, —N(O)ₘ₉, —NR⁴⁴R⁴⁵, —NH—O—R⁴⁴, —C(O)R⁴⁴, —C(O)—OR⁴⁴, —C(O)NR⁴⁴R⁴⁵, —N(R⁴⁴)C(O)R⁴⁵, —O—C(O)NR⁴⁴R⁴⁵, —OR⁴⁴. In embodiments, R²⁹ is R^{29A}-substituted or unsubstituted C₁-C₂₀ (e.g., C₁-C₆) alkyl, R^{29A}-substituted or unsubstituted 2 to 20 membered (e.g., 2 to 6 membered) heteroalkyl, R^{29A}-substituted or unsubstituted C₃-C₈ (e.g., C₅-C₇) cycloalkyl, R^{29A}-substituted or unsubstituted 3 to 8 membered (e.g., 3 to 6 membered) heterocycloalkyl, R^{29A}-substituted or unsubstituted C₅-C₁₀ (e.g., C₅-C₆) aryl, or R^{29A}-substituted or unsubstituted 5 to 10 membered (e.g., 5 to 6 membered) heteroaryl.

R^{29A} may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{29B}-substituted or unsubstituted alkyl, R^{29B}-substituted or unsubstituted heteroalkyl, R^{29B}-substituted or unsubstituted cycloalkyl, R^{29B}-substituted or unsubstituted heterocycloalkyl, R^{29B}-substituted or unsubstituted aryl, or R^{29B}-substituted or unsubstituted heteroaryl. In embodiments, where R^{29A} is ═O or ═S, R²⁹ is not aryl or heteroaryl. In embodiments, R^{29A} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂. In other embodiments, R^{29A} is R^{29B}-substituted or unsubstituted alkyl, R^{29B}-substituted or unsubstituted heteroalkyl, R^{29B}-substituted or unsubstituted cycloalkyl, R^{29B}-substituted or unsubstituted heterocycloalkyl, R^{29B}-substituted or unsubstituted aryl, or R^{29B}-substituted or unsubstituted heteroaryl. R^{29A} may be R^{29B}-substituted or unsubstituted C₁-C₂₀ (e.g., C₁-C₆) alkyl, R^{29B}-substituted or unsubstituted 2 to 20 membered (e.g., 2 to 6 membered) heteroalkyl, R^{29B}-substituted or unsubstituted C₃-C₈ (e.g., C₅-C₇) cycloalkyl, R^{29B}-substituted or unsubstituted 3 to 8 membered (e.g., 3 to 6 membered) heterocycloalkyl, R^{29B}-substituted or unsubstituted C₅-C₁₀ (e.g., C₅-C₆) aryl, or R^{29B}-substituted or unsubstituted 5 to 10 membered (e.g., 5 to 6 membered) heteroaryl.

R^{29B} may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{29C}-substituted or unsubstituted alkyl, R^{29C}-substituted or unsubstituted heteroalkyl, R^{29C}-substituted or unsubstituted cycloalkyl, R^{29C}-substituted or unsubstituted heterocycloalkyl, R^{29C}-substituted or unsubstituted aryl, or R^{29C}-substituted or unsubstituted heteroaryl. In embodiments, where R^{29B} is ═O or ═S, R^{29A} is not aryl or heteroaryl. In embodiments, R^{29B} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂. In other embodiments, R^{29B} is R^{29C}-substituted or unsubstituted alkyl, R^{29C}-substituted or unsubstituted heteroalkyl, R^{29C}-substituted or unsubstituted cycloalkyl, R^{29C}-substituted or unsubstituted heterocycloalkyl, R^{29C}-substituted or unsubstituted aryl, or R^{29C}-substituted or unsubstituted heteroaryl. R^{29B} may be R^{29C}-substituted or unsubstituted C₁-C₂₀ (e.g., C₁-C₆) alkyl, R^{29C}-substituted or unsubstituted 2 to 20 membered (e.g., 2 to 6 membered) heteroalkyl, R^{29C}-substituted or unsubstituted C₃-C₈ (e.g., C₅-C₇) cycloalkyl, R^{29C}-substituted or unsubstituted 3 to 8 membered (e.g., 3 to 6 membered) heterocycloalkyl, R^{29C}-substituted or unsubstituted C₅-C₁₀ (e.g., C₅-C₆) aryl, or R^{29C}-substituted or unsubstituted 5 to 10 membered (e.g., 5 to 6 membered) heteroaryl.

R^{29C} may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{29D}-substituted or unsubstituted alkyl, R^{29D}-substituted or unsubstituted heteroalkyl, R^{29D}-substituted or unsubstituted cycloalkyl, R^{29D}-substituted or unsubstituted heterocycloalkyl, R^{29D}-substituted or unsubstituted aryl, or R^{29D}-substituted or unsubstituted heteroaryl. In embodiments, where R^{29C} is ═O or ═S, R^{29B} is not aryl or heteroaryl. In embodiments, R^{29C} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂. In other embodiments, R^{29C} is R^{29D}-substituted or unsubstituted alkyl, R^{29D}-substituted or unsubstituted heteroalkyl, R^{29D}-substituted or unsubstituted cycloalkyl, R^{29D}-substituted or unsubstituted heterocycloalkyl, R^{29D}-substituted or unsubstituted aryl, or R^{29D}-substituted or unsubstituted heteroaryl. R^{29C} may be R^{29D}-substituted or unsubstituted C₁-C₂₀ (e.g., C₁-C₆) alkyl, R^{29D}-substituted or unsubstituted 2 to 20 membered (e.g., 2 to 6 membered) heteroalkyl, R^{29D}-substituted or unsubstituted C₃-C₈ (e.g., C₅-C₇) cycloalkyl, R^{29D}-substituted or unsubstituted 3 to 8 membered (e.g., 3 to 6 membered) heterocycloalkyl, R^{29D}-substituted or unsubstituted C₅-C₁₀ (e.g., C₅-C₆) aryl, or R^{29D}-substituted or unsubstituted 5 to 10 membered (e.g., 5 to 6 membered) heteroaryl.

R^{29D} may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. In embodiments, where R^{29D} is ═O or ═S, R^{29C} is not aryl or heteroaryl. In embodiments, R^{29D} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂. In other embodiments, R^{29D} is unsubstituted alkyl, unsubstituted heteroalkyl, unsubstituted cycloalkyl, unsubstituted heterocycloalkyl, unsubstituted aryl, or unsubstituted heteroaryl. R^{29D} may be unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cycloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

L³ may be independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted C₁-C₂₀ (e.g., C₁-C₆) alkylene, substituted or unsubstituted 2 to 20 membered (e.g., 2 to 6 membered) heteroalkylene, substituted or unsubstituted C₃-C₈ (e.g., C₅-C₇) cycloalkylene, substituted or unsubstituted 3 to 8 membered (e.g., 3 to 6 membered) heterocycloalkylene, substituted or unsubstituted C₅-C₁₀ (e.g., C₅-C₆) arylene, or substituted or unsubstituted 5 to 10 membered (e.g., 5 to 6 membered) heteroarylene.

In embodiments, L³ is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, R⁴⁶-substituted or unsubstituted alkylene, R⁴⁶-substituted or unsubstituted heteroalkylene, R⁴⁶-substituted or unsubstituted cycloalkylene, R⁴⁶-substituted or unsubstituted heterocycloalkylene, R⁴⁶-substituted or unsubstituted arylene, or R⁴⁶-substituted or unsubstituted heteroarylene. In embodiments, L³ is independently a bond, —S(O)—, —S(O)₂NH—, —NHS(O)₂—, —C(O)O—, —OC(O)—, —C(O)—, —C(O)NH—, —NH—, —NHC(O)—, —O—, or —S—. In embodiments, L³ is independently R⁴⁶-substituted or unsubstituted alkylene, R⁴⁶-substituted or unsubstituted heteroalkylene, R⁴⁶-substituted or unsubstituted cycloalkylene, R⁴⁶-substituted or unsubstituted heterocycloalkylene, R⁴⁶-substituted or unsubstituted arylene, or R⁴⁶-substituted or unsubstituted heteroarylene. L³ may be independently R⁴⁶-substituted or unsubstituted C₁-C₂₀ (e.g., C₁-C₆) alkylene, R⁴⁶-substituted or unsubstituted 2 to 20 membered (e.g., 2 to 6 membered) heteroalkylene, R⁴⁶-substituted or unsubstituted C₃-C₈ (e.g., C₅-C₇) cycloalkylene, R⁴⁶-substituted or unsubstituted 3 to 8 membered (e.g., 3 to 6 membered) heterocycloalkylene, R⁴⁶-substituted or unsubstituted C₅-C₁₀ (e.g., C₅-C₆) arylene, or R⁴⁶-substituted or unsubstituted 5 to 10 membered (e.g., 5 to 6 membered) heteroarylene.

R⁴⁶ may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R⁴⁷-substituted or unsubstituted alkyl, R⁴⁷-substituted or unsubstituted heteroalkyl, R⁴⁷-substituted or unsubstituted cycloalkyl, R⁴⁷-substituted or unsubstituted heterocycloalkyl, R⁴⁷-substituted or unsubstituted aryl, or R⁴⁷-substituted or unsubstituted heteroaryl. In embodiments, where R⁴⁶ is ═O or ═S, L³ is not arylene or heteroarylene. In embodiments, R⁴⁶ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂ or —NHC═(O)NHNH₂. In embodiments, R⁴⁶ is independently R⁴⁷-substituted or unsubstituted alkyl, R⁴⁷-substituted or unsubstituted heteroalkyl, R⁴⁷-substituted or unsubstituted cycloalkyl, R⁴⁷-substituted or unsubstituted heterocycloalkyl, R⁴⁷-substituted or unsubstituted aryl, or R⁴⁷-substituted or unsubstituted heteroaryl. R⁴⁶ may be independently R⁴⁷-substituted or unsubstituted C₁-C₂₀ (e.g., C₁-C₆) alkyl, R⁴⁷-substituted or unsubstituted 2 to 20 membered (e.g., 2 to 6 membered) heteroalkyl, R⁴⁷-substituted or unsubstituted C₃-C₈ (e.g., C₅-C₇) cycloalkyl, R⁴⁷-substituted or unsubstituted 3 to 8 membered (e.g., 3 to 6 membered) heterocycloalkyl, R⁴⁷-substituted or unsubstituted C₅-C₁₀ (e.g., C₅-C₆) aryl, or R⁴⁷-substituted or unsubstituted 5 to 10 membered (e.g., 5 to 6 membered) heteroaryl.

R⁴⁷ may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R⁴⁸-substituted or unsubstituted alkyl, R⁴⁸-substituted or unsubstituted heteroalkyl, R⁴⁸-substituted or unsubstituted cycloalkyl, R⁴⁸-substituted or unsubstituted heterocycloalkyl, R⁴⁸-substituted or unsubstituted aryl, or R⁴⁸-substituted or unsubstituted heteroaryl. In embodiments, where R⁴⁷ is ═O or ═S, R⁴⁶ is not aryl or heteroaryl. In embodiments, R⁴⁷ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂ or —NHC—(O)NHNH₂. In embodiments, R⁴⁷ is independently R⁴⁸-substituted or unsubstituted alkyl, R⁴⁸-substituted or unsubstituted heteroalkyl, R⁴⁸-substituted or unsubstituted cycloalkyl, R⁴⁸-substituted or unsubstituted heterocycloalkyl, R⁴⁸-substituted or unsubstituted aryl, or R⁴⁸-substituted or unsubstituted heteroaryl. R⁴⁷ may be independently R⁴⁸-substituted or unsubstituted C₁-C₂₀ (e.g., C₁-C₆) alkyl, R⁴⁸-substituted or unsubstituted 2 to 20 membered (e.g., 2 to 6 membered) heteroalkyl, R⁴⁸-substituted or unsubstituted C₃-C₈ (e.g., C₅-C₇) cycloalkyl, R⁴⁸-substituted or unsubstituted 3 to 8 membered (e.g., 3 to 6 membered) heterocycloalkyl, R⁴⁸-substituted or unsubstituted C₅-C₁₀ (e.g., C₅-C₆) aryl, or R⁴⁸-substituted or unsubstituted 5 to 10 membered (e.g., 5 to 6 membered) heteroaryl.

R⁴⁸ may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R⁴⁹-substituted or unsubstituted alkyl, R⁴⁹-substituted or unsubstituted heteroalkyl, R⁴⁹-substituted or unsubstituted cycloalkyl, R⁴⁹-substituted or unsubstituted heterocycloalkyl, R⁴⁹-substituted or unsubstituted aryl, or R⁴⁹-substituted or unsubstituted heteroaryl. In embodiments, where R⁴⁸ is ═O or ═S, R⁴⁷ is not aryl or heteroaryl. In embodiments, R⁴⁸ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂ or —NHC═(O)NHNH₂. In embodiments, R⁴⁸ is independently R⁴⁹-substituted or unsubstituted alkyl, R⁴⁹-substituted or unsubstituted heteroalkyl, R⁴⁹-substituted or unsubstituted cycloalkyl, R⁴⁹-substituted or unsubstituted heterocycloalkyl, R⁴⁹-substituted or unsubstituted aryl, or R⁴⁹-substituted or unsubstituted heteroaryl. R⁴⁸ may be independently R⁴⁹-substituted or unsubstituted C₁-C₂₀ (e.g., C₁-C₆) alkyl, R⁴⁹-substituted or unsubstituted 2 to 20 membered (e.g., 2 to 6 membered) heteroalkyl, R⁴⁹-substituted or unsubstituted C₃-C₈ (e.g., C₅-C₇) cycloalkyl, R⁴⁹-substituted or unsubstituted 3 to 8 membered (e.g., 3 to 6 membered) heterocycloalkyl, R⁴⁹-substituted or unsubstituted C₅-C₁₀ (e.g., C₅-C₆) aryl, or R⁴⁹-substituted or unsubstituted 5 to 10 membered (e.g., 5 to 6 membered) heteroaryl.

R⁴⁹ may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R⁵⁰-substituted or unsubstituted alkyl, R⁵⁰-substituted or unsubstituted heteroalkyl, R⁵⁰-substituted or unsubstituted cycloalkyl, R⁵⁰-substituted or unsubstituted heterocycloalkyl, R⁵⁰-substituted or unsubstituted aryl, or R⁵⁰-substituted or unsubstituted heteroaryl. In embodiments, where R⁴⁹ is ═O or ═S, R⁴⁸ is not aryl or heteroaryl. In embodiments, R⁴⁹ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂ or —NHC═(O)NHNH₂. In embodiments, R⁴⁹ is independently R⁵⁰-substituted or unsubstituted alkyl, R⁵⁰-substituted or unsubstituted heteroalkyl, R⁵⁰-substituted or unsubstituted cycloalkyl, R⁵⁰-substituted or unsubstituted heterocycloalkyl, R⁵⁰-substituted or unsubstituted aryl, or R⁵⁰-substituted or unsubstituted heteroaryl. R⁴⁹ may be independently R⁵⁰-substituted or unsubstituted C₁-C₂₀ (e.g., C₁-C₆) alkyl, R⁵⁰-substituted or unsubstituted 2 to 20 membered (e.g., 2 to 6 membered) heteroalkyl, R⁵⁰-substituted or unsubstituted C₃-C₈ (e.g., C₅-C₇) cycloalkyl, R⁵⁰-substituted or unsubstituted 3 to 8 membered (e.g., 3 to 6 membered) heterocycloalkyl, R⁵⁰-substituted or unsubstituted C₅-C₁₀ (e.g., C₅-C₆) aryl, or R⁵⁰-substituted or unsubstituted 5 to 10 membered (e.g., 5 to 6 membered) heteroaryl.

R⁵⁰ may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. In embodiments, where R⁵⁰ is ═O or ═S, R⁴⁹ is not aryl or heteroaryl. In embodiments, R⁵⁰ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂ or —NHC═(O)NHNH₂. In embodiments, R⁵⁰ is independently unsubstituted alkyl, unsubstituted heteroalkyl, unsubstituted cycloalkyl, unsubstituted heterocycloalkyl, unsubstituted aryl, or unsubstituted heteroaryl. R⁵⁰ may be independently unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cycloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

In embodiments, L³ is independently R⁴⁶-substituted or unsubstituted alkylene, R⁴⁶-substituted or unsubstituted heteroalkylene, R⁴⁶-substituted or unsubstituted cycloalkylene, R⁴⁶-substituted or unsubstituted heterocycloalkylene, R⁴⁶-substituted or unsubstituted arylene, or R⁴⁶-substituted or unsubstituted heteroarylene. R⁴⁶ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R⁴⁷-substituted or unsubstituted alkyl, R⁴⁷-substituted or unsubstituted heteroalkyl, R⁴⁷-substituted or unsubstituted cycloalkyl, R⁴⁷-substituted or unsubstituted heterocycloalkyl, R⁴⁷-substituted or unsubstituted aryl, or R⁴⁷-substituted or unsubstituted heteroaryl. R⁴⁷ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R⁴⁸-substituted or unsubstituted alkyl, R⁴⁸-substituted or unsubstituted heteroalkyl, R⁴⁸-substituted or unsubstituted cycloalkyl, R⁴⁸-substituted or unsubstituted heterocycloalkyl, R⁴⁸-substituted or unsubstituted aryl, or R⁴⁸-substituted or unsubstituted heteroaryl. R⁴⁸ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R⁴⁹-substituted or unsubstituted alkyl, R⁴⁹-substituted or unsubstituted heteroalkyl, R⁴⁹-substituted or unsubstituted cycloalkyl, R⁴⁹-substituted or unsubstituted heterocycloalkyl, R⁴⁹-substituted or unsubstituted aryl, or R⁴⁹-substituted or unsubstituted heteroaryl. R⁴⁹ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R⁵⁰-substituted or unsubstituted alkyl, R⁵⁰-substituted or unsubstituted heteroalkyl, R⁵⁰-substituted or unsubstituted cycloalkyl, R⁵⁰-substituted or unsubstituted heterocycloalkyl, R⁵⁰-substituted or unsubstituted aryl, or R⁵⁰-substituted or unsubstituted heteroaryl. And R⁵⁰ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl.

In embodiments, R³⁰, R³¹, R³², R³³, R³⁴, R³⁵ R³⁶, R³⁷, R³⁸, R³⁹, R⁴⁰, R⁴¹, R⁴², R⁴³, R⁴⁴ and R⁴⁵ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂ or —NHC═(O)NHNH₂. In embodiments, R³⁰, R³¹, R³², R³³, R³⁴, R³⁵ R³⁶, R³⁷, R³⁸, R39, R⁴⁰, R⁴¹, R⁴², R⁴³, R⁴⁴ and R⁴⁵ are independently substituted or unsubstituted C₁-C₂₀ (e.g., C₁-C₆) alkyl, substituted or unsubstituted 2 to 20 membered (e.g., 2 to 6 membered) heteroalkyl, substituted or unsubstituted C₃-C₈ (e.g., C₅-C₇) cycloalkyl, substituted or unsubstituted 3 to 8 membered (e.g., 3 to 6 membered) heterocycloalkyl, substituted or unsubstituted C₅-C₁₀ (e.g., C₅-C₆) aryl, or substituted or unsubstituted 5 to 10 membered (e.g., 5 to 6 membered) heteroaryl.

In embodiments, R³⁰, R³¹, R³², R³³, R³⁴, R³⁵ R³⁶, R³⁷, R³⁸, R³⁹, R⁴⁰, R⁴¹, R⁴², R⁴³, R⁴⁴ and R⁴⁵ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R⁵⁵-substituted or unsubstituted alkyl, R⁵⁵-substituted or unsubstituted heteroalkyl, R⁵⁵-substituted or unsubstituted cycloalkyl, R⁵⁵-substituted or unsubstituted heterocycloalkyl, R⁵⁵-substituted or unsubstituted aryl, or R⁵⁵-substituted or unsubstituted heteroaryl. In embodiments, R³⁰, R³¹, R³², R³³, R³⁴, R³⁵ R³⁶, R³⁷, R³⁸, R³⁹, R⁴⁰, R⁴¹, R⁴², R⁴³, R⁴⁴ and R⁴⁵ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂ or —NHC═(O)NHNH₂. In embodiments, R³⁰, R³¹, R³², R³³, R³⁴, R³⁵ R³⁶, R³⁷, R³⁸, R³⁹, R⁴⁰, R⁴¹, R⁴², R⁴³, R44 and R⁴⁵ are independently R⁵⁵-substituted or unsubstituted alkyl, R⁵⁵-substituted or unsubstituted heteroalkyl, R⁵⁵-substituted or unsubstituted cycloalkyl, R⁵⁵-substituted or unsubstituted heterocycloalkyl, R⁵⁵-substituted or unsubstituted aryl, or R⁵⁵-substituted or unsubstituted heteroaryl. R³⁰, R³¹, R³², R³³, R³⁴, R³⁵ R³⁶, R³⁷, R³⁸, R³⁹, R⁴⁰, R⁴¹, R⁴², R⁴³, R44 and R⁴⁵ may be independently R⁵⁵-substituted or unsubstituted C₁-C₂₀ (e.g., C₁-C₆) alkyl, R⁵⁵-substituted or unsubstituted 2 to 20 membered (e.g., 2 to 6 membered) heteroalkyl, R⁵⁵-substituted or unsubstituted C₃-C₈ (e.g., C₅-C₇) cycloalkyl, R⁵⁵-substituted or unsubstituted 3 to 8 membered (e.g., 3 to 6 membered) heterocycloalkyl, R⁵⁵-substituted or unsubstituted C₅-C₁₀ (e.g., C₅-C₆) aryl, or R⁵⁵-substituted or unsubstituted 5 to 10 membered (e.g., 5 to 6 membered) heteroaryl.

R⁵⁵ as provided herein may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R⁵⁶-substituted or unsubstituted alkyl, R⁵⁶-substituted or unsubstituted heteroalkyl, R⁵⁶-substituted or unsubstituted cycloalkyl, R⁵⁶-substituted or unsubstituted heterocycloalkyl, R⁵⁶-substituted or unsubstituted aryl, or R⁵⁶-substituted or unsubstituted heteroaryl. In embodiments, R⁵⁵is independently hydrogen, halogen, ═O, ═S, —CF₃, —CN, —CCl₃, —COOH, —CH₂COOH, —CONH₂, —OH, —SH, —SO₂Cl, —SO₃, —SO₄H, —SO₂NH₂, —NO₂, —NH₂, —NHNH₂, —ONH₂ or —NHC═(O)NHNH₂. In embodiments, where R⁵⁵ is ═O or ═S, R³⁰, R³¹, R³², R³³, R³⁴, R³⁵ R³⁶, R³⁷, R³⁸, R³⁹, R⁴⁰, R⁴¹, R⁴², R⁴³, R⁴⁴and R⁴⁵ is not aryl or heteroaryl. In embodiments, R⁵⁵ is independently R⁵⁶-substituted or unsubstituted alkyl, R⁵⁶-substituted or unsubstituted heteroalkyl, R⁵⁶-substituted or unsubstituted cycloalkyl, R⁵⁶-substituted or unsubstituted heterocycloalkyl, R⁵⁶-substituted or unsubstituted aryl, or R⁵⁶-substituted or unsubstituted heteroaryl. R⁵⁵ may be independently R⁵⁶-substituted or unsubstituted C₁-C₂₀ (e.g., C₁-C₆) alkyl, R⁵⁶-substituted or unsubstituted 2 to 20 membered (e.g., 2 to 6 membered) heteroalkyl, R⁵⁶-substituted or unsubstituted C₃-C₈ (e.g., C₅-C₇) cycloalkyl, R⁵⁶-substituted or unsubstituted 3 to 8 membered (e.g., 3 to 6 membered) heterocycloalkyl, R⁵⁶-substituted or unsubstituted C₅-C₁₀ (e.g., C₅-C₆) aryl, or R⁵⁶-substituted or unsubstituted 5 to 10 membered (e.g., 5 to 6 membered) heteroaryl.

R⁵⁶ as provided herein may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R⁵⁷-substituted or unsubstituted alkyl, R⁵⁷-substituted or unsubstituted heteroalkyl, R⁵⁷-substituted or unsubstituted cycloalkyl, R⁵⁷-substituted or unsubstituted heterocycloalkyl, R⁵⁷-substituted or unsubstituted aryl, or R⁵⁷-substituted or unsubstituted heteroaryl. In embodiments, R⁵⁶is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂ or —NHC═(O)NHNH₂. In embodiments, where R⁵⁶ is ═O or ═S, R⁵⁵ is not aryl or heteroaryl. In embodiments, R⁵⁶ is independently R⁵⁷-substituted or unsubstituted alkyl, R⁵⁷-substituted or unsubstituted heteroalkyl, R⁵⁷-substituted or unsubstituted cycloalkyl, R⁵⁷-substituted or unsubstituted heterocycloalkyl, R⁵⁷-substituted or unsubstituted aryl, or R⁵⁷-substituted or unsubstituted heteroaryl. R⁵⁶ may be independently R⁵⁷-substituted or unsubstituted C₁-C₂₀ (e.g., C₁-C₆) alkyl, R⁵⁷-substituted or unsubstituted 2 to 20 membered (e.g., 2 to 6 membered) heteroalkyl, R⁵⁷-substituted or unsubstituted C₃-C₈ (e.g., C₅-C₇) cycloalkyl, R⁵⁷-substituted or unsubstituted 3 to 8 membered (e.g., 3 to 6 membered) heterocycloalkyl, R⁵⁷-substituted or unsubstituted C₅-C₁₀ (e.g., C₅-C₆) aryl, or R⁵⁷-substituted or unsubstituted 5 to 10 membered (e.g., 5 to 6 membered) heteroaryl.

R⁵⁷ as provided herein may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R⁵⁸-substituted or unsubstituted alkyl, R⁵⁸-substituted or unsubstituted heteroalkyl, R⁵⁸-substituted or unsubstituted cycloalkyl, R⁵⁸-substituted or unsubstituted heterocycloalkyl, R⁵⁸-substituted or unsubstituted aryl, or R⁵⁸-substituted or unsubstituted heteroaryl. In embodiments, R⁵⁷ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂ or —NHC═(O)NHNH₂. In embodiments, where R⁵⁷ is ═O or ═S, R⁵⁶ is not aryl or heteroaryl. In embodiments, R⁵⁷ is independently R⁵⁸-substituted or unsubstituted alkyl, R⁵⁸-substituted or unsubstituted heteroalkyl, R⁵⁸-substituted or unsubstituted cycloalkyl, R⁵⁸-substituted or unsubstituted heterocycloalkyl, R⁵⁸-substituted or unsubstituted aryl, or R⁵⁸-substituted or unsubstituted heteroaryl. R⁵⁷ may be independently R⁵⁸-substituted or unsubstituted C₁-C₂₀ (e.g., C₁-C₆) alkyl, R⁵⁸-substituted or unsubstituted 2 to 20 membered (e.g., 2 to 6 membered) heteroalkyl, R⁵⁸-substituted or unsubstituted C₃-C₈ (e.g., C₅-C₇) cycloalkyl, R⁵⁸-substituted or unsubstituted 3 to 8 membered (e.g., 3 to 6 membered) heterocycloalkyl, R⁵⁸-substituted or unsubstituted C₅-C₁₀ (e.g., C₅-C₆) aryl, or R⁵⁸-substituted or unsubstituted 5 to 10 membered (e.g., 5 to 6 membered) heteroaryl.

R⁵⁸ as provided herein may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl or unsubstituted heteroaryl. In embodiments, R⁵⁸ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂ or —NHC═(O)NHNH₂. In embodiments, where R⁵⁸ is ═O or ═S, R⁵⁷ is not aryl or heteroaryl. In embodiments, R⁵⁸ is independently unsubstituted alkyl, unsubstituted heteroalkyl, unsubstituted cycloalkyl, unsubstituted heterocycloalkyl, unsubstituted aryl or unsubstituted heteroaryl. R⁵⁸ may be independently unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cycloalkyl, unsubstituted 3 to 8 membered (e.g., 3 to 6 membered) heterocycloalkyl, unsubstituted C₅-C₁₀ (e.g., C₅-C₆) aryl or unsubstituted 5 to 10 membered (e.g., 5 to 6 membered) heteroaryl.

In embodiments, the compound of formula (VI) including embodiments thereof may include multiple instances of R⁵⁵, R⁵⁶, R⁵⁷, and/or R⁵⁸ (e.g., R²² may be —ONR³⁰R³¹ and R³⁰ and R³¹ may be independently R⁵⁵-substituted). In such embodiments, each variable may optional be different and be appropriately labeled to distinguish each group for greater clarity. For example, where each R⁵⁵, R⁵⁶, R⁵⁷, and/or R⁵⁸ is different, they may be referred to, for example as R^{55.1}, R^{55.2}, R^{55.3}, R^{55.4}, R^{56.1}, R^{56.2}, R^{56.3}, R^{56.4}, R^{57.1}, R^{57.2}, R^{57.3}, R^{57.4}, R^{58.1}, R^{58.2}, R^{58.3}, and/or R^{58.4}, respectively, wherein the definition of R⁵⁵ is assumed by R^{55.1}, R^{55.2}, R^{55.3}, and/or R^{55.4}, the definition of R⁵⁶ is assumed by R^{56.1}, R^{56.2}, R^{56.3}, and/or R^{56.4}, the definition of R⁵⁷ is assumed by R^{57.1}, R^{57.2}, R^{57.3}, and/or R^{57.4}, the definition of R⁵⁸ is assumed by R^{58.1}, R^{58.2}, R^{58.3}, and/or R^{58.4}. The variables used within a definition of R⁵⁵, R⁵⁶, R⁵⁷, and/or R⁵⁸ and/or other variables that appear at multiple instances and are different may similarly be appropriately labeled to distinguish each group for greater clarity.

In formula (VI) R²¹ is independently hydrogen, halogen, —CX^{a}₃, —CN, —SO₂Cl, —SOₙ₁R²⁸, —SOᵥ₁NR²⁸R²⁹, —NHNH₂, —ONR²⁸R²⁹, —NHC═(O)NHNH₂, —NHC═(O)NR²⁸R²⁹, —NHC═(O)R²⁸, —N(O)ₘ₁, —NR²⁸R²⁹, —NH—O—R²⁸, —C(O)R²⁸, —C(O)—OR²⁸, —C(O)NR²⁸R²⁹, —N(R²⁸)C(O)R²⁹, —O—R²⁸, —O—C(O)NR²⁸R²⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R²¹ is substituted or unsubstituted (e.g., C₁-C₈) alkyl, substituted or unsubstituted (e.g., 2 to 10 membered) heteroalkyl, substituted or unsubstituted (e.g., C₅-C₆) aryl, substituted or unsubstituted (e.g., 5 to 10 membered) heteroaryl, —SOᵥ₁NR²⁸R²⁹, —NHC═(O)R²⁸, —NR²⁸R²⁹, —C(O)NR²⁸R²⁹, or —O—C(O)NR²⁸R²⁹.

In embodiments, R²¹ is substituted or unsubstituted C₁-C₈ alkyl. In embodiments, R²¹ is substituted or unsubstituted C₁-C₇ alkyl. In embodiments, R²¹ is substituted or unsubstituted C₁-C₆ alkyl. In embodiments, R²¹ is substituted or unsubstituted C₁-C₅ alkyl. In embodiments, R²¹ is substituted or unsubstituted C₁-C₄ alkyl. In embodiments, R²¹ is substituted or unsubstituted C₁-C₃ alkyl. In embodiments, R²¹ is substituted or unsubstituted ethyl or methyl. In embodiments, R²¹ is R^{21A}-substituted or unsubstituted C₁-C₈ alkyl. In embodiments, R²¹ is R^{21A}-substituted or unsubstituted C₁-C₇ alkyl. In embodiments, R²¹ is R^{21A}-substituted or unsubstituted C₁-C₆ alkyl. In embodiments, R²¹ is R^{21A}-substituted or unsubstituted C₁-C₅ alkyl. In embodiments, R²¹ is R^{21A}-substituted or unsubstituted C₁-C₄ alkyl. In embodiments, R²¹ is R^{21A}-substituted or unsubstituted C₁-C₃ alkyl. In embodiments, R²¹ is R^{21A}-substituted or unsubstituted ethyl or methyl.

In embodiments, R²¹ is R^{21A}-substituted or unsubstituted branched C₁-C₈ alkyl. In embodiments, R²¹ is R^{21A}-substituted or unsubstituted branched C₁-C₇ alkyl. In embodiments, R²¹ is R^{21A}-substituted or unsubstituted branched C₁-C₆ alkyl. In embodiments, R²¹ is R^{21A}-substituted or unsubstituted branched C₁-C₅ alkyl. In embodiments, R²¹ is R^{21A}-substituted or unsubstituted branched C₁-C₄ alkyl. In embodiments, R²¹ is R^{21A}-substituted or unsubstituted branched C₁-C₃ alkyl. In embodiments, R²¹ is unsubstituted branched C₁-C₈ alkyl. In embodiments, R²¹ is unsubstituted branched C₁-C₇ alkyl. In embodiments, R²¹ is unsubstituted branched C₁-C₆ alkyl. In embodiments, R²¹ is unsubstituted branched C₁-C₅ alkyl. In embodiments, R²¹ is unsubstituted branched C₁-C₄ alkyl. In embodiments, R²¹ is unsubstituted branched C₁-C₃ alkyl. In embodiments, R²¹ is unsubstituted branched C₅ alkyl.

In embodiments, R²¹ is R^{21A}-substituted C₁-C₃ alkyl. In embodiments, R21 is R^{21A}-substituted methyl. In embodiments, R^{21A} is R^{21B}-substituted or unsubstituted 5 to 10 membered heterocycloalkyl or R^{21B}-substituted or unsubstituted 5 to 10 membered heteroaryl. In embodiments, R^{21A} is R^{21B}-substituted or unsubstituted 5 to 10 membered heterocycloalkyl. In embodiments, R^{21A} is R^{21B}-substituted or unsubstituted 5 to 10 membered heteroaryl. In embodiments, R^{21A} is R^{21B}-substituted or unsubstituted 5 membered heteroaryl. In embodiments, R^{21A} is R^{21B}-substituted or unsubstituted 6 membered heteroaryl. In embodiments, R^{21A} is R^{21B}-substituted or unsubstituted 10 membered heteroaryl. In embodiments, R^{21A} is R^{21B}-substituted 10 membered heteroaryl. In embodiments, R^{21B} is independently NH₂ or C₁-C₈ alkyl. In embodiments, R^{21B} is independently —NH₂ or C₁-C₅ alkyl. In embodiments, R^{21B} is independently —NH₂ or C₁-C₃ alkyl. In embodiments, R^{21B} is independently —NH₂ or methyl. In embodiments, R^{21A} is R^{21B}-substituted or unsubstituted 5 to 10 membered heterocycloalkyl. In embodiments, R^{21A} is R^{21B}-substituted or unsubstituted 5 membered heterocycloalkyl. In embodiments, R^{21A} is R^{21B}-substituted or unsubstituted 6 membered heterocycloalkyl. In embodiments, R^{21A} is R^{21B}-substituted or unsubstituted 10 membered heterocycloalkyl. In embodiments, R^{21A} is unsubstituted 5 to 10 membered heterocycloalkyl. In embodiments, R^{21A} is unsubstituted 5 membered heterocycloalkyl. In embodiments, R^{21A} is unsubstituted 6 membered heterocycloalkyl. In embodiments, R^{21A} is unsubstituted 10 membered heterocycloalkyl.

In embodiments, R²¹ is R^{21A}-substituted C₁-C₃ alkyl. In embodiments, R²¹ is R^{21A}-substituted ethyl. In embodiments, R^{21A} is R^{21B}-substituted or unsubstituted 5 to 10 membered heterocycloalkyl. In embodiments, R^{21A} is R^{21B}-substituted or unsubstituted 5 membered heterocycloalkyl. In embodiments, R^{21A} is R^{21B}-substituted or unsubstituted 6 membered heterocycloalkyl. In embodiments, R^{21A} is R^{21B}-substituted 5 membered heterocycloalkyl. In embodiments, R^{21A} is R^{21B}-substituted 6 membered heterocycloalkyl. In embodiments, R^{21B} is R^{21C}-substituted or unsubstituted C₁-C₈ alkyl. In embodiments, R^{21B} is R^{21C}-substituted or unsubstituted C₁-C₅ alkyl. In embodiments, R^{21B} is R^{21C}-substituted or unsubstituted C₁-C₃ alkyl. In embodiments, R^{21B} is unsubstituted C₁-C₈ alkyl. In embodiments, R^{21B} is unsubstituted C₁-C₅ alkyl. In embodiments, R^{21B} is unsubstituted C₁-C₃ alkyl. In embodiments, R^{21B} is unsubstituted C₃ alkyl.

In embodiments, R²¹ is substituted or unsubstituted 2 to 10 membered heteroalkyl. In embodiments, R²¹ is substituted or unsubstituted 2 to 8 membered heteroalkyl. In embodiments, R²¹ is substituted or unsubstituted 2 to 6 membered heteroalkyl. In embodiments, R²¹ is substituted or unsubstituted 2 to 4 membered heteroalkyl. In embodiments, R²¹ is substituted or unsubstituted 4 to 10 membered heteroalkyl. In embodiments, R²¹ is substituted or unsubstituted 4 to 8 membered heteroalkyl. In embodiments, R²¹ is substituted or unsubstituted 4 to 6 membered heteroalkyl. In embodiments, R²¹ is substituted or unsubstituted 6 to 10 membered heteroalkyl. In embodiments, R²¹ is substituted or unsubstituted 6 to 8 membered heteroalkyl. In embodiments, R²¹ is substituted or unsubstituted 2 membered heteroalkyl. In embodiments, R²¹ is substituted or unsubstituted 3 membered heteroalkyl. In embodiments, R²¹ is substituted or unsubstituted 4 membered heteroalkyl. In embodiments, R²¹ is substituted or unsubstituted 5 membered heteroalkyl. In embodiments, R²¹ is substituted or unsubstituted 6 membered heteroalkyl. In embodiments, R²¹ is substituted or unsubstituted 7 membered heteroalkyl. In embodiments, R²¹ is substituted or unsubstituted 8 membered heteroalkyl. In embodiments, R²¹ is substituted or unsubstituted 9 membered heteroalkyl. In embodiments, R²¹ is substituted or unsubstituted 10 membered heteroalkyl.

In embodiments, R²¹ is substituted or unsubstituted 4 to 8 membered heteroalkyl. In embodiments, R^{21A} is independently C₁-C₅ alkyl or ═O. In embodiments, R^{21A} is independently C₁-C₃ alkyl or ═O. In embodiments, R^{21A} is independently methyl or ═O.

In embodiments, R²¹ is substituted or unsubstituted C₅-C₆ aryl. In embodiments, R²¹ is substituted or unsubstituted phenyl. In embodiments, R²¹ is R^{21A}-substituted or unsubstituted aryl. In embodiments, R²¹ is R^{21A}-substituted (e.g., C₅-C₆) aryl. In embodiments, R^{21A} is halogen. In embodiments, R²¹ is R^{21A}-substituted phenyl and R^{21A} is —Cl.

In embodiments, R²¹ is substituted (e.g., R^{21A}-substituted) or unsubstituted 5 to 10 membered heteroaryl. In embodiments, R²¹ is substituted or unsubstituted 5 membered heteroaryl. In embodiments, R²¹ is substituted or unsubstituted 6 membered heteroaryl. In embodiments, R²¹ is substituted or unsubstituted 10 membered heteroaryl. In embodiments, R²¹ is R^{21A}-substituted or unsubstituted (e.g., 5 to 10 membered) heteroaryl. In embodiments, R²¹ is R^{21A}-substituted 5 membered heteroaryl. In embodiments, R²¹ is R^{21A}-substituted 6 membered heteroaryl. In embodiments, R²¹ is R^{21A}-substituted 10 membered heteroaryl. In embodiments, R^{21A} is substituted or unsubstituted C₁-C₅ alkyl. In embodiments, R^{21A} is substituted or unsubstituted C₁-C₃ alkyl. In embodiments, R^{21A} unsubstituted C₁-C₅ alkyl. In embodiments, R^{21A} is unsubstituted C₁-C₃ alkyl. In embodiments, R^{21A} is unsubstituted methyl or ethyl. In embodiments, R^{21A} is C₁-C₅ alkyl. In embodiments, R²¹ is R^{21A}-substituted 5 membered heteroaryl and R^{21A} is ethyl. In embodiments, R²¹ is R^{21A}-substituted 5 membered heteroaryl and R^{21A} is unsubstituted ethyl. In embodiments, R²¹ is R^{21A}-substituted 5 membered heteroaryl and R^{21A} is substituted ethyl. In embodiments, R²¹ is R^{21A}-substituted 5 membered heteroaryl and R^{21A} is unsubstituted C₁-C₅ alkyl. In embodiments, R²¹ is R^{21A}-substituted 5 membered heteroaryl and R^{21A} is substituted C₁-C₅ alkyl.

In embodiments, R²¹ is SOᵥ₁NR²⁸R²⁹, —NHC═(O)R²⁸, —NR²⁸R²⁹, —C(O)NR²⁸R²⁹, or —O—C(O)NR²⁸R²⁹. In embodiments, R²¹ is SOᵥ₁NR²⁸R²⁹. In embodiments, v₁ is 2 and R²⁸ and R²⁹ are independently hydrogen. In embodiments, R²¹ is SO₂NH₂.

In embodiments, R²¹ is —NHC═(O)R²⁸. In embodiments, R28 is substituted (e.g., R^{28A}-substituted) or unsubstituted 5 to 10 membered heteroaryl. In embodiments, R²⁸ is substituted or unsubstituted 5 membered heteroaryl. In embodiments, R²⁸ is substituted or unsubstituted 6 membered heteroaryl. In embodiments, R²⁸ is substituted or unsubstituted 8 membered heteroaryl. In embodiments, R²⁸ is unsubstituted indolyl.

In embodiments, R²¹ is —NR²⁸R²⁹. In embodiments, R²⁸ and R²⁹ are independently hydrogen, substituted (e.g., R^{28A}-substituted or R^{29A}-substituted) or unsubstituted (e.g., 5 to 10 membered) heterocycloalkyl or substituted (e.g., R ^{28A}-substituted or R^{29A}-substituted) or unsubstituted (5 to 10 membered) heteroaryl. In embodiments, R²⁸ is hydrogen and R²⁹ is substituted or unsubstituted 6 membered heterocycloalkyl. In embodiments, R²⁸ is hydrogen and R²⁹ is unsubstituted piperazinyl.

In embodiments, R²¹ is —NR²⁸R²⁹. In embodiments, R²⁸ is hydrogen and R²⁹ is substituted (e.g., R^{29A}-substituted) or unsubstituted 5 to 10 membered heteroaryl. In embodiments, R²⁸ is hydrogen and R²⁹ is R^{29A}-substituted or unsubstituted 5 membered heteroaryl. In embodiments, R²⁸ is hydrogen and R²⁹ is R^{29A}-substituted or unsubstituted 6 membered heteroaryl. In embodiments, R²⁸ is hydrogen and R²⁹ is R^{29A}-substituted or unsubstituted 10 membered heteroaryl. In embodiments, R²⁸ is hydrogen and R²⁹ is unsubstituted 10 membered heteroaryl. In embodiments, R²⁸ is hydrogen and R²⁹ is R^{29A}-substituted 10 membered heteroaryl. In embodiments, R²⁸ is hydrogen and R²⁹ is unsubstituted quinolinyl. In embodiments, R²⁸ is hydrogen and R²⁹ is R^{29A}-substituted quinolinyl. In embodiments, R^{29A} is halogen. In embodiments, R^{29A} is —Cl. In embodiments, R²⁸ is hydrogen, R²⁹ is R^{29A}-substituted quinolinyl and R^{29A} is —Cl.

In embodiments, R²¹ is —C(O)NR²⁸R²⁹. In embodiments, R²⁸ and R²⁹ are independently hydrogen, substituted (e.g., R^{28A}-substituted or R^{29A}-substituted) or unsubstituted 4 to 6 membered heteroalkyl or substituted (e.g., R^{28A}-substituted or R^{29A}-substituted) or unsubstituted (e.g., C₅-C₆) aryl.

In embodiments, R²⁸ is hydrogen and R²⁹ is R^{29A}-substituted 2 to 10 membered heteroalkyl. In embodiments, R²⁸ is hydrogen and R²⁹ is R^{29A}-substituted 2 to 8 membered heteroalkyl. In embodiments, R²⁸ is hydrogen and R²⁹ is R^{29A}-substituted 2 to 6 membered heteroalkyl. In embodiments, R²⁸ is hydrogen and R²⁹ is R^{29A}-substituted 2 to 4 membered heteroalkyl. In embodiments, R²⁸ is hydrogen and R²⁹ is R^{29A}-substituted 4 to 10 membered heteroalkyl. In embodiments, R²⁸ is hydrogen and R²⁹ is R^{29A}-substituted 4 to 8 membered heteroalkyl. In embodiments, R²⁸ is hydrogen and R²⁹ is R^{29A}-substituted 4 to 6 membered heteroalkyl. In embodiments, R²⁸ is hydrogen and R²⁹ is R^{29A}-substituted 2 membered heteroalkyl. In embodiments, R²⁸ is hydrogen and R²⁹ is R^{29A}-substituted 4 membered heteroalkyl. In embodiments, R²⁸ is hydrogen and R²⁹ is R^{29A}-substituted 6 membered heteroalkyl. In embodiments, R²⁸ is hydrogen and R²⁹ is R^{29A}-substituted 8 membered heteroalkyl. In embodiments, R^{29A} is substituted or unsubstituted C₁-C₈ alkyl. In embodiments, R^{29A} is substituted or unsubstituted C₁-C₆ alkyl. In embodiments, R^{29A} is substituted or unsubstituted C₁-C₄ alkyl. In embodiments, R^{29A} is unsubstituted C₁-C₄ alkyl. In embodiments, R^{29A} is unsubstituted methyl or ethyl. In embodiments, R²⁸ is hydrogen and R²⁹ is R^{29A}-substituted 4 membered heteroalkyl and R^{29A} is methyl. In embodiments, R²⁸ is hydrogen and R²⁹ is R^{29A}-substituted 6 membered heteroalkyl and R^{29A} is methyl.

In embodiments, R²⁸ is hydrogen and R²⁹ is substituted (e.g., R^{29A}-substituted) C₅-C₆ aryl. In embodiments, R²⁸ is hydrogen and R²⁹ is R^{29A}-substituted phenyl. In embodiments, R^{29A} is —NHR^{29B} and NR^{29B} is unsubstituted (e.g., 5 to10 membered) heteroaryl. In embodiments, R^{29A} is —NHR^{29B} and NR^{29B} is unsubstituted 6 membered heteroaryl. In embodiments, R^{29A} is —NHR^{29B} and NR^{29B} is unsubstituted pyridinyl.

In embodiments, R²¹ is —O—C(O)NR²⁸R²⁹. In embodiments, R²⁸ and R²⁹ are independently hydrogen or substituted or unsubstituted (e.g., R^{28A}-substituted or R^{29A}-substituted) C₁-C₅ alkyl. In embodiments, R²⁸ and R²⁹ are independently hydrogen or unsubstituted methyl.

In formula (VI) R²² may be independently hydrogen, halogen, —CX^{b}₃, —CN, —SO₂Cl, —SOₙ₂R³⁰, —SOᵥ₂NR³⁰R³¹, —NHNH₂, —ONR³⁰R³¹, —NHC═(O)NHNH₂, —NHC═(O)NR³⁰R³¹, —NHC═(O)R³⁰, —N(O)ₘ₂, —NR³⁰R³¹, —NH—O—R³⁰, —C(O)R³⁰, —C(O)—OR³⁰, —C(O)NR³⁰R³¹, —N(R³⁰)C(O)R³¹, —O—C(O)NR³⁰R³¹, —OR³⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R²² is independently hydrogen or OR³⁰. In embodiments, R³⁰ is substituted (e.g., R⁵⁵-substituted) or unsubstituted C₁-C₁₀ alkyl. In embodiments, R³⁰ is substituted or unsubstituted C₁-C₈ alkyl. In embodiments, R³⁰ is substituted or unsubstituted C₁-C₇ alkyl. In embodiments, R³⁰ is substituted or unsubstituted C₁-C₆ alkyl. In embodiments, R³⁰ is substituted or unsubstituted C₁-C₅ alkyl. In embodiments, R³⁰ is substituted or unsubstituted C₁-C₃ alkyl. In embodiments, R³⁰ is unsubstituted C₁-C₁° alkyl. In embodiments, R³⁰ is unsubstituted C₁-C₈ alkyl. In embodiments, R³⁰ is unsubstituted C₁-C₇ alkyl. In embodiments, R³⁰ is unsubstituted C₁-C₆ alkyl. In embodiments, R³⁰ is unsubstituted C₁-C₅ alkyl. In embodiments, R³⁰ is unsubstituted C₁-C₃ alkyl. In embodiments, R³⁰ is unsubstituted methyl.

In formula (VI) R²³ may be independently hydrogen, halogen, —CX^{c}₃, —CN, —SO₂Cl, —SOₙ₃R³², —SOᵥ₃NR³²R³³, —NR³²SOᵥ₃R³³, —NHNH₂, —ONR³²R³³, —NHC═(O)NHNH₂, —NHC═(O)NR³²R³³, —NHC═(O)R³², —N(O)ₘ₃, —NR³²R³³, —NH—O—R³², —R³²NR³³NH₂, —C(O)R³², —C(O)—OR³², —C(O)NR³²R³³, —N(R³²)C(O)R³³, —O—C(O)NR³²R³³, —OR³², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R²³ is independently hydrogen, halogen, —NR³²SOᵥ₃R³³, —R³²NR³³NH₂, OR³² or substituted or unsubstituted alkyl. In embodiments, R²³ is independently hydrogen, or halogen. In embodiments, R²³ is independently —NR³²SOᵥ₃R³³, —R³²NR³³NH₂, OR³² or substituted or unsubstituted (e.g., C₁-C₈) alkyl. In embodiments, R³² and R³³ are independently hydrogen or unsubstituted C₁-C₅ alkyl and v₃ is 2. In embodiments, R³² and R³³ are independently hydrogen or methyl.

In formula (VI) R²⁴ may be independently hydrogen, halogen, —CX^{d}₃, —CN, —SO₂Cl, —SOₙ₄R³⁴, —SOᵥ₄NR³⁴R³⁵, —NHNH₂, —ONR³⁴R³⁵, —NHC═(O)NHNH₂, —NHC═(O)NR³⁴R³⁵, —NHC═(O)R³⁴, —N(O)ₘ₄, —NR³⁴R³⁵, —NH—O—R³⁴, —C(O)R³⁴, —C(O)—OR³⁴, —C(O)NR³⁴R³⁵, —N(R³⁴)C(O)R³⁵, —O—C(O)NR³⁴R³⁵, —OR³⁴, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R²⁴ is independently hydrogen, halogen, substituted (e.g., R^{24A}-substituted) or unsubstituted (e.g., 2 to 10 membered) heteroalkyl or substituted (e.g., R^{24A}-substituted) or unsubstituted (e.g., 5 to 10 membered) heterocycloalkyl. In embodiments, R²⁴ is substituted (e.g., R^{24A}-substituted) or unsubstituted 2 to 5 membered heteroalkyl or substituted (e.g., R^{24A}-substituted) or unsubstituted 6 membered heterocycloalkyl. In embodiments, R²⁴ is substituted (e.g., R^{24A}-substituted) or unsubstituted 2 to 5 membered heteroalkyl. In embodiments, R²⁴ is R^{24A-}substituted or unsubstituted 2 to 5 membered heteroalkyl and R^{24A} is methyl. In embodiments, R²⁴ is substituted (e.g., R^{24A}-substituted) or unsubstituted 6 membered heterocycloalkyl. In embodiments, R²⁴ is R^{24A}-substituted 6 membered heterocycloalkyl and R^{24A} is C₁-C₈ alkyl. In embodiments, R²⁴ is R^{24A}-substituted 6 membered heterocycloalkyl and R^{24A} is C₁-C₅ alkyl. In embodiments, R²⁴ is R^{24A}-substituted 6 membered heterocycloalkyl and R^{24A} is C₁-C₃ alkyl. In embodiments, R²⁴ is R^{24A}-substituted 6 membered heterocycloalkyl and R^{24A} is methyl. In embodiments, R²⁴ is R^{24A}-substituted 6 membered heterocycloalkyl and R^{24A} is ethyl.

In formula (VI) R²⁵ may be independently hydrogen, halogen, —CX^{e}₃, —CN, —SO₂Cl, —SOₙ₅R³⁶, —SOᵥ₅NR³⁶R³⁷, —NHNH₂, —ONR³⁶R³⁷, —NHC═(O)NHNH₂, —NHC═(O)NR³⁶R³⁷, —NHC═(O)R³⁶, —N(O)ₘ₅, —NR³⁶R³⁷, —NH—O—R³⁶, —C(O)R³⁶, —C(O)—OR³⁶, —C(O)NR³⁶R³⁷, —N(R³⁶)C(O)R³⁷, —O—C(O)NR³⁶R³⁷, —OR³⁶, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R²⁵ is hydrogen.

In embodiments, W¹ is CR²⁶ and R²⁶ is hydrogen. In embodiments, W² is CR²⁷ and R²⁷ is independently hydrogen, halogen or NR⁴⁰R⁴¹. In embodiments, R⁴⁰ and R⁴¹ are independently hydrogen.

In formula (VI) L³ may be independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L³ is independently a bond, —C(O)—, substituted (e.g., R⁴⁶-substituted) or unsubstituted C₁-C₈ alkylene or substituted (e.g., R⁴⁶-substituted) or unsubstituted 5 to 10 membered arylene. In embodiments, L³ is independently a bond, —C(O)—, substituted (e.g., R⁴⁶-substituted) or unsubstituted C₁-C₅ alkylene or substituted (e.g., R⁴⁶-substituted) or unsubstituted 5 to 10 membered arylene.

In embodiments, L³ is R⁴⁶-substituted or unsubstituted C₁-C₈ alkylene. In embodiments, L³ is R⁴⁶-substituted or unsubstituted C₁-C₇ alkylene. In embodiments, L³ is R⁴⁶-substituted or unsubstituted C₁-C₆ alkylene. In embodiments, L³ is R⁴⁶-substituted or unsubstituted C₁-C₅ alkylene. In embodiments, L³ is R⁴⁶-substituted or unsubstituted C₁-C₄ alkylene. In embodiments, L³ is R⁴⁶-substituted or unsubstituted C₁-C₃ alkylene. In embodiments, L³ is R⁴⁶-substituted or unsubstituted methylene. In embodiments, L³ is R⁴⁶-substituted or unsubstituted ethylene. In embodiments, L³ is R⁴⁶-substituted or unsubstituted propylene. In embodiments, R⁴⁶ is —OH.

In embodiments, L³ is unsubstituted C₁-C₈ alkylene. In embodiments, L³ is unsubstituted C₁-C₇ alkylene. In embodiments, L³ is unsubstituted C₁-C₆ alkylene. In embodiments, L³ is unsubstituted C₁-C₅ alkylene. In embodiments, L³ is unsubstituted C₁-C₄ alkylene. In embodiments, L³ is unsubstituted C₁-C₃ alkylene. In embodiments, L³ is unsubstituted methylene. In embodiments, L³ is ethylene. In embodiments, L³ is unsubstituted propylene.

In embodiments, L³ is substituted (e.g., R⁴⁶-substituted) or unsubstituted 5 to 10 membered arylene. In embodiments, L³ is R⁴⁶-substituted or unsubstituted 5 to 10 membered arylene. In embodiments, L³ is R⁴⁶-substituted or unsubstituted 5 membered arylene. In embodiments, L³ is R⁴⁶-substituted or unsubstituted 6 membered arylene. In embodiments, L³ is R⁴⁶-substituted or unsubstituted 10 membered arylene. In embodiments, L³ is R⁴⁶-substituted or unsubstituted (e.g., R⁴⁶-substituted) phenylene. In embodiments, R⁴⁶ is hydrogen, —OH, or —NO₂.

In embodiments, the compound has the structure:

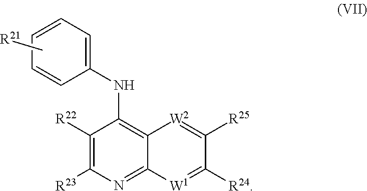

In embodiments, the compound has the structure:

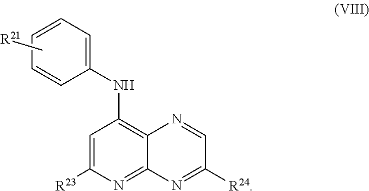

In embodiments, the compound has the structure:

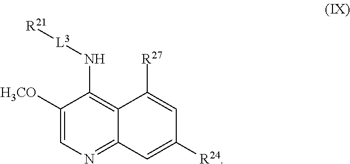

In some embodiments, a compound of formula (VI), (VII). (VIII), or (IX) is one or more compounds set forth in Table 2 below.

Further to any of Formulae (I) to (IX), in some embodiments a substituent is a size-limited substituent. For example without limitation, in some embodiments each substituted or unsubstituted alkyl may be a substituted or unsubstituted C₁-C₂₀, C₁-C₁₀, C₁-C₆, or even C₁ alkyl. In some embodiments each substituted or unsubstituted heteroalkyl may be a substituted or unsubstituted 2-20 membered, 2-10 membered, or 2-6 membered heteroalkyl. In some embodiments, each substituted or unsubstituted cycloalkyl is a substituted or unsubstituted C₃-C₈, C₄-C₈, C₅-C₇ cycloalkyl. In some embodiments, each substituted or unsubstituted heterocycloalkyl is a substituted or unsubstituted 3-8 membered, 4-8 membered, or 3-6 membered heterocycloalkyl. In some embodiments, each substituted or unsubstituted heteroaryl is a substituted or unsubstituted 4-14 membered, 4-10 membered, 5-8 membered, 4-6 membered, 5-6 membered or 6-membered heteroaryl. In some embodiments, each substituted or unsubstituted aryl is a substituted or unsubstituted C₄-C₁₄, C₄-C₁₀, C₆-C₁₀, C₅-C₈, C₅-C₆, or C₆ aryl (phenyl). In other embodiments each substituted or unsubstituted alkylene may be a substituted or unsubstituted C₁-C₂₀, C₁-C₁₀, C₁-C₆, or even C₁ alkylene. In some embodiments each substituted or unsubstituted heteroalkylene may be a substituted or unsubstituted 2-20 membered, 2-10 membered, or 2-6 membered heteroalkylene. In some embodiments, each substituted or unsubstituted cycloalkylene is a substituted or unsubstituted C₃-C₈, C₄-C₈, C₅-C₇ cycloalkylene. In some embodiments, each substituted or unsubstituted heterocycloalkylene is a substituted or unsubstituted 3-8 membered, 4-8 membered, or 3-6 membered heterocycloalkylene. In some embodiments, each substituted or unsubstituted heteroarylene is a substituted or unsubstituted 4-14 membered, 4-10 membered, 5-8 membered, 4-6 membered, 5-6 membered or 6-membered heteroarylene. In some embodiments, each substituted or unsubstituted arylene is a substituted or unsubstituted C₄-C₁₄, C₄-C₁₀, C₆-C₁₀, C₅-C₈, C₅-C₆, or C₆ arylene (phenylene).

In another aspect a compound having the formula:

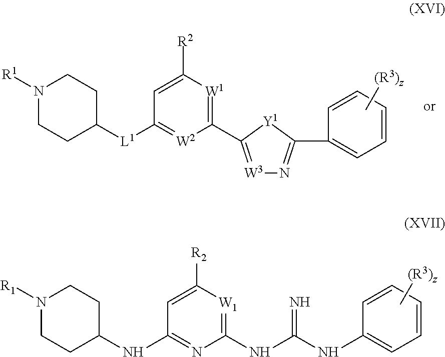

is provided. In formula (XVI) or (XVII) R¹ is independently hydrogen, halogen, —CX^{a}₃, —CN, —SO₂Cl, —SOₙR⁷, —SOᵥNR⁴R⁵, —NHNH₂, —ONR⁴R⁵, —NHC═(O)NHNH₂, —NHC═(O)NR⁴R⁵, —N(O)ₘ, —NR⁴R⁵, —C(O)R⁶, —C(O)—OR⁶, —C(O)NR⁴R⁵, —OR⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R² is independently hydrogen, halogen, —CX^{b}₃, —CN, —SO₂Cl, —SOₙR¹¹, —SOᵥNR⁸R⁹, —NHNH₂, —ONR⁸R⁹, —NHC═(O)NHNH₂, —NHC═(O)NR⁸R⁹, —N(O)ₘ, —NR⁸R⁹, —C(O)R¹⁰, —C(O)—OR¹⁰, —C(O)NR⁸R⁹, —OR¹¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R³ is independently hydrogen, halogen, —CX^{c}₃, —CN, —SO₂Cl, —SOₙR¹⁵, —SOᵥNR12R¹³, —NHNH₂, —ONR¹²R¹³, —NHC═(O)NHNH₂, —NHC═(O)NR¹²R¹³, —N(O)ₘ, —NR¹²R¹³, —C(O)R¹⁴, —C(O)—OR¹⁴, —C(O)NR¹²R¹³, —OR¹⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁴, R⁵, R⁶, R⁷, R⁸, R⁹, R¹⁰, R¹¹, R¹², R¹³, R¹⁴, R¹⁵ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. W¹, W², and W³ are independently —CH— or —N—. L¹ is independently —CH₂— or —NH—. Y¹ is independently —O—, —S—, or —NH—. X^{a}, X^{b}, and X^{c} are independently —F, —Cl, —Br, or —I. The symbol n is an integer from 0 to 4. The symbol m is an integer from 1 to 2. The symbol v is an integer from 1 to 2. The symbol z is an integer from 0 to 5.

In another aspect a compound having the formula:

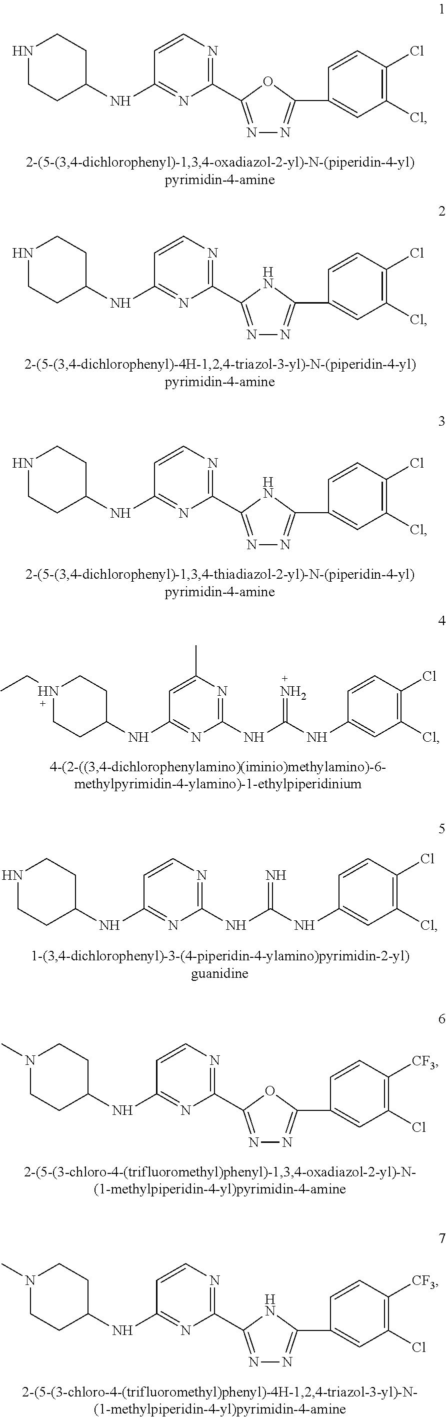

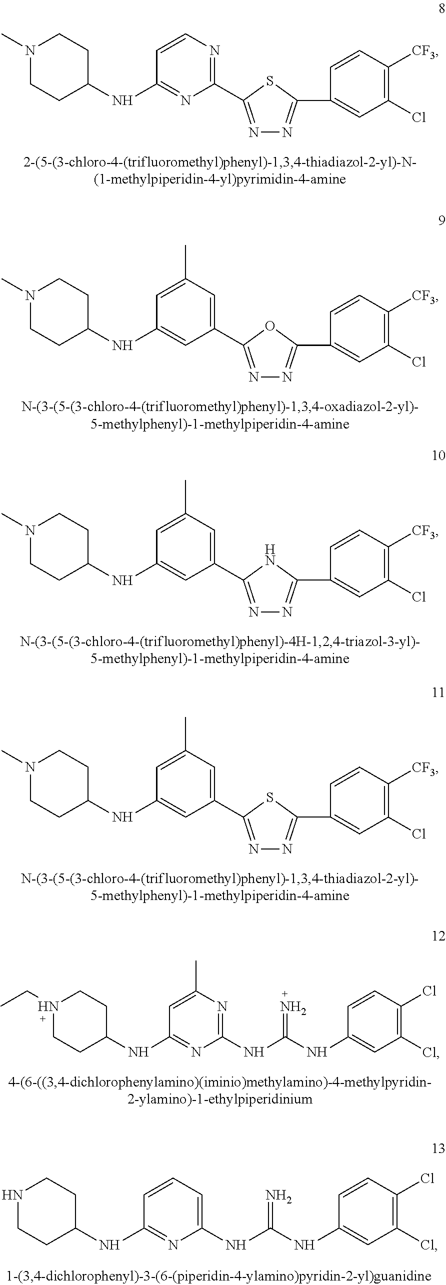 [SMILES: CN1CCC(Nc2ccnc(-c3nnc(-c4ccc(C(F)(F)F)c(Cl)c4)s3)n2)CC1 ; Cc1cc(NC2CCN(C)CC2)cc(-c2nnc(-c3ccc(C(F)(F)F)c(Cl)c3)o2)c1 ; Cc1cc(NC2CCN(C)CC2)cc(-c2nnc(-c3ccc(C(F)(F)F)c(Cl)c3)[nH]2)c1 ; Cc1cc(NC2CCN(C)CC2)cc(-c2nnc(-c3ccc(C(F)(F)F)c(Cl)c3)s2)c1 ; CCN1CCC(Nc2cc(C)nc(NC(=N)Nc3ccc(Cl)c(Cl)c3)n2)CC1 ; N=C(Nc1ccc(Cl)c(Cl)c1)Nc1cccc(NC2CCNCC2)n1]

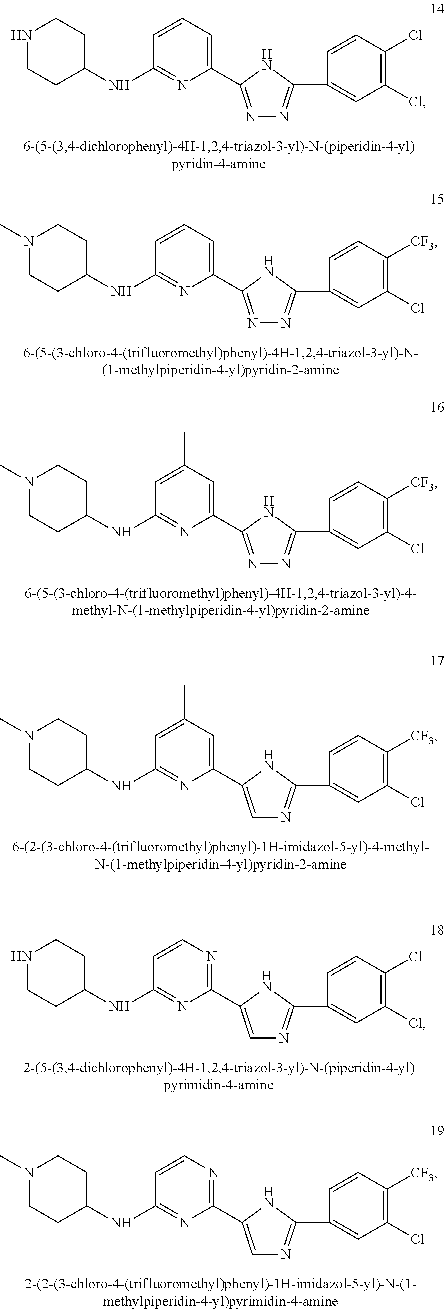 [SMILES: Clc1ccc(-c2nnc(-c3cccc(NC4CCNCC4)n3)[nH]2)cc1Cl ; CN1CCC(Nc2cccc(-c3nnc(-c4ccc(C(F)(F)F)c(Cl)c4)[nH]3)n2)CC1 ; Cc1cc(NC2CCN(C)CC2)nc(-c2nnc(-c3ccc(C(F)(F)F)c(Cl)c3)[nH]2)c1 ; Cc1cc(NC2CCN(C)CC2)nc(-c2cnc(-c3ccc(C(F)(F)F)c(Cl)c3)[nH]2)c1 ; Clc1ccc(-c2ncc(-c3nccc(NC4CCNCC4)n3)[nH]2)cc1Cl ; CN1CCC(Nc2ccnc(-c3cnc(-c4ccc(C(F)(F)F)c(Cl)c4)[nH]3)n2)CC1]

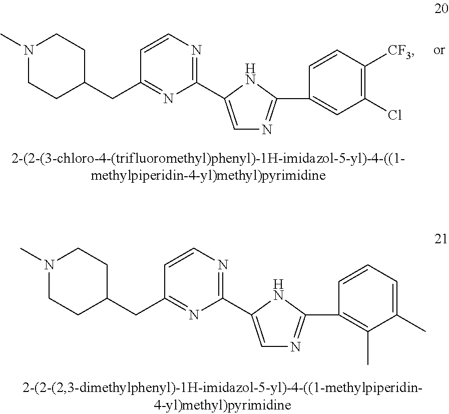

is provided.

In another aspect a compound having the formula:

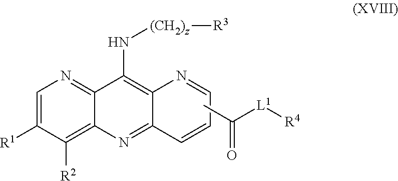

is provided. In formula (XVIII) R¹ is independently hydrogen, halogen, —CX^{a}₃, —CN, —SO₂Cl, —SOₙR⁸, —SOᵥNR⁵R⁶, —NHNH₂, —ONR⁵R⁶, —NHC═(O)NHNH₂, —NHC═(O)NR⁵R⁶, —N(O)ₘ, —NR⁵R⁶, —C(O)R⁷, —C(O)—OR⁷, —C(O)NR⁵R⁶, —OR⁸, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R² is independently hydrogen, halogen, —CX^{b}₃, —CN, —SO₂Cl, —SOₙR¹², —SOᵥNR⁹R¹⁰, —NHNH₂, —ONR⁹R¹⁰, —NHC═(O)NHNH₂, —NHC═(O)NR⁹R¹⁰, —N(O)ₘ, —NR⁹R¹⁰, —C(O)R¹¹, —C(O)—OR¹¹, —C(O)NR⁹R¹⁰, —OR¹², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R³ is independently hydrogen, halogen, —CX^{c}₃, —CN, —SO₂Cl, —SOₙR¹⁶, —SOᵥNR¹³R¹⁴, —NHNH₂, —ONR¹³R¹⁴, —NHC═(O)NHNH₂, —NHC═(O)NR¹³R¹⁴, —N(O)ₘ, —NR¹³R¹⁴, —C(O)R¹⁵, —C(O)—OR¹⁵, —C(O)NR¹³R¹⁴, —OR¹⁶, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁴ is independently hydrogen, halogen, —CX^{d}₃, —CN, —SO₂Cl, —SOₙR²⁰, —SOᵥNR¹⁷R¹⁸, —NHNH₂, —ONR¹⁷R¹⁸, —NHC═(O)NHNH₂, —NHC═(O)NR¹⁷R¹⁸, —N(O)ₘ, —NR¹⁷R¹⁸, —C(O)R¹⁹, —C(O)—OR¹⁹, —C(O)NR¹⁷R¹⁸, —OR²⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. L¹ is independently —O— or —NH—. X^{a}, X^{b}, X^{c} and X^{d} are independently —F, —Cl, —Br, or —I. The symbol n is an integer from 0 to 4. The symbol m is an integer from 1 to 2. The symbol v is an integer from 1 to 2. The symbol z is an integer from 1 to 25.

In another aspect, a compound having the formula:

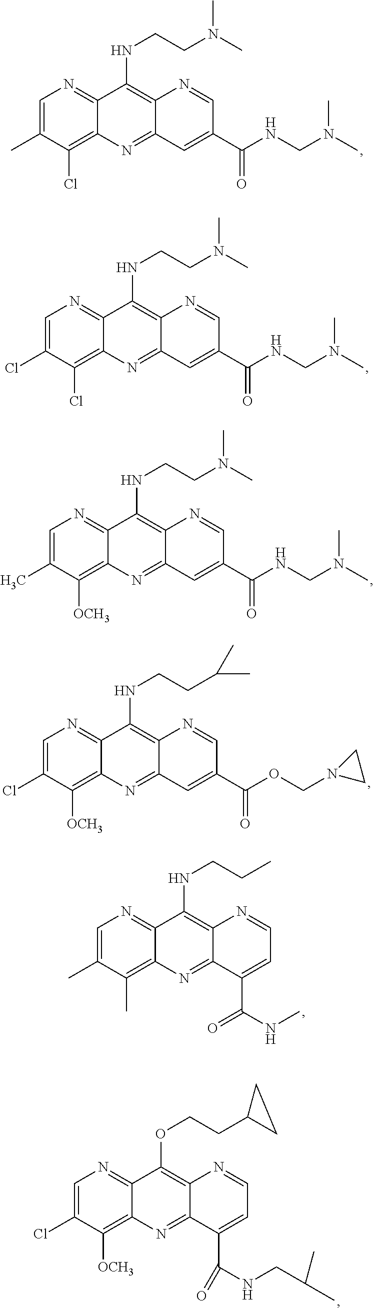

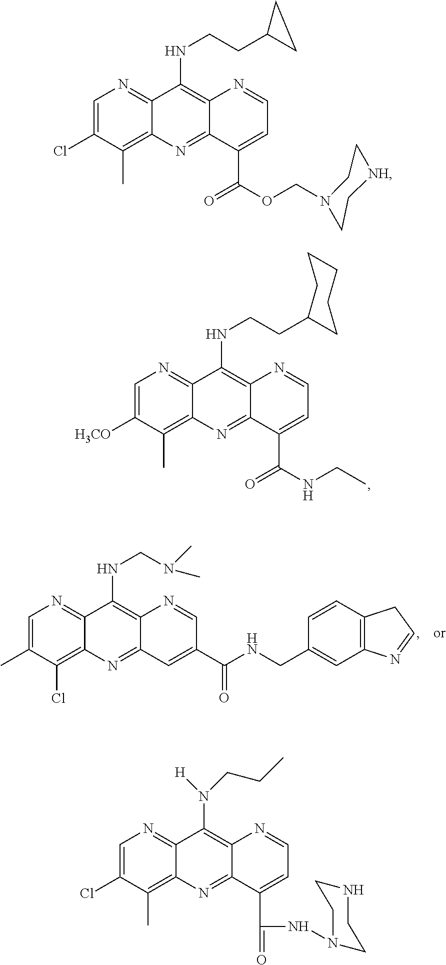

is provided.

In another aspect, a compound having the formula:

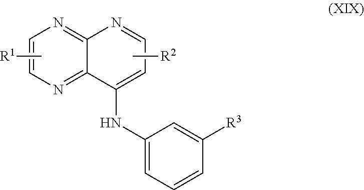

is provided. In formula (XIX) R¹ is independently hydrogen, halogen, —CX^{a}₃, —CN, —SO₂Cl, —SOₙR⁷, —SOᵥNR⁴R⁵, —NHNH₂, —ONR⁴R⁵, —NHC═(O)NHNH₂, —NHC═(O)NR⁴R⁵, —N(O)ₘ, —NR⁴R⁵, —C(O)R⁶, —C(O)—OR⁶, —C(O)NR⁴R⁵, —OR⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R² is independently hydrogen, halogen, —CX^{b}₃, —CN, —SO₂Cl, —SOₙR¹¹, —SOᵥNR⁸R⁹, —NHNH₂, —ONR⁸R⁹, —NHC═(O)NHNH₂, —NHC═(O)NR⁸R⁹, —N(O)ₘ, —NR⁸R⁹, —C(O)R¹⁰, —C(O)—OR¹⁰, —C(O)NR⁸R⁹, —OR¹¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R³ is independently hydrogen, halogen, —CX^{c}₃, —CN, —SO₂Cl, —SOₙR¹⁵, —SOᵥNR¹²R¹³, —NHNH₂, —ONR¹²R¹³, —NHC═(O)NHNH₂, —NHC═(O)NR¹²R¹³, —N(O)ₘ, —NR¹²R¹³, —C(O)R¹⁴, —C(O)—OR¹⁴, —C(O)NR¹²R¹³, —OR¹⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁴, R⁵, R⁶, R⁷, R⁸, R⁹, R¹⁰, R¹¹, R¹², R¹³, R¹⁴, R¹⁵, are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. X^{a}, X^{b}, and X^{c} are independently —F, —Cl, —Br, or —I. The symbol n is an integer from 0 to 4. The symbol m is independently an integer from 1 to 2. The symbol v is an integer from 1 to 2. The symbol z is an integer from 0 to 5.

In another aspect, a compound having the formula:

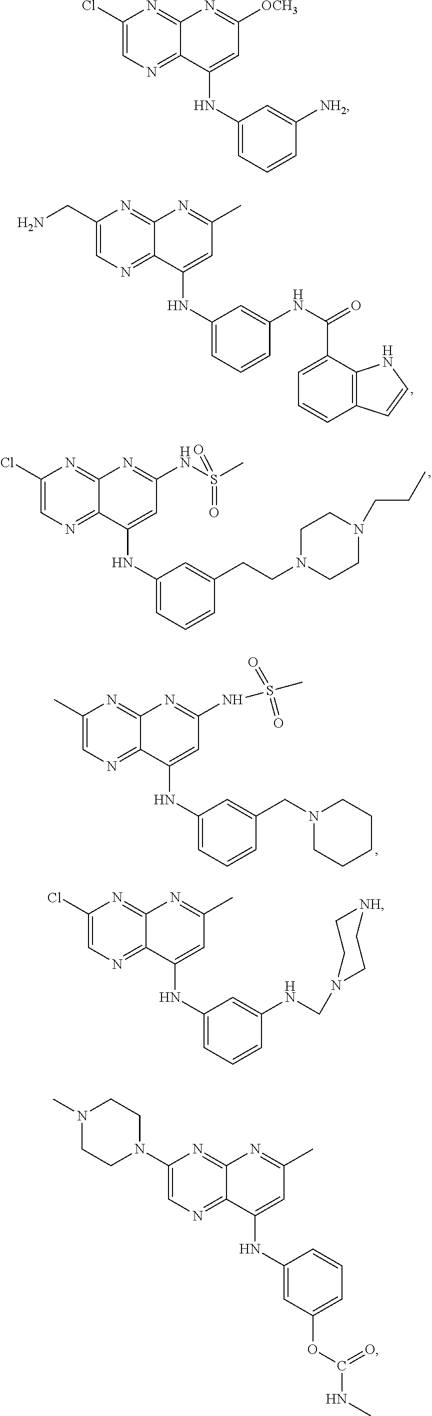

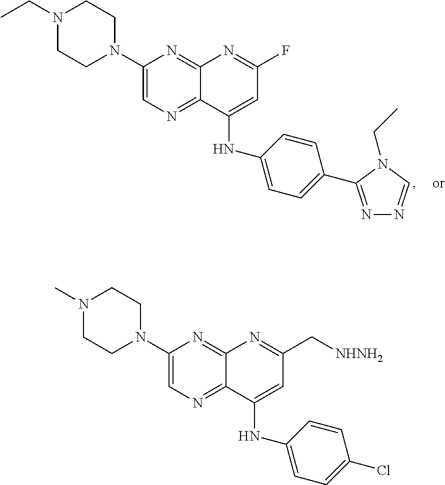 [SMILES: CCN1CCN(c2cnc3c(Nc4ccc(-c5nncn5CC)cc4)cc(F)nc3n2)CC1 ; CN1CCN(c2cnc3c(Nc4ccc(Cl)cc4)cc(CNN)nc3n2)CC1]

is provided.

### III. Peptides and Peptidomimetics

In another aspect, a peptide, peptidomimetic, cyclic peptidomimetic, or cyclic peptide, wherein the peptide, peptidomimetic, cyclic peptidomimetic, or cyclic peptide capable of binding to Olig2 is provided. In some embodiments, the peptide or cyclic peptide include a sequence of seven to 57 amino acids, wherein the seven to 57 amino acids are substantially identical to the sequence:

(SEQ ID NO: 14)
RLKINSRERKRMHDLNIAMDGLREVMPYAHGPSVRKLSKIATLLLARNYI
LMLTNSL.

In some embodiments, the peptide or cyclic peptide includes a sequence of seven to 57 amino acids, wherein the seven to 57 amino acids are identical to the sequence:

(SEQ ID NO: 14)
RLKINSRERKRMHDLNIAMDGLREVMPYAHGPSVRKLSKIATLLLARNYI
LMLTNSL.

In some embodiments, the peptidomimetic or cyclic peptidomimetic competes for binding to Olig2 with a peptide or cyclic peptide including a sequence of seven to 57 amino acids, wherein the seven to 57 amino acids are identical to the sequence:

(SEQ ID NO: 14)
RLKINSRERKRMHDLNIAMDGLREVMPYAHGPSVRKLSKIATLLLARNYI
LMLTNSL.

In some embodiments, the peptide or cyclic peptide includes a sequence selected from DLNIAMDGLREVM (SEQ ID NO:1), DLNIAMDGLRE (SEQ ID NO:2), DLNIAMDGLR (SEQ ID NO:3), DLNIAMD (SEQ ID NO:4), AMDGLREVM (SEQ ID NO:5), DGLREVM (SEQ ID NO:6), YAHGPSVRKLSKIATLLLARNYILMLTN (SEQ ID NO:7), YAHGPSVRKLSKIATLLLAR (SEQ ID NO:8), KLSKIATLLLARNYILMLTN (SEQ ID NO:9), TLLLARNYILMLTN (SEQ ID NO:10), RKLSKIATLLLAR (SEQ ID NO:11), YAHGPSVRKLSK (SEQ ID NO:12), or RNYILMLTN (SEQ ID NO:13). In some embodiments, the peptide, peptidomimetic, cyclic peptidomimetic, or cyclic peptide binds the hinge region of the dimerization loop of Olig2.

### IV. Pharmaceutical Compositions and Methods of Treatment

In another aspect, a pharmaceutical composition is provided. The pharmaceutical composition includes a pharmaceutically acceptable excipient and a compound as provided herein (e.g. a compound of Formula (I), (II), (III), (IV) and (V)) including embodiments thereof).

In another aspect, a pharmaceutical composition is provided. The pharmaceutical composition includes a pharmaceutically acceptable excipient and a compound as provided herein (e.g. a compound of Formula (VI), (VII), (VIII), and (IX)) including embodiments thereof).

In another aspect, a pharmaceutical composition is provided. The pharmaceutical composition includes a pharmaceutically acceptable excipient and a compound of Table 1, 2, or 3.

In another aspect, a pharmaceutical composition is provided. The pharmaceutical composition includes a pharmaceutically acceptable excipient and a peptide, peptidomimetic, cyclic peptidomimetic, or cyclic peptide as provided herein.

In another aspect, a method of treating a disease in a patient in need of such treatment is provided. The method includes administering a therapeutically effective amount of a compound as provided herein (e.g. Formula (I), (II), (III), (IV) and (V)).

In another aspect, a method of treating a disease in a patient in need of such treatment is provided. The method includes administering a therapeutically effective amount of a compound as provided herein (e.g. Formula (VI), (VII), (VIII), and (IX)).

In another aspect, a method of treating a disease in a patient in need of such treatment is provided. The method includes administering a therapeutically effective amount of a compound of Table 1, 2, or 3.

In another aspect, a method of treating a disease in a patient in need of such treatment is provided. The method includes administering a therapeutically effective amount of a peptide, peptidomimetic, cyclic peptidomimetic, or cyclic peptide as provided herein.

In embodiments of the method of treating a disease, the disease is cancer. In embodiments, the cancer is brain cancer, glioblastoma multiforme, medulloblastoma, astrocytomas, brain stem gliomas, meningiomas, oligodendrogliomas, melanoma, lung cancer, breast cancer, or leukemia. In embodiments, the disease is Down's Syndrome.

The pharmaceutical compositions include optical isomers, diastereomers, or pharmaceutically acceptable salts of the modulators disclosed herein. The compound included in the pharmaceutical composition may be covalently attached to a carrier moiety, as described above. Alternatively, the compound included in the pharmaceutical composition is not covalently linked to a carrier moiety.

The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation).

The compounds of the present invention can be prepared and administered in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The compounds of the present invention can also be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present invention can be administered transdermally. It is also envisioned that multiple routes of administration (e.g., intramuscular, oral, transdermal) can be used to administer the compounds of the invention. Accordingly, the present invention also provides pharmaceutical compositions comprising a pharmaceutically acceptable excipient and one or more compounds of the invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substance, that may also act as diluents, flavoring agents, binders, preservatives, tablet disintegrating agents, or an encapsulating material.

In powders, the carrier is a finely divided solid in a mixture with the finely divided active component (e.g. a compound provided herein). In tablets, the active component is mixed with the carrier having the necessary binding properties in suitable proportions and compacted in the shape and size desired. The powders and tablets preferably contain from 5% to 70% of the active compound.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. The compounds of the invention can also be incorporated into liposomes or administered via transdermal pumps or patches. Pharmaceutical admixtures suitable for use in the present invention are well-known to those of skill in the art and are described, for example, in Pharmaceutical Sciences (17th Ed., Mack Pub. Co., Easton, Pa.) and WO 96/05309, the teachings of both of which are hereby incorporated by reference.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60 and 80; Pluronic F-68, F-84 and P-103; cyclodextrin; polyoxyl 35 castor oil; or other agents known to those skilled in the art. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, combinations of the foregoing, and other agents known to those skilled in the art. Such agents are typically employed at a level between about 0.01% and about 2% by weight. Determination of acceptable amounts of any of the above adjuvants is readily ascertained by one skilled in the art.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

Pharmaceutical compositions provided by the present invention include compositions wherein the active ingredient is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule (e.g. Olig2), and/or reducing, eliminating, or slowing the progression of disease symptoms (e.g. cancer growth or metastasis). Determination of a therapeutically effective amount of a compound of the invention is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g. brain cancer or Down's Syndrome), kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. In one embodiment, the dosage range is 0.001% to 10% w/v. In another embodiment, the dosage range is 0.1% to 5% w/v.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. See, e.g. Fingl et al., In: THE PHARMACOLOGICAL BASIS OF THERAPEUTICS, Ch.1, p.1, 1975. The exact formulation, route of administration and dosage can be chosen by the individual physician in view of the patient's condition and the particular method in which the compound is used.

### V. Administration

The compositions of the present invention can be delivered by transdermally, by a topical route, formulated as applicator sticks, solutions, suspensions, emulsions, gels, creams, ointments, pastes, jellies, paints, powders, and aerosols. For therapeutic applications, the compounds or drugs of the present invention can be administered alone or co-administered in combination with conventional chemotherapy, radiotherapy, hormonal therapy, and/or immunotherapy.

The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

The pharmaceutical compositions of the present invention can be provided as a salt and can be formed with many acids, including but not limited to hydrochloric, sulfuric, acetic, lactic, tartaric, malic, succinic, etc. Pharmaceutical compositions described herein may be salts of a compound or composition which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

Certain compositions described herein or kinase inhibitor compounds of the present invention can exist in unsolvated forms as well as solvated forms, including hydrated forms. In general, the solvated forms are equivalent to unsolvated forms and are intended to be encompassed within the scope of the present invention. Certain kinase inhibitor compounds of the present invention may exist in multiple crystalline or amorphous forms. In general, all physical forms are equivalent for the uses contemplated by the present invention and are intended to be within the scope of the present invention.

In another embodiment, the compositions of the present invention are useful for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The compounds described herein can be used in combination with one another, with other active agents known to be useful in treating a disease associated with cells expressing a Olig2 (e.g. cancer), or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

As a non-limiting example, the Olig2 inhibitor compounds described herein can be co-administered with conventional chemotherapeutic agents including alkylating agents (e.g., cyclophosphamide, ifosfamide, chlorambucil, busulfan, melphalan, mechlorethamine, uramustine, thiotepa, nitrosoureas, etc.), anti-metabolites (e.g., 5-fluorouracil, azathioprine, methotrexate, leucovorin, capecitabine, cytarabine, floxuridine, fludarabine, gemcitabine, pemetrexed, raltitrexed, etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin, etc.), and the like.

The Olig2 inhibitor compounds described herein can also be co-administered with conventional hormonal therapeutic agents including, but not limited to, steroids (e.g., dexamethasone), finasteride, aromatase inhibitors, tamoxifen, and gonadotropin-releasing hormone agonists (GnRH) such as goserelin.

Additionally, the Olig2 inhibitor compounds described herein can be co-administered with conventional immunotherapeutic agents including, but not limited to, immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), and radioimmunotherapy (e.g., anti-CD20 monoclonal antibody conjugated to ¹¹¹In, ⁹⁰Y, or ¹³¹I, etc.).

In a further embodiment, the kinase inhibitor compounds described herein can be co-administered with conventional radiotherapeutic agents including, but not limited to, radionuclides such as ⁷⁴Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ^{117m}Sn, ¹⁴⁹Pm, ¹⁵³Sm, ¹⁶⁶Ho, ¹⁷⁷Lu, ¹⁸⁶Re, ¹⁸⁸Re, ²¹¹At, and ²¹²Bi, optionally conjugated to antibodies directed against tumor antigens.

The pharmaceutical compositions of the present invention may be sterilized by conventional, well-known sterilization techniques or may be produced under sterile conditions. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents, wetting agents, and the like, e.g., sodium acetate, sodium lactate, sodium chloride, potassium chloride, calcium chloride, sorbitan monolaurate, and triethanolamine oleate.

Formulations suitable for oral administration can comprise: (a) liquid solutions, such as an effective amount of a packaged kinase inhibitor compound or drug suspended in diluents, e.g., water, saline, or PEG 400; (b) capsules, sachets, or tablets, each containing a predetermined amount of an Olig2 inhibitor compound or drug, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise an Olig2 inhibitor compound or drug in a flavor, e.g., sucrose, as well as pastilles comprising the Olig2 inhibitor compound in an inert base, such as gelatin and glycerin or sucrose and acacia emulsions, gels, and the like, containing, in addition to the Olig2 inhibitor, carriers known in the art.

The Olig2 inhibitor compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which comprises an effective amount of a packaged Olig2 inhibitor compound or drug with a suppository base. Suitable suppository bases include natural or synthetic triglycerides or paraffin hydrocarbons. In addition, it is also possible to use gelatin rectal capsules which contain a combination of the Olig2 inhibitor compound or drug of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can also be prepared from sterile powders, granules, and tablets. In the practice of the present invention, compositions can be administered, for example, by intravenous infusion, orally, topically, intraperitoneally, intravesically, or intrathecally. Parenteral administration, oral administration, and intravenous administration are the preferred methods of administration. The formulations of compounds can be presented in unit-dose or multi-dose sealed containers, such as ampoules and vials.

The pharmaceutical preparation is preferably in unit dosage form. In such form the preparation is subdivided into unit doses containing appropriate quantities of the active component, e.g., a kinase inhibitor compound. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

In therapeutic use for the treatment of cancer, Olig2 inhibitor compound utilized in the pharmaceutical compositions of the present invention are administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the Olig2 inhibitor compound or drug being employed. For example, dosages can be empirically determined considering the type and stage of cancer diagnosed in a particular patient. The dose administered to a patient, in the context of the present invention, should be sufficient to affect a beneficial therapeutic response in the patient over time. The size of the dose will also be determined by the existence, nature, and extent of any adverse side-effects that accompany the administration of an Olig2 inhibitor compound in a particular patient. Determination of the proper dosage for a particular situation is within the skill of the practitioner. Generally, treatment is initiated with smaller dosages which are less than the optimum dose of the Olig2 inhibitor compound. Thereafter, the dosage is increased by small increments until the optimum effect under circumstances is reached. For convenience, the total daily dosage may be divided and administered in portions during the day, if desired.

The compounds described herein can be used in combination with one another, with other active agents known to be useful in treating cancer or with adjunctive agents that may not be effective alone, but may contribute to the efficacy of the active agent. In some embodiments, the compounds described herein may be used in combination with compounds or therapies known to be useful for treating brain cancers, including Temozolomide (TMZ-Temodar); radiation; cyclophosphamide; carboplatin, or Avastin (bevacizumab).

### VI. Methods of Inhibiting OLIG2

In another aspect, methods of inhibiting the activity of OLIG2 are provided. The methods include contacting a Olig2 protein with an effective amount of a compound provided herein (e.g., a compound of formula (I), (II), (III), (IV), (V), (VI), (VII), (VIII), (IX). The compound may have the structure of the Formulae provided herein (or any of the embodiments thereof described above). In some embodiments, the methods of inhibiting a Olig2 protein are conducted within a cell. Thus, in certain embodiments, methods of inhibiting the activity of Olig2 within a cell are provided. The method includes contacting a cell with an effective amount of a compound provided herein. The compound may have the structure of the Formulae provided herein (or any of the embodiments thereof described above). In some embodiments, the cell is a prokaryote or eukaryote. The cell may be a eukaryote (e.g. protozoan cell, fungal cell, plant cell or an animal cell). In some embodiments, the cell is a mammalian cell such as a human cell, cow cell, pig cell, horse cell, dog cell and cat cell, mouse cell, or rat cell. In some embodiments, the cell is a human cell. The cell may form part of an organ or an organism. In certain embodiments, the cell does not form part of an organ or an organism.

In another aspect, a method of inhibiting the activity of Olig2 in a cell is provided. The method includes contacting the cell with a compound as provided herein (e.g. Formula (I), (II), (III), (IV), (V) (VI), (VII), (VIII), and (IX)). In some embodiments the compound binds the hinge region of the dimerization loop of Olig2. In some embodiments, the compound inhibits dimerization of Olig2.

In another aspect, a method of inhibiting the activity of Olig2 in a cell is provided. The method includes contacting the cell with a peptide, peptidomimetic, cyclic peptidomimetic, or cyclic peptide as provided herein.

### VII. Methods of Identifying OLIG2 Inhibitors

In another aspect, a method of identifying an inhibitor of protein dimerization is provided. The method includes constructing in silico a computer readable peptide including a steric feature and an electronic feature, wherein the steric feature and the electronic feature form part of a first protein and wherein the steric feature and the electronic feature participate in dimerization of the first protein with a second protein. A level of binding of the computer readable peptide to a compound is determined in silico. The level is compared to a control level, wherein an increase of the level compared to the control level indicates the compound is an inhibitor compound of protein dimerization.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

The compounds described herein are modulators or inhibitors of the neural and GBM (glioblastoma multiforme) stem cell transcriptional repressor OLIG2 (e.g. NM_005806, NP_005797 for human). OLIG2 (also written herein as Olig2) is the oligodendrocyte transcription factor 2. This protein is a member of the bHLH (basic helix-loop-helix) family. The bHLH family is a family of transcription factors that contain the structure motif characterized by two alpha helices connected by a loop. The transcription factors containing bLHL domains are generally dimeric. Generally one of the helices contains basic amino acid residues that are facilitate binding to DNA. OLIG2 is normally restricted to the central nervous system (CNS) in non-disease states, where it is an essential regulator of progenitor cell fate. OLIG2 homodimerizes and hetereodimerizes with the E12 or E47 proteins to then bind and repress the p21 gene promoter among other effects. P21 is a stem cell and tumor suppressor, and is directly repressed by OLIG2. P21 is activated by the tumor suppressor p53. p53 occurs in the intact, wild type form in nearly 70% of primary GBM patient samples. OLIG2 is highly expressed in all diffuse gliomas, and is found in virtually 100% of GBM cells positive for the CD133 stem cell marker. Importantly, OLIG2 is typically not found in normal brain and in tissues outside the CNS unless they are malignant, such as T-cell leukemia, melanoma, lung and breast cancer. No other neural or glial marker gene, and no other transcriptional repressor displays as consistent a link to brain cancers. In contrast, membrane receptors (EGFR, PDGFR, etc) are not uniformly expressed among patients, and various approaches targeting them has met with limited success in GBM treatment. Applicants have shown that genetic silencing of OLIG2 completely ablates the malignant potential of an array of GBM CSC lines in relevant stem cell GBM models and human-derived GBM tumorsphere cultures, indicating that targeting OLIG2 may have a significant anti-tumor effect in GBM. Inhibition of protein dimerization with a small molecule agent has been achieved for the Myc/Max dimer, which along with other examples of protein-protein interaction interventions, indicates that this general approach is practicable. The unique structure of the OLIG2 hinge region compared to other bHLH proteins is allowing us to develop an inhibitor with high specificity.

The expression of Olig2 in diffuse gliomas likely results from the transformed stem cell origin of these tumors. It has been found that a small cohort of the cells present in patient GBM expresses neural stem cell markers including CD133 and nestin, among others. The CD133(+) cells isolated from existing GBM are highly tumorigenic when orthotopically implanted into mice. In one study, as few as 100 of the CD133(+) cells extracted from a patient GBM produced an invasive tumor when transplanted into the brain of a recipient mouse, while 100,000 CD133(−) GBM cells were unable to generate a tumor. Consistent with these findings, a strikingly high percentage of GBM occur in close proximity to the neural stem cell germinal zones in the brain, i.e., neural stem cells undergo malignant transformation and migrate some distance from the germinal zones and establish a GBM.

Another significant finding with respect to GBM cancer stem cells (CSCs) is that the CD133(+) cells are significantly more resistant to radiation and cytotoxic agents used to treat GBM than the bulk of the tumor mass which is comprised of CD133(−) cells. This suggests that conventional radio/chemotherapy spares the CSCs within a GBM, and unless these cells are targeted, the tumor invariably is resurgent, with lethal effect. Moreover, the very few patients that survive GBM suffer lifelong morbidity from chemo- and radio-toxicity, in terms of cognition, endocrine balance, and other functions.

OLIG2 is highly expressed in GBM CSCs, but is only expressed in low levels by normal brain and is not detected in tissues outside the nervous system. OLIG2 inhibitors would offer a therapeutic margin superior to conventional chemotherapy. Low systemic toxicity would be much more compatible with long-term clinical management of GBM than is the case with currently used treatment approaches.

High rates of mortality for patients with brain cancers make this disease a leading cause of cancer related death in men, women and children. Primary brain tumors are actually the most common solid tumor of childhood and the second leading cause of cancer death after leukemia. The toxicity of current treatments causes serious life-long morbidity in the few patients that survive. The development of small molecule, orally available drugs with low toxicity effective in brain cancers would represent a significant advance. Moreover, the compounds may also be effective in other cancers that are stem cell driven and which highly express OLIG2. These cancers include T-cell leukemias, skin cancers, small cell lung cancers, and breast cancers. Moreover, these cancers often metastasize to the brain. This would be relevant to millions of patients worldwide.

Described herein are small molecules that inhibit Olig2 which is a transcription factor critical for survival and proliferation of glioblastoma and other brain cancers, i.e., medulloblastoma, astrocytomas, brain stem gliomas, meningiomas, and oligodendrogliomas. Olig2 especially is detected primarily in the brain, generally not outside the nervous system, and it is highly expressed in glioblastoma tumors. This means that OLIG2 inhibition should have relatively low toxicity to a patient. Olig2 is also over-expressed in melanomas, lung cancers, breast cancer and T-cell leukemias, so an Olig2 inhibitor may also be applicable to the treatment of these cancers.

No other transcription factor or marker displays as consistent a link to brain cancer as does Olig2, so Olig2 inhibition should compare favorably to other signaling pathway inhibitors in glioblastoma. Olig2 is a robust target in that the hinge region of its dimerization loop is unique compared to other proteins of its class (basic helix-loop-helix proteins).

The Olig2 targeted inhibitors described herein should prove unique in terms of efficacy and toxicity.

The existing agents, therapeutics, and methods used to treat brain cancers include Temozolomide (TMZ-Temodar); radiation; cyclophosphamide; carboplatin, and occasional supplementation with Avastin. All these are only somewhat effective standard brain cancer therapeutic agents, and they are very toxic. No brain cancer stem cell inhibitors currently exist for brain tumors.

### A. Example 1

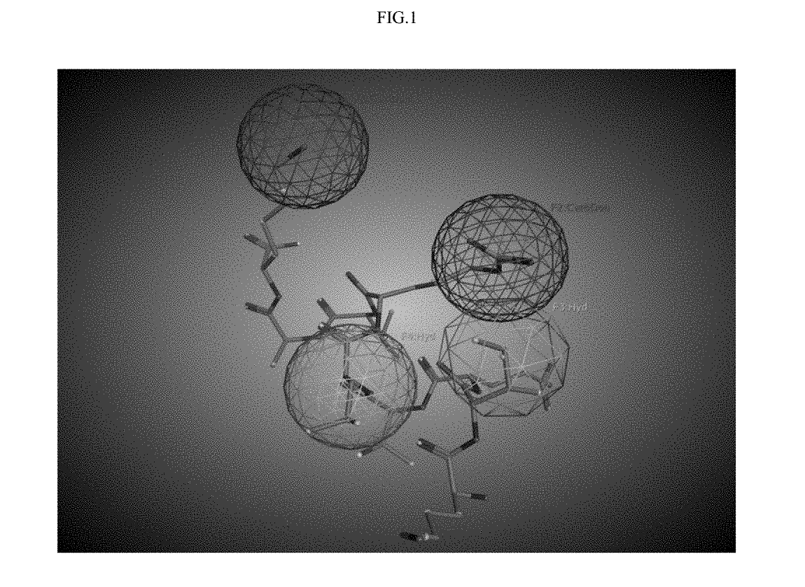

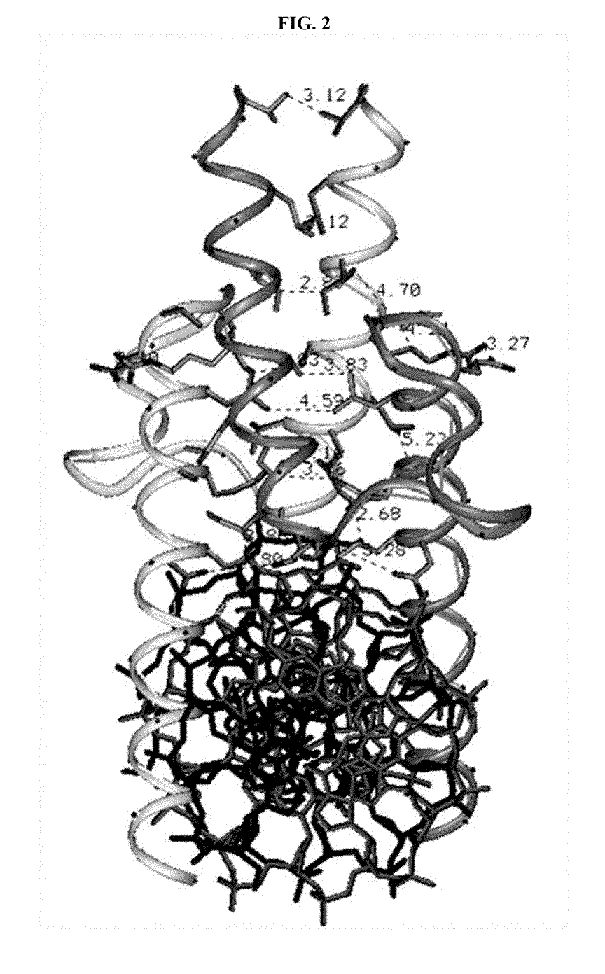

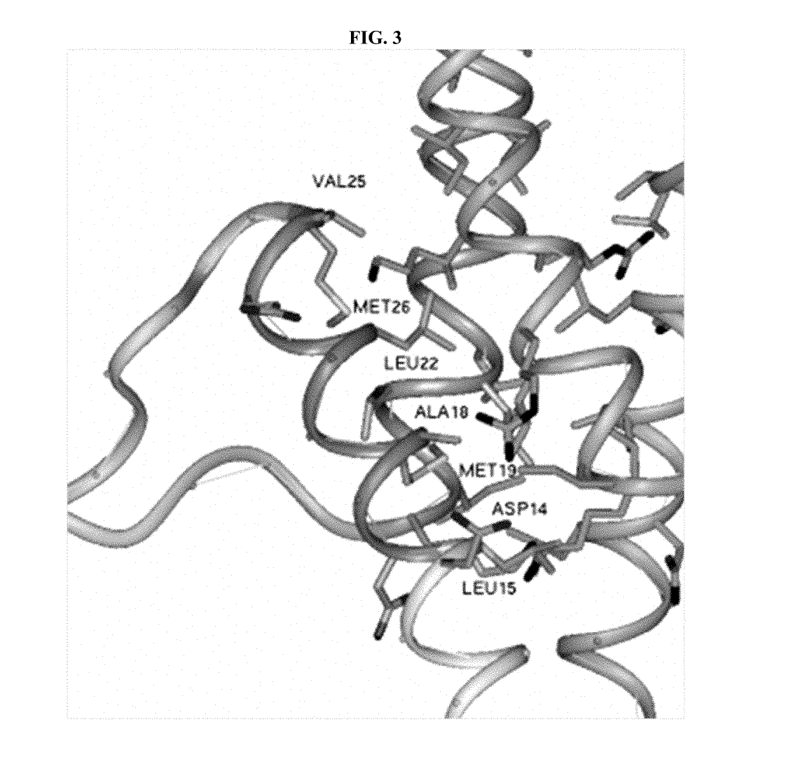

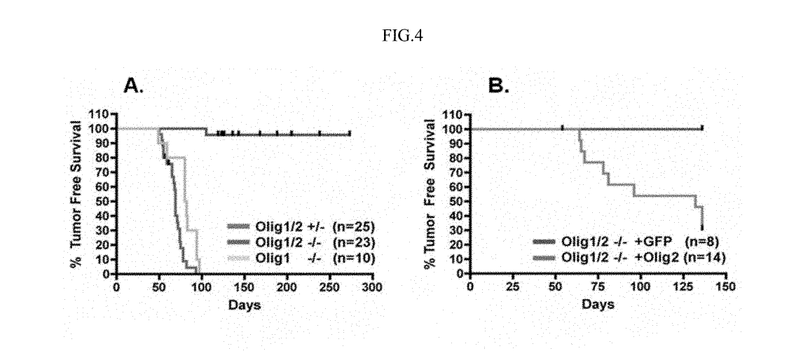

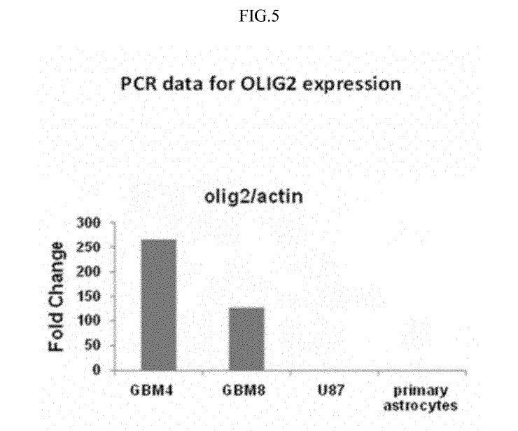

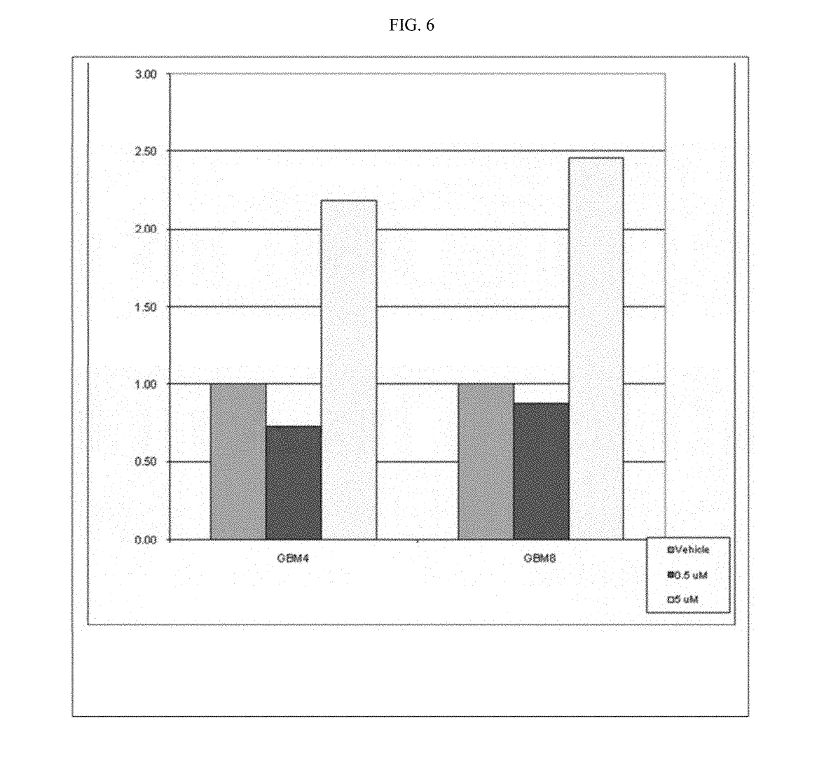

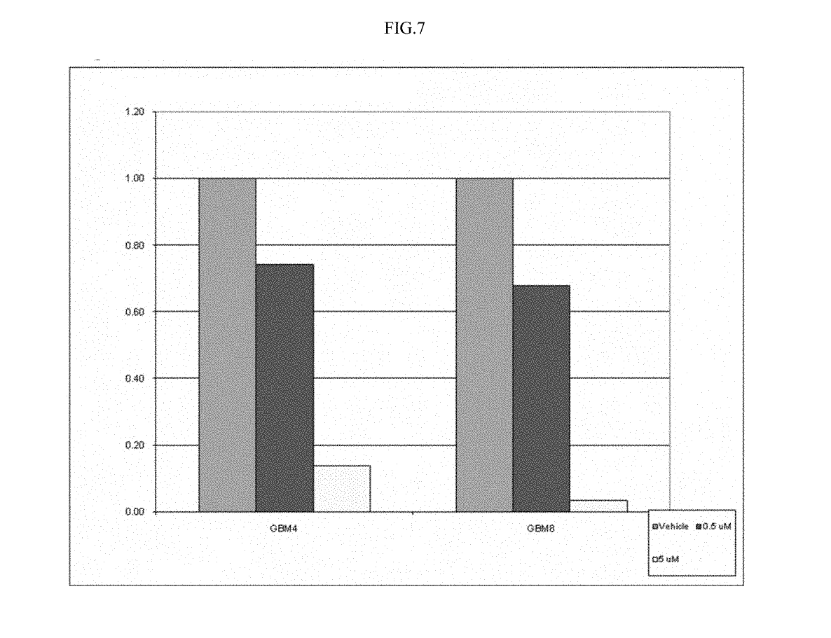

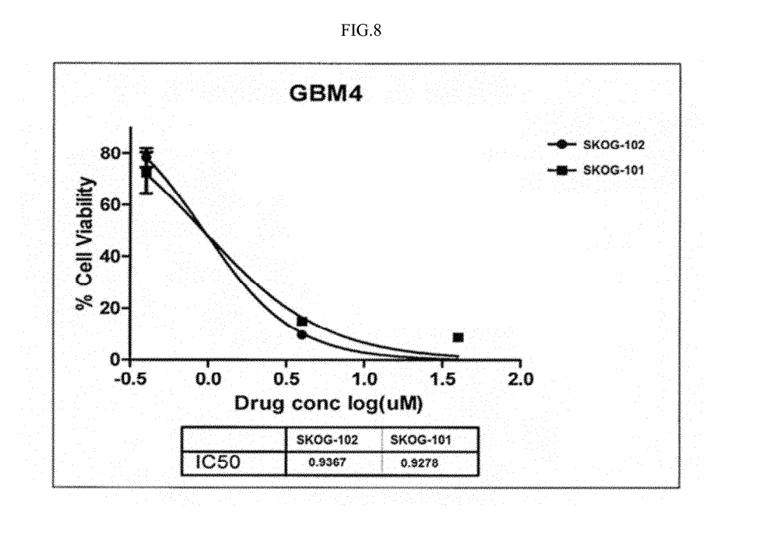

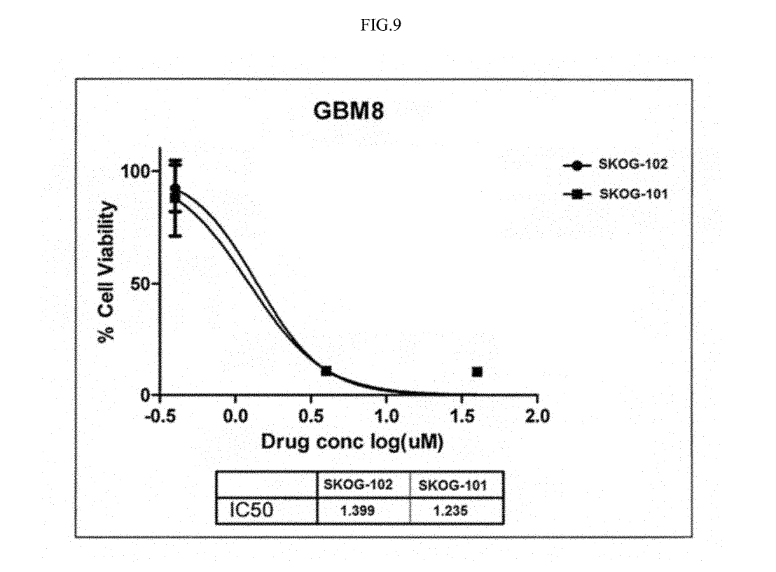

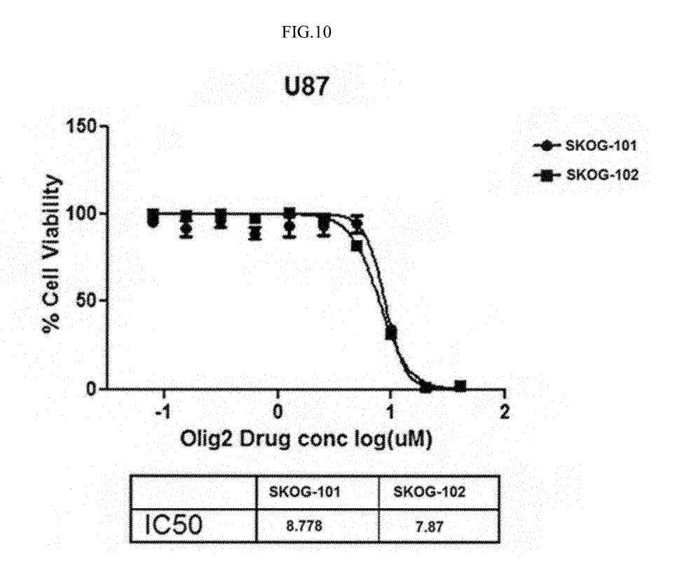

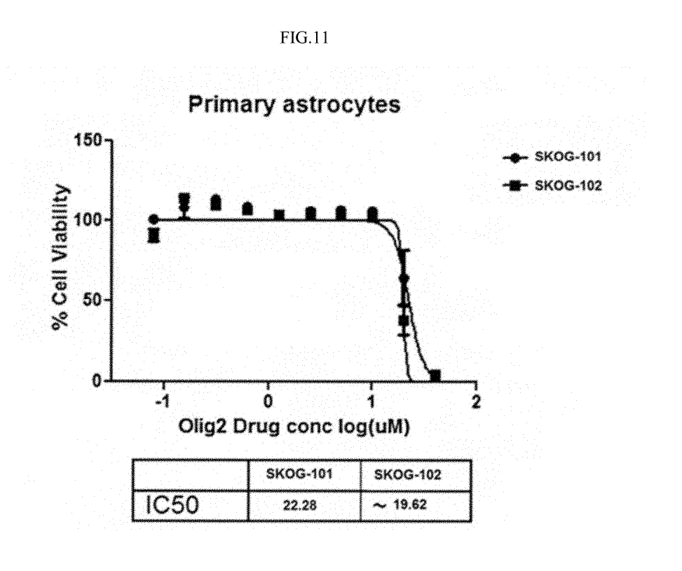

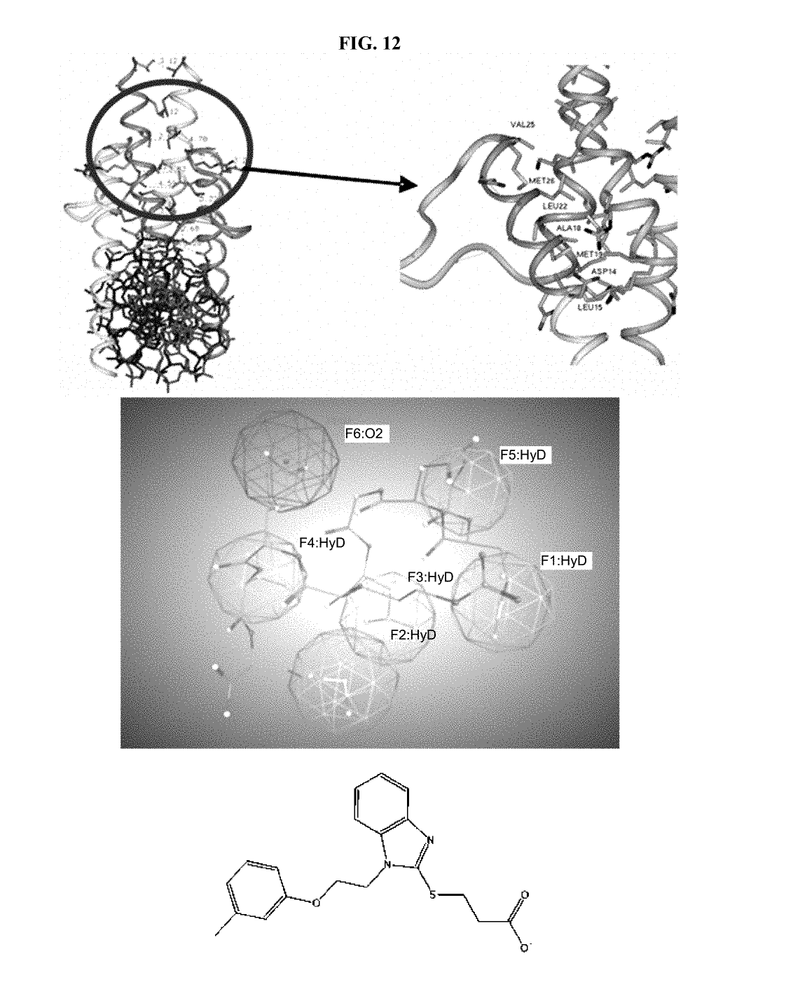

In some embodiments, the compounds (e.g., compounds of formula (I), (II), (III), (IV), (V), (VI), (VII), (VIII), or (IX)) described herein interfere with Olig2 dimerization with itself and partner proteins by blocking binding hotspots within the dimerization region. The pharmacophore for this region is defined herein (FIG. 1). The pharmacophore shown in FIG. 1 was used in generating an in silico compound search of small molecules described herein (e.g., in Table 1 or 2). The inhibitors in Table 3 were screen in a cell-based glioblastoma assay. These molecules were used in the design of additional inhibitors (Tables 1 and 2).

Table 3 left column in table is compound number, second column is chemical registry number, middle column shows the structure, and the last column indicates the IC50 in micromoles (μM) for human glioblastoma cells in culture.

### B. Example 2

From the dimerization region of Olig2 specific peptide sequences have been identified, which are shown in Table 4. These sequences are important in generating peptide probes for testing dimerization inhibition and are the basis of making peptidomimetic molecules. The sequence of the OLIG2 molecule directly involved in dimerization has been identified:

RLKINSRERKRMHDLNIAMDGLREVMPYAHGPSVRKLSKIATLLLARNYI
LMLTNSL

Table 4 lists peptide sequences predicted to bind the brown and gray colored regions which are most important for dimerization. Some sequences overlap both the loop and the direct contact sites which may increase target affinity. Sequence number 12 is a probe for the loop region, as this may block dimerization. The peptide RNYILMLTN has been synthesized.

Applicants have performed homology modeling of E47 and OLIG2 based on the crystal structure of E47 and the amino acid sequence of both proteins, and have identified the dimerization region of OLIG2.

Applicants have performed extensive computer modeling and have designed peptide probes for OLIG2, and Applicants have synthesized two probes for OLIG2 inhibition. Applicants have also defined the pharmacophore for both molecules.

Applicants have designed and synthesized probe compounds to confirm the pharmacophore and design peptides, peptidomimetics, and cyclic peptides and small organics.

Applicants have demonstrated experimentally that Olig2 silencing abolishes the ability of human glioblastomas to grow in mouse models. Applicants have suppressed human GBMs with inhibitors described herein, in vitro.

### C. Example 3

Applicants have optimized the design of several novel druggable Olig2 inhibitors. These new compounds are shown in Table 1 and 2 below. Tables 1 and 2 show analogues of Olig2 inhibitors from a pharmacophore similarity search with improved druggability profiles.

### D. Example 4

Transcription factors (TFs) are a major class of protein signaling molecules that play critical cellular roles in cancers, such as the highly lethal glioblastoma (GBM) brain tumors. Though TFs are promising targets for drug intervention, the development of specific TF inhibitors has proved difficult owing to expansive protein-protein interfaces and absence of hydrophobic pockets. In silico modeling is increasingly being used in drug design but has met with limited success in the context of TF inhibitors. Hence Applicants sought to redefine and expand computational strategies for TF inhibitor design. Applicants' case study was the OLIG2 protein, which is a TF essential for the survival and expansion of GBM. Applicants' analyses revealed that single residues or small foci, called binding hotspots that have typically been the focus of previous TF modeling efforts, do not adequately represent the total active dimerization interface. Rather, the TF dimerization surface appears to involve a general, parental pharmacophore, which in turn comprises several regional daughter pharmacophores, or subpharmacophores. Applicants hypothesized that small molecules able to fit each subpharmacophore in the OLIG2 dimerization region could selectively inhibit activating dimerization. Applicants identified candidate compound structures from library screens guided by Applicants' combined subpharmacophore hypotheses that demonstrated OLIG2 pathway inhibition and in vitro anti-GBM potency in biochemical and cell-based screens. These data may provide the basis for the discovery of novel small molecule inhibitors of TF dimerization, and potentially lead to the development of novel cancer therapeutics.

Transcription factors (TFs) comprise a large class of proteins that bind specific DNA regions and regulate gene expression to control key processes such as differentiation, cell cycle, survival, and apoptosis (Pabo C O & Sauer R T, Annual Review of Biochemistry 61:1053-1095 (1992); Riley T et al., Nature Reviews. Molecular Cell Biology 9:402-412 (2008)). Dysregulation of these proteins drives a range of disorders including various cancers, where they can act either as tumor suppressors or oncogenes (Nebert D W, Toxicology 181-182, 131-141 (2002); Papavassiliou A G, Anticancer Research 15:891-894 (1995)). Clearly, TFs represent a major therapeutic target (Darnell J E, Jr., Nature Reviews. Cancer 2:740-749 (2002); Karamouzis M V et al., Clinical Cancer Research: An Official Journal of the American Association for Cancer Research 8:949-961 (2002)), but the realization of specific inhibitors has proved notoriously difficult owing to large TF protein-protein dimerization interfaces, the absence of hydrophobic pockets, and different TF conformations between the free and dimerized states (Arkin M R & Wells J A, Nature Reviews. Drug discovery 3:301-317 (2004)).

Direct inhibition of TFs would ideally involve a small molecule able to interfere with TF-DNA interaction or with TF dimerization. Importantly, some success has been achieved using these two avenues. For example, a small molecule has been shown to inhibit the binding of the TF C/EBPa to DNA (Berg T, Current Opinion in Chemical Biology 12:464-471 (2008); Rishi V et al., Analytical Biochemistry 340:259-271 (2005)). Similarly, compounds have been shown to inhibit c-Myc/Max heterodimerization (Lu X et al., Oncology Reports 19:825-830 (2008); Xu Y et al., Bioorganic & Medicinal Chemistry 14:2660-2673 (2006)). Despite the fact that the activities of these compounds are not sufficient to justify further development as drug candidates, the success so far attained suggests that TFs can be directly inhibited.

Applicants were motivated to redefine and expand computational strategies to design small-molecule TF inhibitors owing to the considerable biological importance of TFs, and particularly due to their role in cancer stem cell growth and proliferation (Bao S et al., Nature 444:756-760 (2006); Cheng L et al., Biochem Pharmacol 80:654-665 (2010); Ikushima H et al., Cell Stem Cell 5:504-514 (2009)). Additionally, in vitro high-throughput screening for potential inhibitor structures is very costly and requires extensive resources. However, a well-known obstacle in designing compounds that inhibit TF dimerization is the presence of a large interface area between the TFs in the dimer. Moreover, TFs may acquire specific conformations during dimerization that may be very different from the conformations they exist in during the unbound state. These conditions necessitate developing new computational strategies for the design of compounds to bind TF interfaces and inhibit TF dimerization. Applicants' preliminary computational analyses prompted us to question previous TF inhibitor modeling efforts, which presumed that important sites in the dimerization surface are small foci or single residues, termed binding hotspots (Clackson T & Wells J A, Science 267:383-386 (1995); Jubb H et al., Trends Pharmacol Sci. (2012)). Rather, Applicants' results suggest that the TF dimerization surface includes a comparatively large, parental pharmacophore comprising several discrete but substantial daughter regions, or subpharmacophores.

Using the concept of a combined pharmacophore, ie., parental pharmacophore with multiple daughters, Applicants targeted OLIG2, a basic helix-loop-helix (bHLH) TF that is critical in tumorigenesis and regulates the survival and expansion of glioblastoma (GBM) (Dietrich J, Imitola J, & Kesari S, Nature Clinical Practice. Oncology 5:393-404 (2008); Ligon K L et al., Journal of Neuropathology and Experimental Neurology 63:499-509 (2004); Ligon K L et al., Neuron 53:503-517 (2007); Mehta S et al., Cancer Cell 19:359-371 (2011); Soda Y et al., Proceedings of the National Academy of Sciences of the United States of America 108:4274-4280 (2011); Wen P Y & Kesari S, The New England Journal of Medicine 359: 492-507 (2008)). Applicants' objectives were to define the pharmacophores related to the OLIG2-E47 interface and search existing chemical structure libraries for scaffolds predicted to bind the combined pharmacophores. Further, Applicants aimed to validate Applicants' approach by demonstrating both in vitro potency against human GBM, and OLIG2 selectivity of in silico identified compounds.

An initial challenge was that high-resolution crystal structures for OLIG2 and its dimers have not been reported. However, OLIG2 is known to bind E47, an E2A class TF whose crystal structure is resolved. In addition, OLIG2 has close sequence identity to a number of other TFs that bind the E2A group, and whose crystal structures have been resolved (Ellenberger T et al., Genes & Development 8:970-980 (1994)). Based on this information, Applicants analyzed the intermolecular contacts between OLIG2 and E2A TFs. In addition, using the E2A-NeuroD1 complex as a template (Longo A, Guanga G P, & Rose R B, Biochemistry 47: 218-229 (2008)), Applicants modeled both OLIG2 and the OLIG2E47 heterodimer. This allowed us to develop a new computational strategy to elucidate a set of possible “parent” and “daughter” pharmacophore hypotheses for targeting the OLIG2-E47 heterodimer interface. Applicants' analysis led us to three different four-feature daughter hypotheses, derived from the parental five-feature pharmacophore hypothesis. Further Applicants found that the OLIG2 hinge region within the binding domain has a unique structure among bHLH TFs.

The OLIG2 parental and daughter pharmacophore hypotheses guided 3D-structure searches of the Open NCI Database (http://cactvs.nci.nih.gov/download/nci/) that identified compounds with potential OLIG2 inhibitory activity. Those compounds predicted to engage all three daughter pharmacophores were acquired and screened in vitro against patient-derived GBM neurospheres. Applicants screened 103 chemical structures and found 23 compounds from various structural classes that exhibited activity in the low micromolar (μM) to high nanomolar (nM) range. The most potent compound was subjected to more comprehensive testing and was found to exhibit selectivity, as it only had significant activity in OLIG2-expressing cell lines, and it affected genetic targets directly regulated by OLIG2.

Computational Modeling of the Specific OLIG2-E47/12 Dimer Interface

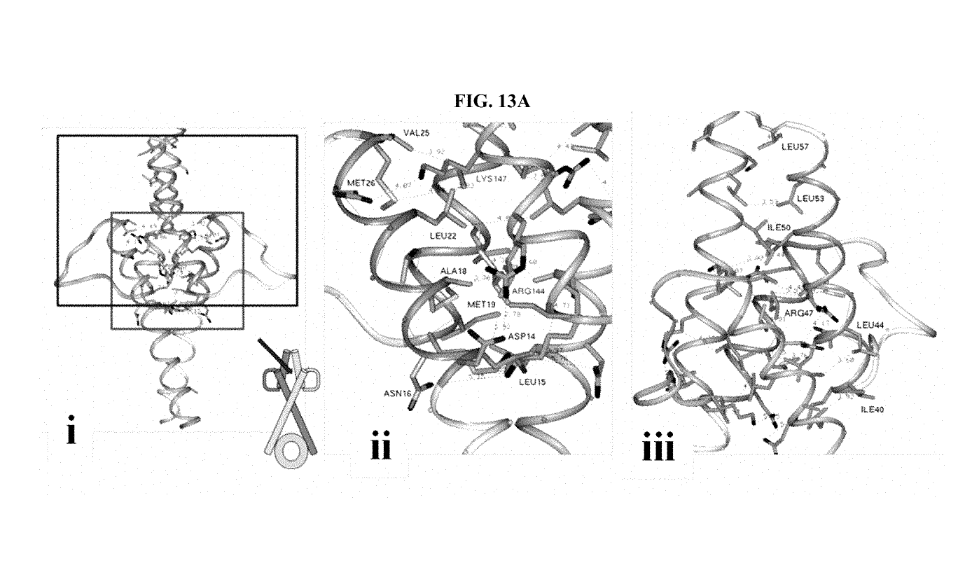

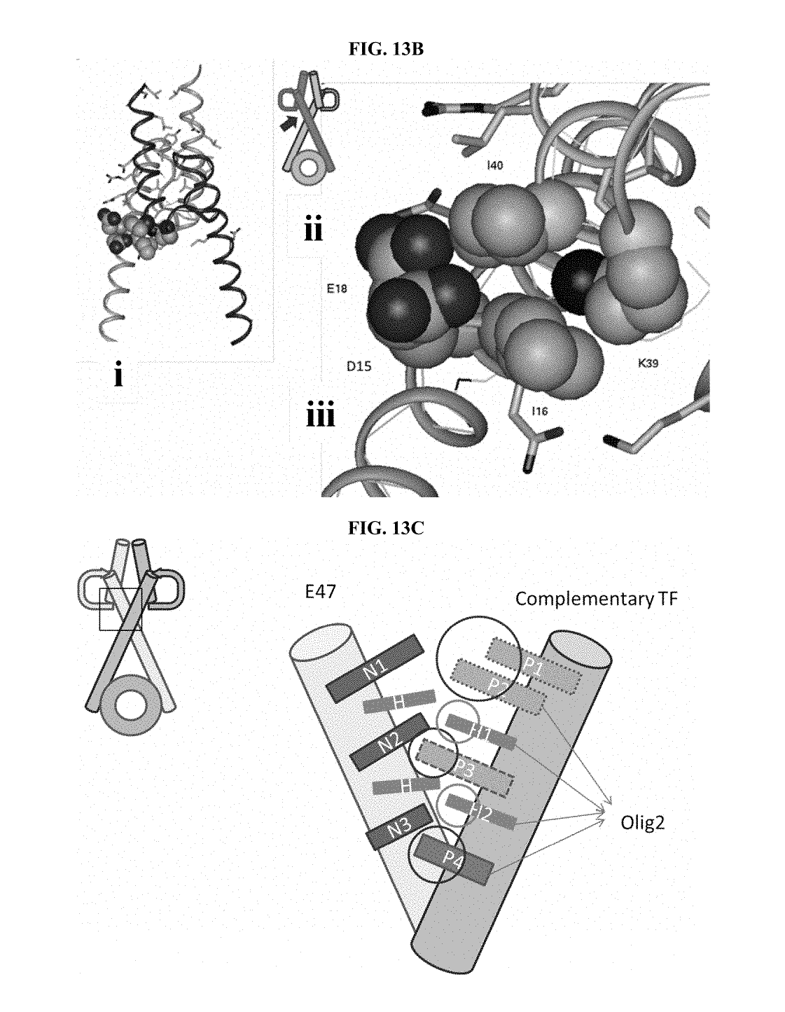

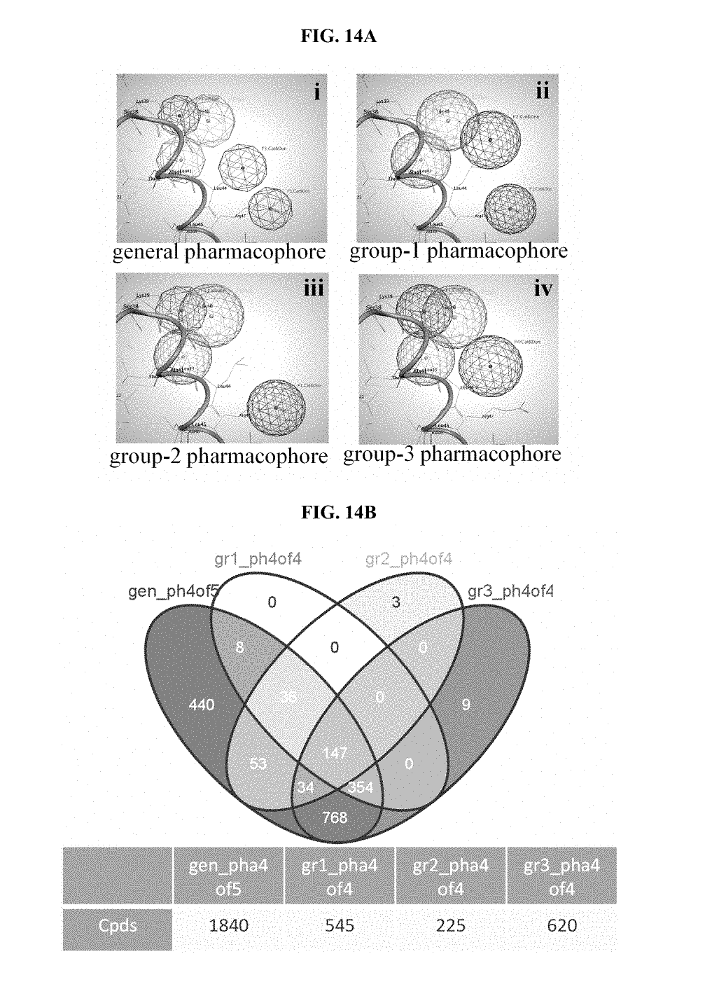

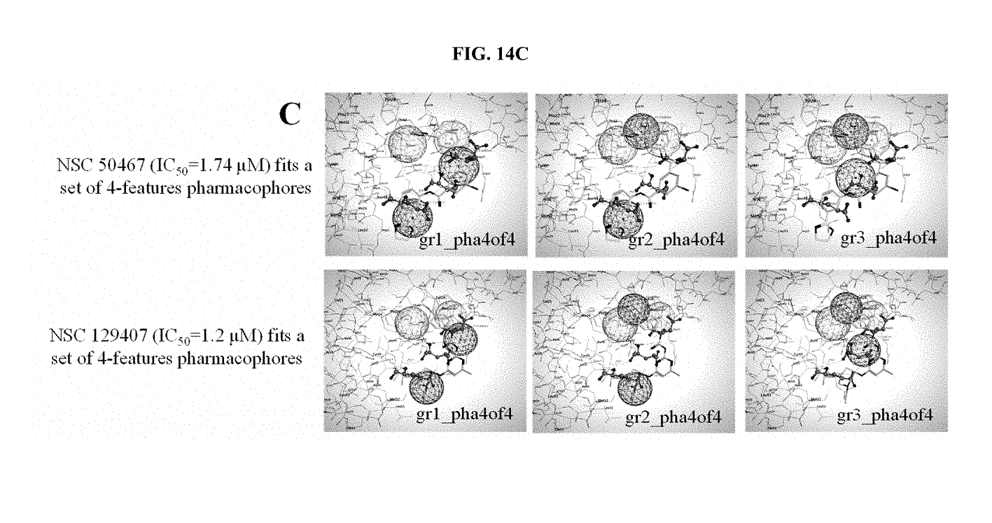

Based on a strong homology between OLIG2 and NeuroD1, Applicants modeled the 3-D structure of OLIG2-E47 heterodimer using as template, the crystallographic structure of NeuroD1-E47 heterodimer (PDB ID: 2ql2; FIG. 14A; (Longo A, Guanga G P, & Rose R B, Biochemistry 47: 218-229 (2008))). The modeled OLIG2-E47 dimer structure is depicted in FIG. 13A, with the inset illustrating the general topology of the heterodimer which contains unique sequences and pharmacophore features. The red arrow indicates the important OLIG2-E47 interface region that was used for pharmacophore design. The interface negative residues (E18 and D15) from E2A interact with the positive (K39) residue on OLIG2, and this triad is encompassed by two hydrophobic residues preventing water molecules from entering this contact zone (FIG. 13B). Such an arrangement significantly lowers the dielectric constant in the contact region and thus markedly increases attractive electrostatic energy between apposed residues.

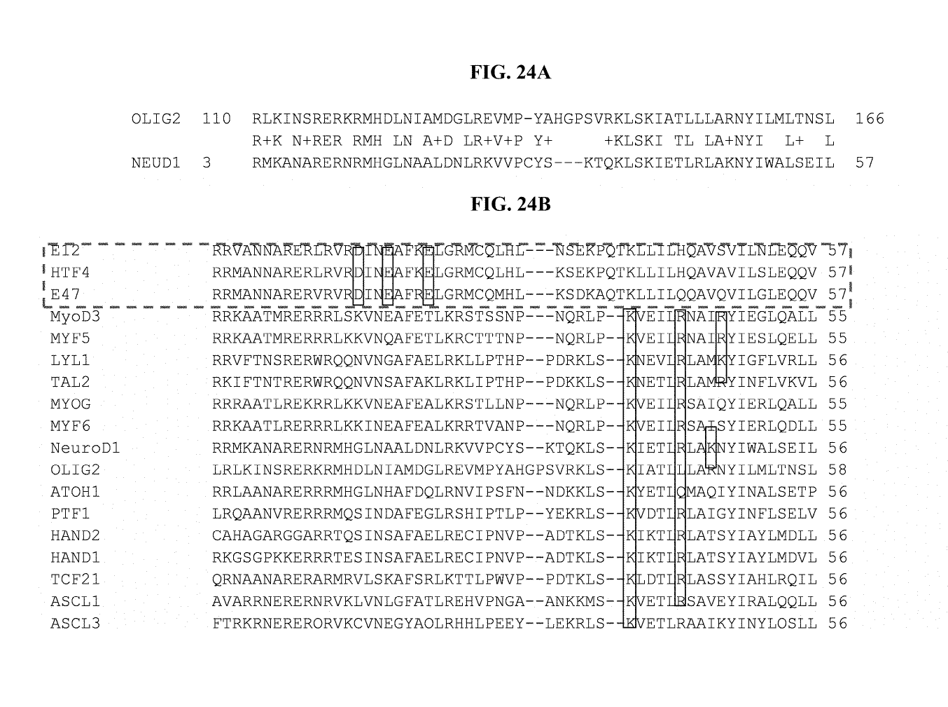

After modeling of OLIG2-E47 heterodimer Applicants conducted an analysis of possible structures of the heterodimers of E47 with the other TFs similar to OLIG2 included in the alignment (FIG. 24B). The general scheme of the interface between the group containing E2A and HTF4 TFs and the group of the other proteins is clearly dependent on complementary interactions between the positive and negative residues and formed the basis for subsequent pharmacophore development. The interface has a unique sequence and is amenable to the design of selective agents, although there are three conserved negative residues—one aspartic acid and two glutamic acid residues in the E2A group—and two positive residues—lysine and arginine—conserved among most of the other TFs, and a third positive site located further in the sequence and conserved in specific groups of TFs. All these residues are outlined by the rectangles on the alignment depicted in FIG. 24B. The scheme of the E47 interface with the complementary TF, which includes OLIG2 is shown in FIG. 13C. There is evident complementarity of the cationic negative residues on the E47 side (N1-N3) to the anionic positive residues (P1-P4) on the partner TF. Importantly, two possible positions of less conserved anionic residues for groups 1 and 2 are not far from each other in the 3D structure. Their side chains can be reasonably adjusted to be in the attracting energy range, as exemplified by the anionic residue N1 belonging to E2A.

Pharmacophore Hypotheses Development

Parental pharmacophore: The previous section outlines the basis for the development of pharmacophores for TFs complementary to E47 and this approach was used for OLIG2. The purple spheres shown in FIG. 14A represent cationic/donor features, while the green spheres denote hydrophobic features of the pharmacophore. The parental five-feature pharmacophore hypothesis (FIG. 14A, panel i) included all cationic/donor and hydrophobic features. TFs similar to OLIG2 (see FIG. 14) can be separated into three groups.

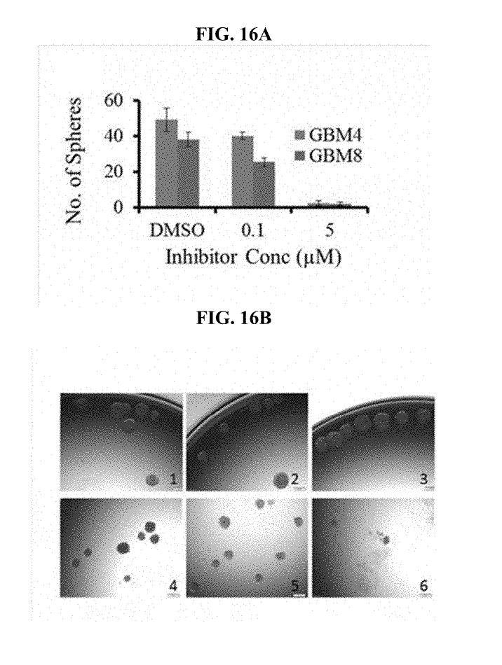

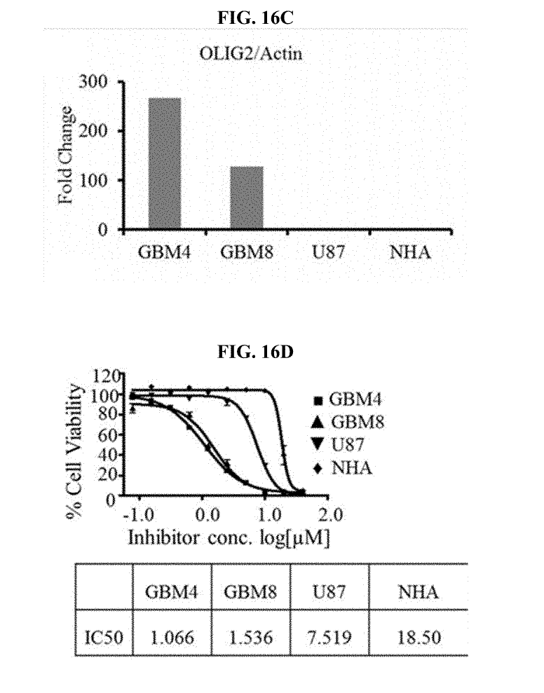

Subpharmacophores (daughter pharmacophores): Binding of E47 with TFs of each group is directed only by two positive-negative contacts of all three such possible contacts, so Applicants created set of four-feature daughter pharmacophore hypotheses containing the various combinations of pairs of cationic/donor features with the same two hydrophobic regions (FIG. 16A).

Pharmacophore-Guided Structure Similarity Search of Conformational Databases

The parental and daughter pharmacophores were used to search Applicants' conformational databases derived from the Open NCI Chemical Structure Database. The search yielded 1840 compounds predicted to fit 4 of 5 features belonging to the parental pharmacophore; only 4 features were searched to avoid search narrowing. Subsequent searches based on the 3 daughter pharmacophores, utilizing 4 features, yielded sets of 545, 273, and 1312 compounds, termed group-1, group-2, and group-3, respectively. The four set Venn diagram in FIG. 14B reveals that there are 147 compounds predicted to fit the four-of-five features of the parental and all features of daughter pharmacophores, 354 compounds that satisfy the four-of-five features of the parental and all features of two daughter pharmacophores (gr 1 and gr 3), another 36 that satisfy a separate combination of parental and daughter pharmacophores (gr 1 and gr 2), and 8 that satisfy the parental and one daughter pharmacophore (gr 1).

It is noteworthy that the compounds selected from the Open NCI Database set, which showed the most potent activity in subsequent GBM cell-based screens were mainly those that were predicted to fit all four hypothesized pharmacophores. Applicants found that ⅗ of the most potent compounds bound all the pharmacophores, and one compound bound ⅘ parental features, which showed some overlap with the three daughter pharmacophores. One compound bound all pharmacophores except one. This result is consistent with Applicants' contention that the OLIG2 TF dimerization active region may be operationally regarded as including a parental and daughter pharmacophores. The most potent compounds generally had conformations (different for each pharmacophore) that fit all, or nearly all four features of each pharmacophore. This means that a compound that fits all subpharmacophores has three separate opportunities to dock with the TF interface. This greatly increases the probability of binding to the dimerization interface and interfering with OLIG2E2A dimerization. FIG. 14C shows the possible configurations of two selected compounds that fit all four-feature subpharmacophores. Both of these compounds exhibited comparatively potent anti-GBM activity.

Definition of Compound Structure Classes

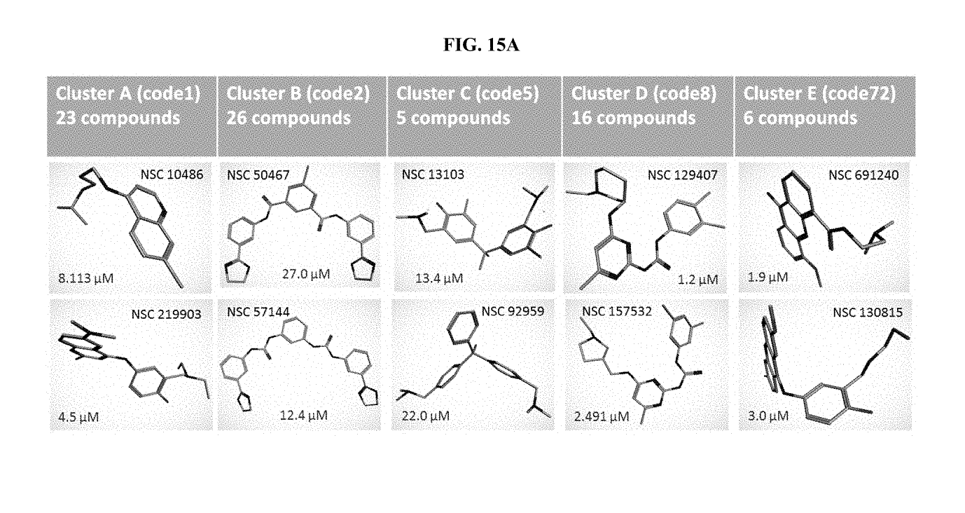

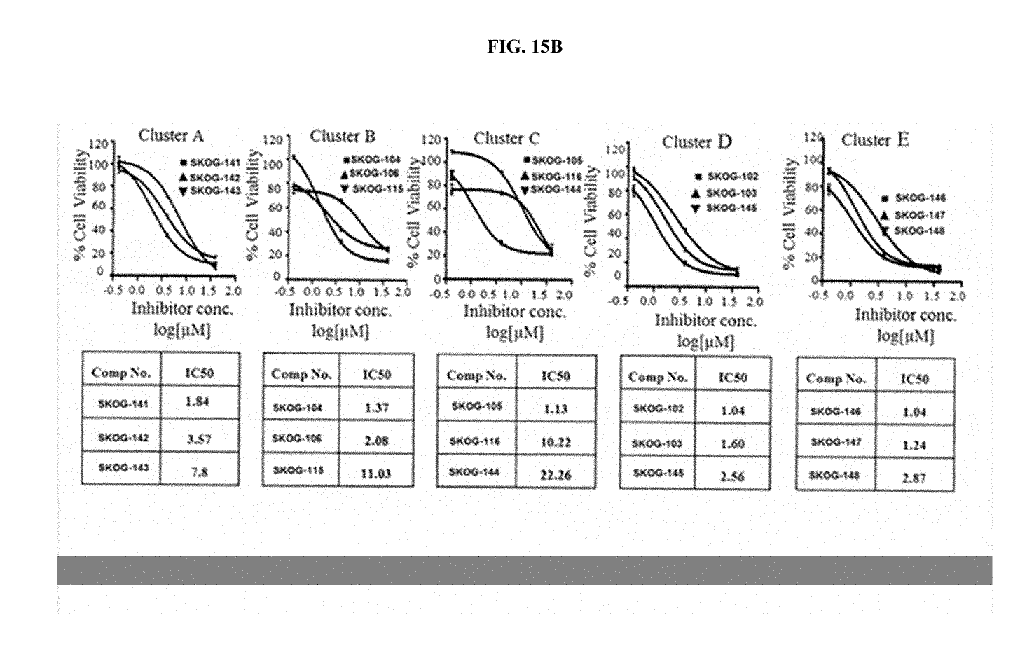

The next step was to confirm the chemical stability and features of compounds for in vitro validation of modeling/in silico search results and for the subsequent development as GBM therapeutics, For this purpose, Applicants further subdivided the compounds that fit all four-feature pharmacophores, depicted by the gray zone on the Venn diagram (FIG. 16B). These compounds were clustered into nine structural classes using MOE (Molecular Operating Environment, Chemical Computing Group—CCG, Montreal) software. Then Applicants refined the clusters based on size, structure and functional groups, charge, druggability, and chemical tractability criteria, removing unstable and highly charged compounds. Based on this analysis, Applicants selected five clusters (A-E) for in-vitro screening. Compounds that were not available from the NIH collections were eliminated from the final clusters depicted in FIG. 15A and listed below:

Cluster A—Includes 23 compounds that have the quininoline moiety in common. These compounds are frequently asymmetric, having an aliphatic or alicyclic tail terminating in a substituted amino group.

Cluster B—Contains 26 compounds all of which are either aromatic amides or ureas and most frequently have terminal dihydroimidazole ring structures.

Cluster C—All 5 compounds from this cluster can be classified as polyphenolic and are terminated by trisubstituted amino groups.

Cluster D—All 16 compounds in this cluster have either terminal substituted guanidine groups or disubstituted guanidine groups in the center of the molecules.

Cluster E—All 6 compounds in this cluster have the acridine moiety as a central scaffold and are substituted with hydrogen bond donating amino groups.

Biochemical and Cell-Based Validation of Compounds Identified by Pharmacophore Hypotheses

Several compounds from among the 103 tested, exhibited considerable potency against GBM cells in vitro. FIG. 15B shows the most potent compounds according to structural class (cluster). FIGS. 16A and 16B reveal that the most potent compound (SKOG-102) was active against two patient-derived GBM lines cultured as neurospheres. These GBM cancer stem cell derived lines are highly representative of actual tumors and furthermore, can recapitulate accurately the invasive natural history of GBM when implanted into immunocompromised mice.

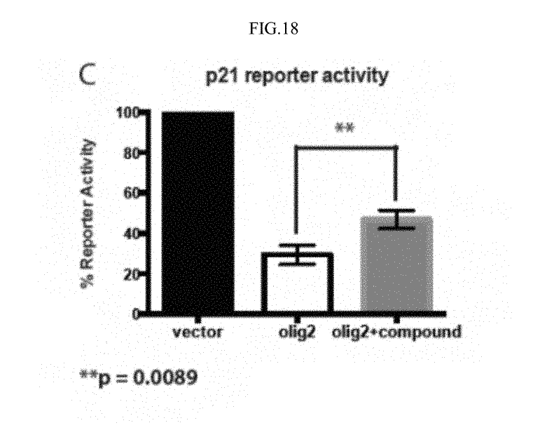

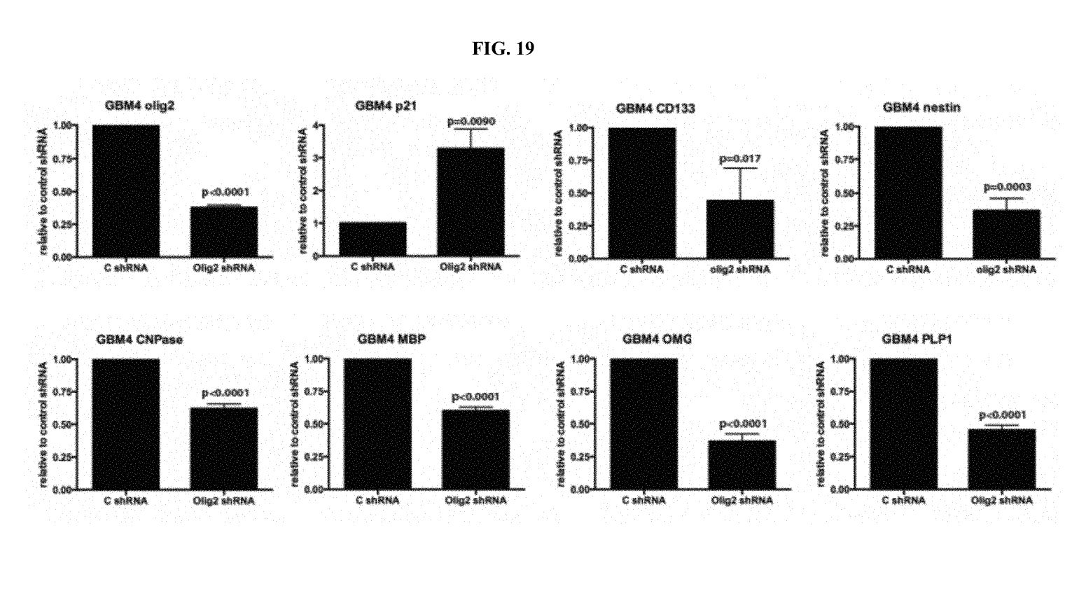

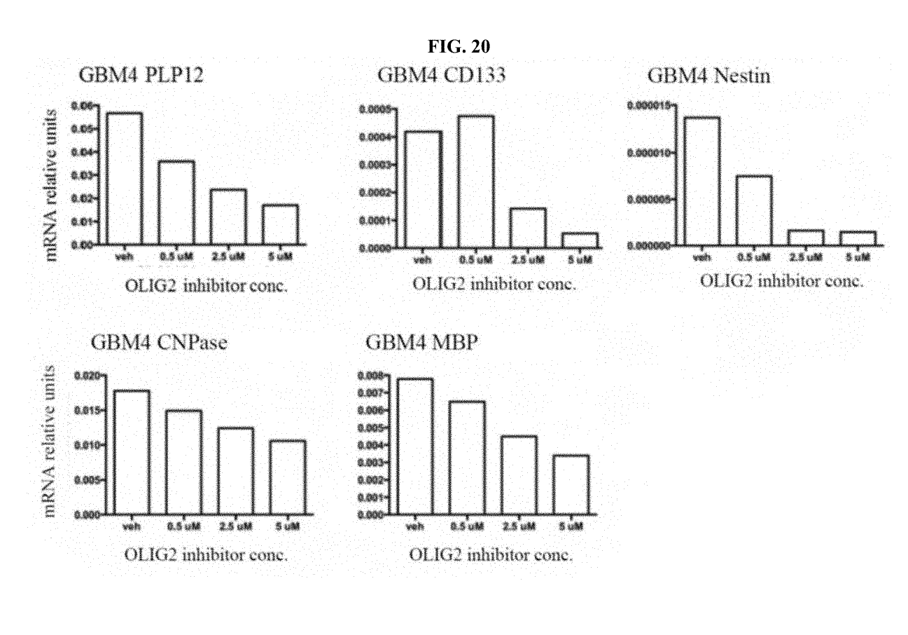

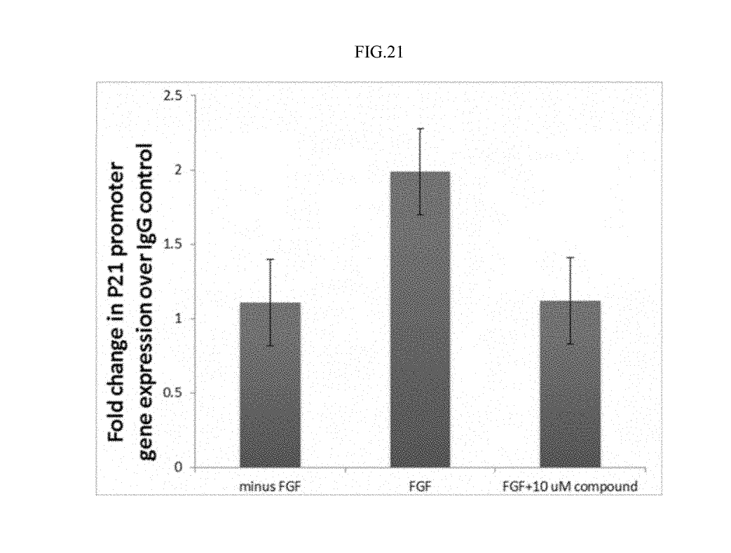

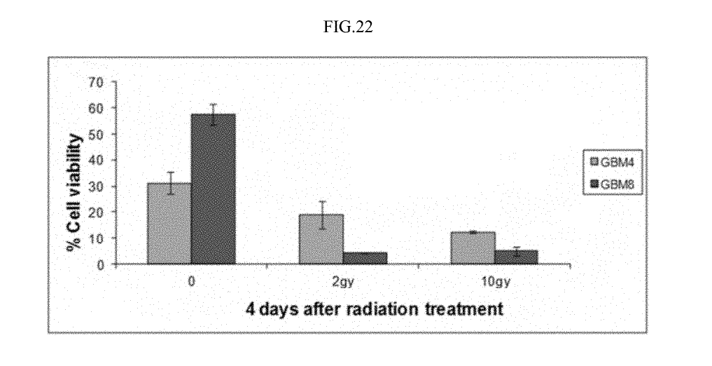

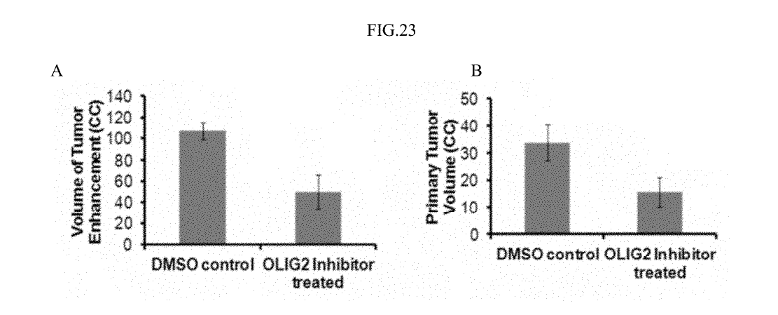

The most potent inhibitor appeared to be selective for OLIG2. qPCR analysis showed that GBM4/8 cells expressed more OLIG2 than U87 cells and that normal human astrocytes (NHA) expressed no OLIG2, as depicted in FIG. 16C. The graphs in FIG. 18D clearly show that the IC₅₀ for GBM4/8 cells was much lower than in U87 cells, and NHA required a very high dose of the inhibitor before showing toxicity.

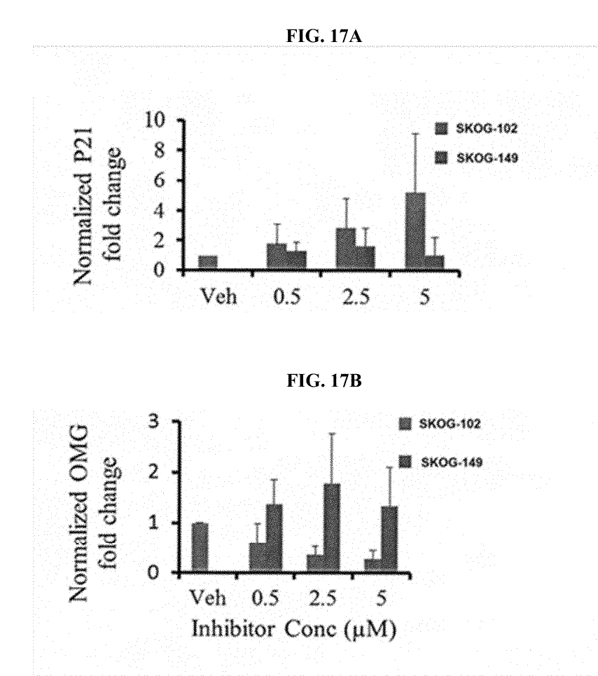

In FIG. 17, expression of direct genetic targets of OLIG2 (p21 and OMG) was examined following exposure to the OLIG2 inhibitor compound (SKOG-102). OLIG2 binds the p21 gene promoter and decreases its expression. Significantly, when the OLIG2 inhibitor was added in escalating doses, the levels of p21 increased in a dose-dependent manner (FIG. 17A). Conversely, OMG is upregulated by OLIG2 and addition of the inhibitor caused OMG levels to decline (FIG. 17B). These data strongly suggest that the inhibitor compound mainly acts via OLIG2, although off-target effects clearly occur at higher doses.

Discussion and Conclusions

The development of protein interface pharmacophores is considered to be one of most difficult and least tractable drug design tasks and this reality has limited the development of TF inhibitors (REF). This is particularly evident when compared with most typical drug design scenarios, which involves designing so-called “pocket” inhibitors that fit within the well-defined boundaries of a protein pocket. Such a pocket encompasses all possible positions of the designed compound, critical reference points relating to shape and all necessary pharmacophore features corresponding to the residues of the pocket. In contrast, large interfaces such as those presented by TFs contain no such boundaries and no shape limitations. Moreover, often the protein surfaces involved in the interface are wide open in terms of a solution and have a comparatively “flat” shape that is not amenable to the stable binding of any peptide or compound (REF). In consideration of the foregoing, Applicants developed a novel approach which enhances the probability of robust and stable inhibitor binding to TF interfaces, and Applicants' case study was the bHLH family of transcription factors, specifically the OLIG2-E47 TF heterodimer.

Applicants introduce the concept of a “combined pharmacophore hypotheses” which is embodied by various computationally derived sets, each one of which includes a “parental pharmacophore” and multiple “daughter subpharmacophores”. These combined pharmacophores were used to search for structures potentially able to bind all pharmacophores and thus avidly bind to the target protein and overcome the “flat” configuration of OLIG2. This methodology may potentially be generalized to various TFs, other protein-protein interface targets, and Applicants' work elucidates the salient features not only of the OLIG2-E47 interface, but of a number of bHLH heterodimers having related sequence and structural identities. These general structural features as well as those features unique to OLIG2-E47 were taken into consideration during OLIG2 pharmacophore design, specifically to culminate in the identification of OLIG2 selective compounds.

The parental and daughter pharmacophore hypotheses were used for the selection of possible inhibitors from the NCI open database. Validation of the combined pharmacophore approach was achieved by the identification and screening of compounds that suppressed human GBM in vitro and suppressed OLIG2 target genes. Moreover, inhibitor suppression of the levels of direct OLIG2 genetic targets, together with the comparatively weak cell killing of cells expressing little or no OLIG2, pointed to the likelihood that Applicants' most potent inhibitor did selectively bind OLIG2.

Applicants' combined pharmacophore approach is potentially applicable to other important TFs previously regarded as undruggable, and to other protein-protein interactions. The data presented herein supports further comprehensive investigation and validation of OLIG2 selective binding and interference with dimerization, using additional biochemical and x-ray crystallographic methods. Applicants' putative OLIG2/E2A dimerization inhibitors may be further assessed with in vivo GBM models, structurally optimized, and evaluated on a preclinical basis for subsequent development as potential GBM therapeutics.

Materials and Methods

Homology Modeling

As the first step in Applicants' analyses, Applicants selected TFs that bind to E2A using the APID program (Prieto C & De Las Rivas J, Nucleic Acids Research 34:W298-302 (2006)). The sequences of the selected TFs were aligned with the program Clustal W 2.1 (Larkin M A et al., Bioinformatics 23:2947-2948 (2007)). The next step involved preparing a homology model of OLIG2 and its heterodimer with E47, using the InsightII package (Accelrys, San Diego, Calif.). As a basis for this modeling, Applicants used the known structure of the E47-NeuroD1 dimer (PDB ID 2ql2). Applicants modeled only the specified region of interest in OLIG2 that was previously selected on the basis of alignment (FIG. 14A). In this region the sequence homology of OLIG2 to NeuroD1 was found to be ˜55% using the homology module of InsightII® modeling software. The resultant modeled OLIG2-E47 structure then underwent 10,000 iterations according to molecular mechanics minimization using the Discover (Accelrys, 2008) program.

Pharmacophore Development

Applicants applied an interface-based method for pharmacophore development, which implies that the 3D positions of the important residues of interacting proteins defined the pharmacophore hypothesis relevant to a compound for the inhibition of OLIG2-E2A (E47) dimerization. The analysis of the interface between E47 and “OLIG2-like transcription factors” led us to a five-feature parental pharmacophore that encompasses the general scheme of interaction between E2A and different classes of E2A-binding transcription factors including OLIG2 (see FIG. 15). This design was conducted using the Pharmacophore Editor module from the MOE 2011.10 program (CCG, Montreal, Canada). The designed pharmacophore hypothesis included two hydrophobic and three cationic/donor features. Based on this parent hypothesis, three different four-feature daughter hypotheses were designed, each of them containing the same two hydrophobes and different combinations of two of three cations/donors.

The Open NCI Database (http://cactvs.nci.nih.gov/download/nci/) containing 3D structures of over 250,000 compounds was searched for compounds that would fit all pharmacophores. First, Applicants created conformations for each of the NCI compounds using the Conformational Import module of MOE. Then, Applicants extensively searched the resultant conformational database, using the MOE Pharmacophore Search module, with the parental and each daughter pharmacophore. The resulting four sets of compounds able to variously fit the different pharmacophores were used to create the four-set Venn diagram, using the VENNY server (Oliveros J C, http://bioinfogp.cnb.csic.es/tools/venny/index.html (2007)). Fingerprints for the compounds from the intersection zone common to all four sets were calculated and the compounds were clusterized using the similarity method (MOE Fingerprints module) (Williams, C, private communication). This process involved a three-point-pharmacophore-based fingerprint calculated from the 2D molecular graph, GpiDAPH3 (Graph of pi-system-donor-acceptor-polar-hydrophobe 3-point pharmacophore), as a fingerprint scheme. Then Applicants applied nearest-neighbor Jarvis-Patrick clustering with the both similarity (S) and overlap (O) parameters: SO=0.45 (Jarvis R A & Patrick E A, Ieee Transactions on Computers C-22:1025-1034 (1973); Williams C, Molecular diversity 10:311-332 (2006)), and Tanimoto coefficient as a similarity metrics. This SO value has been shown to be optimal for GpiDAPH3-fingerprint schemes (Williams, C, private communication). The Tanimoto similarity coefficient S(i,j) estimates similarities between two compounds (Thorner D A et al., Journal of Computer-aided Molecular Design 11:163-174 (1997); Willett P, J Chem Inf Comput Sci 38:983-996 (1998)). For two compounds i and j with fingerprints of length lᵢ and lⱼ, respectively, S(i,j)=l_{i,j}/(lᵢ+lⱼ−l_{i,j}), where lᵢ is the number of bits in molecule i, lⱼ is the number of bits in molecule j, and l_{i,j} is the number of common bits between i and j. The similarity scores between the reference molecule and each molecule in the database are computed and ranked, thus creating clusters.

Cell Culture and Cell Viability Assays

Culture and assay system: Ink4a/arfEGFR-VIII mouse cells and U87 human GBM cells were cultured in DMEM medium with 10% FBS. GBM4 and GBM8 patient-derived tumor neurosphere lines were cultured in stem cell medium supplemented with FGF and EGF. Primary normal human astrocytes (NHA) were cultured in astrocyte medium (Life Technologies, Grand Island N.Y.) with EGF. Cytotoxicity of the compounds was assessed by 3-(4,5-dimethylthiazol-2-yl)-2,5-diphenyltetrazolium bromide (MTT) reduction assay (for Ink4a/arfEGFR-VIII cells) as described earlier (Rajesh M et al., Journal of the American Chemical Society 129:11408-11420 (2007)) or by Alamar Blue assay (for all other cell types). For the MTT reduction assay, Ink4a/arfEGFR-VIII cells were grown in 96-well plates at a concentration of ˜2000 cells per well overnight and all assays were conducted in DMEM medium with 10% FBS and additives. For studies with inhibitor compounds, 10 mM stocks were serially diluted in dimethyl sulfoxide and further diluted into culture media to give appropriate concentrations while minimizing precipitation associated with serial dilutions in medium alone. Inhibitors were added to cells and left in the media for 72 hours. Cell viability for Ink4a/arfEGFR VIII cells was quantified at 540 nm after the addition of MTT (Sigma-Aldrich). Results were expressed as:

Percent viability=[A540(treated cells)−background/A540(untreated cells)−background]×100

For GBM4, GBM8, U87, and NHA cell lines, viability was quantified by Alamar Blue Assay. In this assay, cells were treated with inhibitor compounds as described and Alamar Blue added after 72 hours. Emission values at 590 nm were measured after the addition of Alamar Blue. Dose-response curves for MTT assays and Alamar Blue Assays were plotted and IC₅₀ values were calculated by using GraphPad Prism (GraphPad Prism Software, Inc., La Jolla, Calif.).

Statistical analysis: Classical statistical considerations were used, ANOVA and t-test. OLIG2 inhibition in GBM cells and NHA was assessed by dividing the average number of viable cells from three treatment plate replicates per dose by the average of three controls. At a type I error rate of 0.05, using a one-sided t-test, Applicants had 80% power to evaluate whether a decrease in mean percent viable cells was significantly lower than 100%.

Chemosensitivity of GBM Neurospheres

GBM4 cells were plated in 96-well plates and cultured as neurospheres (Sunayama J et al., Neuro-Oncology 12:1205-1219 (2010)). The active compound—129407 [1-(3,4-dichlorophenyl)-3-(4-((1-ethyl-3-piperidyl)amino)-6-methyl-2-pyrimidinyl)guanidine] and the inactive compound—305831 [3-(4,5-dimethylthiazol-2-yl)-2,5-diphenyltetrazolium bromide] were added at varying concentrations 12 h after plating. Neurospheres were viewed and photographed under Nikon microscope 4× objective after 72 h of incubation.

Quantitation of OLIG2 and OLIG2-Target mRNA Expression of Cell Lines

mRNA was extracted from different cell lines with the AllPrep DNA/RNA Mini Kit (Qiagen, Inc.), followed by cDNA synthesis using the iScript cDNA Synthesis Kit (Bio-Rad, Inc). To investigate single-gene expression patterns, individual gene primers were purchased from Allele Biotechnology and Pharmaceuticals Inc. SYBR Green Real Time PCR master mixes were purchased from Roche Corporation. qPCR was performed with primers specific for OLIG2, p21, and OMG genes. Individual gene expression was normalized to expression of beta-Actin. Histograms show mean and standard error of the mean (SEM) from three separate experiments.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### E. Example 5

Provided below are exemplary synthesis schemes for compounds provided herein. The schemes provided below may be generalized as appropriate using chemical synthetic techniques known in the art.

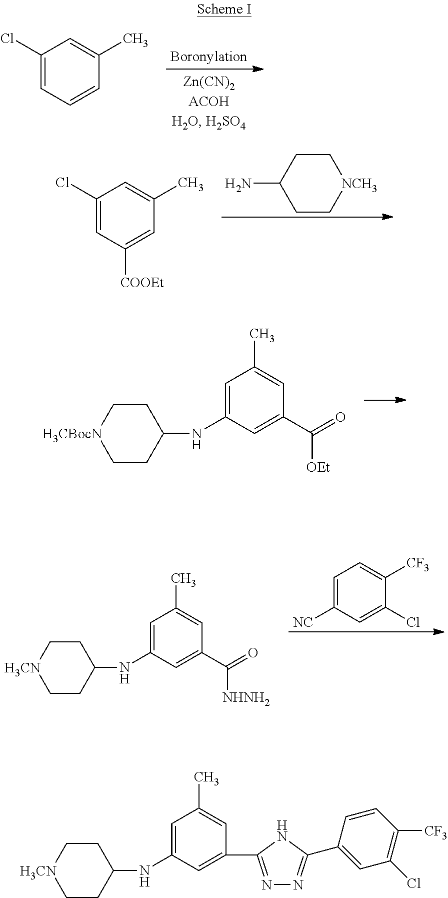

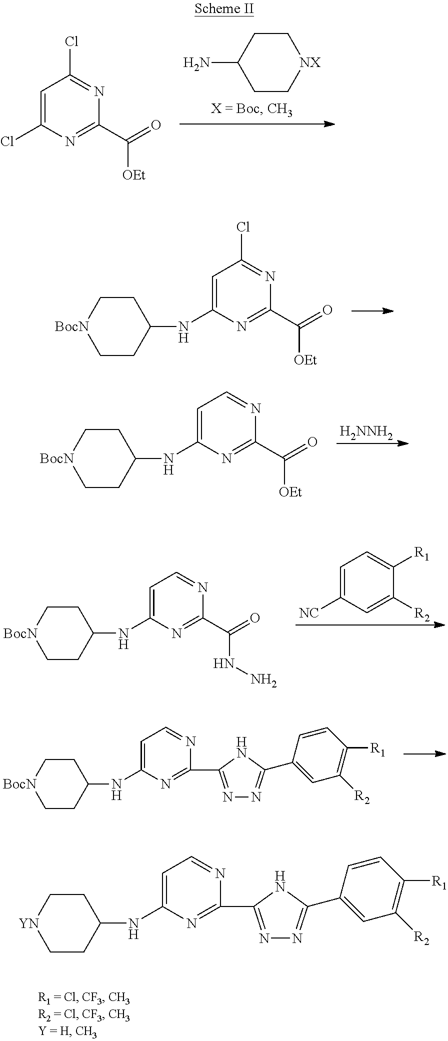

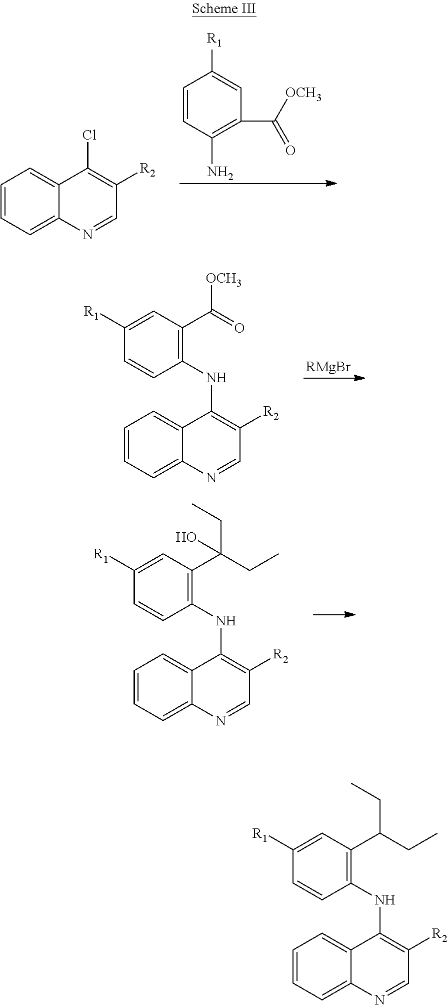

### REFERENCES

1. Dietrich J, Imitola J, Kesari S. Mechanisms of Disease: the role of stem cells in the biology and treatment of gliomas. Nature Clinical Practice Oncology 2008; 5(29): 9547-9556.

2. Sanai, N., A. Alvarez-Buylla, and M. S. Berger. Neural stem cells and the origin of gliomas. N Engl J Med 2005; 353:811-22.

3. Ligon K L, Huillard E, Mehta S, Kesari S, Liu H, Alberta J A et al., Olig2-regulated lineage-restricted pathway controls replication competence in neural stem cells and malignant glioma. Neuron. 2007 Feb 15;53(4):503-17.

4. Rhee W, Ray S, Yokoo H, Hoane M E, Lee C C, Mikheev A M, et al., Quantitative analysis of mitotic Olig2 cells in adult human brain and gliomas: implications for glioma histogenesis and biology. Glia. 2009 Apr 1;57(5):510-23.

5. Ligon K L, Fancy S P, Franklin R J, Rowitch D H. Olig gene function in CNS development and disease. Glia. 2006 Jul;54(1):1-10.

6. Lee S K, Lee B, Ruiz E C, Pfaff S L. Olig2 and Ngn2 function in opposition to modulate gene expression in motor neuron progenitor cells. Genes Dev. 2005 Jan 15;19(2):282-94.

7. Doukas J, Wrasidlo W, Noronha G, Dneprovskaia E, Hood J, Soll R. Isoform-selective PI3K inhibitors as novel therapeutics for the treatment of acute myocardial infarction. Biochem Soc Trans. 2007 Apr;35(Pt 2):204-6.

8. Noronha G, Barrett K, Cao J, Dneprovskaia E, Fine R, Gong X, et al., Discovery and preliminary structure-activity relationship studies of novel benzotriazine based compounds as Src inhibitors. Bioorg Med Chem Lett. 2006 Nov 1;16(21):5546-50.

9. Lange B, Schroeder U, Huebener N, Jikai J, Wenkel J, Strandsby A, et al., Rationally designed hydrolytically activated etoposide prodrugs, a novel strategy for the treatment of neuroblastoma. Cancer Lett. 2003 Jul 18;197(1-2):225-30.

10. Murphy E A, Shields D J, Stoletov K, Dneprovskaia E, McElroy M, Greenberg J I, et al., Disruption of angiogenesis and tumor growth with an orally active drug that stabilizes the inactive state of PDGFR{beta}/B-RAF. Proc Natl Acad Sci USA. 2010;107(9):4299-304.

11. Doukas J, Eide L, Stebbins K, Racanelli-Layton A, Dellamary L, Martin M, et al., Aerosolized phosphoinositide 3-kinase gamma/delta inhibitor TG100-115 [3-[2,4-diamino-6-(3-hydroxyphenyl)pteridin-7-yl]phenol] as a therapeutic candidate for asthma and chronic obstructive pulmonary disease. J Pharmacol Exp Ther. 2009 Mar;328(3):758-65.

12. Tsigelny I, Sharikov Y, Ten Eyck L F. Hidden Markov models-based system (HMMSPECTR) for detecting structural homologies on the basis of sequential information. Protein Eng. 2002; 15(5):347-52.

13. Lathia J D, Gallagher J, Heddleston J M, Wang J, Eyler C E, Macswords J, et al., Integrin alpha 6 regulates glioblastoma stem cells. Cell Stem Cell. 2010 May 7;6(5):421-32.

14. Chen R, Nishimura M C, Bumbaca S M, Kharbanda S, Forrest W F, Kasman I M, et al., A hierarchy of self-renewing tumor-initiating cell types in glioblastoma. Cancer Cell. 2010 Apr 13;17(4):362-75.

15. Hambardzumyan D, Squatrito M, Holland E C. Radiation resistance and stem-like cells in brain tumors. Cancer Cell. 2006 Dec;10(6):454-6

16. Bao S, Wu Q, McLendon R E, Hao Y, Shi Q, Hjelmeland A B, Dewhirst M W, Bigner D D, Rich J N. Glioma stem cells promote radioresistance by preferential activation of the DNA damage response. Nature. 2006 Dec 7;444(7120):756-60.

17. Rich J N. Cancer stem cells in radiation resistance. Cancer Res. 2007 Oct 1;67(19):8980-4.

18. Rhee W, Ray S, Yokoo H, Hoane M E, Lee C C, Mikheev A M, Horner P J, Rostomily R C. Quantitative analysis of mitotic Olig2 cells in adult human brain and gliomas: implications for glioma histogenesis and biology. Glia. 2009 Apr 1;57(5):510-23.

19. Macdonald D R, Cascino T L, Schold S C Jr, et al: Response criteria for phase II studies of supratentorial malignant glioma. J Clin Oncol 8:1277-1280, 1990.

20. Xu Z, Nagashima K, Sun D, Rush T, Northrup A, Andersen J N, Kariv I, Bobkova E V. Development of high-throughput TR-FRET and AlphaScreen assays for identification of potent inhibitors of PDK1. J Biomol Screen. 2009 Dec;14(10):1257-62.

21. Lee Y R, Park J H, Hahm S H, Kang L W, Chung J H, Nam K H, Hwang K Y, Kwon I C, Han Y S. Development of Bimolecular Fluorescence Complementation Using Dronpa for Visualization of Protein-Protein Interactions in Cells. Mol Imaging Biol. 2010 Apr 7.

22. C. A. Lipinski; F. Lombardo; B. W. Dominy and P. J. Feeney (1997). “Experimental and computational approaches to estimate solubility and permeability in drug discovery and development settings”. Adv Drug Del Rev 23: 3-25.

23. Cancer Genome Atlas Research Network. Comprehensive genomic characterization defines human glioblastoma genes and core pathways. Nature. 2008 Oct 23;455(7216): 1061-8.

24. Brennan C, Momota H, Hambardzumyan D, Ozawa T, Tandon A, Pedraza A, Holland E. Glioblastoma subclasses can be defined by activity among signal transduction pathways and associated genomic alterations. PLoS One. 2009 Nov 13;4(11):e7752.

25. Wright C W, Duckett C S. New insights into the function of IAP proteins: modulation of the MYC/MAX/MAD network. Dev Cell. 2008 Jan;14(1):3-4.

26. Strojnik T, Rosland G V, Sakariassen P O, Kavalar R, Lah T. Neural stem cell markers, nestin and musashi proteins, in the progression of human glioma: correlation of nestin with prognosis of patient survival. Surg Neurol. 2007 Aug;68(2):133-43; discussion 143-4.

27. Rebetz J, Tian D, Persson A, Widegren B, Salford L G, Englund E, Gisselsson D, Fan X. Glial progenitor-like phenotype in low-grade glioma and enhanced CD133-expression and neuronal lineage differentiation potential in high-grade glioma. PLoS One. 2008.

28. Zhang M, Song T, Yang L, Chen R, Wu L, Yang Z, Fang J. Nestin and CD133: valuable stem cell-specific markers for determining clinical outcome of glioma patients. J Exp Clin Cancer Res. 2008; 24;27:85.

29. Beier D, Hau P, Proescholdt M, Lohmeier A, Wischhusen J, Oefner P J, Aigner L, Brawanski A, Bogdahn U, Beier C P. CD133(+) and CD133(−) glioblastoma-derived cancer stem cells show differential growth characteristics and molecular profiles. Cancer Res. 2007:67(9):4010-5.

30. Shu Q, Wong K K, Su J M, Adesina A M, Yu L T, Tsang Y T, et al., Direct orthotopic transplantation of fresh surgical specimen preserves CD133+ tumor cells in clinically relevant mouse models of medulloblastoma and glioma. Stem Cells. 2008 26(6):1414-24.

31. Nayeem A, Sitkoff D, Krystek S. A comparative study of available software for high-accuracy homology modeling: from sequence alignments to structural models. Protein Sci. 2006;15(4):808-24

32. Wen P Y, Kesari S. Malignant gliomas in adults. N Engl J Med. 2008: 359(5):492-507.

33. Duffner P K. Risk factors for cognitive decline in children treated for brain tumors. Eur J Paediatr Neurol. 2010;14(2):106-15.

34. Longo A, Guanga G P, Rose R B. Crystal structure of E47-NeuroD1/beta2 bHLH domain-DNA complex: heterodimer selectivity and DNA recognition. Biochemistry. 2008: 8;47(1):218-29.

35. Klekota, J., E. Brauner, et al. (2005). “Identifying biologically active compound classes using phenotypic screening data and sampling statistics.” J Chem Inf Model 45(6): 1824-36.

36. Yan, S. F., H. Asatryan, et al. (2005). “Novel statistical approach for primary high-throughput screening hit selection.” J Chem Inf Model 45(6): 1784-90.

37. Tanimoto, T. T. (1957) IBM Internal Report 17th Nov.

38. Benjamini, Y., and Hochberg, Y. (1995). Controlling the false discovery rate: a practical and powerful approach to multiple testing. Journal of the Royal Statistical Society Series B, 57, 289-300.

39. Silverman R B. The organic chemistry of drug design and drug action: Chapter 2.2—Lead modification. Elsevier, San Diego, 2004, p17-65.

### VIII. Tables

TABLE 1
Pyrimidine-based Olig2 inhibitors.

| Compound structure | Compound name |
|---|---|
| 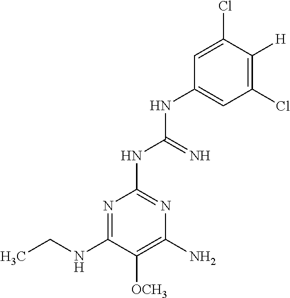 [SMILES: CCNc1nc(NC(=N)Nc2cc(Cl)cc(Cl)c2)nc(N)c1OC] | 1-(4-amino-6-(ethylamino)-5- methoxypyrimidin-2-yl)-3-(3,5- dichlorophenyl)guanidine |
| 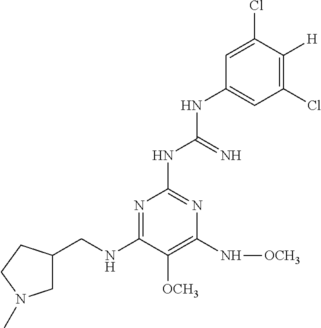 [SMILES: CONc1nc(NC(=N)Nc2cc(Cl)cc(Cl)c2)nc(NCC2CCN(C)C2)c1OC] | 1-(3,5-dichlorophenyl)-3-(5- methoxy-4-(methoxy- amino)-6-((1-methyl- pyrrolidin-3-yl)methyl- amino)pyrimidin-2- yl)guanidine |
| 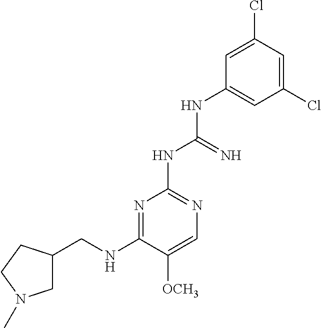 [SMILES: COc1cnc(NC(=N)Nc2cc(Cl)cc(Cl)c2)nc1NCC1CCN(C)C1] | 1-(3,5-dichlorophenyl)-3- (5-methoxy-4-((1-methyl- pyrrolidin-3- yl)methylamino)pyrimidin- 2-yl)guanidine |
| 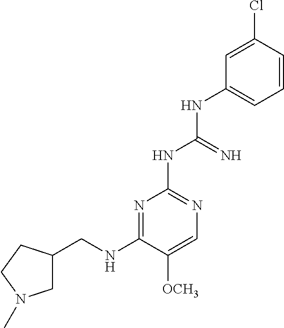 [SMILES: COc1cnc(NC(=N)Nc2cccc(Cl)c2)nc1NCC1CCN(C)C1] | 1-(3-chlorophenyl)-3-(5-methoxy- 4-((1-methylpyrrolidin-3- yl)methylamino)pyrimidin- 2-yl)guanidine |
| 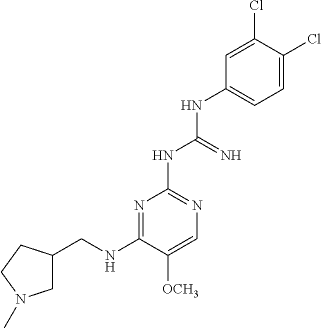 [SMILES: COc1cnc(NC(=N)Nc2ccc(Cl)c(Cl)c2)nc1NCC1CCN(C)C1] | 1-(3,4-dichlorophenyl)-3-(5- methoxy-4-((1-methyl- pyrrolidin-3- yl)methylamino)pyrimidin- 2-yl)guanidine |
| 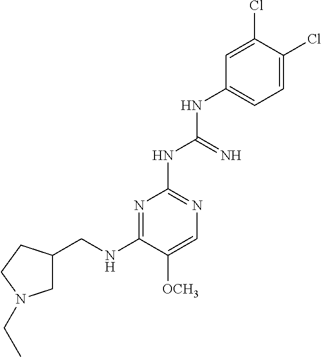 [SMILES: CCN1CCC(CNc2nc(NC(=N)Nc3ccc(Cl)c(Cl)c3)ncc2OC)C1] | 1-(3,4-dichlorophenyl)-3-(4- ((1-ethylpyrrolidin-3- yl)methylamino)-5- methoxypyrimidin-2- yl)guanidine |
| 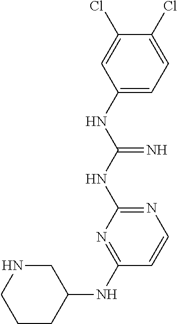 [SMILES: N=C(Nc1ccc(Cl)c(Cl)c1)Nc1nccc(NC2CCCNC2)n1] | 1-(3,4-dichlorophenyl)-3-(4- (piperidin-3- ylamino)pyrimidin-2- yl)guanidine |
| 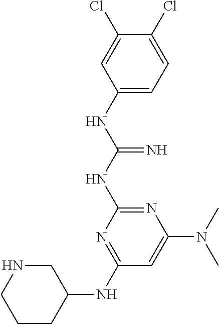 [SMILES: CN(C)c1cc(NC2CCCNC2)nc(NC(=N)Nc2ccc(Cl)c(Cl)c2)n1] | 1-(3,4-dichlorophenyl)-3-(4- (dimethylamino)-6-(piperidin-3- ylamino)pyrimidin-2-yl)guanidine |
| 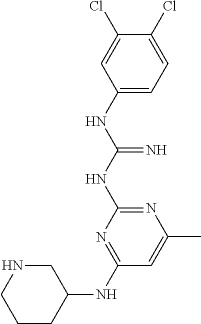 [SMILES: Cc1cc(NC2CCCNC2)nc(NC(=N)Nc2ccc(Cl)c(Cl)c2)n1] | 1-(3,4-dichlorophenyl)-3-(4- methyl-6-(piperidin-3- ylamino)pyrimidin-2- yl)guanidine |
| 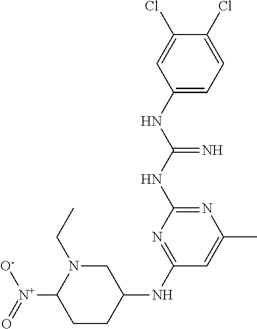 [SMILES: CCN1CC(Nc2cc(C)nc(NC(=N)Nc3ccc(Cl)c(Cl)c3)n2)CCC1[N+](=O)[O-]] | 5-(2-(3-(3,4-dichloro- phenyl)guanidino)-6- methylpyrimidin-4-ylamino)-1- ethyl-N-methyl-N- oxopiperidin-2-aminium |
| 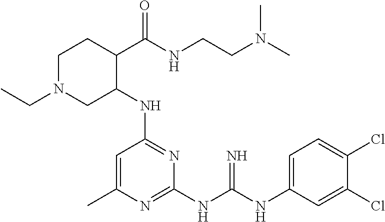 [SMILES: CCN1CCC(C(=O)NCCN(C)C)C(Nc2cc(C)nc(NC(=N)Nc3ccc(Cl)c(Cl)c3)n2)C1] | 3-(2-(3-(3,4-dichloro- phenyl)guanidino)-6- methylpyrimidin-4-ylamino)- N-(2-(dimethylamino)ethyl)- 1-ethylpiperidine-4- carboxamide |
| 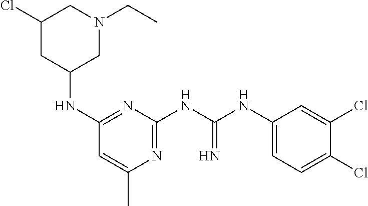 [SMILES: CCN1CC(Cl)CC(Nc2cc(C)nc(NC(=N)Nc3ccc(Cl)c(Cl)c3)n2)C1] | 1-(4-(5-chloro-1-ethylpiperidin- 3-ylamino)-6-methyl- pyrimidin-2-yl)-3-(3,4- dichlorophenyl)guanidine |
| 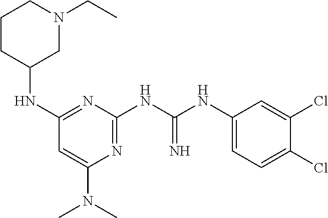 [SMILES: CCN1CCCC(Nc2cc(N(C)C)nc(NC(=N)Nc3ccc(Cl)c(Cl)c3)n2)C1] | 1-(3,4-dichlorophenyl)-3-(4- (dimethylamino)-6-(1-ethyl- piperidin-3- ylamino)pyrimidin-2- yl)guanidine |
| 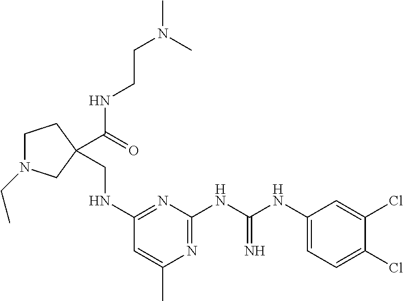 [SMILES: CCN1CCC(CNc2cc(C)nc(NC(=N)Nc3ccc(Cl)c(Cl)c3)n2)(C(=O)NCCN(C)C)C1] | 3-((2-(3-(3,4-dichloro- phenyl)guanidino)-6- methylpyrimidin-4- ylamino)methyl)-N-(2- (dimethylamino)ethyl)-1- ethylpyrrolidine-3-carboxamide |
| 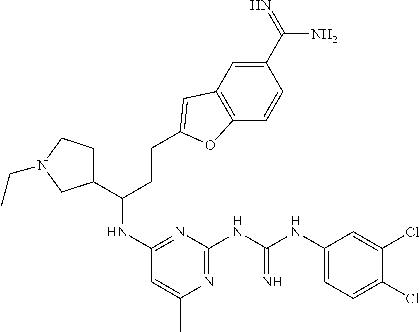 [SMILES: CCN1CCC(C(CCc2cc3cc(C(=N)N)ccc3o2)Nc2cc(C)nc(NC(=N)Nc3ccc(Cl)c(Cl)c3)n2)C1] | 2-(3-(2-(3-(3,4-dichloro- phenyl)guanidino)-6- methylpyrimidin-4- ylamino)-3-(1- ethylpyrrolidin-3- yl)propyl)benzofuran-5- carboximidamide |
| 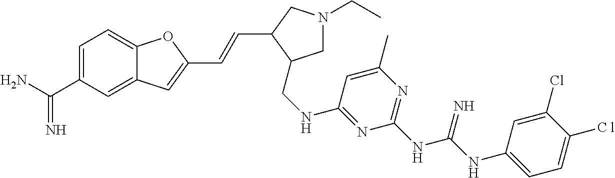 | (E)-2-(2-(4-((2-(3-(3,4- dichlorophenyl)guanidino)-6- methylpyrimidin-4- ylamino)methyl)-1- ethylpyrrolidin-3- yl)vinyl)benzofuran-5- carboximidamide |
| 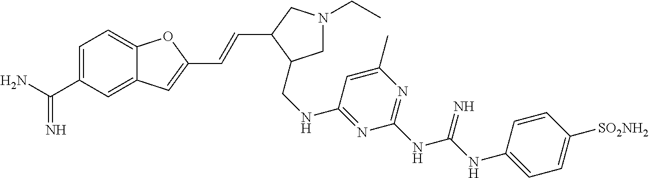 [SMILES: CCN1CC(/C=C/c2cc3cc(C(=N)N)ccc3o2)C(CNc2cc(C)nc(NC(=N)Nc3ccc(S(N)(=O)=O)cc3)n2)C1] | (E)-2-(2-(1-ethyl-4-((6-methyl-2- (3-(4-sulfamoyl- phenyl)guanidino)pyrimidin-4- ylamino)methyl)pyrrolidin-3- yl)vinyl)benzofuran-5- carboximidamide |
| 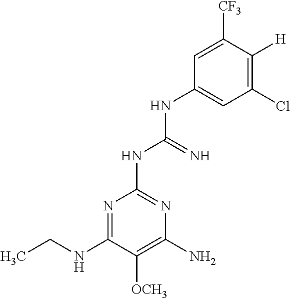 [SMILES: CCNc1nc(NC(=N)Nc2cc(Cl)cc(C(F)(F)F)c2)nc(N)c1OC] | 1-(4-amino-6-(ethylamino)-5- methoxypyrimidin-2-yl)-3- (3-chloro-5-(trifluoro- methyl)phenyl)guanidine |
| 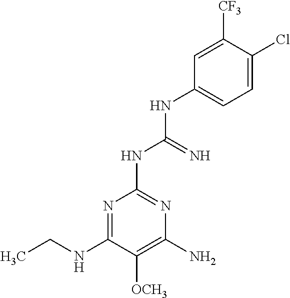 [SMILES: CCNc1nc(NC(=N)Nc2ccc(Cl)c(C(F)(F)F)c2)nc(N)c1OC] | 1-(4-amino-6-(ethylamino)-5- methoxypyrimidin-2-yl)-3- (4-chloro-3-(trifluoro- methyl)phenyl)guanidine |
| 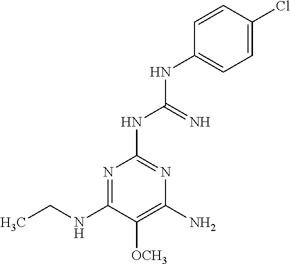 [SMILES: CCNc1nc(NC(=N)Nc2ccc(Cl)cc2)nc(N)c1OC] | 1-(4-amino-6-(ethylamino)-5- methoxypyrimidin-2-yl)-3-(4- chlorophenyl)guanidine |
| 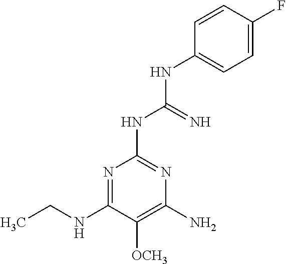 [SMILES: CCNc1nc(NC(=N)Nc2ccc(F)cc2)nc(N)c1OC] | 1-(4-amino-6-(ethylamino)-5- methoxypyrimidin-2-yl)-3-(4- fluorophenyl)guanidine |
| 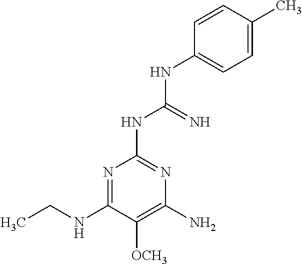 [SMILES: CCNc1nc(NC(=N)Nc2ccc(C)cc2)nc(N)c1OC] | 1-(4-amino-6-(ethylamino)-5- methoxypyrimidin-2-yl)-3- p-tolylguanidine |
| 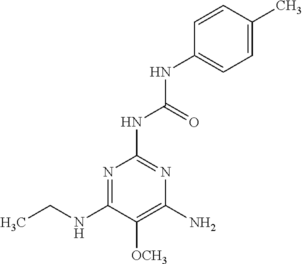 [SMILES: CCNc1nc(NC(=O)Nc2ccc(C)cc2)nc(N)c1OC] | 1-(4-amino-6-(ethylamino)-5- methoxypyrimidin-2-yl)-3- p-tolylurea |
| 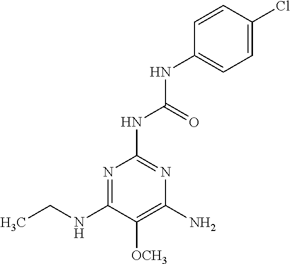 [SMILES: CCNc1nc(NC(=O)Nc2ccc(Cl)cc2)nc(N)c1OC] | 1-(4-amino-6-(ethylamino)-5- methoxypyrimidin-2-yl)-3-(4- chlorophenyl)urea |
| 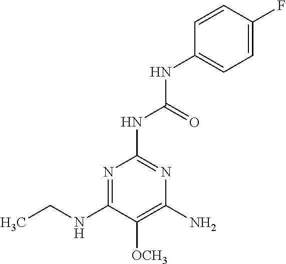 [SMILES: CCNc1nc(NC(=O)Nc2ccc(F)cc2)nc(N)c1OC] | 1-(4-amino-6-(ethylamino)-5- methoxypyrimidin-2-yl)-3-(4- fluorophenyl)urea |
| 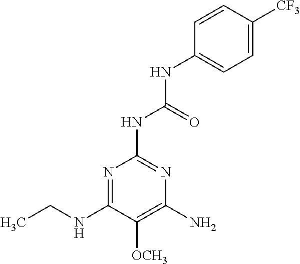 [SMILES: CCNc1nc(NC(=O)Nc2ccc(C(F)(F)F)cc2)nc(N)c1OC] | 1-(4-amino-6-(ethylamino)-5- methoxypyrimidin-2-yl)-3-(4- (trifluoromethyl)phenyl)urea |
| 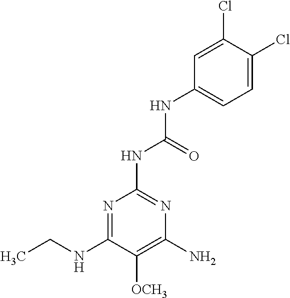 [SMILES: CCNc1nc(NC(=O)Nc2ccc(Cl)c(Cl)c2)nc(N)c1OC] | 1-(4-amino-6-(ethylamino)-5- methoxypyrimidin-2-yl)-3-(3,4- dichlorophenyl)urea |
| 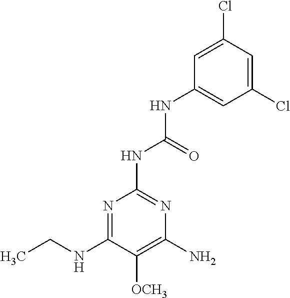 [SMILES: CCNc1nc(NC(=O)Nc2cc(Cl)cc(Cl)c2)nc(N)c1OC] | 1-(4-amino-6-(ethylamino)-5- methoxypyrimidin-2-yl)-3-(3,5- dichlorophenyl)urea |
| 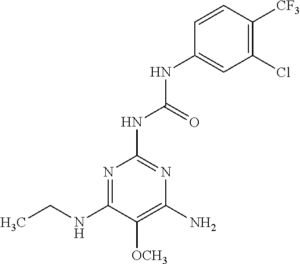 [SMILES: CCNc1nc(NC(=O)Nc2ccc(C(F)(F)F)c(Cl)c2)nc(N)c1OC] | 1-(4-amino-6-(ethylamino)-5- methoxypyrimidin-2-yl)- 3-(3-chloro-4-(trifluoro- methyl)phenyl)urea |
| 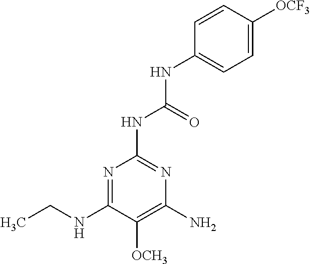 [SMILES: CCNc1nc(NC(=O)Nc2ccc(OC(F)(F)F)cc2)nc(N)c1OC] | 1-(4-amino-6-(ethylamino)-5- methoxypyrimidin-2-yl)-3-(4- (trifluoromethoxy)phenyl)urea |
| 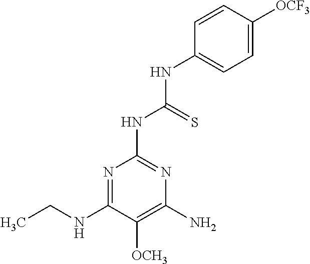 [SMILES: CCNc1nc(NC(=S)Nc2ccc(OC(F)(F)F)cc2)nc(N)c1OC] | 1-(4-amino-6-(ethylamino)-5- methoxypyrimidin-2-yl)-3-(4- (trifluoromethoxy)phenyl)thiourea |
| 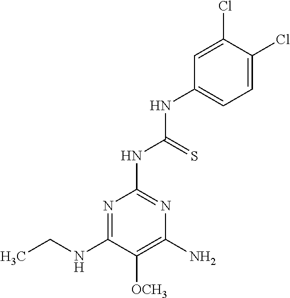 [SMILES: CCNc1nc(NC(=S)Nc2ccc(Cl)c(Cl)c2)nc(N)c1OC] | 1-(4-amino-6-(ethylamino)-5- methoxypyrimidin-2-yl)-3-(3,4- dichlorophenyl)thiourea |
| 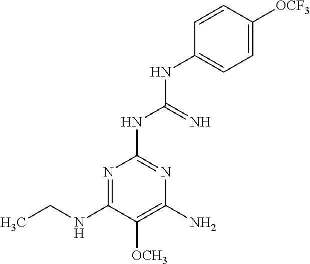 [SMILES: CCNc1nc(NC(=N)Nc2ccc(OC(F)(F)F)cc2)nc(N)c1OC] | 1-(4-amino-6-(ethylamino)-5- methoxypyrimidin-2-yl)-3- (4-(trifluoro- methoxy)phenyl)guanidine |
| 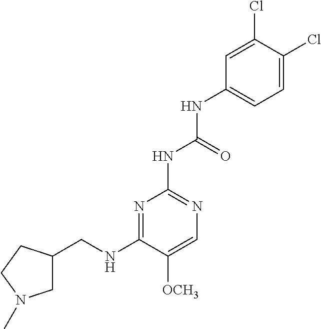 [SMILES: COc1cnc(NC(=O)Nc2ccc(Cl)c(Cl)c2)nc1NCC1CCN(C)C1] | 1-(3,4-dichlorophenyl)-3-(5- methoxy-4-((1-methyl- pyrrolidin-3-yl)methyl- amino)pyrimidin-2-yl)urea |
| 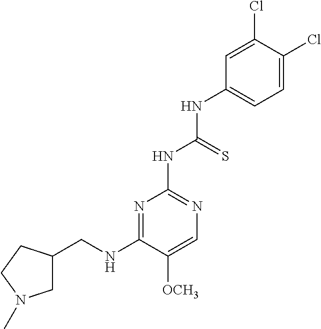 [SMILES: COc1cnc(NC(=S)Nc2ccc(Cl)c(Cl)c2)nc1NCC1CCN(C)C1] | 1-(3,4-dichlorophenyl)-3- (5-methoxy-4-((1- methylpyrrolidin-3- yl)methylamino)pyrimidin- 2-yl)thiourea |
| 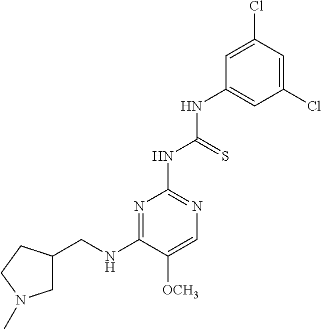 [SMILES: COc1cnc(NC(=S)Nc2cc(Cl)cc(Cl)c2)nc1NCC1CCN(C)C1] | 1-(3,5-dichlorophenyl)-3-(5- methoxy-4-((1- methylpyrrolidin-3- yl)methylamino)pyrimidin- 2-yl)thiourea |
| 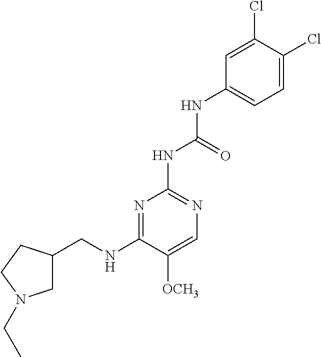 [SMILES: CCN1CCC(CNc2nc(NC(=O)Nc3ccc(Cl)c(Cl)c3)ncc2OC)C1] | l-(3,4-dichlorophenyl)-3-(4-((1- ethylpyrrolidin-3-yl)methyl- amino)-5- methoxypyrimidin-2-yl)urea |
| 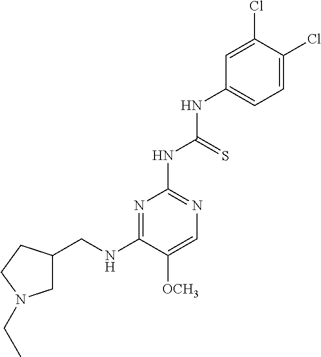 [SMILES: CCN1CCC(CNc2nc(NC(=S)Nc3ccc(Cl)c(Cl)c3)ncc2OC)C1] | 1-(3,4-dichlorophenyl)-3-(4-((1- ethylpyrrolidin-3-yl)methyl- amino)-5-methoxy- pyrimidin-2-yl)thiourea |
| 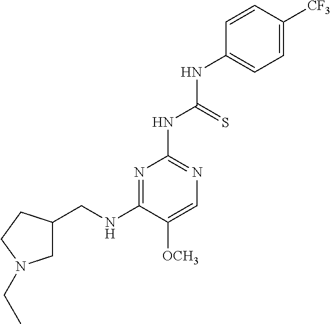 [SMILES: CCN1CCC(CNc2nc(NC(=S)Nc3ccc(C(F)(F)F)cc3)ncc2OC)C1] | 1-(4-((1-ethylpyrrolidin-3- yl)methylamino)-5- methoxypyrimidin-2-yl)-3- (4-(trifluoro- methyl)phenyl)thiourea |
| 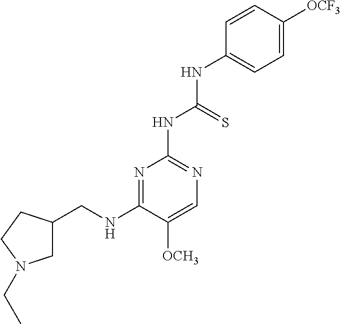 [SMILES: CCN1CCC(CNc2nc(NC(=S)Nc3ccc(OC(F)(F)F)cc3)ncc2OC)C1] | 1-(4-((1-ethylpyrrolidin-3- yl)methylamino)-5- methoxypyrimidin-2- yl)-3-(4-(trifluoro- methoxy)phenyl)thiourea |
| 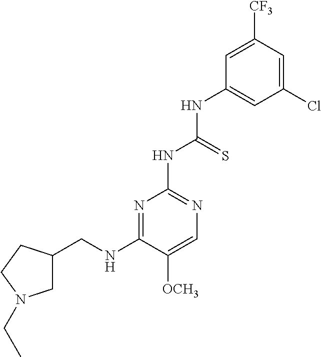 [SMILES: CCN1CCC(CNc2nc(NC(=S)Nc3cc(Cl)cc(C(F)(F)F)c3)ncc2OC)C1] | 1-(3-chloro-5-(trifluoro- methyl)phenyl)-3-(4- ((1-ethylpyrrolidin-3- yl)methylamino)-5- methoxypyrimidin-2- yl)thiourea |
| 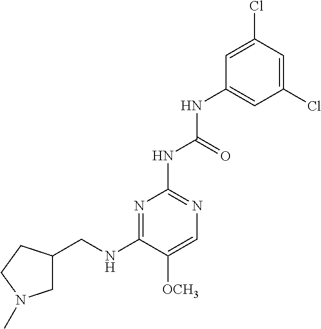 [SMILES: COc1cnc(NC(=O)Nc2cc(Cl)cc(Cl)c2)nc1NCC1CCN(C)C1] | 1-(3,5-dichlorophenyl)-3-(5- methoxy-4-((1- methylpyrrolidin-3- yl)methylamino)pyrimidin- 2-yl)urea |
| 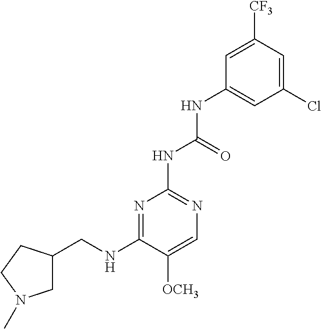 [SMILES: COc1cnc(NC(=O)Nc2cc(Cl)cc(C(F)(F)F)c2)nc1NCC1CCN(C)C1] | 1-(3-chloro-5-(trifluoro- methyl)phenyl)-3-(5- methoxy-4-((1-methyl- pyrrolidin-3- yl)methylamino)pyrimidin- 2-yl)urea |
| 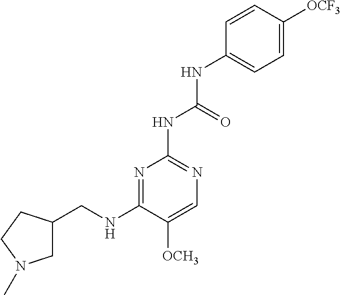 [SMILES: COc1cnc(NC(=O)Nc2ccc(OC(F)(F)F)cc2)nc1NCC1CCN(C)C1] | 1-(5-methoxy-4-((1-methyl- pyrrolidin-3- yl)methylamino)pyrimidin- 2-yl)-3-(4-(trifluoro- methoxy)phenyl)urea |
| 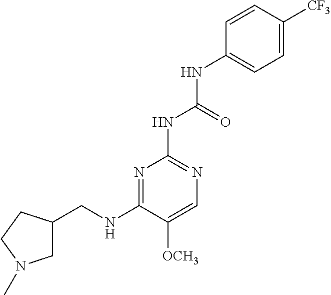 [SMILES: COc1cnc(NC(=O)Nc2ccc(C(F)(F)F)cc2)nc1NCC1CCN(C)C1] | 1-(5-methoxy-4-((1-methyl- pyrrolidin-3- yl)methylamino)pyrimidin- 2-yl)-3-(4-(trifluoro- methyl)phenyl)urea |
| 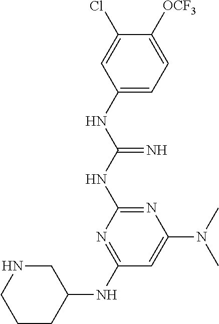 [SMILES: CN(C)c1cc(NC2CCCNC2)nc(NC(=N)Nc2ccc(OC(F)(F)F)c(Cl)c2)n1] | 1-(3-chloro-4-(trifluoro- methoxy)phenyl)-3- (4-(dimethylamino)- 6-(piperidin-3- ylamino)pyrimidin-2- yl)guanidine |
| 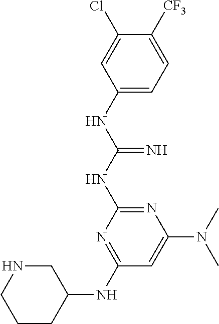 [SMILES: CN(C)c1cc(NC2CCCNC2)nc(NC(=N)Nc2ccc(C(F)(F)F)c(Cl)c2)n1] | 1-(3-chloro-4-(trifluoro- methyl)phenyl)-3-(4- (dimethylamino)-6- (piperidin-3- ylamino)pyrimidin-2- yl)guanidine |
| 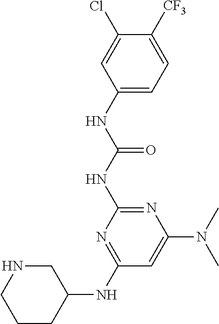 [SMILES: CN(C)c1cc(NC2CCCNC2)nc(NC(=O)Nc2ccc(C(F)(F)F)c(Cl)c2)n1] | 1-(3-chloro-4-(trifluoro- methyl)phenyl)-3-(4- (dimethylamino)-6-(piperidin- 3-ylamino)pyrimidin- 2-yl)urea |
| 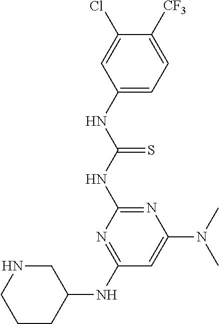 [SMILES: CN(C)c1cc(NC2CCCNC2)nc(NC(=S)Nc2ccc(C(F)(F)F)c(Cl)c2)n1] | 1-(3-chloro-4-(trifluoro- methyl)phenyl)-3-(4- (dimethylamino)-6-(piperidin-3- ylamino)pyrimidin-2-yl)thiourea |
| 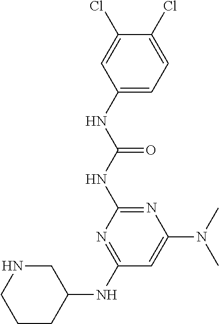 [SMILES: CN(C)c1cc(NC2CCCNC2)nc(NC(=O)Nc2ccc(Cl)c(Cl)c2)n1] | 1-(3,4-dichlorophenyl)-3-(4- (dimethylamino)-6-(piperidin-3- ylamino)pyrimidin-2-yl)urea |
| 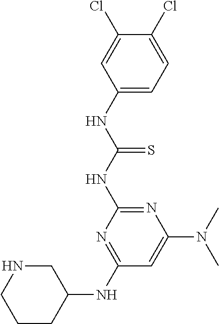 [SMILES: CN(C)c1cc(NC2CCCNC2)nc(NC(=S)Nc2ccc(Cl)c(Cl)c2)n1] | 1-(3,4-dichlorophenyl)-3-(4- (dimethylamino)-6-(piperidin-3- ylamino)pyrimidin-2-yl)thiourea |
| 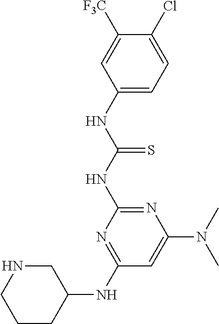 [SMILES: CN(C)c1cc(NC2CCCNC2)nc(NC(=S)Nc2ccc(Cl)c(C(F)(F)F)c2)n1] | 1-(4-chloro-3-(trifluoro- methyl)phenyl)-3-(4- (dimethylamino)-6-(piperidin- 3-ylamino)pyrimidin-2- yl)thiourea |
| 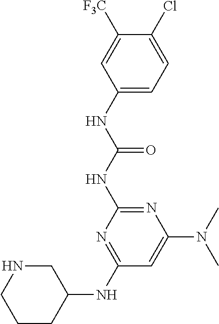 [SMILES: CN(C)c1cc(NC2CCCNC2)nc(NC(=O)Nc2ccc(Cl)c(C(F)(F)F)c2)n1] | 1-(4-chloro-3-(trifluoro- methyl)phenyl)-3-(4- (dimethylamino)-6- (piperidin-3- ylamino)pyrimidin-2- yl)urea |
| 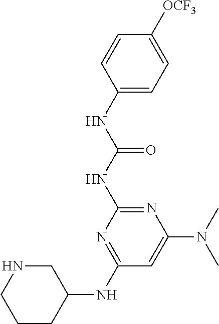 [SMILES: CN(C)c1cc(NC2CCCNC2)nc(NC(=O)Nc2ccc(OC(F)(F)F)cc2)n1] | 1-(4-(dimethylamino)-6- (piperidin-3- ylamino)pyrimidin-2-yl)-3- (4-(trifluoro- methoxy)phenyl)urea |
| 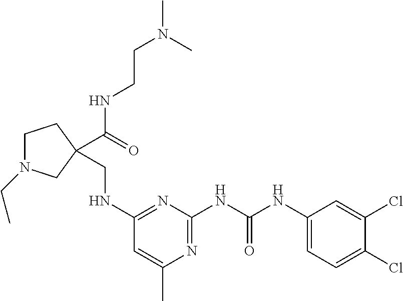 [SMILES: CCN1CCC(CNc2cc(C)nc(NC(=O)Nc3ccc(Cl)c(Cl)c3)n2)(C(=O)NCCN(C)C)C1] | 3-((2-(3-(3,4-dichloro- phenyl)ureido)-6-methyl- pyrimidin-4- ylamino)methyl)-N-(2- (dimethylamino)ethyl)-1- ethylpyrrolidine-3- carboxamide |
| 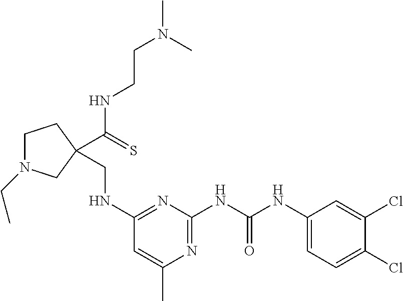 [SMILES: CCN1CCC(CNc2cc(C)nc(NC(=O)Nc3ccc(Cl)c(Cl)c3)n2)(C(=S)NCCN(C)C)C1] | 3-((2-(3-(3,4-dichloro- phenyl)ureido)-6-methyl- pyrimidin-4- ylamino)methyl)-N-(2- (dimethylamino)ethyl)-1- ethylpyrrolidine-3- carbothioamide |
| 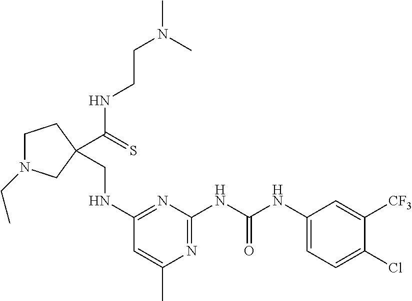 [SMILES: CCN1CCC(CNc2cc(C)nc(NC(=O)Nc3ccc(Cl)c(C(F)(F)F)c3)n2)(C(=S)NCCN(C)C)C1] | 3-((2-(3-(4-chloro-3- (trifluoro- methyl)phenyl)ureido)-6- methylpyrimidin-4- ylamino)methyl)-N-(2- (dimethylamino)ethyl)-1-ethyl- pyrrolidine-3-carbothioamide |
| 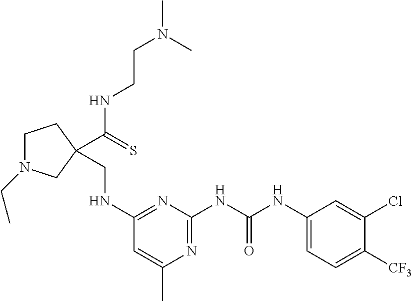 [SMILES: CCN1CCC(CNc2cc(C)nc(NC(=O)Nc3ccc(C(F)(F)F)c(Cl)c3)n2)(C(=S)NCCN(C)C)C1] | 3-((2-(3-(3-chloro-4- (trifluoro- methyl)phenyl)ureido)- 6-methylpyrimidin-4- ylamino)methyl)-N-(2- (dimethylamino)ethyl)-1- ethylpyrrolidine-3- carbothioamide |
| 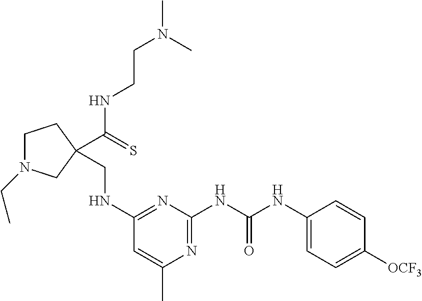 [SMILES: CCN1CCC(CNc2cc(C)nc(NC(=O)Nc3ccc(OC(F)(F)F)cc3)n2)(C(=S)NCCN(C)C)C1] | N-(2-(dimethylamino)ethyl)-1- ethyl-3-((6-methyl-2-(3-(4- (trifluoromethoxy)phenyl) ureido)pyrimidin-4- ylamino)methyl)pyrrolidine- 3-carbothioamide |
| 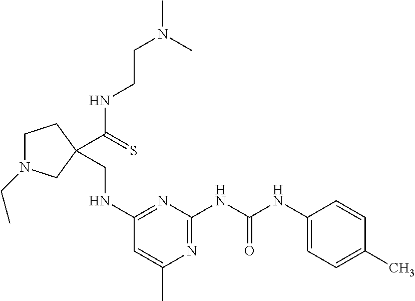 [SMILES: CCN1CCC(CNc2cc(C)nc(NC(=O)Nc3ccc(C)cc3)n2)(C(=S)NCCN(C)C)C1] | N-(2-(dimethylamino)ethyl)-1- ethyl-3-((6-methyl-2-(3- p-tolylureido)pyrimidin-4- ylamino)methyl)pyrrolidine- 3-carbothioamide |
| 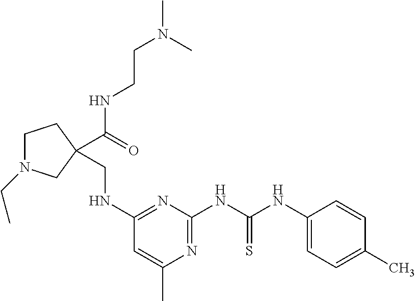 [SMILES: CCN1CCC(CNc2cc(C)nc(NC(=S)Nc3ccc(C)cc3)n2)(C(=O)NCCN(C)C)C1] | N-(2-(dimethylamino)ethyl)- 1-ethyl-3-((6-methyl- 2-(3-p-tolylthioureido) pyrimidin-4- ylamino)methyl)pyrrolidine- 3-carboxamide |
| 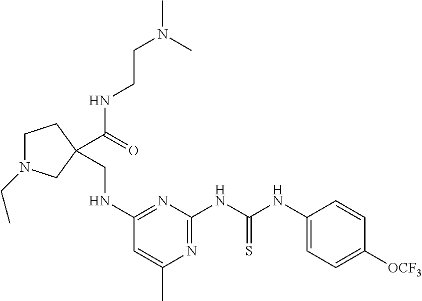 [SMILES: CCN1CCC(CNc2cc(C)nc(NC(=S)Nc3ccc(OC(F)(F)F)cc3)n2)(C(=O)NCCN(C)C)C1] | N-(2-(dimethylamino)ethyl)-1- ethyl-3-((6-methyl-2-(3-(4- (trifluoromethoxy(phenyl) thioureido)pyrimidin-4- ylamino)methyl)pyrrolidine- 3-carboxamide |
| 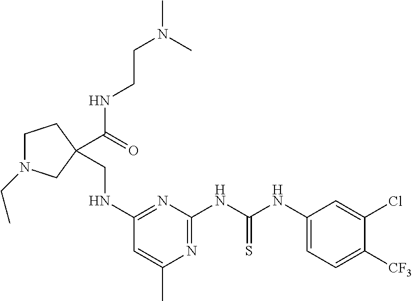 [SMILES: CCN1CCC(CNc2cc(C)nc(NC(=S)Nc3ccc(C(F)(F)F)c(Cl)c3)n2)(C(=O)NCCN(C)C)C1] | 3-((2-(3-(3-chloro-4- (trifluoro- methyl)phenyl)thioureido)- 6-methylpyrimidin-4- ylamino)methyl)-N-(2- (dimethylamino)ethyl)-1- ethylpyrrolidine-3- carboxamide |
| 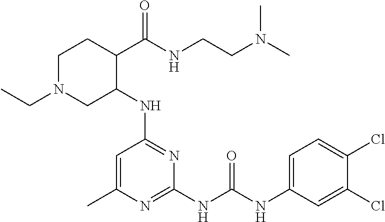 [SMILES: CCN1CCC(C(=O)NCCN(C)C)C(Nc2cc(C)nc(NC(=O)Nc3ccc(Cl)c(Cl)c3)n2)C1] | 3-(2-(3-(3,4-dichloro- phenyl)ureido)-6- methylpyrimidin-4-ylamino)- N-(2-(dimethylamino)ethyl)- 1-ethylpiperidine-4- carboxamide |
| 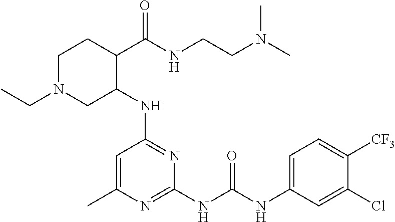 [SMILES: CCN1CCC(C(=O)NCCN(C)C)C(Nc2cc(C)nc(NC(=O)Nc3ccc(C(F)(F)F)c(Cl)c3)n2)C1] | 3-(2-(3-(3-chloro-4- (trifluoromethyl)phenyl)ureido)- 6-methylpyrimidin-4- ylamino)-N-(2- (dimethylamino)ethyl)-1- ethylpiperidine-4- carboxamide |
| 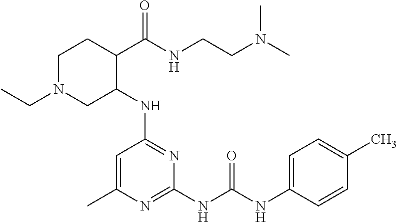 [SMILES: CCN1CCC(C(=O)NCCN(C)C)C(Nc2cc(C)nc(NC(=O)Nc3ccc(C)cc3)n2)C1] | N-(2-(dimethylamino)ethyl)-1- ethyl-3-(6-methyl-2- (3-p-tolylureido)pyrimidin- 4-ylamino)piperidine- 4-carboxamide |
| 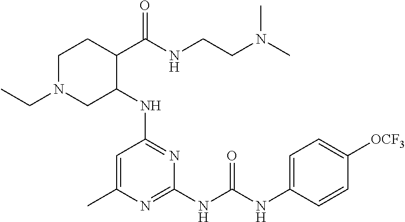 [SMILES: CCN1CCC(C(=O)NCCN(C)C)C(Nc2cc(C)nc(NC(=O)Nc3ccc(OC(F)(F)F)cc3)n2)C1] | N-(2-(dimethylamino)ethyl)-1- ethyl-3-(6-methyl-2-(3-(4- (trifluoromethoxy)phenyl) ureido)pyrimidin-4- ylamino)piperidine-4- carboxamide |
| 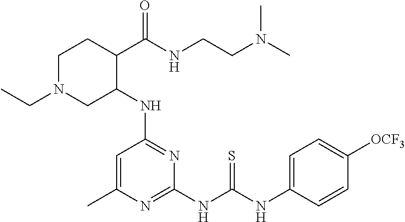 [SMILES: CCN1CCC(C(=O)NCCN(C)C)C(Nc2cc(C)nc(NC(=S)Nc3ccc(OC(F)(F)F)cc3)n2)C1] | N-(2-(dimethylamino)ethyl)- 1-ethyl-3-(6-methyl-2-(3- (4-(trifluoro- methoxy)phenyl)thio- ureido)pyrimidin-4- ylamino)piperidine-4- carboxamide |
| 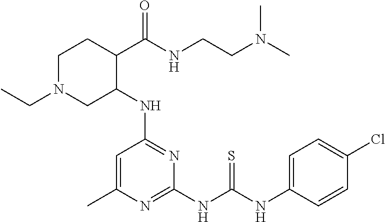 [SMILES: CCN1CCC(C(=O)NCCN(C)C)C(Nc2cc(C)nc(NC(=S)Nc3ccc(Cl)cc3)n2)C1] | 3-(2-(3-(4-chloro- phenyl)thioureido)-6- methylpyrimidin-4- ylamino)-N-(2-(dimethyl- amino)ethyl)-1-ethyl- piperidine-4-carboxamide |
| 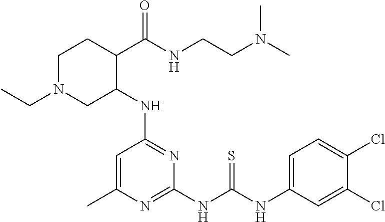 [SMILES: CCN1CCC(C(=O)NCCN(C)C)C(Nc2cc(C)nc(NC(=S)Nc3ccc(Cl)c(Cl)c3)n2)C1] | 3-(2-(3-(3,4-dichloro- phenyl)thioureido)-6- methylpyrimidin-4- ylamino)-N-(2- (dimethylamino)ethyl)- 1-ethylpiperidine-4- carboxamide |
| 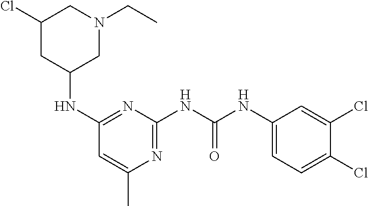 [SMILES: CCN1CC(Cl)CC(Nc2cc(C)nc(NC(=O)Nc3ccc(Cl)c(Cl)c3)n2)C1] | 1-(4-(5-chloro-1-ethyl- piperidin-3-ylamino)- 6-methylpyrimidin-2-yl)- 3-(3,4-dichlorophenyl)urea |
| 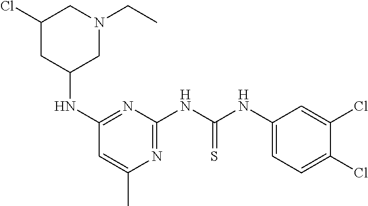 [SMILES: CCN1CC(Cl)CC(Nc2cc(C)nc(NC(=S)Nc3ccc(Cl)c(Cl)c3)n2)C1] | 1-(4-(5-chloro-1-ethyl- piperidin-3-ylamino)- 6-methylpyrimidin-2-yl)- 3-(3,4-dichloro- phenyl)thiourea |
| 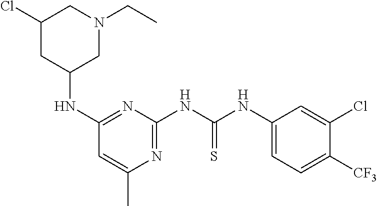 [SMILES: CCN1CC(Cl)CC(Nc2cc(C)nc(NC(=S)Nc3ccc(C(F)(F)F)c(Cl)c3)n2)C1] | 1-(4-(5-chloro-1-ethyl- piperidin-3-ylamino)- 6-methylpyrimidin-2- yl)-3-(3-chloro-4-(trifluoro- methyl)phenyl)thiourea |
| 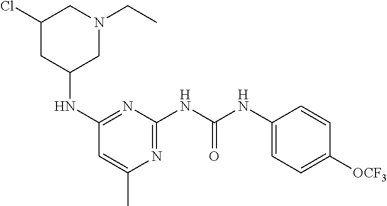 [SMILES: CCN1CC(Cl)CC(Nc2cc(C)nc(NC(=O)Nc3ccc(OC(F)(F)F)cc3)n2)C1] | 1-(4-(5-chloro-1-ethyl- piperidin-3-ylamino)- 6-methylpyrimidin-2-yl)- 3-(4-(trifluoro- methoxy)phenyl)urea |
| 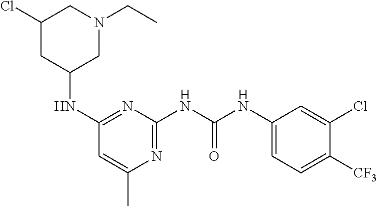 [SMILES: CCN1CC(Cl)CC(Nc2cc(C)nc(NC(=O)Nc3ccc(C(F)(F)F)c(Cl)c3)n2)C1] | 1-(4-(5-chloro-1-ethyl- piperidin-3-ylamino)- 6-methylpyrimidin-2- yl)-3-(3-chloro-4-(trifluoro- methyl)phenyl)urea |
| 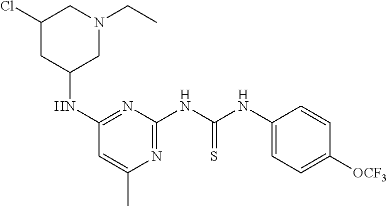 [SMILES: CCN1CC(Cl)CC(Nc2cc(C)nc(NC(=S)Nc3ccc(OC(F)(F)F)cc3)n2)C1] | 1-(4-(5-chloro-1-ethyl- piperidin-3-ylamino)- 6-methylpyrimidin-2-yl)- 3-(4-(trifluoro- methoxy)phenyl)thiourea |
| 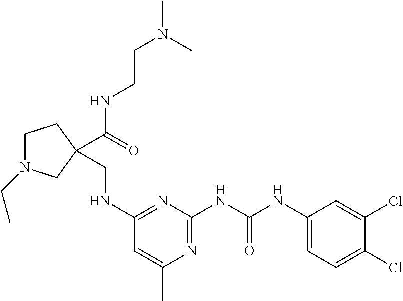 [SMILES: CCN1CCC(CNc2cc(C)nc(NC(=O)Nc3ccc(Cl)c(Cl)c3)n2)(C(=O)NCCN(C)C)C1] | 3-((2-(3-(3,4-dichloro- phenyl)ureido)-6- methylpyrimidin-4- ylamino)methyl)-N-(2- (dimethylamino)ethyl)-1- ethylpyrrolidine-3- carboxamide |
| 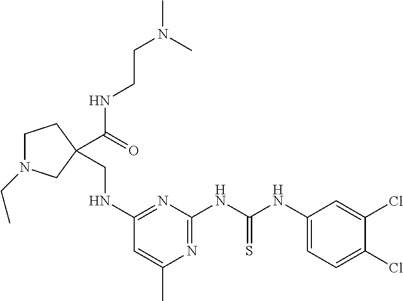 [SMILES: CCN1CCC(CNc2cc(C)nc(NC(=S)Nc3ccc(Cl)c(Cl)c3)n2)(C(=O)NCCN(C)C)C1] | 3-((2-(3-(3,4-dichloro- phenyl)thioureido)-6- methylpyrimidin-4- ylamino)methyl)-N-(2- (dimethylamino)ethyl)- 1-ethylpyrrolidine-3- carboxamide |
| 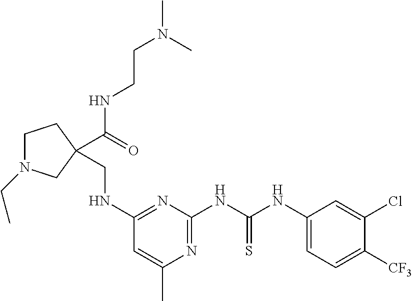 [SMILES: CCN1CCC(CNc2cc(C)nc(NC(=S)Nc3ccc(C(F)(F)F)c(Cl)c3)n2)(C(=O)NCCN(C)C)C1] | 3-((2-(3-(3-chloro-4- (trifluoromethyl)phenyl) thioureido)-6-methyl- pyrimidin-4- ylamino)methyl)-N-(2- (dimethylamino)ethyl)-1- ethylpyrrolidine-3- carboxamide |
| 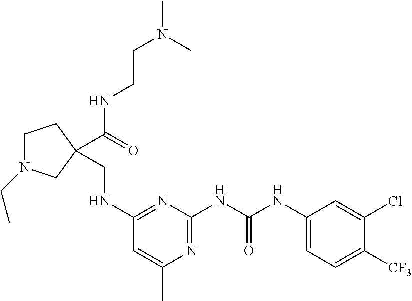 [SMILES: CCN1CCC(CNc2cc(C)nc(NC(=O)Nc3ccc(C(F)(F)F)c(Cl)c3)n2)(C(=O)NCCN(C)C)C1] | 3-((2-(3-(3-chloro-4- (trifluoromethyl)phenyl) ureido)-6-methyl- pyrimidin-4-ylamino) methyl)-N-(2-(dimethyl- amino)ethyl)-1-ethyl- pyrrolidine-3- carboxamide |
| 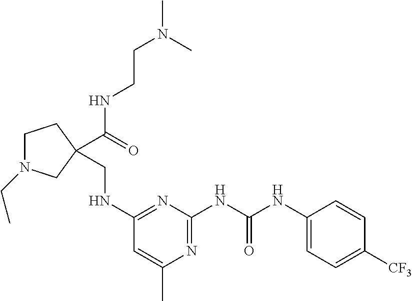 [SMILES: CCN1CCC(CNc2cc(C)nc(NC(=O)Nc3ccc(C(F)(F)F)cc3)n2)(C(=O)NCCN(C)C)C1] | N-(2-(dimethylamino)ethyl)- 1-ethyl-3-((6-methyl-2-(3- (4-(trifluoro- methyl)phenyl) ureido)pyrimidin-4- ylamino)methyl)pyrrolidine- 3-carboxamide |
| 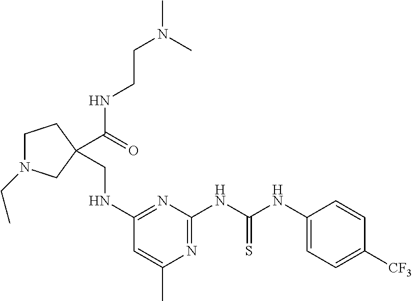 [SMILES: CCN1CCC(CNc2cc(C)nc(NC(=S)Nc3ccc(C(F)(F)F)cc3)n2)(C(=O)NCCN(C)C)C1] | N-(2-(dimethylamino)ethyl)-1- ethyl-3-((6-methyl-2-(3-(4- (trifluoromethyl)phenyl) thioureido)pyrimidin-4- ylamino)methyl) pyrrolidine-3- carboxamide |
| 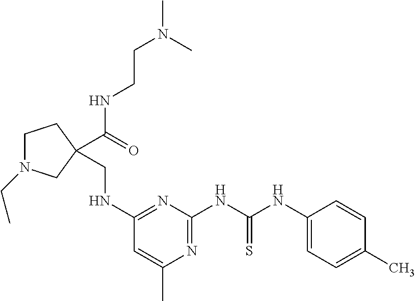 [SMILES: CCN1CCC(CNc2cc(C)nc(NC(=S)Nc3ccc(C)cc3)n2)(C(=O)NCCN(C)C)C1] | N-(2-(dimethyl- amino)ethyl)-1-ethyl-3- ((6-methyl-2-(3-p-tolyl- thioureido)pyrimidin-4- ylamino)methyl)pyrrolidine- 3-carboxamide |
| 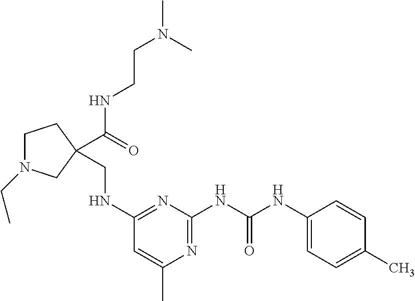 [SMILES: CCN1CCC(CNc2cc(C)nc(NC(=O)Nc3ccc(C)cc3)n2)(C(=O)NCCN(C)C)C1] | N-(2-(dimethylamino)ethyl)- 1-ethyl-3-((6-methyl-2- (3-p-tolylureido)pyrimidin- 4-ylamino)methyl) pyrrolidine-3- carboxamide |
| 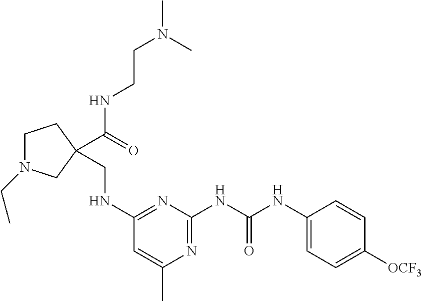 [SMILES: CCN1CCC(CNc2cc(C)nc(NC(=O)Nc3ccc(OC(F)(F)F)cc3)n2)(C(=O)NCCN(C)C)C1] | N-(2-(dimethyl- amino)ethyl)-1-ethyl-3- ((6-methyl-2-(3-(4- (trifluoromethoxy)phenyl) ureido)pyrimidin-4- ylamino)methyl)pyrrolidine- 3-carboxamide |
| 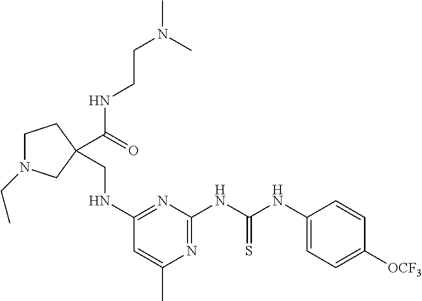 [SMILES: CCN1CCC(CNc2cc(C)nc(NC(=S)Nc3ccc(OC(F)(F)F)cc3)n2)(C(=O)NCCN(C)C)C1] | N-(2-(dimethyl- amino)ethyl)-1-ethyl-3-((6- methyl-2-(3-(4-(trifluoro- methoxy)phenyl)thio- ureido)pyrimidin-4- ylamino)methyl) pyrrolidine-3- carboxamide |
| 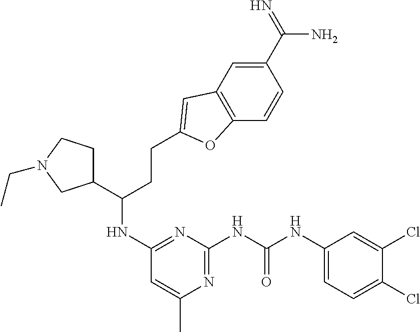 [SMILES: CCN1CCC(C(CCc2cc3cc(C(=N)N)ccc3o2)Nc2cc(C)nc(NC(=O)Nc3ccc(Cl)c(Cl)c3)n2)C1] | 2-(3-(2-(3-(3,4-dichloro- phenyl)ureido)-6-methyl- pyrimidin-4-ylamino)-3-(1- ethylpyrrolidin-3- yl)propyl)benzofuran-5- carboximidamide |
| 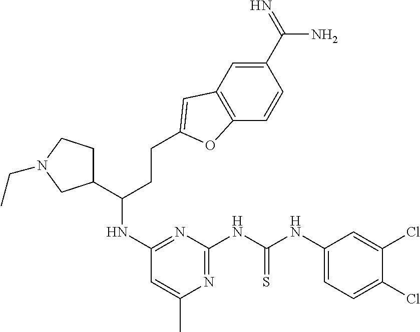 [SMILES: CCN1CCC(C(CCc2cc3cc(C(=N)N)ccc3o2)Nc2cc(C)nc(NC(=S)Nc3ccc(Cl)c(Cl)c3)n2)C1] | 2-(3-(2-(3-(3,4-dichloro- phenyl)thioureido)- 6-methylpyrimidin-4- ylamino)-3-(1-ethyl- pyrrolidin-3-yl)propyl) benzofuran-5- carboximidamide |
| 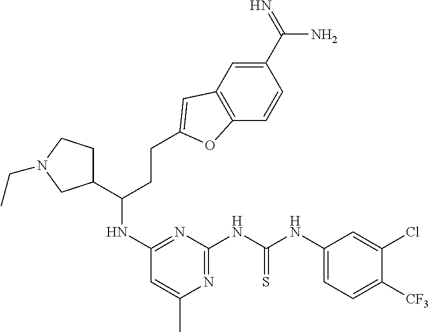 [SMILES: CCN1CCC(C(CCc2cc3cc(C(=N)N)ccc3o2)Nc2cc(C)nc(NC(=S)Nc3ccc(C(F)(F)F)c(Cl)c3)n2)C1] | 2-(3-(2-(3-(3-chloro- 4-(trifluoro- methyl)phenyl)thioureido)- 6-methylpyrimidin-4- ylamino)-3-(1-ethyl- pyrrolidin-3-yl)propyl) benzofuran-5- carboximidamide |
| 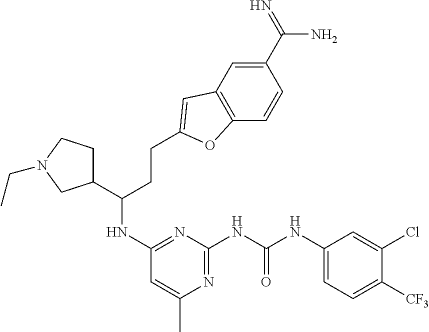 [SMILES: CCN1CCC(C(CCc2cc3cc(C(=N)N)ccc3o2)Nc2cc(C)nc(NC(=O)Nc3ccc(C(F)(F)F)c(Cl)c3)n2)C1] | 2-(3-(2-(3-(3-chloro- 4-(trifluoro- methyl)phenyl)ureido)- 6-methylpyrimidin-4- ylamino)-3-(1-ethyl- pyrrolidin-3- yl)propyl)benzofuran-5- carboximidamide |
| 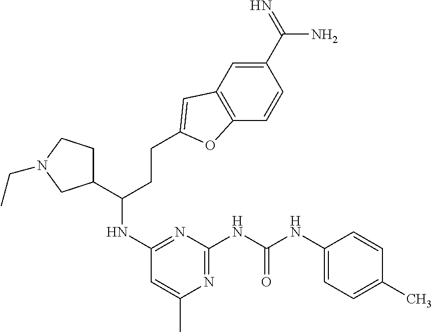 [SMILES: CCN1CCC(C(CCc2cc3cc(C(=N)N)ccc3o2)Nc2cc(C)nc(NC(=O)Nc3ccc(C)cc3)n2)C1] | 2-(3-(1-ethylpyrrolidin- 3-yl)-3-(6-methyl-2- (3-p-tolylureido)pyrimidin- 4-ylamino)propyl) benzofuran-5- carboximidamide |
| 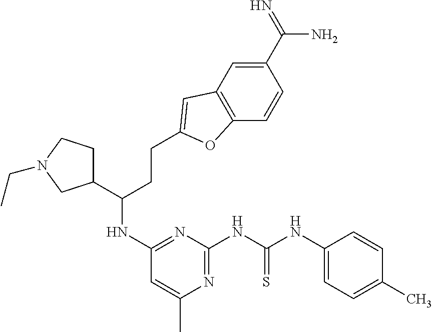 [SMILES: CCN1CCC(C(CCc2cc3cc(C(=N)N)ccc3o2)Nc2cc(C)nc(NC(=S)Nc3ccc(C)cc3)n2)C1] | 2-(3-(1-ethylpyrrolidin-3- yl)-3-(6-methyl-2- (3-p-tolylthio- ureido)pyrimidin-4- ylamino)propyl)benzofuran- 5-carboximidamide |
| 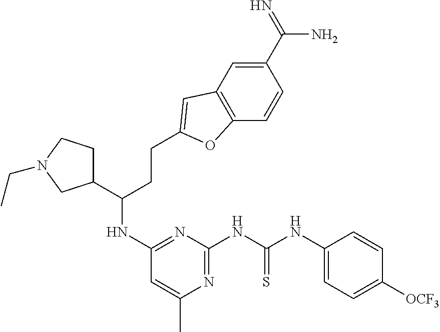 [SMILES: CCN1CCC(C(CCc2cc3cc(C(=N)N)ccc3o2)Nc2cc(C)nc(NC(=S)Nc3ccc(OC(F)(F)F)cc3)n2)C1] | 2-(3-(1-ethylpyrrolidin-3- yl)-3-(6-methyl-2-(3- (4-(trifluoro- methoxy(phenyl)thio- ureido)pyrimidin-4- ylamino)propyl)benzofuran- 5-carboximidamide |
| 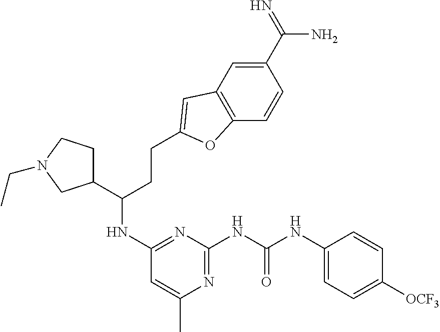 [SMILES: CCN1CCC(C(CCc2cc3cc(C(=N)N)ccc3o2)Nc2cc(C)nc(NC(=O)Nc3ccc(OC(F)(F)F)cc3)n2)C1] | 2-(3-(1-ethylpyrrolidin-3-yl)-3- (6-methyl-2-(3- (4-(trifluoro- methoxy)phenyl)ureido) pyrimidin-4- ylamino)propyl)benzofuran- 5-carboximidamide |
| 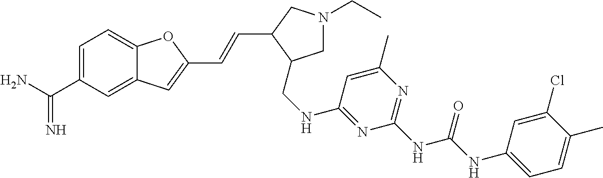 [SMILES: CCN1CC(/C=C/c2cc3cc(C(=N)N)ccc3o2)C(CNc2cc(C)nc(NC(=O)Nc3ccc(C)c(Cl)c3)n2)C1] | (E)-2-(2-(4-((2-(3-(3,4- dichlorophenyl)ureido)-6- methylpyrimidin-4- ylamino)methyl)-1- ethylpyrrolidin-3- yl)vinyl)benzofuran- 5-carboximidamide |
| 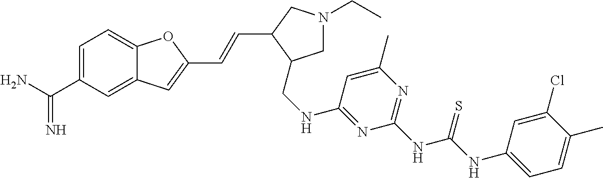 [SMILES: CCN1CC(/C=C/c2cc3cc(C(=N)N)ccc3o2)C(CNc2cc(C)nc(NC(=S)Nc3ccc(C)c(Cl)c3)n2)C1] | (E)-2-(2-(4-((2-(3- (3,4-dichloro- phenyl)thioureido)-6- methylpyrimidin-4- ylamino)methyl)-1- ethylpyrrolidin-3- yl)vinyl)benzofuran-5- carboximidamide |
| 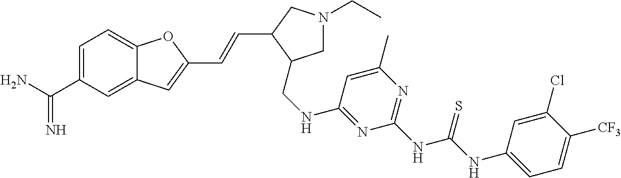 [SMILES: CCN1CC(/C=C/c2cc3cc(C(=N)N)ccc3o2)C(CNc2cc(C)nc(NC(=S)Nc3ccc(C(F)(F)F)c(Cl)c3)n2)C1] | (E)-2-(2-(4-((2-(3-(3- chloro-4-(trifluoro- methyl)phenyl)thio- ureido)-6-methyl- pyrimidin-4- ylamino)methyl)-1- ethylpyrrolidin-3- yl)vinyl)benzofuran-5- carboximidamide |
| 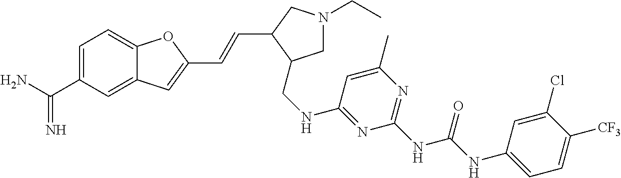 [SMILES: CCN1CC(/C=C/c2cc3cc(C(=N)N)ccc3o2)C(CNc2cc(C)nc(NC(=O)Nc3ccc(C(F)(F)F)c(Cl)c3)n2)C1] | (E)-2-(2-(4-((2-(3-(3- chloro-4-(trifluoro- methyl)phenyl)ureido)- 6-methylpyrimidin-4- ylamino)methyl)-1- ethylpyrrolidin-3- yl)vinyl)benzofuran-5- carboximidamide |
| 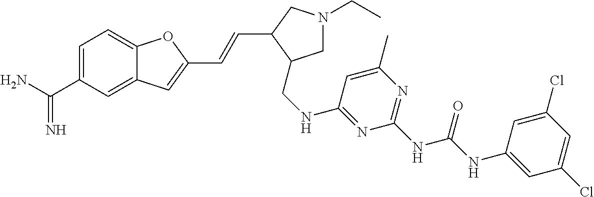 [SMILES: CCN1CC(/C=C/c2cc3cc(C(=N)N)ccc3o2)C(CNc2cc(C)nc(NC(=O)Nc3cc(Cl)cc(Cl)c3)n2)C1] | (E)-2-(2-(4-((2-(3- (3,5-dichloro- phenyl)ureido)-6-methyl- pyrimidin-4- ylamino)methyl)-1- ethylpyrrolidin-3- yl)vinyl)benzofuran- 5-carboximidamide |
| 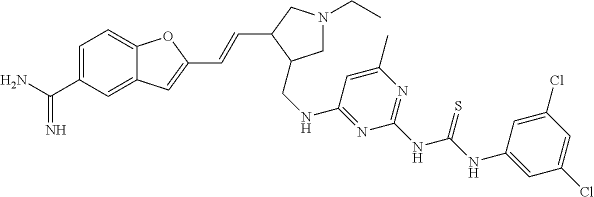 [SMILES: CCN1CC(/C=C/c2cc3cc(C(=N)N)ccc3o2)C(CNc2cc(C)nc(NC(=S)Nc3cc(Cl)cc(Cl)c3)n2)C1] | (E)-2-(2-(4-((2-(3- (3,5-dichloro- phenyl)thioureido)- 6-methylpyrimidin- 4-ylamino)methyl)-1- ethylpyrrolidin-3- yl)vinyl)benzofuran-5- carboximidamide |
| 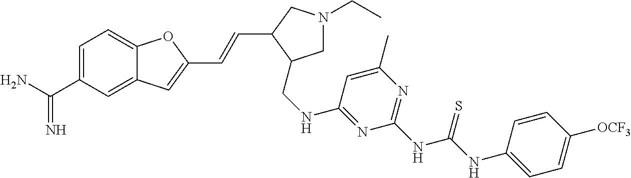 [SMILES: CCN1CC(/C=C/c2cc3cc(C(=N)N)ccc3o2)C(CNc2cc(C)nc(NC(=S)Nc3ccc(OC(F)(F)F)cc3)n2)C1] | (E)-2-(2-(1-eihyl-4- ((6-methyl-2-(3- (4-(trifluoro- methoxy)phenyl)thio- ureido)pyrimidin-4- ylamino)methyl)pyrrolidin- 3-yl)vinyl)benzofuran- 5-carboximidamide |
| 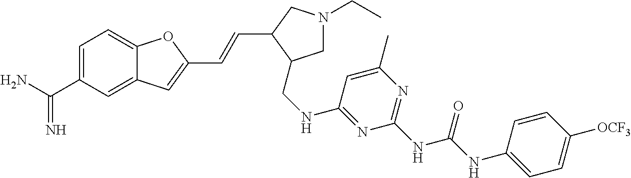 [SMILES: CCN1CC(/C=C/c2cc3cc(C(=N)N)ccc3o2)C(CNc2cc(C)nc(NC(=O)Nc3ccc(OC(F)(F)F)cc3)n2)C1] | (E)-2-(2-(1-ethyl-4-((6- methyl-2-(3-(4-(trifluoro- methoxy)phenyl)ureido) pyrimidin-4- ylamino)methyl) pyrrolidin-3- yl)vinyl)benzofuran- 5-carboximidamide |
| 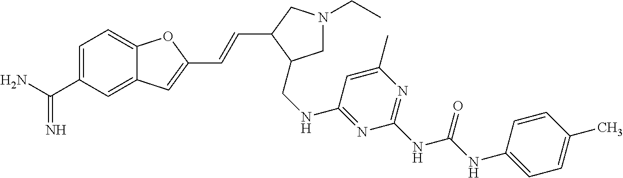 [SMILES: CCN1CC(/C=C/c2cc3cc(C(=N)N)ccc3o2)C(CNc2cc(C)nc(NC(=O)Nc3ccc(C)cc3)n2)C1] | (E)-2-(2-(1-ethyl-4- ((6-methyl-2-(3-p- tolylureido)pyrimidin-4- ylamino)methyl)pyrrolidin- 3-yl)vinyl)benzofuran- 5-carboximidamide |
| 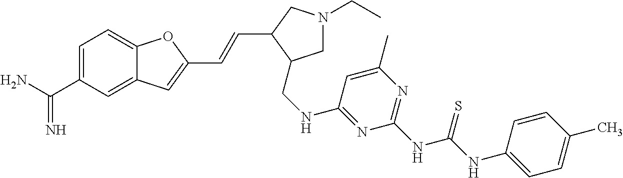 [SMILES: CCN1CC(/C=C/c2cc3cc(C(=N)N)ccc3o2)C(CNc2cc(C)nc(NC(=S)Nc3ccc(C)cc3)n2)C1] | (E)-2-(2-(1-ethyl-4- ((6-methyl-2-(3- p-tolylthio- ureido)pyrimidin- 4-ylamino)methyl) pyrrolidin-3- yl)vinyl)benzofuran- 5-carboximidamide |
| 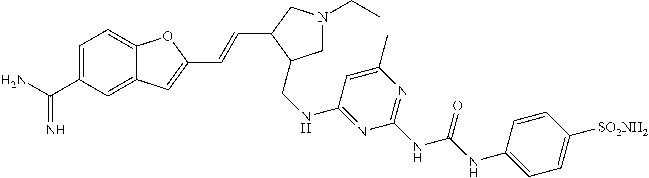 [SMILES: CCN1CC(/C=C/c2cc3cc(C(=N)N)ccc3o2)C(CNc2cc(C)nc(NC(=O)Nc3ccc(S(N)(=O)=O)cc3)n2)C1] | (E)-2-(2-(1-ethyl-4- ((6-methyl-2-(3-(4- sulfamoylphenyl)ureido) pyrimidin-4-yl- amino)methyl) pyrrolidin-3- yl)vinyl)benzofuran- 5-carboximidamide |
| 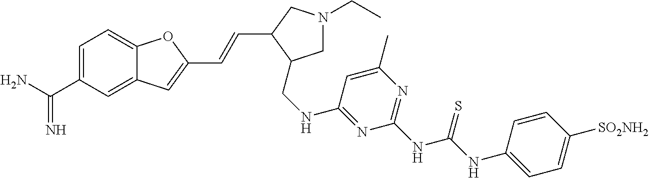 [SMILES: CCN1CC(/C=C/c2cc3cc(C(=N)N)ccc3o2)C(CNc2cc(C)nc(NC(=S)Nc3ccc(S(N)(=O)=O)cc3)n2)C1] | (E)-2-(2-(1-ethyl-4- ((6-methyl-2-(3- (4-sulfamoyl- phenyl)thioureido) pyrimidin-4- ylamino)methyl) pyrrolidin-3- yl)vinyl)benzofuran- 5-carboximidamide |
| 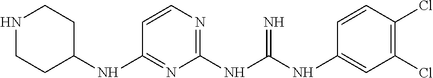 [SMILES: N=C(Nc1ccc(Cl)c(Cl)c1)Nc1nccc(NC2CCNCC2)n1] | |
| 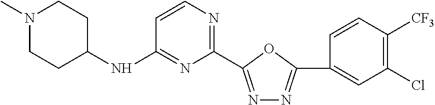 [SMILES: CN1CCC(Nc2ccnc(-c3nnc(-c4ccc(C(F)(F)F)c(Cl)c4)o3)n2)CC1] | |
| 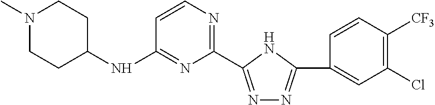 [SMILES: CN1CCC(Nc2ccnc(-c3nnc(-c4ccc(C(F)(F)F)c(Cl)c4)[nH]3)n2)CC1] | |
| 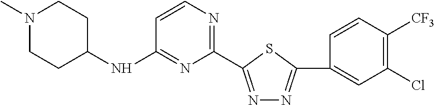 [SMILES: CN1CCC(Nc2ccnc(-c3nnc(-c4ccc(C(F)(F)F)c(Cl)c4)s3)n2)CC1] | |
| 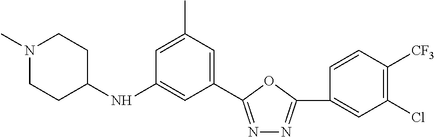 [SMILES: Cc1cc(NC2CCN(C)CC2)cc(-c2nnc(-c3ccc(C(F)(F)F)c(Cl)c3)o2)c1] | |
| 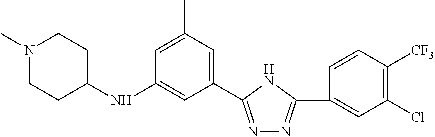 [SMILES: Cc1cc(NC2CCN(C)CC2)cc(-c2nnc(-c3ccc(C(F)(F)F)c(Cl)c3)[nH]2)c1] | |
| 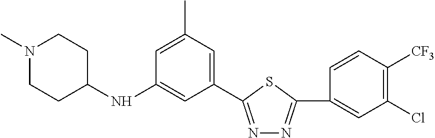 [SMILES: Cc1cc(NC2CCN(C)CC2)cc(-c2nnc(-c3ccc(C(F)(F)F)c(Cl)c3)s2)c1] | |
| 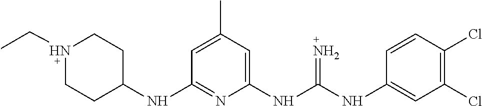 [SMILES: CCN1CCC(Nc2cc(C)cc(NC(=N)Nc3ccc(Cl)c(Cl)c3)n2)CC1] | |
| 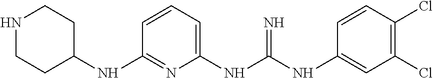 [SMILES: N=C(Nc1ccc(Cl)c(Cl)c1)Nc1cccc(NC2CCNCC2)n1] | |
| 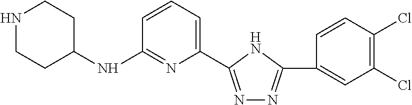 [SMILES: Clc1ccc(-c2nnc(-c3cccc(NC4CCNCC4)n3)[nH]2)cc1Cl] | |
| 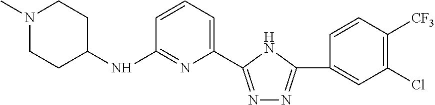 [SMILES: CN1CCC(Nc2cccc(-c3nnc(-c4ccc(C(F)(F)F)c(Cl)c4)[nH]3)n2)CC1] | |
| 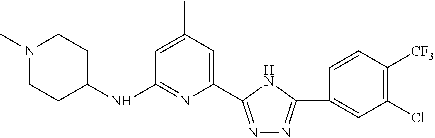 [SMILES: Cc1cc(NC2CCN(C)CC2)nc(-c2nnc(-c3ccc(C(F)(F)F)c(Cl)c3)[nH]2)c1] | |
| 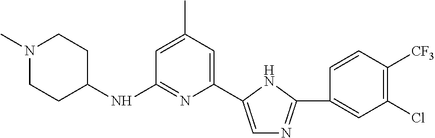 [SMILES: Cc1cc(NC2CCN(C)CC2)nc(-c2cnc(-c3ccc(C(F)(F)F)c(Cl)c3)[nH]2)c1] | |
| 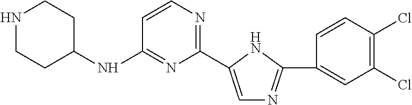 [SMILES: Clc1ccc(-c2ncc(-c3nccc(NC4CCNCC4)n3)[nH]2)cc1Cl] | |
| 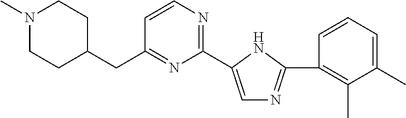 | |
| 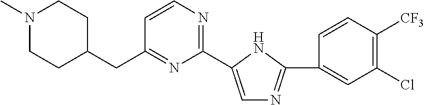 [SMILES: CN1CCC(Cc2ccnc(-c3cnc(-c4ccc(C(F)(F)F)c(Cl)c4)[nH]3)n2)CC1] | |
| 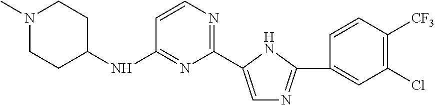 [SMILES: CN1CCC(Nc2ccnc(-c3cnc(-c4ccc(C(F)(F)F)c(Cl)c4)[nH]3)n2)CC1] | |

TABLE 2
Quinoline-based Olig2 inhibitors.

| Compound structure | Compound name |
|---|---|
| 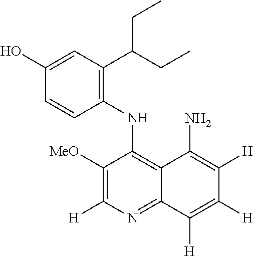 [SMILES: CCC(CC)c1cc(O)ccc1Nc1c(OC)cnc2cccc(N)c12] | 4-(5-amino-3-methoxyquinolin-4- ylamino)-3-(pentan-3-yl)phenol |
| 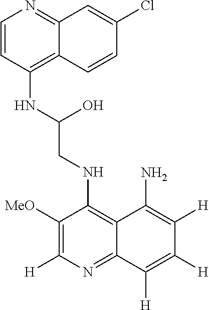 [SMILES: COc1cnc2cccc(N)c2c1NCC(O)Nc1ccnc2cc(Cl)ccc12] | 2-(5-amino-3-methoxyquinolin-4- ylamino)-1-(7-chloroquinolin-4- ylamino)ethanol |
| 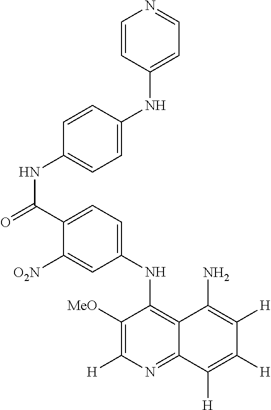 [SMILES: COc1cnc2cccc(N)c2c1Nc1ccc(C(=O)Nc2ccc(Nc3ccncc3)cc2)c([N+](=O)[O-])c1] | 4-(5-amino-3-methoxyquinolin-4- ylamino)-2-nitro-N-(4-(pyridin-4- ylamino)phenyl)benzamide |
| 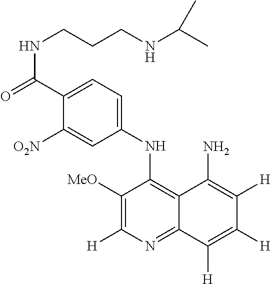 [SMILES: COc1cnc2cccc(N)c2c1Nc1ccc(C(=O)NCCCNC(C)C)c([N+](=O)[O-])c1] | 4-(5-amino-3-methoxyquinolin-4- ylamino)-N-(3- (isopropylamino)propyl)-2- nitrobenzamide |
| 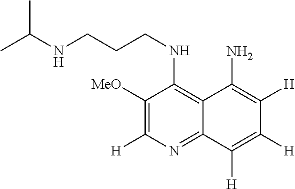 [SMILES: COc1cnc2cccc(N)c2c1NCCCNC(C)C] | N4-(3-(isopropylamino)propyl)-3- methoxyquinoline-4,5-diamine |
| 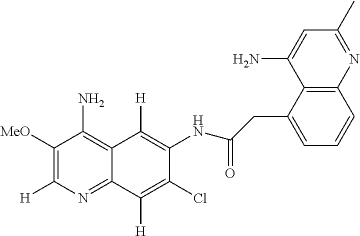 [SMILES: COc1cnc2cc(Cl)c(NC(=O)Cc3cccc4nc(C)cc(N)c34)cc2c1N] | 2-(4-amino-2-methylquinolin-5-yl)-N- (4-amino-7-chloro-3- methoxyquinolin-6-yl)acetamide |
| 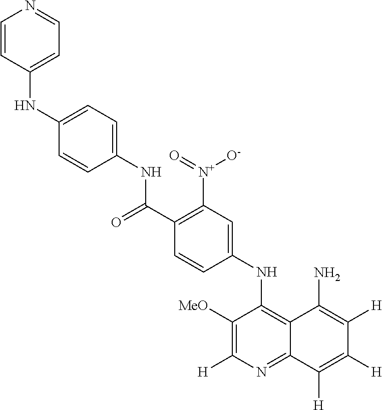 [SMILES: COc1cnc2cccc(N)c2c1Nc1ccc(C(=O)Nc2ccc(Nc3ccncc3)cc2)c([N+](=O)[O-])c1] | 4-(5-amino-3-methoxyquinolin-4- ylamino)-2-nitro-N-(4-(pyridin-4- ylamino)phenyl)benzamide |
| 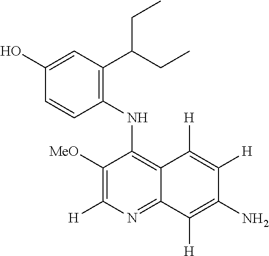 [SMILES: CCC(CC)c1cc(O)ccc1Nc1c(OC)cnc2cc(N)ccc12] | 4-(7-amino-3-methoxyquinolin-4- ylamino)-3-(pentan-3-yl)phenol |
| 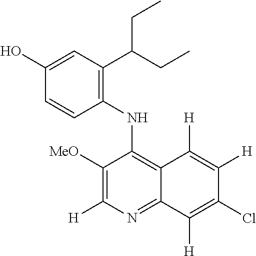 [SMILES: CCC(CC)c1cc(O)ccc1Nc1c(OC)cnc2cc(Cl)ccc12] | 4-(7-chloro-3-methoxyquinolin-4- ylamino)-3-(pentan-3-yl)phenol |
| 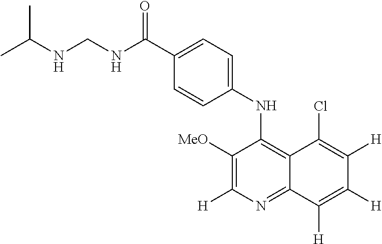 [SMILES: COc1cnc2cccc(Cl)c2c1Nc1ccc(C(=O)NCNC(C)C)cc1] | 4-(5-chloro-3-methoxyquinolin-4- ylamino)-N- ((isopropylamino)methyl)benzamide |
| 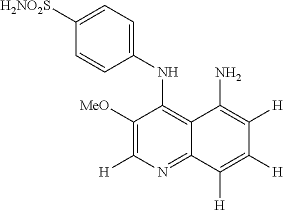 [SMILES: COc1cnc2cccc(N)c2c1Nc1ccc(S(N)(=O)=O)cc1] | 4-(5-amino-3-methoxyquinolin-4- ylamino)benzenesulfonamide |
| 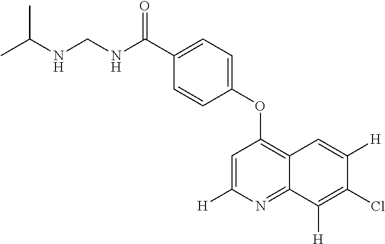 [SMILES: CC(C)NCNC(=O)c1ccc(Oc2ccnc3cc(Cl)ccc23)cc1] | 4-(7-chloroquinolin-4-yloxy)-N- ((isopropylamino)methyl)benzamide |
| 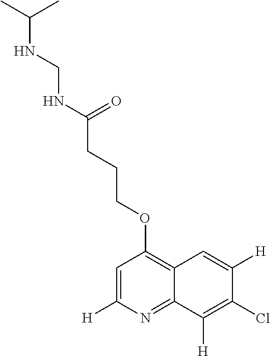 [SMILES: CC(C)NCNC(=O)CCCOc1ccnc2cc(Cl)ccc12] | 4-(7-chloroquinolin-4-yloxy)-N- ((isopropylamino)methyl)butanamide |
| 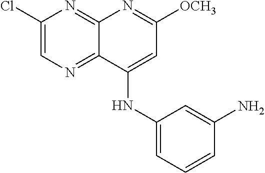 [SMILES: COc1cc(Nc2cccc(N)c2)c2ncc(Cl)nc2n1] | |
| 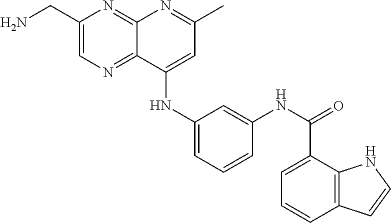 [SMILES: Cc1cc(Nc2cccc(NC(=O)c3cccc4cc[nH]c34)c2)c2ncc(CN)nc2n1] | |
| 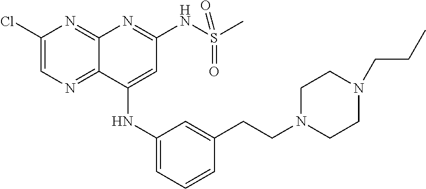 [SMILES: CCCN1CCN(CCc2cccc(Nc3cc(NS(C)(=O)=O)nc4nc(Cl)cnc34)c2)CC1] | |
| 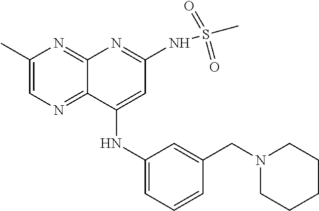 [SMILES: Cc1cnc2c(Nc3cccc(CN4CCCCC4)c3)cc(NS(C)(=O)=O)nc2n1] | |
| 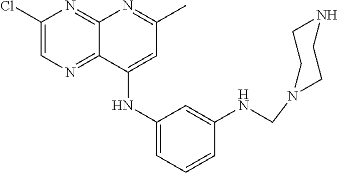 [SMILES: Cc1cc(Nc2cccc(NCN3CCNCC3)c2)c2ncc(Cl)nc2n1] | |
| 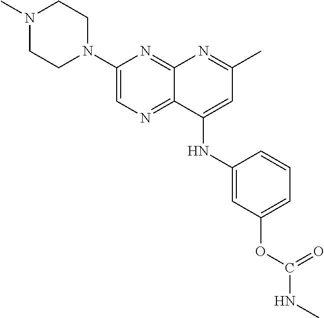 [SMILES: CNC(=O)Oc1cccc(Nc2cc(C)nc3nc(N4CCN(C)CC4)cnc23)c1] | |
| 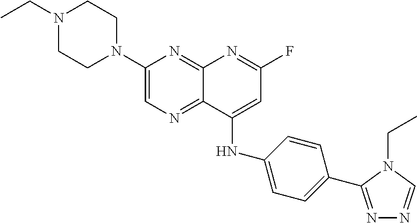 [SMILES: CCN1CCN(c2cnc3c(Nc4ccc(-c5nncn5CC)cc4)cc(F)nc3n2)CC1] | |
| 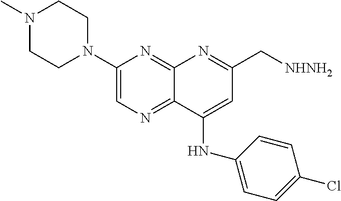 [SMILES: CN1CCN(c2cnc3c(Nc4ccc(Cl)cc4)cc(CNN)nc3n2)CC1] | |
| [symbol] | [symbol] |
| | 2 |

[symbol]

indicates data missing or illegible when filed

TABLE 3
Olig2 inhbitor compounds and activity thereof.

| | | | |
|---|---|---|---|
| 1 | SKOG-101 | 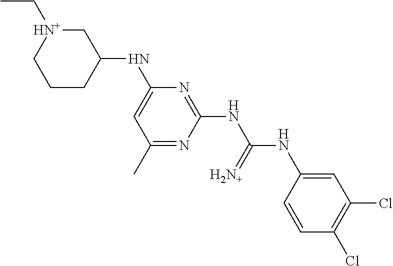 [SMILES: CC[NH+]1CCCC(Nc2cc(C)nc(NC(=[NH2+])Nc3ccc(Cl)c(Cl)c3)n2)C1] | cytotoxic |
| 2 | SKOG-102 | 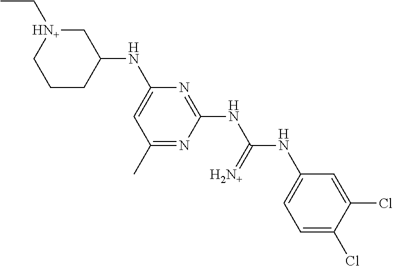 [SMILES: CC[NH+]1CCCC(Nc2cc(C)nc(NC(=[NH2+])Nc3ccc(Cl)c(Cl)c3)n2)C1] | cytotoxic |
| 3 | SKOG-103 | 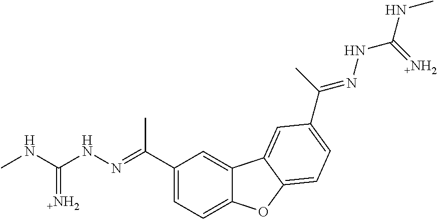 | cytotoxic |
| 4 | SKOG-104 | 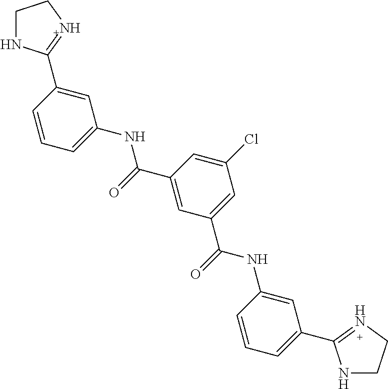 | cytotoxic |
| 5 | SKOG-105 | 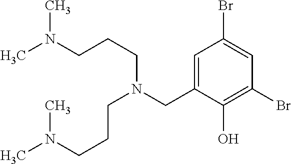 [SMILES: CN(C)CCCN(CCCN(C)C)Cc1cc(Br)cc(Br)c1O] | cytotoxic |
| 6 | SKOG-106 | 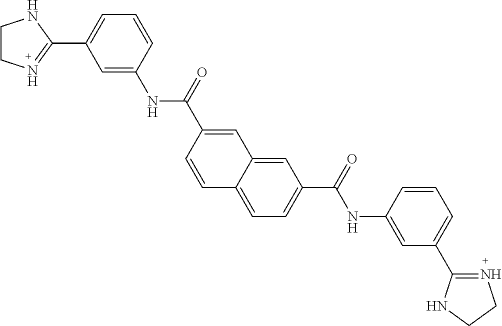 [SMILES: O=C(Nc1cccc(C2=NCCN2)c1)c1ccc2ccc(C(=O)Nc3cccc(C4=NCCN4)c3)cc2c1] | cytotoxic |
| 7 | SKOG-107 | 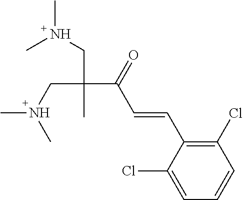 [SMILES: CN(C)CC(C)(CN(C)C)C(=O)/C=C/c1c(Cl)cccc1Cl] | cytotoxic |
| 8 | SKOG-108 | 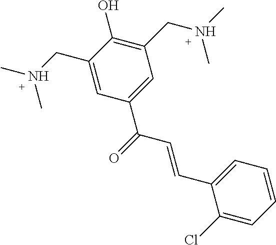 [SMILES: CN(C)Cc1cc(C(=O)/C=C/c2ccccc2Cl)cc(CN(C)C)c1O] | cytotoxic |
| 9 | SKOG-109 | 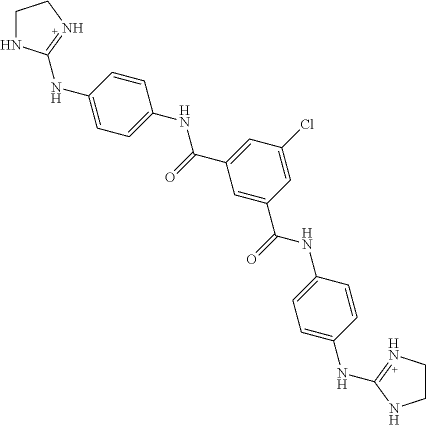 | cytotoxic |
| 10 | SKOG-110 | 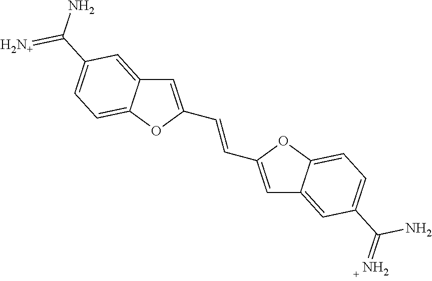 [SMILES: N=C(N)c1ccc2oc(/C=C/c3cc4cc(C(N)=[NH2+])ccc4o3)cc2c1] | cytotoxic |
| 11 | SKOG-111 | 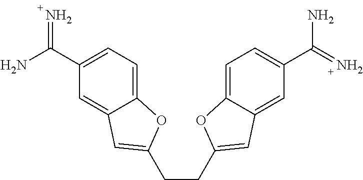 [SMILES: N=C(N)c1ccc2oc(CCc3cc4cc(C(=N)N)ccc4o3)cc2c1] | cytotoxic |
| 12 | SKOG-112 | 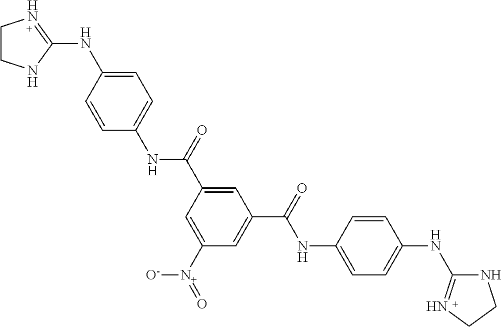 | cytotoxic |
| 13 | SKOG-113 | 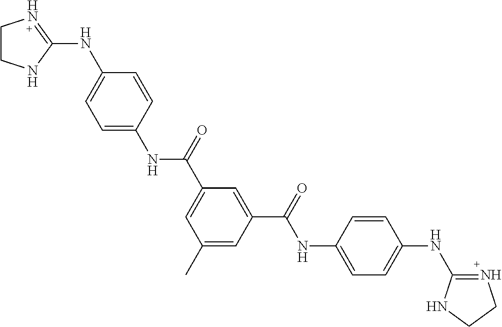 | cytotoxic |
| 14 | SKOG-114 | 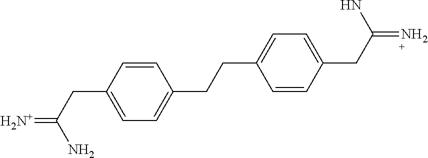 [SMILES: N=C(N)Cc1ccc(CCc2ccc(CC(N)=[NH2+])cc2)cc1] | cytotoxic |
| 15 | SKOG-115 | 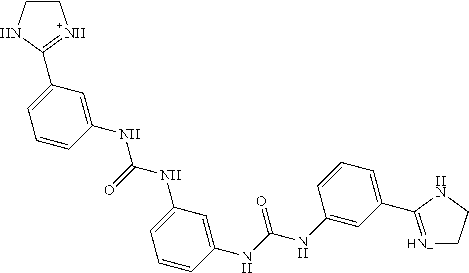 [SMILES: O=C(Nc1cccc(NC(=O)Nc2cccc(C3=[NH+]CCN3)c2)c1)Nc1cccc(C2=NCCN2)c1] | cytotoxic |
| 16 | SKOG-116 | 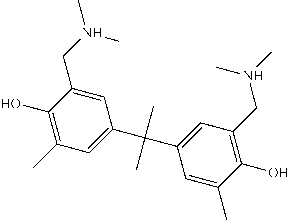 [SMILES: Cc1cc(C(C)(C)c2cc(C)c(O)c(CN(C)C)c2)cc(CN(C)C)c1O] | cytotoxic |
| 17 | SKOG-117 | 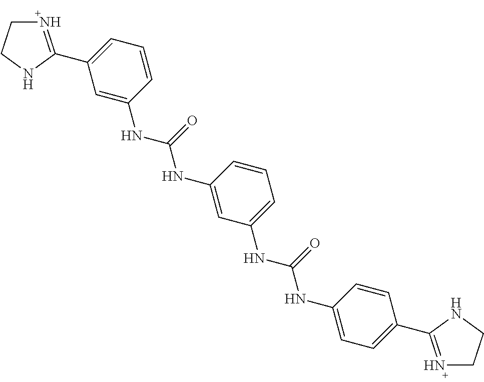 [SMILES: O=C(Nc1ccc(C2=NCCN2)cc1)Nc1cccc(NC(=O)Nc2cccc(C3=NCCN3)c2)c1] | cytotoxic |
| 18 | SKOG-118 | 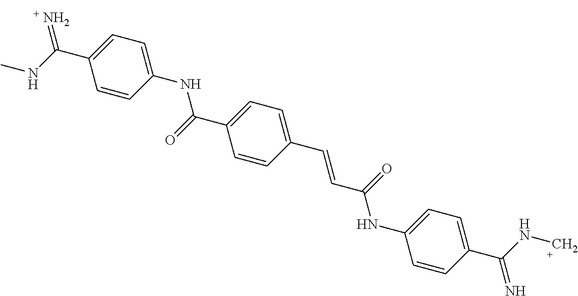 | cytotoxic |
| 19 | SKOG-119 | 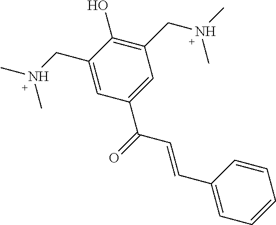 [SMILES: CN(C)Cc1cc(C(=O)/C=C/c2ccccc2)cc(CN(C)C)c1O] | cytotoxic |
| 20 | SKOG-120 | 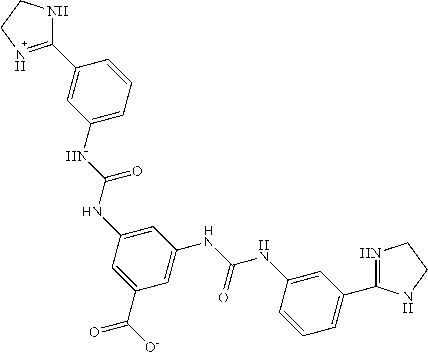 [SMILES: O=C(Nc1cc(NC(=O)Nc2cccc(C3=NCCN3)c2)cc(C(=O)[O-])c1)Nc1cccc(C2=NCCN2)c1] | cytotoxic |
| 21 | SKOG-121 | 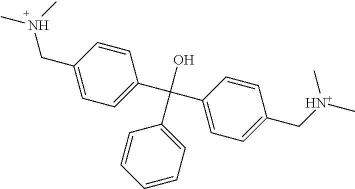 | cytotoxic |
| 22 | SKOG-122 | 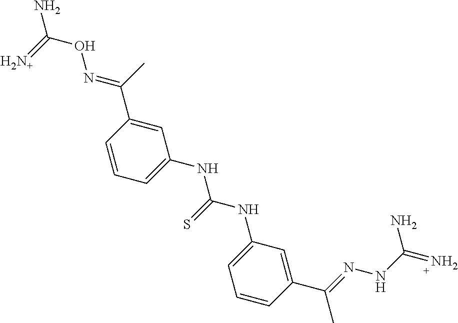 [SMILES: C/C(=N\NC(=N)N)c1cccc(NC(=S)Nc2cccc(/C(C)=N/OC(N)=[NH2+])c2)c1] | cytotoxic |
| 23 | SKOG-123 | 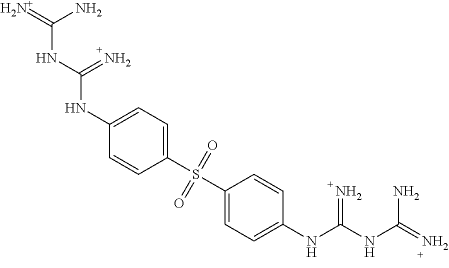 [SMILES: N=C(N)NC(=N)Nc1ccc(S(=O)(=O)c2ccc(NC(=N)NC(N)=[NH2+])cc2)cc1] | cytotoxic |
| 24 | SKOG-124 | 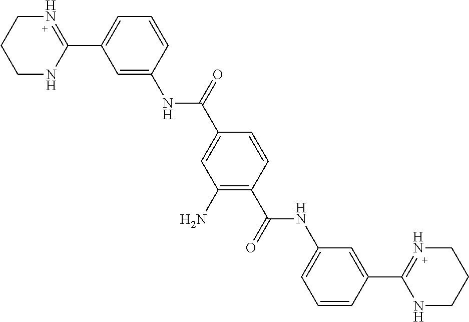 | cytotoxic |
| 25 | SKOG-125 | 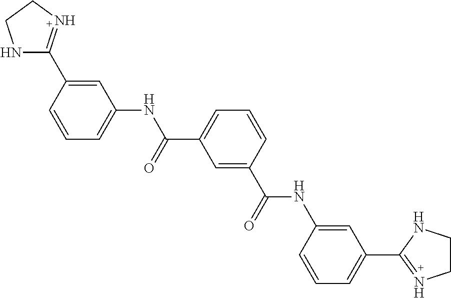 [SMILES: O=C(Nc1cccc(C2=NCCN2)c1)c1cccc(C(=O)Nc2cccc(C3=NCCN3)c2)c1] | cytotoxic |
| 26 | SKOG-126 | 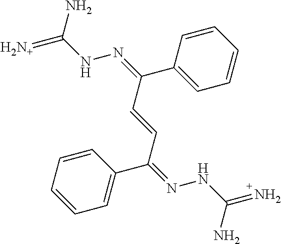 [SMILES: N=C(N)N\N=C(/C=C/C(=N\NC(N)=[NH2+])c1ccccc1)c1ccccc1] | cytotoxic |
| 27 | SKOG-127 | 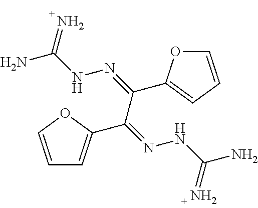 [SMILES: N=C(N)N/N=C(\C(=N/NC(=N)N)c1ccco1)c1ccco1] | cytotoxic |
| 28 | SKOG-128 | 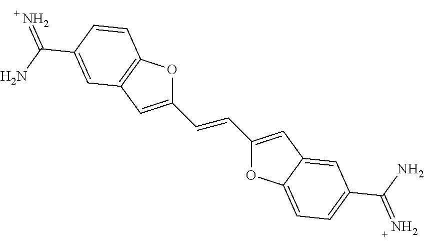 [SMILES: N=C(N)c1ccc2oc(/C=C/c3cc4cc(C(=N)N)ccc4o3)cc2c1] | cytotoxic |
| 29 | SKOG-129 | 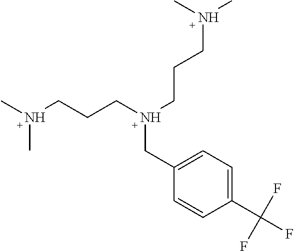 [SMILES: CN(C)CCCN(CCCN(C)C)Cc1ccc(C(F)(F)F)cc1] | cytotoxic |
| 30 | SKOG-130 | 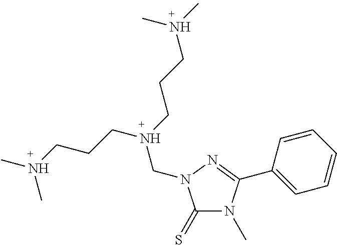 [SMILES: CN(C)CCCN(CCCN(C)C)Cn1nc(-c2ccccc2)n(C)c1=S] | NA |
| 31 | SKOG-131 | 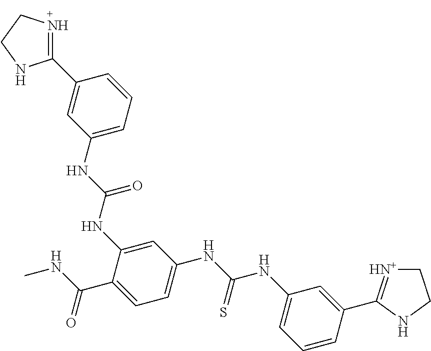 [SMILES: CNC(=O)c1ccc(NC(=S)Nc2cccc(C3=[NH+]CCN3)c2)cc1NC(=O)Nc1cccc(C2=NCCN2)c1] | cytotoxic |
| 32 | SKOG-132 | 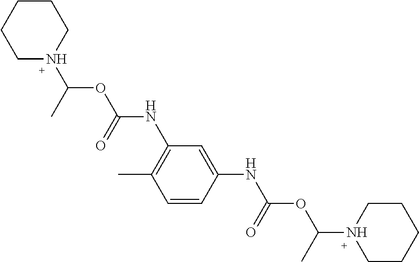 [SMILES: Cc1ccc(NC(=O)OC(C)N2CCCCC2)cc1NC(=O)OC(C)N1CCCCC1] | cytotoxic |
| 33 | SKOG-133 | 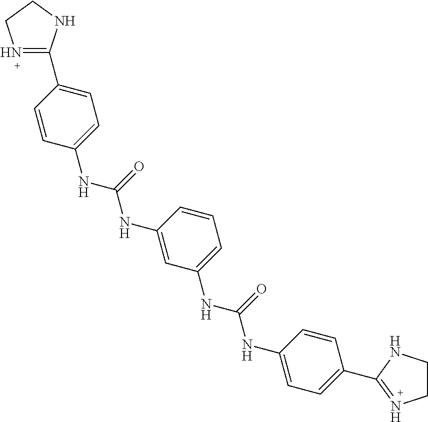 [SMILES: O=C(Nc1ccc(C2=NCCN2)cc1)Nc1cccc(NC(=O)Nc2ccc(C3=NCCN3)cc2)c1] | cytotoxic |
| 34 | SKOG-134 | 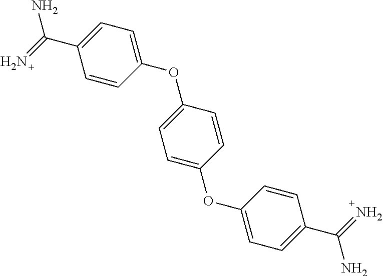 [SMILES: N=C(N)c1ccc(Oc2ccc(Oc3ccc(C(=N)N)cc3)cc2)cc1] | cytotoxic |
| 35 | SKOG-135 | 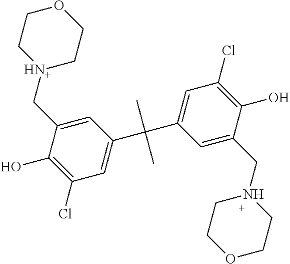 [SMILES: CC(C)(c1cc(Cl)c(O)c(CN2CCOCC2)c1)c1cc(Cl)c(O)c(C[NH+]2CCOCC2)c1] | cytotoxic |
| 36 | SKOG-136 | 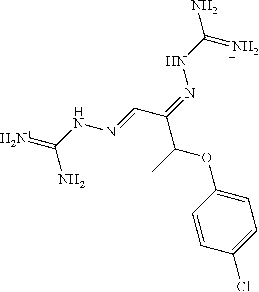 [SMILES: CC(Oc1ccc(Cl)cc1)C(/C=N/NC(N)=[NH2+])=N/NC(=N)N] | cytotoxic |
| 37 | SKOG-137 | 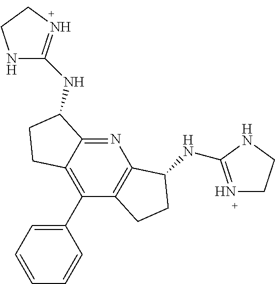 [SMILES: c1ccc(-c2c3c(nc4c2CC[C@H]4NC2=NCCN2)[C@@H](NC2=NCCN2)CC3)cc1] | cytotoxic |
| 38 | SKOG-138 | 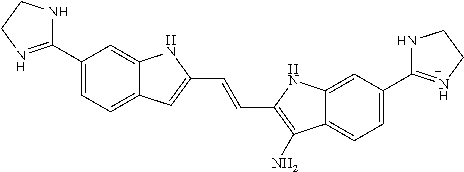 [SMILES: Nc1c(/C=C/c2cc3ccc(C4=NCCN4)cc3[nH]2)[nH]c2cc(C3=NCCN3)ccc12] | cytotoxic |
| 39 | SKOG-139 | 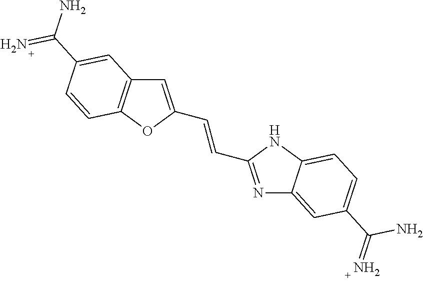 [SMILES: N=C(N)c1ccc2oc(/C=C/c3nc4cc(C(=N)N)ccc4[nH]3)cc2c1] | NA |
| 40 | SKOG-140 | 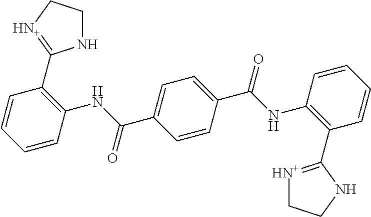 [SMILES: O=C(Nc1ccccc1C1=[NH+]CCN1)c1ccc(C(=O)Nc2ccccc2C2=[NH+]CCN2)cc1] | cytotoxic |

TABLE 4
Peptide probe sequences for OLIG2 pharmacophore
definition

| SEQ | | |
|---|---|---|
| ID | | |
| NO: | PEPTIDE SEQUENCE | TARGET OLIG2 REGIONS |
| 1 | DLNIAMDGLREVM | All contact points for brown |
| | | sequence |
| 2 | DLNIAMDGLRE | Short segment of brown helix |
| 3 | DLNIAMDGLR | Short segment of brown helix |
| 4 | DLNIAMD | Short segment of brown helix |
| 5 | AMDGLREVM | Overlapping brown helix - |
| | | loop segment |
| 6 | DGLREVM | Brown helix segment adjacent |
| | | to loop |
| 7 | YAHGPSVRKLSKIATLL | Loop/C- terminal helix |
| | LARNYILMLTN | |
| 8 | YAHGPSVRKLSKIATLL | Loop/C- terminal helix no |
| | LAR | hydrophobic end |
| 9 | KLSKIATLLLARNYILM | C-terminal helix only |
| | LTN | |
| 10 | TLLLARNYILMLTN | C-terminal helix no hydro- |
| | | phobic end |
| 11 | RKLSKIATLLLAR | End of loop beginning of |
| | | helix |
| 12 | YAHGPSVRKLSK | Loop sequence only |
| 13 | RNYILMLTN | Segment of C- terminal |

### IX. Embodiments

Embodiment 1. A compound having the formula:

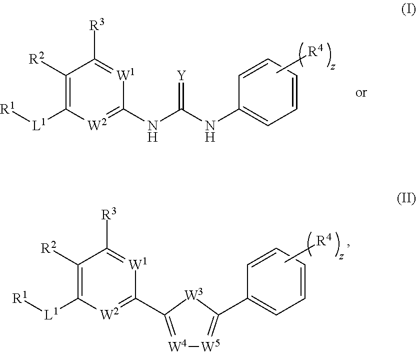

wherein,

R¹ is independently hydrogen, halogen, —CX^{a}₃, —CN, —SO₂Cl, —SOₙ₁R⁵, —SOᵥ₁NR⁵R⁶, —NHNH₂, —ONR⁵R⁶, —NHC═(O)NHNH₂, —NHC═(O)NR⁵R⁶, —N(O)ₘ₁, —NR⁵R⁶, —NH—O—R⁵, —C(O)R⁵, —C(O)—OR⁵, —C(O)NR⁵R⁶, —OR⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R² is independently hydrogen, halogen, —CX^{b}₃, —CN, —SO₂Cl, —SOₙ₂R⁷, —SOᵥ₂NR⁷R⁸, —NHNH₂, —ONR⁷R⁸, —NHC═(O)NHNH₂, —NHC═(O)NR⁷R⁸, —N(O)ₘ₂, —NR⁷R⁸, —NH—O—R⁷, —C(O)R⁷, —C(O)—OR⁷, —C(O)NR⁷R⁸, —OR⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R³ is independently hydrogen, halogen, —CX^{c}₃, —CN, —SO₂Cl, —SOₙ₃R⁹, —SOᵥ₃NR⁹R¹⁰, —NHNH₂, —ONR⁹R¹⁰, —NHC═(O)NHNH₂, —NHC═(O)NR⁹R¹⁰, —N(O)ₘ₃, —NR⁹R¹⁰, —NH—O—R⁹, —C(O)R⁹, —C(O)—OR⁹, —C(O)NR⁹R¹⁰, —OR⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁴ is independently hydrogen, halogen, —CX^{d}₃, —CN, —SO₂Cl, —SOₙ₄R¹¹, —SOᵥ₄NR¹¹R¹², —NHNH₂, —ONR¹¹R¹², —NHC═(O)NHNH₂, —NHC═(O)NR¹¹R¹², —N(O)ₘ₄, —NR¹¹R¹², —NH—O—R¹¹, —C(O)R¹¹, —C(O)—OR¹¹, —C(O)NR¹¹R¹², —OR¹¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

Y is independently O, S or NH;

W¹, W², W⁴ and W⁵ are independently CR¹³ or N;

W³ is O, NR¹⁴, or S;

L¹ is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, —NH-L²-, —NH—R¹⁵—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

L² is independently —C(O)—, —C(O)—NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R⁵, R⁶, R⁷, R⁸, R⁹, R¹⁰, R¹¹, R¹², R¹³, R¹⁴ and R¹⁵ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

X^{a}, X^{b}, X^{c} and X^{d} are independently —F, —Cl, —Br, or —I;

n₁, n₂, n₃ and n₄ are independently an integer from 0 to 4;

m₁, m₂, m₃ and m₄ are independently an integer from 1 to 2;

v₁, v₂, v₃ and v₄ are independently an integer from 1 to 2;

z is independently an integer from 0 to 5.

Embodiment 2 The compound of embodiment 1, wherein R¹ is independently R^{1A}-substituted or unsubstituted alkyl, R^{1A}-substituted or unsubstituted heteroalkyl, R^{1A}-substituted or unsubstituted cycloalkyl, R^{1A}-substituted or unsubstituted heterocycloalkyl, R^{1A}-substituted or unsubstituted aryl, or R^{1A}-substituted or unsubstituted heteroaryl;

R^{1A} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{1B}-substituted or unsubstituted alkyl, R^{1B}-substituted or unsubstituted heteroalkyl, R^{1B}-substituted or unsubstituted cycloalkyl, R^{1B}-substituted or unsubstituted heterocycloalkyl, R^{1B}-substituted or unsubstituted aryl, or R^{1B}-substituted or unsubstituted heteroaryl;

R^{1B} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{1C}-substituted or unsubstituted alkyl, R^{1C}-substituted or unsubstituted heteroalkyl, R^{1C}-substituted or unsubstituted cycloalkyl, R^{1C}-substituted or unsubstituted heterocycloalkyl, R^{1C}-substituted or unsubstituted aryl, or R^{1C}-substituted or unsubstituted heteroaryl;

R^{1C} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{1D}-substituted or unsubstituted alkyl, R^{1D}-substituted or unsubstituted heteroalkyl, R^{1D}-substituted or unsubstituted cycloalkyl, R^{1D}-substituted or unsubstituted heterocycloalkyl, R^{1D}-substituted or unsubstituted aryl, or R^{1D}-substituted or unsubstituted heteroaryl;

R^{1D} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{1E}-substituted or unsubstituted alkyl, R^{1E}-substituted or unsubstituted heteroalkyl, R^{1E}-substituted or unsubstituted cycloalkyl, R^{1E}-substituted or unsubstituted heterocycloalkyl, R^{1E}-substituted or unsubstituted aryl, or R^{1E}-substituted or unsubstituted heteroaryl; and

R^{1E} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl.

Embodiment 3. The compound of embodiment 1, wherein R¹ is substituted or unsubstituted alkyl or substituted or unsubstituted heterocycloalkyl.

Embodiment 4. The compound of embodiment 3, wherein R¹ is substituted or unsubstituted C₁-C₅ alkyl.

Embodiment 5 The compound of embodiment 4, wherein R¹ is unsubstituted ethyl.

Embodiment 6. The compound of embodiment 3, wherein R¹ is substituted or unsubstituted 5 or 6 membered heterocycloalkyl.

Embodiment 7. The compound of embodiment 6, wherein R¹ is substituted or unsubstituted 6 membered heterocycloalkyl.

Embodiment 8. The compound of embodiment 7, wherein R¹ is substituted or unsubstituted piperidinyl.

Embodiment 9 The compound of embodiment 6, wherein R¹ is substituted or unsubstituted 5 membered heterocycloalkyl.

Embodiment 10. The compound of embodiment 9, wherein R¹ is substituted or unsubstituted pyrrolidinyl.

Embodiment 11. The compound of embodiment 2, wherein R¹ is R^{1A}-substituted or unsubstituted 5 or 6 membered heterocycloalkyl.

Embodiment 12 The compound of embodiment 11, wherein R¹ is R^{1A}-substituted or unsubstituted 5 membered heterocycloalkyl.

Embodiment 13. The compound of embodiment 12, wherein R¹ is

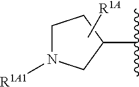

and wherein R^{1A1} is substituted or unsubstituted C₁-C₅ alkyl

Embodiment 14. The compound of embodiment 13, wherein R^{1A1} is unsubstituted methyl or ethyl.

Embodiment 15 The compound of embodiment 14, wherein R^{1A} is hydrogen.

Embodiment 16. The compound of embodiment 13, wherein R^{1A} is R^{1B}-substituted or unsubstituted C₁-C₅ alkyl, R^{1B} is R^{1C}-substituted or unsubstituted 5 to 10 membered heteroaryl, and R^{1C} is substituted or unsubstituted 1 to 5 membered heteroalkyl.

Embodiment 17. The compound of embodiment 16, wherein R^{1A} is R^{1B}-substituted or unsubstituted unsaturated C₁-C₅ alkyl.

Embodiment 18. The compound of embodiment 17, wherein R^{1A} is R^{1B}-substituted or unsubstituted ethenyl.

Embodiment 19 The compound of embodiment 16, wherein R^{1B} is R^{1C}-substituted 9 membered heteroaryl.

Embodiment 20. The compound of embodiment 20, wherein R^{1B} is R^{1C}-substituted benzofuranyl.

Embodiment 21. The compound of embodiment 16, wherein R^{1C} is —C(NH)NH₂.

Embodiment 22 The compound of embodiment 13, wherein R^{1A} is R^{1B}-substituted or unsubstituted 3 to 10 membered heteroalkyl.

Embodiment 23. The compound of embodiment 22, wherein R^{1B} is unsubstituted alkyl, ═O or ═S.

Embodiment 24. The compound of embodiment 22, wherein R^{1A} is R^{1B}-substituted 6 membered heteroalkyl and R^{1B} is independently unsubstituted methyl, ═O or ═S.

Embodiment 25 The compound of embodiment 11, wherein R¹ is R^{1A}-substituted or unsubstituted 6 membered heterocycloalkyl.

Embodiment 26. The compound of embodiment 25, wherein R¹ is

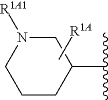

and wherein R^{1A1} is hydrogen or substituted or unsubstituted C₁-C₅ alkyl.

Embodiment 27. The compound of embodiment 26, wherein R^{1A1} is unsubstituted methyl or ethyl.

Embodiment 28. The compound of embodiment 26, wherein R^{1A1} and R^{1A} are independently hydrogen.

Embodiment 29 The compound of embodiment 26, wherein R^{1A} is hydrogen, halogen, —NO₂ or substituted or unsubstituted heteroalkyl.

Embodiment 30. The compound of embodiment 29, wherein R^{1A} is R^{1B}-substituted or unsubstituted 3 to 10 membered heteroalkyl.

Embodiment 31. The compound of embodiment 30, wherein R^{1B} is unsubstituted alkyl, ═O or ═S.

Embodiment 32 The compound of embodiment 30, wherein R^{1A} is R^{1B}-substituted 6 membered heteroalkyl and R^{1B} is independently unsubstituted methyl, ═O or ═S.

Embodiment 33. The compound of embodiment 1, wherein R² is hydrogen, substituted or unsubstituted alkyl or OR⁷.

Embodiment 34. The compound of embodiment 33, wherein R² is hydrogen or —OR′.

Embodiment 35 The compound of embodiment 34, wherein R⁷ is substituted or unsubstituted C₁-C₅ alkyl.

Embodiment 36. The compound of embodiment 35, wherein R⁷ is methyl.

Embodiment 37. The compound of embodiment 1, wherein R³ is substituted or unsubstituted alkyl, —NR⁹R¹⁰ or —NH—OR⁹.

Embodiment 38. The compound of embodiment 37, wherein R³ is hydrogen, methyl, —NR⁹R¹⁰ or —NH—OR⁹.

Embodiment 39 The compound of embodiment 38, wherein R³ is methyl.

Embodiment 40. The compound of embodiment 38, wherein R⁹ and R¹⁰ are independently hydrogen or substituted or unsubstituted C₁-C₅ alkyl.

Embodiment 41. The compound of embodiment 40, wherein R³ is —NR⁹R¹⁰ and R⁹ and R¹⁰ are independently hydrogen.

Embodiment 42 The compound of embodiment 40, wherein R³ is —NR⁹R¹⁰ and R⁹ and R¹⁰ are independently methyl.

Embodiment 43. The compound of embodiment 38, wherein R³ is —NH—OR⁹.

Embodiment 44. The compound of embodiment 43, wherein R⁹ is hydrogen or substituted or unsubstituted alkyl.

Embodiment 45 The compound of embodiment 44, wherein R⁹ is methyl.

Embodiment 46. The compound of embodiment 1, wherein R⁴ is independently hydrogen, halogen, —CX^{d}₃, —SOᵥ₄NR¹¹R¹², —OR¹¹ or substituted or unsubstituted alkyl.

Embodiment 47. The compound of embodiment 46, wherein X^{d} is —F, v₄ is 2, R¹¹ and R¹² are independently hydrogen or substituted or unsubstituted alkyl.

Embodiment 48. The compound of embodiment 47, wherein R⁴ is independently hydrogen, —Cl, —F, —CF₃, —SO₂NH₂ or methyl.

Embodiment 49 The compound of embodiment 48, wherein z is 2.

Embodiment 50. The compound of embodiment 48, wherein R⁴ is methyl and z is 2.

Embodiment 51. The compound of embodiment 47, wherein R¹¹ and R¹² are independently hydrogen and z is 1.

Embodiment 52 The compound of embodiment 48, wherein R⁴ is independently —Cl or —CF₃ and z is 2.

Embodiment 53. The compound of embodiment 48, wherein R⁴ is independently —Cl or hydrogen and z is 2.

Embodiment 54. The compound of embodiment 48, wherein R⁴ is independently —SO₂NH₂ or hydrogen and z is 2.

Embodiment 55 The compound of embodiment 46, wherein R⁴ is —OR¹¹ and z is 1.

Embodiment 56. The compound of embodiment 55, wherein R^{H} is —CF₃.

Embodiment 57. The compound of embodiment 1, wherein W¹, W², W⁴, and W⁵ are independently N or CH.

Embodiment 58. The compound of embodiment 57, wherein W³ is independently O, NH or S.

Embodiment 59 The compound of embodiment 1, wherein L¹ is unsubstituted C₁-C₅ alkylene, —NH— or —NH-L²-.

Embodiment 60. The compound of embodiment 59, wherein L¹ is unsubstituted methylene.

Embodiment 61. The compound of embodiment 59, wherein L² is independently R¹⁶-substituted or unsubstituted alkylene, R¹⁶-substituted or unsubstituted heteroalkylene, R¹⁶-substituted or unsubstituted cycloalkylene, R¹⁶-substituted or unsubstituted heterocycloalkylene, R¹⁶-substituted or unsubstituted arylene, or R¹⁶-substituted or unsubstituted heteroarylene;

R¹⁶ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R¹⁷-substituted or unsubstituted alkyl, R¹⁷-substituted or unsubstituted heteroalkyl, R¹⁷-substituted or unsubstituted cycloalkyl, R¹⁷-substituted or unsubstituted heterocycloalkyl, R¹⁷-substituted or unsubstituted aryl, or R¹⁷-substituted or unsubstituted heteroaryl;

R¹⁷ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R¹⁸-substituted or unsubstituted alkyl, R¹⁸-substituted or unsubstituted heteroalkyl, R¹⁸-substituted or unsubstituted cycloalkyl, R¹⁸-substituted or unsubstituted heterocycloalkyl, R¹⁸-substituted or unsubstituted aryl, or R¹⁸-substituted or unsubstituted heteroaryl;

R¹⁸ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R¹⁹-substituted or unsubstituted alkyl, R¹⁹-substituted or unsubstituted heteroalkyl, R¹⁹-substituted or unsubstituted cycloalkyl, R¹⁹-substituted or unsubstituted heterocycloalkyl, R¹⁹-substituted or unsubstituted aryl, or R¹⁹-substituted or unsubstituted heteroaryl;

R¹⁹ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R²⁰-substituted or unsubstituted alkyl, R²⁰-substituted or unsubstituted heteroalkyl, R²⁰-substituted or unsubstituted cycloalkyl, R²⁰-substituted or unsubstituted heterocycloalkyl, R²⁰-substituted or unsubstituted aryl, or R²⁰-substituted or unsubstituted heteroaryl; and

R²⁰ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl.

Embodiment 62 The compound of embodiment 61, wherein L² is unsubstituted C₁-C₅ alkylene.

Embodiment 63. The compound of embodiment 62, wherein L² is unsubstituted methylene.

Embodiment 64. The compound of embodiment 61, wherein L² is R¹⁶-substituted or unsubstituted C₁-C₅ alkylene.

Embodiment 65 The compound of embodiment 64, wherein L² is R¹⁶-substituted methylene.

Embodiment 66. The compound of embodiment 64, wherein R¹⁶ is R¹⁷-substituted or unsubstituted C₁-C₅ alkyl, R¹⁷ is R¹⁸-substituted or unsubstituted 5 to 10 membered heteroaryl, and R¹⁸ is substituted or unsubstituted 1 to 5 membered heteroalkyl.

Embodiment 67. The compound of embodiment 66, wherein R¹⁶ is R¹⁷-substituted or unsubstituted saturated C₁-C₅ alkyl.

Embodiment 68. The compound of embodiment 67, wherein R¹⁶ is R¹⁷-substituted or unsubstituted ethenyl.

Embodiment 69 The compound of embodiment 66, wherein R¹⁷ is R¹⁸-substituted 9 membered heteroaryl.

Embodiment 70. The compound of embodiment 69, wherein R¹⁷ is R¹⁸-substituted benzofuranyl.

Embodiment 71. The compound of embodiment 66, wherein R¹⁸ is —C(NH)NH₂.

Embodiment 72 The compound of embodiment 66, wherein R¹ is substituted or unsubstituted 5 membered heterocycloalkyl.

Embodiment 73. The compound of embodiment 72, wherein R¹ is pyrrolidinyl.

Embodiment 74. The compound of embodiment 1, wherein the compound has the formula:

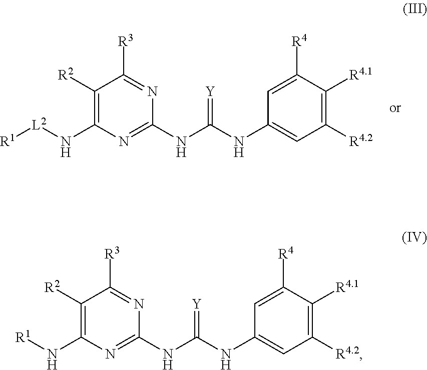

wherein

R⁴, R^{4.1} and R^{4.2} are independently hydrogen, halogen, —CX^{d}₃, —SOᵥ₄NR¹¹R¹², —OR¹¹, or substituted or unsubstituted alkyl.

Embodiment 75 The compound of embodiment 74, wherein R¹ is substituted or unsubstituted piperidinyl or substituted or unsubstituted pyrrolidinyl.

Embodiment 76. The compound of embodiment 1, wherein the compound has the formula:

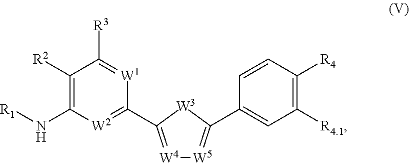

wherein

R⁴ and R^{4.1} are independently halogen or —CF₃

Embodiment 77. The compound of embodiment 76, wherein R¹ is substituted or unsubstituted piperidinyl.

Embodiment 78. A compound having the formula:

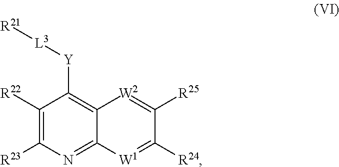

wherein,

R²¹ is independently hydrogen, halogen, —CX^{a}₃, —CN, —SO₂Cl, —SOₙ₁R²⁸, —SOᵥ₁NR²⁸R²⁹, —NHNH₂, —ONR²⁸R²⁹, —NHC═(O)NHNH₂, —NHC═(O)NR²⁸R²⁹, —NHC═(O)R²⁸, —NOₘ₁, —NR²⁸R²⁹, —NH—O—R²⁸, —C(O)R²⁸, —C(O)—OR²⁸, —C(O)NR28R²⁹, —N(R²⁸)C(O)R²⁹, —OR²⁸, —O—C(O)NR²⁸R²⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²² is independently hydrogen, halogen, —CX^{b}₃, —CN, —SO₂Cl, —SOₙ₂R³⁰, —SOᵥ₂NR³⁰R³¹, —NHNH₂, —ONR³⁰R³¹, —NHC═(O)NHNH₂, —NHC═(O)NR³⁰R³¹, —NHC═(O)R³⁰, —N(O)ₘ₂, —NR³⁰R³¹, —NH—O—R³⁰, —C(O)R³⁰, —C(O)—OR³⁰, —C(O)NR³⁰R³¹, —N(R³⁰)C(O)R³¹, —O—C(O)NR³⁰R³¹, —OR³⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²³ is independently hydrogen, halogen, —CX^{c}₃, —CN, —SO₂Cl, —SOₙ₃R³², —SOᵥ₃NR³²R³³, —NR³²SOᵥ₃R³³, —NHNH₂, —ONR³²R³³, —NHC═(O)NHNH₂, —NHC═(O)NR³²R³³, —NHC═(O)R³², —N(O)ₘ₃, —NR³²R³³, —NH—O—R³², —R³²NR³³NH₂, —C(O)R³², —C(O)—OR³², —C(O)NR³²R³³, —N(R³²)C(O)R³³, —O—C(O)NR³²R³³, —OR³², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁴ is independently hydrogen, halogen, —CX^{d}₃, —CN, —SO₂Cl, —SOₙ₄R³⁴, —SOᵥ₄NR³⁴R³⁵, —NHNH₂, —ONR³⁴R³⁵, —NHC═(O)NHNH₂, —NHC═(O)NR³⁴R³⁵, —NHC═(O)R³⁴, —N(O)ₘ₄, —NR³⁴R³⁵, —NH—O—R³⁴, —C(O)R³⁴, —C(O)—OR³⁴, —C(O)NR³⁴R³⁵, —N(R³⁴)C(O)R³⁵, —O—C(O)NR³⁴R³⁵, —OR³⁴, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁵ is independently hydrogen, halogen, —CX^{e}₃, —CN, —SO₂Cl, —SOₙ₅R³⁶, —SOᵥ₅NR³⁶R³⁷, —NHNH₂, —ONR³⁶R³⁷, —NHC═(O)NHNH₂, —NHC═(O)NR³⁶R³⁷, —NHC═(O)R³⁶, —N(O)ₘ₅, —NR³⁶R³⁷, —NH—O—R³⁶, —C(O)R³⁶, —C(O)—OR³⁶, —C(O)NR³⁶R³⁷, —N(R³⁶)C(O)R³⁷, —O—C(O)NR³⁶R³⁷, —OR³⁶, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁶ is independently hydrogen, halogen, —CX^{f}₃, —CN, —SO₂Cl, —SOₙ₆R³⁸, —SOᵥ₆NR³⁸R³⁹, —NHNH₂, —ONR³⁸R³⁹, —NHC═(O)NHNH₂, —NHC═(O)NR³⁸R³⁹, —NHC═(O)R³⁸, —N(O)ₘ₆, —NR³⁸R³⁹, —NH—O—R³⁸, —C(O)R³⁸, —C(O)—OR³⁸, —C(O)NR³⁸R³⁹, —N(R³⁸)C(O)R³⁹, —O—C(O)NR³⁸R³⁹, —OR³⁸, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁷ is independently hydrogen, halogen, —CX^{g}₃, —CN, —SO₂Cl, —SOₙ₇R⁴⁰, —SOᵥ₇NR⁴⁰R⁴¹, —NHNH₂, —ONR⁴⁰R⁴¹, —NHC═(O)NHNH₂, —NHC═(O)NR⁴⁰R⁴¹, —NHC═(O)R⁴⁰, —N(O)ₘ₇, —NR⁴⁰R⁴¹, —NH—O—R⁴⁰, —C(O)R⁴⁰, —C(O)—OR⁴⁰, —C(O)NR⁴⁰R⁴¹, —N(R⁴⁰)C(O)R⁴¹, —O—C(O)NR⁴⁰R⁴¹, —OR⁴⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁸ is independently hydrogen, halogen, —CX^{h}₃, —CN, —SO₂Cl, —SOₙ₈R⁴², —SOᵥ₈NR⁴²R⁴³, —NHNH₂, —ONR⁴²R⁴³, —NHC═(O)NHNH₂, —NHC═(O)NR⁴²R⁴³, —NHC═(O)R⁴², —N(O)ₘ₈, —NR⁴²R⁴³, —NH—O—R⁴², —C(O)R⁴², —C(O)—OR⁴², —C(O)NR⁴²R⁴³, —N(R⁴²)C(O)R⁴³, —O—C(O)NR⁴²R⁴³, —OR⁴², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁹ is independently hydrogen, halogen, —CXⁱ₃, —CN, —SO₂Cl, —SOₙ₉R⁴⁴, —SOᵥ₉NR⁴⁴R⁴⁵, —NHNH₂, —ONR⁴⁴R⁴⁵, —NHC═(O)NHNH₂, —NHC═(O)NR⁴⁴R⁴⁵, —NHC═(O)R⁴⁴, —N(O)ₘ₉, —NR⁴⁴R⁴⁵, —NH—O—R⁴⁴, —C(O)R⁴⁴, —C(O)—OR⁴⁴, —C(O)NR⁴⁴R⁴⁵, —N(R⁴⁴)C(O)R⁴⁵, —O—C(O)NR⁴⁴R⁴⁵, —OR⁴⁴, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

Y is independently O or NH;

W¹ is independently N or CR²⁶;

W² is independently N or CR²⁷;

L³ is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R³⁰, R³¹, R³², R³³, R³⁴, R³⁵ R³⁶, R³⁷, R³⁸, R³⁹, R⁴⁰, R⁴¹, R⁴², R⁴³, R⁴⁴ and R⁴⁵ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

X^{a}, X^{b}, X^{c}, X^{d}, X^{e}, X^{f}, X^{g}, X^{h} and Xⁱ are independently —F, —Cl, —Br, or —I;

n₁, n₂, n₃, n₄, n₅, n₆ an, n₇, n₈ and n₉ are independently an integer from 0 to 4;

m₁, m₂, m₃, m₄, m₅, m₆, m₇, m₈ and m₉ are independently an integer from 1 to 2;

v₁, v₂, v₃, v₄, v₅, v₆, v₇, v₈ and v₉ are independently an integer from 1 to 2;

z is independently an integer from 0 to 5.

Embodiment 79 The compound of embodiment 78, wherein R²¹ is independently R^{21A}-substituted or unsubstituted alkyl, R^{21A}-substituted or unsubstituted heteroalkyl, R^{21A}-substituted or unsubstituted cycloalkyl, R^{21A}-substituted or unsubstituted heterocycloalkyl, R^{21A}-substituted or unsubstituted aryl, or R^{21A}-substituted or unsubstituted heteroaryl;

R^{21A} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{21B}-substituted or unsubstituted alkyl, R^{21B}-substituted or unsubstituted heteroalkyl, R^{21B}-substituted or unsubstituted cycloalkyl, R^{21B}-substituted or unsubstituted heterocycloalkyl, R^{21B}-substituted or unsubstituted aryl, or R^{21B}-substituted or unsubstituted heteroaryl;

R^{21B} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{21C}-substituted or unsubstituted alkyl, R^{21C}-substituted or unsubstituted heteroalkyl, R^{21C}-substituted or unsubstituted cycloalkyl, R^{21C}-substituted or unsubstituted heterocycloalkyl, R^{21C}-substituted or unsubstituted aryl, or R^{21C}-substituted or unsubstituted heteroaryl;

R^{21C} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{21D}-substituted or unsubstituted alkyl, R^{21D}-substituted or unsubstituted heteroalkyl, R^{21D}-substituted or unsubstituted cycloalkyl, R^{21D}-substituted or unsubstituted heterocycloalkyl, R^{21D}-substituted or unsubstituted aryl, or R^{21D}-substituted or unsubstituted heteroaryl;

R^{21D} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{21E}-substituted or unsubstituted alkyl, R^{21E}-substituted or unsubstituted heteroalkyl, R^{21E}-substituted or unsubstituted cycloalkyl, R^{21E}-substituted or unsubstituted heterocycloalkyl, R^{21E}-substituted or unsubstituted aryl, or R^{21E}-substituted or unsubstituted heteroaryl; and

R^{21E} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl

Embodiment 80. The compound of embodiment 79, wherein R²¹ is substituted or unsubstituted alkyl, substituted or unsubstituted heteroalkyl, substituted or unsubstituted aryl, substituted or unsubstituted heteroaryl, —SOᵥ₁NR²⁸R²⁹, —NHC═(O)R²⁸, —NR²⁸R²⁹, —C(O)NR²⁸R²⁹, or —O—C(O)NR²⁸R²⁹.

Embodiment 81. The compound of embodiment 80, wherein R²¹ is R^{21A}-substituted or unsubstituted C₁-C₅ alkyl.

Embodiment 82. The compound of embodiment 81, wherein R²¹ is branched unsubstituted C₅ alkyl.

Embodiment 83. The compound of embodiment 81, wherein R²¹ is R^{21A}-substituted C₁-C₃ alkyl.

Embodiment 84. The compound of embodiment 83, wherein R²¹ is R^{21A}-substituted methyl.

Embodiment 85. The compound of embodiment 84, wherein R^{21A} is R^{21B}-substituted or unsubstituted 5 to 10 membered heterocycloalkyl or R^{21B}-substituted or unsubstituted 5 to 10 membered heteroaryl.

Embodiment 86. The compound of embodiment 85, wherein R^{21A} is R^{21B}-substituted 10 membered heteroaryl.

Embodiment 87. The compound of embodiment 86, wherein R^{21B} is independently —NH₂ or methyl.

Embodiment 88. The compound of embodiment 85, wherein R^{21A} is unsubstituted 6 membered heterocycloalkyl.

Embodiment 89. The compound of embodiment 83, wherein R²¹ is R^{21A}-substituted ethyl.

Embodiment 90. The compound of embodiment 89, wherein R^{21A} is R^{21B}-substituted or unsubstituted 5 to 10 membered heterocycloalkyl.

Embodiment 91. The compound of embodiment 90, wherein R^{21A} is R^{21B}-substituted 6 membered heterocycloalkyl.

Embodiment 92 The compound of embodiment 91, wherein R^{21B} is unsubstituted C₃ alkyl.

Embodiment 93. The compound of embodiment 80, wherein R²¹ is R^{21A}-substituted or unsubstituted 4 to 8 membered heteroalkyl.

Embodiment 94. The compound of embodiment 93, wherein R^{21A} is independently methyl or ═O.

Embodiment 95. The compound of embodiment 80, wherein R²¹ is R^{21A}-substituted or unsubstituted aryl.

Embodiment 96. The compound of embodiment 95, wherein R^{21A} is halogen.

Embodiment 97. The compound of embodiment 96, wherein R²¹ is R^{21A}-substituted phenyl and R^{21A} is —Cl.

Embodiment 98. The compound of embodiment 80, wherein R²¹ is R^{21A}-substituted or unsubstituted heteroaryl.

Embodiment 99. The compound of embodiment 98, wherein R^{21A} is C₁-C₅ alkyl.

Embodiment 100. The compound of embodiment 99, wherein R²¹ is R^{21A}-substituted 5 membered heteroaryl and R^{21A} is ethyl.

Embodiment 101. The compound of embodiment 80, wherein R²¹ is —SOᵥ₁NR²⁸R²⁹.

Embodiment 102. The compound of embodiment 101, wherein v₁ is 2 and R²⁸ and R²⁹ are independently hydrogen.

Embodiment 103. The compound of embodiment 80, wherein R²¹ is —NHC═(O)R²⁸.

Embodiment 104. The compound of embodiment 103, wherein R²⁸ is substituted or unsubstituted 5 to 10 membered heteroaryl.

Embodiment 105. The compound of embodiment 104, wherein R²⁸ is unsubstituted indolyl.

Embodiment 106. The compound of embodiment 80, wherein _{R}21 is —NR²⁸R²⁹.

Embodiment 107. The compound of embodiment 106, wherein R²⁸ and R²⁹ are independently hydrogen, substituted or unsubstituted heterocycloalkyl or substituted or unsubstituted heteroaryl.

Embodiment 108. The compound of embodiment 107, wherein R²⁸ is hydrogen and R²⁹ is substituted or unsubstituted 6 membered heterocycloalkyl.

Embodiment 109. The compound of embodiment 108, wherein R²⁸ is hydrogen and R²⁹ is unsubstituted piperazinyl.

Embodiment 110. The compound of embodiment 107, wherein R²⁸ is hydrogen and R²⁹ is substituted or unsubstituted 10 membered heteroaryl.

Embodiment 111. The compound of embodiment 110, wherein R²⁸ is hydrogen and R²⁹ is quinolinyl.

Embodiment 112. The compound of embodiment 80, wherein R²¹ is —C(O)NR²⁸R²⁹.

Embodiment 113. The compound of embodiment 112, wherein R²⁸ and R²⁹ are independently hydrogen, substituted or unsubstituted 4 to 6 membered heteroalkyl or substituted or unsubstituted aryl.

Embodiment 114. The compound of embodiment 113, wherein R²⁸ is hydrogen and R²⁹ is substituted 4 membered heteroalkyl.

Embodiment 115. The compound of embodiment 113, wherein R²⁸ is hydrogen and R²⁹ is substituted 6 membered heteroalkyl.

Embodiment 116. The compound of embodiment 113, wherein R²⁸ is hydrogen and R²⁹ is substituted phenyl.

Embodiment 117. The compound of embodiment 80, wherein R²¹ is —O—C(O)NR²⁸R²⁹.

Embodiment 118. The compound of embodiment 117, wherein R²⁸ and R²⁹ are independently hydrogen or C₁-C₅ alkyl.

Embodiment 119. The compound of embodiment 118, wherein R²⁸ and R²⁹ are independently hydrogen or methyl.

Embodiment 120. The compound of embodiment 78, wherein R²² is independently hydrogen or OR³⁰.

Embodiment 121. The compound of embodiment 120, wherein R³⁰ is substituted or unsubstituted C₁-C₅ alkyl.

Embodiment 122. The compound of embodiment 121, wherein R³⁰ is unsubstituted methyl.

Embodiment 123. The compound of embodiment 78, wherein R²³ is independently hydrogen, halogen, —NR³²SOᵥ₃R³³, —R³²NR³³NH₂, OR³² or substituted or unsubstituted alkyl.

Embodiment 124. The compound of embodiment 123, wherein R³² and R³³ are independently hydrogen or unsubstituted C₁-C₅ alkyl and v₃ is 2.

Embodiment 125. The compound of embodiment 124, wherein R³² and R³³ are independently hydrogen or methyl.

Embodiment 126. The compound of embodiment 78, wherein R²⁴ is independently hydrogen, halogen, substituted or unsubstituted heteroalkyl or substituted or unsubstituted heterocycloalkyl.

Embodiment 127. The compound of embodiment 126, wherein R²⁴ is substituted 2 to 5 membered heteroalkyl or substituted 6 membered heterocycloalkyl.

Embodiment 128. The compound of embodiment 78, wherein R²⁵ is hydrogen.

Embodiment 129. The compound of embodiment 78, wherein W¹ is CR²⁶ and R²⁶ is hydrogen.

Embodiment 130. The compound of embodiment 78, wherein W² is CR²⁷ and R²⁷ is independently hydrogen, halogen or NR⁴⁰R⁴¹.

Embodiment 131. The compound of embodiment 130, wherein R⁴⁰ and R⁴¹ are independently hydrogen.

Embodiment 132. The compound of embodiment 78, wherein L³ is independently R⁴⁶-substituted or unsubstituted alkylene, R⁴⁶-substituted or unsubstituted heteroalkylene, R⁴⁶-substituted or unsubstituted cycloalkylene, R⁴⁶-substituted or unsubstituted heterocycloalkylene, R⁴⁶-substituted or unsubstituted arylene, or R⁴⁶-substituted or unsubstituted heteroarylene;

R⁴⁶ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R⁴⁷-substituted or unsubstituted alkyl, R⁴⁷-substituted or unsubstituted heteroalkyl, R⁴⁷-substituted or unsubstituted cycloalkyl, R⁴⁷-substituted or unsubstituted heterocycloalkyl, R⁴⁷-substituted or unsubstituted aryl, or R⁴⁷-substituted or unsubstituted heteroaryl;

R⁴⁷ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R⁴⁸-substituted or unsubstituted alkyl, R⁴⁸-substituted or unsubstituted heteroalkyl, R⁴⁸-substituted or unsubstituted cycloalkyl, R⁴⁸-substituted or unsubstituted heterocycloalkyl, R⁴⁸-substituted or unsubstituted aryl, or R⁴⁸-substituted or unsubstituted heteroaryl;

R⁴⁸ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R⁴⁹-substituted or unsubstituted alkyl, R⁴⁹-substituted or unsubstituted heteroalkyl, R⁴⁹-substituted or unsubstituted cycloalkyl, R⁴⁹-substituted or unsubstituted heterocycloalkyl, R⁴⁹-substituted or unsubstituted aryl, or R⁴⁹-substituted or unsubstituted heteroaryl;

R⁴⁹ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R⁵⁰-substituted or unsubstituted alkyl, R⁵⁰-substituted or unsubstituted heteroalkyl, R⁵⁰-substituted or unsubstituted cycloalkyl, R⁵⁰-substituted or unsubstituted heterocycloalkyl, R⁵⁰-substituted or unsubstituted aryl, or R⁵⁰-substituted or unsubstituted heteroaryl; and

R⁵⁰ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl.

Embodiment 133. The compound of embodiment 132, wherein L³ is independently a bond, —C(O)—, substituted or unsubstituted C₁-C₅ alkylene or substituted or unsubstituted 5 to 10 membered arylene.

Embodiment 134. The compound of embodiment 133, wherein L³ is R⁴⁶-substituted or unsubstituted ethylene.

Embodiment 135. The compound of embodiment 133, wherein L³ is unsubstituted ethylene.

Embodiment 136. The compound of embodiment 134, wherein R⁴⁶ is —OH.

Embodiment 137. The compound of embodiment 133, wherein L³ is R⁴⁶-substituted or unsubstituted phenylene.

Embodiment 138. The compound of embodiment 137, wherein R⁴⁶ is independently hydrogen, —OH, or —NO₂.

Embodiment 139. The compound of embodiment 78, wherein the compound has the structure:

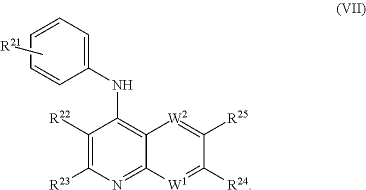

Embodiment 140. The compound of embodiment 139, wherein the compound has the structure:

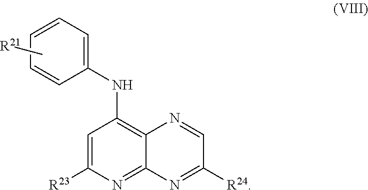

Embodiment 141. The compound of embodiment 78, wherein the compound has the structure:

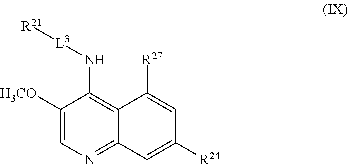

Embodiment 142. A method of treating a disease in a patient in need of such treatment, said method comprising administering a therapeutically effective amount of a compound of any one of embodiments 1-77.

Embodiment 143. The method of embodiment 142, wherein the disease is cancer.

Embodiment 144. The method of embodiment 143, wherein the cancer is brain cancer, glioblastoma multiforme, medulloblastoma, astrocytomas, brain stem gliomas, meningiomas, oligodendrogliomas, melanoma, lung cancer, breast cancer, or leukemia.

Embodiment 145. The method of embodiment 142, wherein the disease is Down's Syndrome.

Embodiment 146. A pharmaceutical composition comprising a pharmaceutically acceptable excipient and a compound of any one of embodiments 1-77.

Embodiment 147. A method of inhibiting the activity of Olig2 in a cell, said method comprising contacting said cell with a compound of any one of embodiments 1-77.

Embodiment 148. A method of treating a disease in a patient in need of such treatment, said method comprising administering a therapeutically effective amount of a compound of any one of embodiments 78-141.

Embodiment 149. The method of embodiment 148, wherein the disease is cancer.

Embodiment 150. The method of embodiment 149, wherein the cancer is brain cancer, glioblastoma multiforme, medulloblastoma, astrocytomas, brain stem gliomas, meningiomas, oligodendrogliomas, melanoma, lung cancer, breast cancer, or leukemia.

Embodiment 151. The method of embodiment 148, wherein the disease is Down's Syndrome.

Embodiment 152. A pharmaceutical composition comprising a pharmaceutically acceptable excipient and a compound of any one of embodiments 78-141.

Embodiment 153. A method of inhibiting the activity of Olig2 in a cell, said method comprising contacting said cell with a compound of any one of embodiments 78-141.

Embodiment 154. A method of treating a disease in a patient in need of such treatment, said method comprising administering a therapeutically effective amount of a compound of Table 1, 2, or 3.

Embodiment 155. The method of embodiment 154, wherein the disease is cancer.

Embodiment 156. The method of embodiment 155, wherein the cancer is brain cancer, glioblastoma multiforme, medulloblastoma, astrocytomas, brain stem gliomas, meningiomas, oligodendrogliomas, melanoma, lung cancer, breast cancer, or leukemia.

Embodiment 157. The method of embodiment 154, wherein the disease is Down's Syndrome.

Embodiment 158. A method of inhibiting the activity of Olig2 in a cell, said method comprising contacting said cell with a compound of Table 1, 2 or 3.

Embodiment 159. A pharmaceutical composition comprising a pharmaceutically acceptable excipient and a compound of Table 1, 2 or 3.

Embodiment 160. A method of identifying an inhibitor of protein dimerization, said method comprising:

(i) constructing in silico a computer readable peptide comprising a steric feature and an electronic feature, wherein said steric feature and said electronic feature form part of a first protein and wherein said steric feature and said electronic feature participate in dimerization of said first protein with a second protein;

(ii) determining in silico a level of binding of said computer readable peptide to a compound;

(iii) comparing said level to a control level, wherein an increase of said level compared to said control level indicates said compound is an inhibitor compound of protein dimerization.

Embodiment 161. A peptide, peptidomimetic, or cyclic peptide, wherein the peptide, peptidomimetic, or cyclic peptide is capable of binding to Olig2.

Embodiment 162. A method of treating a disease in a patient in need of such treatment, said method comprising administering a therapeutically effective amount of a peptide, peptidomimetic, or cyclic peptide of embodiment 161.

Embodiment 163. A pharmaceutical composition comprising a pharmaceutically acceptable excipient and a peptide, peptidomimetic, or cyclic peptide of embodiment 161.

Embodiment 164. A method of inhibiting the activity of Olig2 in a cell, said method comprising contacting said cell with a peptide, peptidomimetic, or cyclic peptide of embodiment 161.

Embodiment 165. A compound having the structure of Formula:

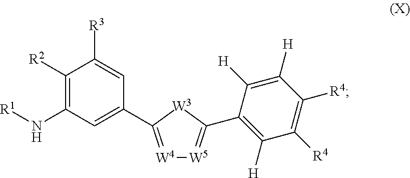

wherein:

W³ is O, S, or NR²⁵;

W⁴ and W⁵ are each independently CH or N;

R¹ is —SOₙ₁R⁵, —SOᵥ₁NR⁵R⁶, —C(O)R⁵, —C(O)—OR⁵, —C(O)NR⁵R⁶, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R² is hydrogen, halogen, —CX^{b}₃, —CN, —SOᵥ₂NR⁷R⁸, —NHNH₂, —ONR⁷R⁸, —NHC(O)NHNH₂, —NHC(O)NR⁷R⁸, —N(O)ₘ₂, —NR⁷R⁸, —NH—O—R⁷, —C(O)R⁷, —C(O)—OR⁷, —C(O)NR⁷R⁸, —OR⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R³ is hydrogen, halogen, —CX^{c}₃, —CN, —SOₙ₃R⁹, —SOᵥ₃NR⁹R¹⁹, —NHNH₂, —ONR⁹R¹⁹, —NHC(O)NHNH₂, —NHC(O)NR⁹R¹⁹, —N(O)ₘ₃, —NR⁹R¹⁰, —NH—O—R⁹, —C(O)R⁹, —C(O)—OR⁹, —C(O)NR⁹R¹⁰, —OR⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

each R⁴ is independently halogen, —CX^{d}₃, —CN, —SOₙ₄R¹¹, —SOᵥ₄NR¹¹R¹², —NHNH₂, —ONR¹¹R¹², —NHC(O)NHNH₂, —NHC(O)NR¹¹R¹², —N(O)ₘ₄, —NR¹¹R¹², —NH—O—R¹¹, —C(O)R¹¹, —C(O)—OR¹¹, —C(O)NR¹¹R¹², —OR¹¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁵ is hydrogen, —CF₃, —CN, —CCl₃, —COOH —CH₂COOH, —CONH₂, —OH, —SH, —SO₃H, —SO₂NH₂, —NO₂, —NH₂, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁶, R⁷, R⁸, R⁹, R¹⁰, R¹¹, and R¹² are independently hydrogen, —CF₃, —CN, —CCl₃, —COOH, —CH₂COOH, —CONH₂, —OH, —SH, —SO₃H, —SO₂NH₂, —NO₂, —NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁵ is H or substituted or unsubstituted C₁-C₆alkyl;

X^{b}, X^{c} and X^{d} are independently —F, —Cl, —Br, or —I;

n₁, n₃ and n₄ are independently an integer from 0 to 4;

m₂, m₃ and m₄ are independently an integer from 1 to 2; and

v₁, v₂, v₃ and v₄ are independently an integer from 1 to 2; or

a pharmaceutically acceptable salt, pharmaceutically acceptable solvate, or pharmaceutically acceptable prodrug thereof.

Embodiment 166. The compound of embodiment 165 wherein each R⁴ is independently halogen, CF₃, OR¹¹, or substituted or unsubstituted alkyl.

Embodiment 167. The compound of embodiment 2 wherein R² is hydrogen; R³ is hydrogen, halogen, —CF₃, or substituted or unsubstituted alkyl; and R²⁵ is H.

Embodiment 168. The compound of embodiment 3 wherein R¹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 169. The compound of embodiment 167 wherein R¹ is substituted or unsubstituted alkyl, substituted or unsubstituted heteroalkyl, substituted or unsubstituted cycloalkyl, or substituted or unsubstituted heterocycloalkyl.

Embodiment 170. The compound of embodiment 5 wherein W⁴ and W⁵ are each N.

Embodiment 171. A compound having the structure of Formula:

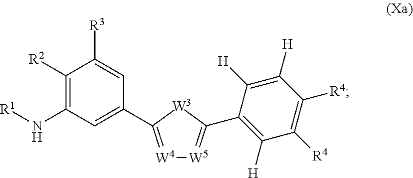

wherein:

W³ is O, S, or NR²⁵;

W⁴ and W⁵ are each independently CH or N;

R¹ is —SOₙ₁R⁵, —SOᵥ₁NR⁵R⁶, —C(O)R⁵, —C(O)—OR⁵, substituted or unsubstituted alkyl, substituted or unsubstituted heteroalkyl, substituted or unsubstituted cycloalkyl, or substituted or unsubstituted heterocycloalkyl;

R² is hydrogen, halogen, —CX^{b}₃, —CN, —SOᵥ₂NR⁷R⁸, —NHNH₂, —ONR⁷R⁸, —NHC(O)NHNH₂, —NHC(O)NR⁷R⁸, —N(O)ₘ₂, —NR⁷R⁸, —C(O)R⁷, —C(O)—OR⁷, —C(O)NR⁷R⁸, —OR⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R³ is hydrogen, halogen, —CX^{c}₃, —CN, —SOₙ₃R⁹, —SOᵥ₃NR⁹R¹⁰, —NHNH₂, —ONR⁹R¹⁰, —NHC(O)NHNH₂, —NHC(O)NR⁹R¹⁰, —N(O)ₘ₃, —NR⁹R¹⁰, —NH—O—R⁹, —C(O)R⁹, —C(O)—OR⁹, —C(O)NR⁹R¹⁰, —OR⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

each R⁴ is independently hydrogen, halogen, —CX^{d}₃, —CN, —SOₙ₄R¹¹, —SOᵥ₄NR¹¹R¹², —NHNH₂, —ONR¹¹R¹², —NHC(O)NHNH₂, —NHC(O)NR¹¹R¹², —N(O)ₘ₄, —NR¹¹R¹², —NH—O—R¹¹, —C(O)R¹¹, —C(O)—OR¹¹, —C(O)NR¹¹R¹², —OR¹¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁵ is hydrogen, —CN, —COOH, —CH₂COOH, —CONH₂, —OH, —SH, —SO₃H, —SO₂NH₂, —NO₂, or —NH₂;

R⁶, R⁷, R⁸, R⁹, R¹⁰, R¹¹, and R¹² are independently hydrogen, —CF₃, —CN, —CCl₃, —COOH, —CH₂COOH, —CONH₂, —OH, —SH, —SO₃H, —SO₂NH₂, —NO₂, —NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁵ is H or unsubstituted C₁-C₆alkyl;

X^{b}, X^{c} and X^{d} are independently —F, —Cl, —Br, or —I;

n₁, n₃ and n₄ are independently an integer from 0 to 4;

m₂, m₃ and m₄ are independently an integer from 1 to 2; and

v₁, v₂, v₃ and v₄ are independently an integer from 1 to 2; or a pharmaceutically acceptable salt, pharmaceutically acceptable solvate, or pharmaceutically acceptable prodrug thereof.

Embodiment 172. A compound having the structure of Formula:

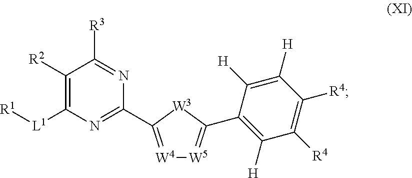

wherein:

L¹ is NH;

W³ is O, S, or NR²⁵;

W⁴ and W⁵ are each independently CH or N;

R¹ is hydrogen, —SOₙ₁R⁵, —SOᵥ₁NR⁵R⁶, —C(O)R⁵, —C(O)—OR⁵, —C(O)NR⁵R⁶, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R² is hydrogen, halogen, —CX^{b}₃, —CN, —SOₙ₂R⁷, —SOᵥ₂NR⁷R⁸, —NHNH₂, —ONR⁷R⁸, —NHC(O)NHNH₂, —NHC(O)NR⁷R⁸, —N(O)ₘ₂, —NR⁷R⁸, —NH—O—R⁷, —C(O)R⁷, —C(O)—OR⁷, —C(O)NR⁷R⁸, —OR′, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R³ is hydrogen, halogen, —CX^{c}₃, —CN, —SOₙ₃R⁹, —SOᵥ₃NR⁹R¹⁰, —NHNH₂, —ONR⁹R¹⁰, —NHC(O)NHNH₂, —NHC(O)NR⁹R¹⁰, —N(O)^{m3}, —NR⁹R¹⁰, —NH—O—R⁹, —C(O)R⁹, —C(O)—OR⁹, —C(O)NR⁹R¹⁰, —OR⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

each R⁴ is independently halogen, —CX^{d}₃, —CN, —SOₙ₄R¹¹, —SOᵥ₄NR¹¹, —NHNH₂, —ONR¹¹R¹², —NHC(O)NHNH₂, —NHC(O)NR¹¹R¹², —NOₘ₄, —NR¹¹R¹², —NH—O—R¹¹, —C(O)R¹¹, —C(O)—OR¹¹, —C(O)NR¹¹R¹², —OR¹¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁵, R⁶, R⁷, R⁸, R⁹, R¹⁰, R¹¹, and R¹² are independently hydrogen, —CF₃, —CN, —CCl₃, —COOH, —CH₂COOH, —OH, —SH, —SO₃H, —SO₂NH₂, —NO₂, —NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁵ is H or substituted or unsubstituted C₁-C₆alkyl;

X^{b}, X^{c} and X^{d} are independently —F, —Cl, —Br, or —I;

n₁, n₂, n₃ and n₄ are independently an integer from 0 to 4;

m₂, m₃ and m₄ are independently an integer from 1 to 2; and

v₁, v₂, v₃ and v₄ are independently an integer from 1 to 2; or

a pharmaceutically acceptable salt, pharmaceutically acceptable solvate, or pharmaceutically acceptable prodrug thereof.

Embodiment 173. The compound of embodiment 172 wherein each R⁴ is independently halogen, CF₃, OR¹¹, or substituted or unsubstituted alkyl.

Embodiment 174. The compound of embodiment 173 wherein R² is hydrogen; R³ is hydrogen, halogen, CF₃, or substituted or unsubstituted alkyl; and R²⁵ is H.

Embodiment 175. The compound of embodiment 10 wherein R¹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 176. The compound of embodiment 10 wherein R¹ is substituted or unsubstituted alkyl, substituted or unsubstituted heteroalkyl, substituted or unsubstituted cycloalkyl, or substituted or unsubstituted heterocycloalkyl.

Embodiment 177. The compound of embodiment 12 wherein W⁴ and W⁵ are each N.

Embodiment 178. A compound having the structure of Formula:

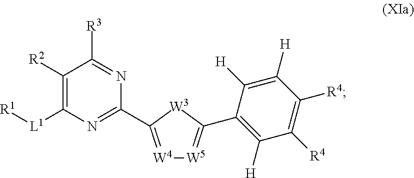

wherein:

L¹ is NH;

W³ is O, S, or NR²⁵;

W⁴ and W⁵ are each independently CH or N;

R¹ is —SOₙ₁R⁵, —SOᵥ₁NR⁵R⁶, —C(O)R⁵, —C(O)—OR⁵, —C(O)NR⁵R⁶, substituted or unsubstituted heteroalkyl, substituted or unsubstituted cycloalkyl, substituted or unsubstituted heterocycloalkyl, or substituted or unsubstituted heteroaryl;

R² is hydrogen, halogen, —CX^{b}₃, —CN, —SOₙ₂R⁷, —SOᵥ₂NR⁷R⁸, —NHNH₂, —ONR⁷R⁸, —NHC(O)NHNH₂, —NHC(O)NR⁷R⁸, —N(O)ₘ₂, —NR⁷R⁸, —NH—O—R⁷, —C(O)R⁷, —C(O)—OR⁷, —C(O)NR⁷R⁸, —OR⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R³ is hydrogen, halogen, —CX^{c}₃, —CN, —SOₙ₃R⁹, —SOᵥ₃NR⁹R¹⁰, —NHNH₂, —ONR⁹R¹⁰, —NHC(O)NHNH₂, —NHC(O)NR⁹R¹⁰, —N(O)ₘ₃, —NR⁹R¹⁰, —NH—O—R⁹, —C(O)R⁹, —C(O)—OR⁹, —C(O)NR⁹R¹⁰, —OR⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

each R⁴ is independently hydrogen, halogen, —CX^{d}₃, —CN, —SOₙ₄R¹¹, —SOᵥ₄NR¹¹R¹² —NHNH₂, —ONR¹¹R¹², —NHC(O)NHNH₂, —NHC(O)NR¹¹R¹², —N(O)ₘ₄, —NR¹¹R¹², —NH—O—R¹¹, —C(O)R¹¹, —C(O)—OR¹¹, —C(O)NR¹¹R¹², —OR¹¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁵, R⁶, R⁷, R⁸, R⁹, R¹⁰, R¹¹, and R¹² are independently hydrogen, —CF₃, —CN, —CCl₃, —COOH, —CH₂COOH, —CONH₂, —OH, —SH, —SO₃H, —SO₂NH₂, —NO₂, —NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁵ is H or substituted or unsubstituted C₁-C₆alkyl;

X^{b}, X^{c} and X^{d} are independently —F, —Cl, —Br, or —I;

n₁, n₂, n₃ and n₄ are independently an integer from 0 to 4;

m₂, m₃ and m₄ are independently an integer from 1 to 2; and

v₁, v₂, v₃ and v₄ are independently an integer from 1 to 2; or

a pharmaceutically acceptable salt, pharmaceutically acceptable solvate, or pharmaceutically acceptable prodrug thereof.

Embodiment 179. A compound having the structure of Formula:

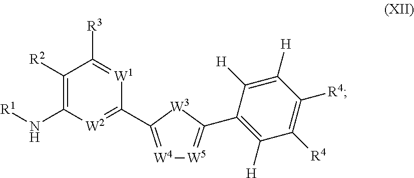

wherein:

one of W¹ and W² is N and the other is CH;

W³ is O, S, or NR²⁵;

W⁴ and W⁵ are each independently CH or N;

R¹ is hydrogen, —SOₙ₁R⁵, —SOᵥ₁NR⁵R⁶, —C(O)R⁵, —C(O)—OR⁵, —C(O)NR⁵R⁶, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R² is hydrogen, halogen, —CX^{b}₃, —CN, —SOₙ₂R⁷, —SOᵥ₂NR⁷R⁸, —NHNH₂, —ONR⁷R⁸, —NHC(O)NHNH₂, —NHC(O)NR⁷R⁸, —N(O)ₘ₂, —NR⁷R⁸, —NH—O—R⁷, —C(O)R⁷, —C(O)—OR⁷, —C(O)NR⁷R⁸, —OR⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R³ is hydrogen, halogen, —CX^{c}₃, —CN, —SOₙ₃R⁹, —SOᵥ₃NR⁹R¹⁰, —NHNH₂, —ONR⁹R¹⁰, —NHC(O)NHNH₂, —NHC(O)NR⁹R¹⁰, —N(O)ₘ₃R⁹, —NR⁹R¹⁰, —NH—O—R⁹, —C(O)R⁹, —C(O)—OR⁹, —C(O)NR⁹R¹⁰, —OR⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

each R⁴ is independently halogen, —CX^{d}₃, —CN, —SOₙ₄R¹¹, —SOᵥ₄NR¹¹R¹², —NHNH₂, —ONR¹¹R¹², —NHC(O)NHNH₂, —NHC(O)NR¹¹R¹², —N(O)ₘ₄, —NR¹¹R¹², —NH—O—R¹¹, —C(O)R¹¹, —C(O)—OR¹¹, —C(O)NR¹¹R¹², —OR¹¹ substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁵, R⁶, R⁷, R⁸, R⁹, R¹⁰, R¹¹ and R¹² are independently hydrogen, —CF₃, —CN, —CCl₃, —COOH, —CH₂COOH, —CONH₂, —OH, —SH, —SO₃H, —SO₂NH₂, —NO₂, —NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁵ is H or substituted or unsubstituted C₁-C₆alkyl;

X^{b}, X^{c} and X^{d} are independently —F, —Cl, —Br, or —I;

n₁, n₂, n₃ and n₄ are independently an integer from 0 to 4;

m₂, m₃ and m₄ are independently an integer from 1 to 2; and

v₁, v₂, v₃ and v₄ are independently an integer from 1 to 2; or

a pharmaceutically acceptable salt, pharmaceutically acceptable solvate, or pharmaceutically acceptable prodrug thereof.

Embodiment 180. The compound of embodiment 15 wherein each R⁴ is independently halogen, —CF₃, —OR¹¹, or substituted or unsubstituted alkyl.

Embodiment 181. The compound of embodiment 16 wherein R² is hydrogen; R³ is hydrogen, halogen, —CF₃, or substituted or unsubstituted alkyl; and R²⁵ is H.

Embodiment 182. The compound of embodiment 17 wherein R¹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 183. The compound of embodiment 17 wherein R¹ is substituted or unsubstituted alkyl, substituted or unsubstituted heteroalkyl, substituted or unsubstituted cycloalkyl, or substituted or unsubstituted heterocycloalkyl.

Embodiment 184. The compound of embodiment 19 wherein W⁴ and W⁵ are each N.

Embodiment 185. A compound having the structure of Formula:

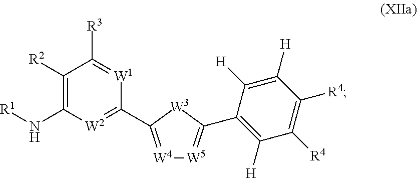

wherein:

one of W¹ and W² is N and the other is CH;

W³ is O, S, or NR²⁵;

W⁴ and W⁵ are each independently CH or N;

R¹ is —SOₙ₁R⁵, —SOᵥ₁NR⁵R⁶, —C(O)R⁵, —C(O)—OR⁵, —C(O)NR⁵R⁶, substituted or unsubstituted alkyl, substituted or unsubstituted heteroalkyl, substituted or unsubstituted cycloalkyl, or substituted or unsubstituted heterocycloalkyl;

R² is hydrogen, halogen, —CX^{b}₃, —CN, —SOₙ₂R⁷, —SOᵥ₂NR⁷R⁸, —NHNH₂, —ONR⁷R⁸, —NHC(O)NHNH₂, —NHC(O)NR⁷R⁸, —N(O)ₘ₂, —NR⁷R⁸, —NH—O—R⁷, —C(O)R⁷, —C(O)—OR⁷, —C(O)NR⁷R⁸, —OR⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R³ is hydrogen, halogen, —CX^{c}₃, —CN, —SOₙ₃R⁹, —SOᵥ₃NR⁹R¹⁰, —NHNH₂, —ONR⁹R¹⁰, —NHC(O)NHNH₂, —NHC(O)NR⁹R¹⁰, —N(O)ₘ₃, —NR⁹R¹⁰, —NH—O—R⁹, —C(O)R⁹, —C(O)—OR⁹, —C(O)NR⁹R¹⁰, —OR⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

each R⁴ is independently halogen, —CX^{d}₃, —CN, —SOₙ₄NR¹¹R¹², —NHNH₂, —ONR¹¹R¹², —NHC(O)NHNH₂, —NHC(O)NR¹¹R¹², —N(O)ₘ₄, —NR¹¹R¹², —NH—O—R¹¹, —C(O)R¹¹, —C(O)—OR¹¹, —C(O)NR¹¹R¹², —OR¹¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁵, R⁶, R⁷, R⁸, R⁹, R¹⁰, R¹¹, and R¹² are independently hydrogen, —CF₃, —CN, —CCl₃, —COOH, —CH₂COOH, —CONH₂, —OH, —SH, —SO₃H, —SO₂NH₂, —NO₂, —NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁵ is H or substituted or unsubstituted C₁-C₆alkyl;

X^{b}, X^{c} and X^{d} are independently —F, —Cl, —Br, or —I;

n₁, n₂, n₃ and n₄ are independently an integer from 0 to 4;

m₂, m₃ and m₄ are independently an integer from 1 to 2; and

v₁, v₂, v₃ and v₄ are independently an integer from 1 to 2; or a pharmaceutically acceptable salt, pharmaceutically acceptable solvate, or pharmaceutically acceptable prodrug thereof.

Embodiment 186. A compound having the structure of Formula:

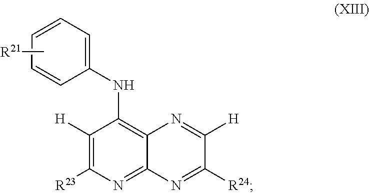

wherein:

R²¹ is hydrogen, F, Br, I, —CN, —SOₙ₁R²⁸, —SOᵥ₁NR²⁸R²⁹, —NHNH₂, —ONR²⁸R²⁹, —NHC(O)NHNH₂, —NHC(O)NR²⁸R²⁹, —NHC(O)R²⁸, —N(O)ₘ₁, —NR²⁸R²⁹, —NH—O—R²⁸, —C(O)R²⁸, —C(O)—OR²⁸, —C(O)NR²⁸R²⁹, —N(R²⁸)C(O)R²⁹, —OR²⁸, —O—C(O)NR²⁸R²⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²³ is hydrogen, halogen, —CX^{c}₃, —CN, —SOₙ₃R³², —SOᵥ₃NR³²R³³, —NR³²SOᵥ₃R³³, —NHNH₂, —CH₂NHNH₂, —ONR³²R³³, —NHC(O)NHNH₂, —NHC(O)NR³²R³³, —NHC(O)R³², —N(O)ₘ₃, —NR³²R³³, —NH—O—R³², —C(O)R³², —C(O)—OR³², —C(O)NR³²R³³ —N(R³²)C(O)R³³, —O—C(O)NR³²R³³, —OR³², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁴ is hydrogen, halogen, —CX^{d}₃, —CN, —SOₙ₄R³⁴, —SOᵥ₄NR³⁴R³⁵, —NHNH₂, —ONR³⁴R³⁵, —NHC(O)NHNH₂, —NHC(O)NR³⁴R³⁵, —NHC(O)R³⁴, —N(O)ₘ₄, —NR³⁴R³⁵, —NH—O—R³⁴, —C(O)R³⁴, —C(O)—OR³⁴, —C(O)NR³⁴R³⁵, —N(R³⁴)C(O)R³⁵, —O—C(O)NR³⁴R³⁵, —OR³⁴, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁸ is hydrogen, halogen, —CX^{h}₃, —CN, —SO₂Cl, —SOₙ₈R⁴², —SOᵥ₈NR⁴²R⁴³, —NHC(O)NR⁴²R⁴³, —NHC(O)R⁴², —N(O)ₘ₈, —NR⁴²R⁴³, —C(O)R⁴², —C(O)—OR⁴², —C(O)NR⁴²R⁴³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁹ is hydrogen, halogen, —CXⁱ₃, —CN, —SO₂Cl, —SOₙ₉R⁴⁴, —SOᵥ₉NR⁴⁴R⁴⁵, —NHC(O)NR⁴⁴R⁴⁵, —NHC(O)R⁴⁴, —N(O)ₘ₉, —NR⁴⁴R⁴⁵, —C(O)R⁴⁴, —C(O)—OR⁴⁴, —C(O)NR⁴⁴R⁴⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R³², R³³, R³⁴, R³⁵, R⁴², R⁴³, R⁴⁴ and R⁴⁵ are independently hydrogen, —CF₃, —CN, —CCl₃, —COOH, —CH₂COOH, —CONH₂, —OH, —SH, —SO₃H, —SO₂NH₂, —NO₂, —NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

X^{c}, X^{d}, X^{h} and Xⁱ are independently —F, —Cl, —Br, or —I;

n₁, n₃, n₄, n₈ and n₉ are independently an integer from 0 to 4;

m₁, m₃, m₄, m₈ and m₉ are independently an integer from 1 to 2; and

v₁, v₂, v₃, v₄, v₈, and v₉ are independently an integer from 1 to 2; or a pharmaceutically acceptable salt, pharmaceutically acceptable solvate, or pharmaceutically acceptable prodrug thereof.

Embodiment 187. The compound of embodiment 22 wherein R²³ is hydrogen, halogen, —CX^{c}₃, —CN, —SOₙ₃R³², —SOᵥ₃NR³²R³³, —NR³²SOᵥ₃R³³, —CH₂NHNH₂, —NHC(O)NR³²R³³, —NHC(O)R³², —NR³²R³³, —C(O)R³², —C(O)—OR³², —C(O)NR³²R³³, —OR³², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 188. The compound of embodiment 23 wherein R²⁴ is hydrogen, halogen, —CX^{d}₃, —CN, —SOₙ₄R³⁴, —SOᵥ₄NR³⁴R³⁵, —NHC(O)NR³⁴R³⁵, —NHC(O)R³⁴, —NR³⁴R³⁵, —C(O)R³⁴, —C(O)—OR³⁴, —C(O)NR³⁴R³⁵, —OR³⁴, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 189. The compound of embodiment 24 wherein R²³ is halogen, —SOᵥ₃NR³²R³³, —NR³²SOᵥ₃R³³, —CH₂NHNH₂, —NHC(O)R³², —C(O)NR³²R³³, —OR³², substituted or unsubstituted alkyl, substituted or unsubstituted heteroalkyl, substituted or unsubstituted cycloalkyl, or substituted or unsubstituted heterocycloalkyl.

Embodiment 190. The compound of embodiment 25 wherein R²⁴ is halogen, —NR³⁴R³⁵, —OR³⁴, substituted or unsubstituted alkyl, substituted or unsubstituted heteroalkyl, substituted or unsubstituted cycloalkyl, or substituted or unsubstituted heterocycloalkyl.

Embodiment 191. The compound of embodiment 26 wherein R²¹ is hydrogen, F, Br, —CN, —SOₙ₁R²⁸, SOᵥ₁NR²⁸R²⁹, —NHC(O)NR²⁸R²⁹, —NHC(O)R²⁸, —NR²⁸R²⁹, —C(O)R²⁸, —C(O)—OR²⁸, —C(O)NR²⁸R²⁹, —OR²⁸, —O—C(O)NR²⁸R²⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 192. The compound of embodiment 27 wherein R²¹ is F, Br, —NHC(O)NR²⁸R²⁹, —NHC(O)R²⁸, —NR²⁸R²⁹, —O—C(O)NR²⁸R²⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 193. A compound having the structure of Formula:

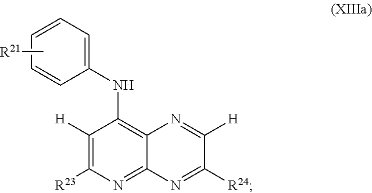

wherein:

R²¹ is hydrogen, halogen, —CX^{a}₃, —CN, —SOₙ₁R²⁸, —SOₙ₁NR²⁸R²⁹, —NHNH₂, —ONR²⁸R²⁹, —NHC(O)NHNH₂, —NHC(O)NR²⁸R²⁹, —NHC(O)R²⁸, —N(O)ₘ₁, —NR²⁸R²⁹, —NH—O—R²⁸, —C(O)R²⁸, —C(O)—OR²⁸, —C(O)NR²⁸R²⁹, —N(R²⁸)C(O)R²⁹, —OR²⁸, —O—C(O)NR²⁸R²⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²³ is hydrogen, halogen, —CX^{c}₃, —CN, —SOₙ₃R³², —SOᵥ₃NR³²R³³, —NR³²SOᵥ₃R³³, —NHNH₂, —CH₂NHNH₂, —ONR³²R³³, —NHC(O)NHNH₂, —NHC(O)NR³²R³³, —N(O)ₘ₃, —NH—O—R³², —C(O)R³², —C(O)—OR³², —C(O)NR³²R³³, —N(R³²)C(O)R³³, —O—C(O)NR³²R³³, —OR³², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁴ is hydrogen, halogen, —CX^{d}₃, —CN, —SOₙ₄R³⁴, —SOᵥ₄NR³⁴R³⁵, —NHNH₂, —ONR³⁴R³⁵, —NHC(O)NHNH₂, —NHC(O)NR³⁴R³⁵, —NHC(O)R³⁴, —N(O)ₘ₄, —NR³⁴R³⁵, —NH—O—R³⁴, —C(O)R³⁴, —C(O)—OR³⁴, —C(O)NR³⁴R³⁵, —N(R³⁴)C(O)R³⁵, —O—C(O)NR³⁴R³⁵, —OR³⁴, substituted or unsubstituted alkyl, substituted or unsubstituted heteroalkyl, substituted or unsubstituted cycloalkyl, substituted or unsubstituted heterocycloalkyl, or substituted or unsubstituted aryl;

R²⁸ is hydrogen, halogen, —CX^{h}₃, —CN, —SO₂Cl, —SOₙ₈R⁴², —SOᵥ₈NR⁴²R⁴³, —NHC(O)NR⁴²R⁴³, —NHC(O)R⁴², —N(O)ₘ₈, —NR⁴²R⁴³, —C(O)R⁴², —C(O)—OR⁴², —C(O)NR⁴²R⁴³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁹ is hydrogen, halogen, —CXⁱ₃, —CN, —SO₂Cl, —SOₙ₉R⁴⁴, —SOᵥ₉NR⁴⁴R⁴⁵, —NHC(O)NR⁴⁴R⁴⁵, —NHC(O)R⁴⁴, —N(O)ₘ₉, —NR⁴⁴R⁴⁵, —C(O)R⁴⁴, —C(O)—OR⁴⁴, —C(O)NR⁴⁴R⁴⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R³², R³³, R³⁴, R³⁵, R⁴², R⁴³, R⁴⁴ and R⁴⁵ are independently hydrogen, —CF₃, —CN, —CCl₃, —COOH, —CH₂COOH, —CONH₂, —OH, —SH, —SO₃H, —SO₂NH₂, —NO₂, —NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

X^{a}, X^{c}, X^{d}, X^{h} and Xⁱ are independently —F, —Cl, —Br, or —I;

n₁, n₃, n₄, n₈ and n₉ are independently an integer from 0 to 4;

m₁, m₃, m₄, m₈ and m₉ are independently an integer from 1 to 2; and

v₁, v₂, v₃, v₄, v₈, and v₉ are independently an integer from 1 to 2; or a pharmaceutically acceptable salt, pharmaceutically acceptable solvate, or pharmaceutically acceptable prodrug thereof.

Embodiment 194. A compound having the structure of Formula:

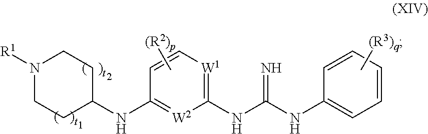

wherein:

one of W¹ and W² is N and the other is CH;

R¹ is hydrogen, —SOₙ₁R⁵, —SOᵥ₁NR⁵R⁶, —C(O)R⁵, —C(O)—OR⁵, —C(O)NR⁵R⁶, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

each R² is independently hydrogen, halogen, —CX^{b}₃, —CN, —SOₙ₂R⁵, —SOᵥ₂NR⁷R⁸, —NHNH₂, —ONR⁷R⁸, —NHC(O)NHNH₂, —NHC(O)NR⁷R⁸, —N(O)ₘ₂, —NR⁷R⁸, —NH—O—R⁷, —C(O)R⁷, —C(O)—OR⁷, —C(O)NR⁷R⁸, —OR⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

each R³ is independently hydrogen, halogen, —CX^{c}₃, —CN, —SOₙ₃R⁹, —SOᵥ₃NR⁹R¹⁶, —NHNH₂, —ONR⁹R¹⁶, —NHC(O)NHNH₂, —NHC(O)NR⁹R¹⁶, —N(O)ₘ₃, —NR⁹R¹⁰, —NH—O—R⁹, —C(O)R⁹, —C(O)—OR⁹, —C(O)NR⁹R¹⁰, —OR⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁵ is hydrogen, —CF₃, —CN, —CCl₃, —COOH, —CH₂COOH, —CONH₂, —OH, —SH, —SO₃H, —SO₂NH₂, —NO₂, —NH₂, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁶, R⁷, R⁸, R⁹, and R¹⁰ are independently hydrogen, —CF₃, —CN, —CCl₃, —COOH, —CH₂COOH, —CONH₂, —OH, —SH, —SO₃H, —SO₂NH₂, —NO₂, —NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

X^{b} and X^{c} are independently —F, —Cl, —Br, or —I;

n₁, n₂, and n₃ are independently an integer from 0 to 4;

m₂ and m₃ are independently an integer from 1 to 2; and

v₁, v₂, and v₃ are independently an integer from 1 to 2;

p is an integer from 1 to 2;

q is an integer from 1 to 4;

t₁ is an integer from 0 to 2;

t₂ is an integer from 1 to 2; or a pharmaceutically acceptable salt, pharmaceutically acceptable solvate, or pharmaceutically acceptable prodrug thereof,

Embodiment 195. The compound of embodiment 194 wherein p is 1 and q is 2.

Embodiment 196. The compound of embodiment 31 wherein each R³ is independently hydrogen, halogen, —CF₃, —OR⁹, or substituted or unsubstituted alkyl.

Embodiment 197. The compound of embodiment 32 wherein R² is hydrogen, halogen, —CF₃, or substituted or unsubstituted alkyl.

Embodiment 198. The compound of embodiment 33 wherein R¹ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 199. The compound of embodiment 34 wherein R¹ is hydrogen, substituted or unsubstituted alkyl, substituted or unsubstituted heteroalkyl, substituted or unsubstituted cycloalkyl, or substituted or unsubstituted heterocycloalkyl.

Embodiment 200. The compound of embodiment 35 wherein t₁ is 0 and t₂ is 1.

Embodiment 201. The compound of embodiment 35 wherein t₁ is 1 and t₂ is 1.

Embodiment 202. The compound of embodiment 199 wherein t1 is 0 and t2 is 2.

Embodiment 203. A compound having the structure of Formula:

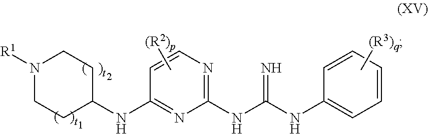

wherein:

R¹ is hydrogen, —SOₙ₁R⁵, —SOᵥ₁NR⁵R⁶, —C(O)R⁵, —C(O)—OR⁵, —C(O)NR⁵R⁶, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

each R² is independently hydrogen, halogen, —CX^{b}₃, —CN, —SOₙ₂R⁵, —SOᵥ₂NR⁷R⁸, —NHNH₂, —ONR⁷R⁸, —NHC(O)NHNH₂, —NHC(O)NR⁷R⁸, —N(O)ₘ₂, —NR⁷R⁸, —NH—O—R⁷, —C(O)R⁷, —C(O)—OR⁷, —C(O)NR⁷R⁸, —OR⁷, substituted or unsubstituted C₂-C₈alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

each R³ is independently hydrogen, halogen, —CX^{c}₃, —CN, —SOₙ₃R⁹, —SOᵥ₃NR⁹R¹⁰, —NHNH₂, —ONR⁹R¹⁰, —NHC(O)NHNH₂, —NHC(O)NR⁹R¹⁰, —N(O)ₘ₃, —NR⁹R¹⁰, —NH—O—R⁹, —C(O)R⁹, —C(O)—OR⁹, —C(O)NR⁹R¹⁰, —OR⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁵ is hydrogen, —CF₃, —CN, —CCl₃, —COOH, —CH₂COOH, —CONH₂, —OH, —SH, —SO₃H, —SO₂NH₂, —NO₂, —NH₂, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁶, R⁷, R⁸, R⁹, and R¹⁰ are independently hydrogen, —CF₃, —CN, —CCl₃, —COOH, —CH₂COOH, —CONH₂, —OH, —SH, —SO₃H, —SO₂NH₂, —NO₂, —NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

X^{b} and X^{c} are independently —F, —Cl, —Br, or —I;

n₁, n₂, and n₃ are independently an integer from 0 to 4;

m₂ and m₃ are independently an integer from 1 to 2; and

v₁, v₂, and v₃ are independently an integer from 1 to 2;

p is an integer from 1 to 2;

q is an integer from 1 to 4;

t₁ is an integer from 0 to 2;

t₂ is an integer from 1 to 2; or a pharmaceutically acceptable salt, pharmaceutically acceptable solvate, or pharmaceutically acceptable prodrug thereof.

Embodiment 204. The compound of embodiment 203 wherein p is 1 and q is 2.

Embodiment 205. The compound of embodiment 40 wherein each R³ is independently hydrogen, halogen, —CF₃, —OR⁹, or substituted or unsubstituted alkyl.

Embodiment 206. The compound of embodiment 41 wherein R² is hydrogen, halogen, —CF₃, or substituted or unsubstituted C₂-C₈alkyl.

Embodiment 207. The compound of embodiment 42 wherein R¹ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 208. The compound of embodiment 43 wherein R¹ is hydrogen, substituted or unsubstituted alkyl, substituted or unsubstituted heteroalkyl, substituted or unsubstituted cycloalkyl, or substituted or unsubstituted heterocycloalkyl.

Embodiment 209. The compound of embodiment 208 wherein t₁ is 0 and t₂ is 1.

Embodiment 210. The compound of embodiment 208 wherein t₁ is 1 and t₂ is 1.

Embodiment 211. The compound of embodiment 44 wherein t₁ is 0 and t₂ is 2.

Embodiment 212. A compound having the structure of Formula:

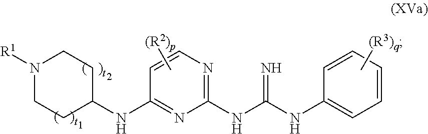

wherein:

R¹ is hydrogen, —SOₙ₁R⁵, —SOᵥ₁NR⁵R⁶, —C(O)R⁵, —C(O)—OR⁵, —C(O)NR⁵R⁶, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

each R² is independently hydrogen, halogen, —CX^{b}₃, —CN, —SOₙ₂R⁵, —SOᵥ₂NR⁷R⁸, —NHNH₂, —ONR⁷R⁸, —NHC(O)NHNH₂, —NHC(O)NR⁷R⁸, —N(O)ₘ₂, —NR⁷R⁸, —NH—O—R⁷, —C(O)R⁷, —C(O)—OR⁷, —C(O)NR⁷R⁸, —OR′, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

each R³ is independently hydrogen, halogen, —CX^{c}₃, —CN, —SOₙ₃R⁹, —SOᵥ₃NR⁹R¹⁰ —NHNH₂, —ONR⁹R¹⁰, —NHC(O)NHNH₂, —NHC(O)NR⁹R¹⁰, —N(O)ₘ₃, —NR⁹R¹⁰, —NH—O—R⁹, —C(O)R⁹, —C(O)—OR⁹, —C(O)NR⁹R¹⁰, —OR⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁵ is hydrogen, —CF₃, —CN, —CCl₃, —COOH, —CH₂COOH, —CONH₂, —OH, —SH, —SO₃H, —SO₂NH₂, —NO₂, —NH₂, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁶, R⁷, R⁸, R⁹, and R¹⁰ are independently hydrogen, —CF₃, —CN, —CCl₃, —COOH, —CH₂COOH, —CONH₂, —OH, —SH, —SO₃H, —SO₂NH₂, —NO₂, —NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

X^{b} and X^{c} are independently —F, —Cl, —Br, or —I;

n₁, n₂, and n₃ are independently an integer from 0 to 4;

m₂ and m₃ are independently an integer from 1 to 2; and

v₁, v₂, and v₃ are independently an integer from 1 to 2;

p is an integer from 1 to 2;

q is an integer from 1 to 4;

t₁ is an integer from 0 to 2;

t₂ is an integer from 1 to 2; or a pharmaceutically acceptable salt, pharmaceutically acceptable solvate, or pharmaceutically acceptable prodrug thereof.

Embodiment 213. A pharmaceutical composition comprising a pharmaceutically acceptable diluent, excipient, or binder, and a compound of any one of embodiments 1-48 or a pharmaceutically acceptable salt, pharmaceutically acceptable prodrug, or pharmaceutically acceptable solvate thereof.

Embodiment 214. A compound having the formula:

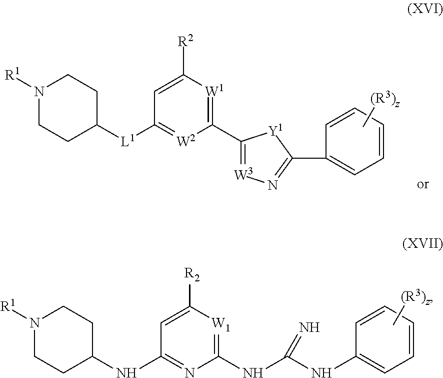

wherein,

R¹ is independently hydrogen, halogen, —CX^{a}₃, —CN, —SO₂Cl, —SOₙR⁷, —SOᵥNR⁴R⁵, —NHNH₂, —ONR⁴R⁵, —NHC═(O)NHNH₂, —NHC═(O)NR⁴R⁵, —N(O)ₘ, —NR⁴R⁵, —C(O)R⁶, —C(O)—OR⁶, —C(O)NR⁴R⁵, —OR⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R² is independently hydrogen, halogen, —CX^{b}₃, —CN, —SO₂Cl, —SOₙR¹¹, —SOᵥNR⁸R⁹, —NHNH₂, —ONR⁸R⁹, —NHC═(O)NHNH₂, —NHC═(O)NR⁸R⁹, —N(O)ₘ, —NR⁸R⁹, —C(O)R¹⁰, —C(O)—OR¹⁰, —C(O)NR⁸R⁹, —OR¹¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R³ is independently hydrogen, halogen, —CX^{c}₃, —CN, —SO₂Cl, —SOₙR¹⁵, —SOᵥNR¹²R¹³, —NHNH₂, —ONR¹²R¹³, —NHC═(O)NHNH₂, —NHC═(O)NR¹²R¹³, —N(O)ₘ, —NR¹²R¹³, —C(O)R¹⁴, —C(O)—OR¹⁴, —C(O)NR¹²R¹³, —OR¹⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁴, R⁵, R⁶, R⁷, R⁸, R⁹, R¹⁰, R¹¹, R¹², R¹³, R¹⁴, R¹⁵ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

W¹, W², and W³ are independently —CH— or —N—;

L¹ is independently —CH₂— or —NH—;

Y¹ is independently —O—, —S—, or —NH—;

X^{a}, X^{b}, and X^{c} are independently —F, —Cl, —Br, or —I;

n is independently an integer from 0 to 4;

m is independently an integer from 1 to 2;

v is independently an integer from 1 to 2;

z is independently an integer from 0 to 5.

Embodiment 215. A compound having the formula:

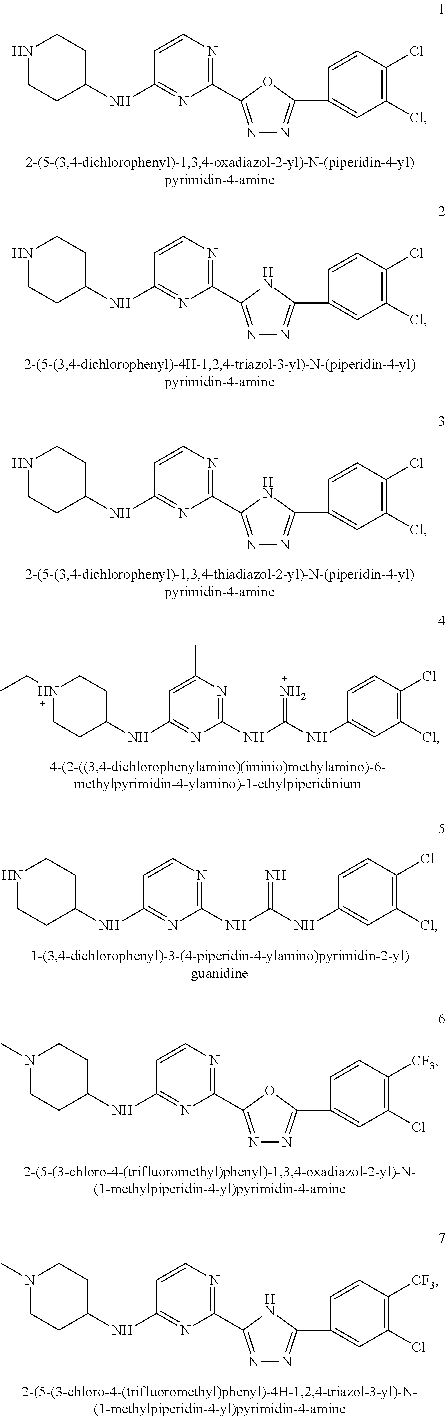

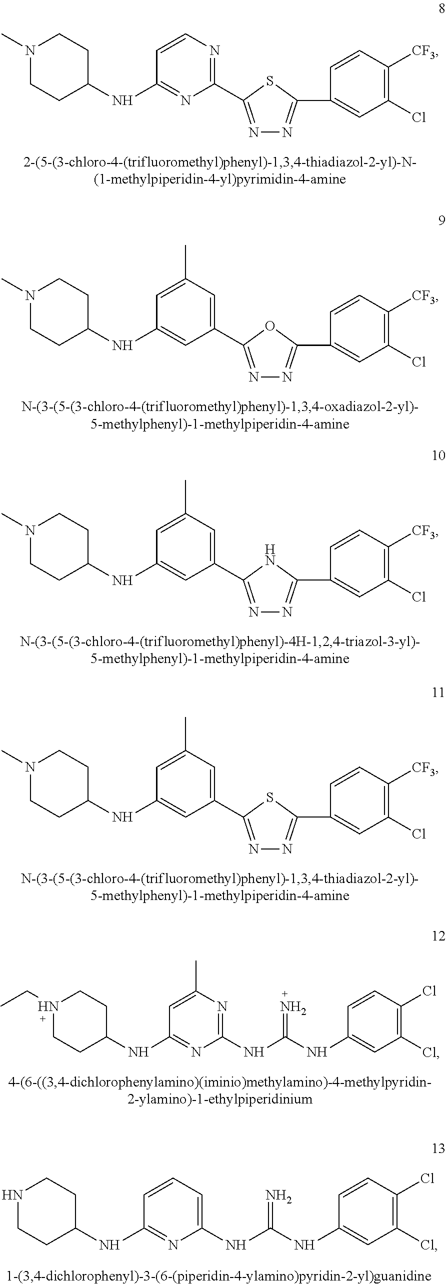 [SMILES: CN1CCC(Nc2ccnc(-c3nnc(-c4ccc(C(F)(F)F)c(Cl)c4)s3)n2)CC1 ; Cc1cc(NC2CCN(C)CC2)cc(-c2nnc(-c3ccc(C(F)(F)F)c(Cl)c3)o2)c1 ; Cc1cc(NC2CCN(C)CC2)cc(-c2nnc(-c3ccc(C(F)(F)F)c(Cl)c3)[nH]2)c1 ; Cc1cc(NC2CCN(C)CC2)cc(-c2nnc(-c3ccc(C(F)(F)F)c(Cl)c3)s2)c1 ; CCN1CCC(Nc2cc(C)nc(NC(=N)Nc3ccc(Cl)c(Cl)c3)n2)CC1 ; N=C(Nc1ccc(Cl)c(Cl)c1)Nc1cccc(NC2CCNCC2)n1]

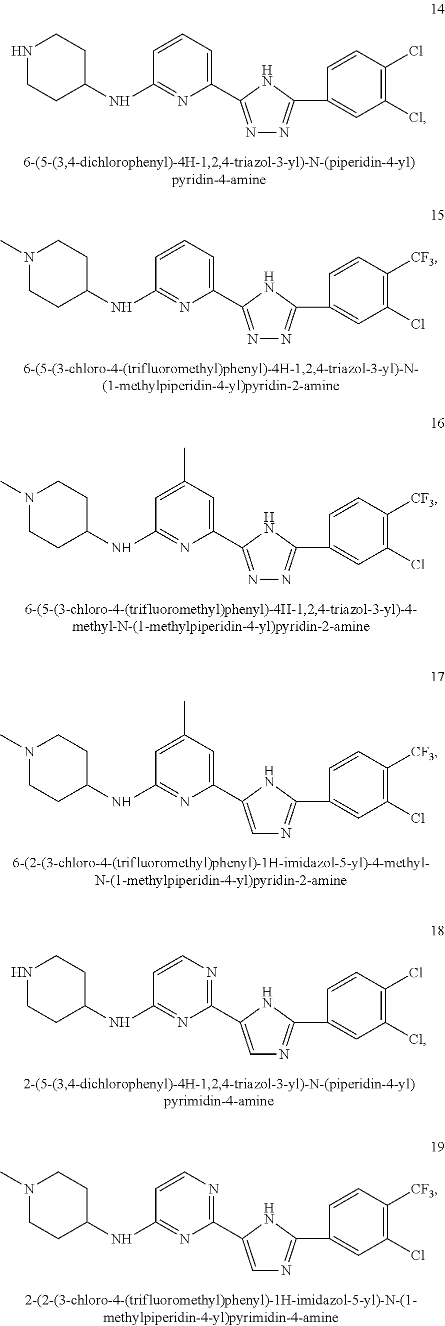 [SMILES: Clc1ccc(-c2nnc(-c3cccc(NC4CCNCC4)n3)[nH]2)cc1Cl ; CN1CCC(Nc2cccc(-c3nnc(-c4ccc(C(F)(F)F)c(Cl)c4)[nH]3)n2)CC1 ; Cc1cc(NC2CCN(C)CC2)nc(-c2nnc(-c3ccc(C(F)(F)F)c(Cl)c3)[nH]2)c1 ; Cc1cc(NC2CCN(C)CC2)nc(-c2cnc(-c3ccc(C(F)(F)F)c(Cl)c3)[nH]2)c1 ; Clc1ccc(-c2ncc(-c3nccc(NC4CCNCC4)n3)[nH]2)cc1Cl ; CN1CCC(Nc2ccnc(-c3cnc(-c4ccc(C(F)(F)F)c(Cl)c4)[nH]3)n2)CC1]

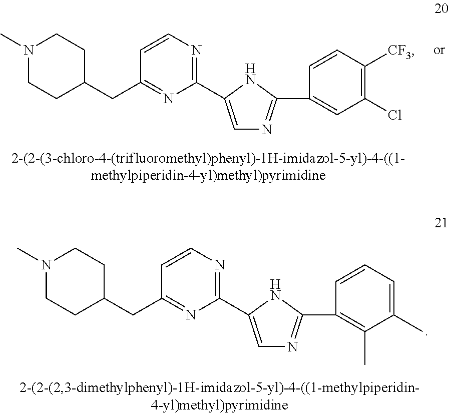

Embodiment 216. A compound having the formula:

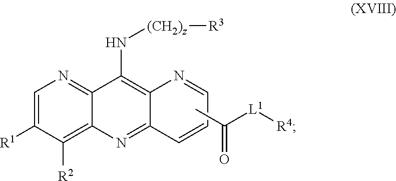

wherein,

R¹ is independently hydrogen, halogen, —CX^{a}₃, —CN, —SO₂Cl, —SOₙR⁸, —SOᵥNR⁵R⁶, —NHNH₂, —ONR⁵R⁶, —NHC═(O)NHNH₂, —NHC═(O)NR⁵R⁶, —N(O)ₘ, —NR⁵R⁶, —C(O)R⁷, —C(O)—OR⁷, —C(O)NR⁵R⁶, —OR⁸, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R² is independently hydrogen, halogen, —CX^{b}₃, —CN, —SO₂Cl, —SOₙR¹², —SOᵥNR⁹R¹⁰, —NHNH₂, —ONR⁹R¹⁰, —NHC═(O)NHNH₂, —NHC═(O)NR⁹R¹⁰, —N(O)ₘ, —NR⁹R¹⁰, —C(O)R¹¹, —C(O)—OR¹¹, —C(O)NR⁹R¹⁰, —OR¹², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R³ is independently hydrogen, halogen, —CX^{c}₃, —CN, —SO₂Cl, —SOₙR¹⁶, —SOᵥNR¹³R¹⁴, —NHNH₂, —ONR¹³R¹⁴, —NHC═(O)NHNH₂, —NHC═(O)NR¹³R¹⁴, —N(O)ₘ, —NR¹³R¹⁴, —C(O)R¹⁵, —C(O)—OR¹⁵, —C(O)NR¹³R¹⁴, —OR¹⁶, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁴ is independently hydrogen, halogen, —CX^{d}₃, —CN, —SO₂Cl, —SOₙR²⁰, —SOᵥNR¹⁷R¹⁸, —NHNH₂, —ONR¹⁷R¹⁸, —NHC═(O)NHNH₂, —NHC═(O)NR¹⁷R¹⁸, —N(O)ₘ, —NR¹⁷R¹⁸, —C(O)R¹⁹, —C(O)—OR¹⁹, —C(O)NR¹⁷R¹⁸, —OR²⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L¹ is independently —O— or —NH—;

X^{a}, X^{b}, X^{c}, and X^{d} are independently —F, —Cl, —Br, or —I;

n is independently an integer from 0 to 4;

m is independently an integer from 1 to 2;

v is independently an integer from 1 to 2;

z is independently an integer from 1 to 25.

Embodiment 217. A compound having the formula:

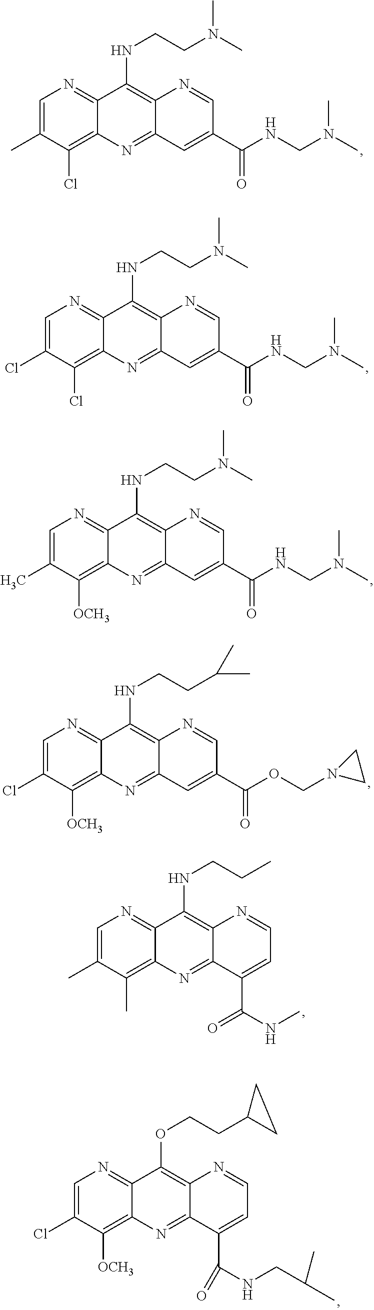

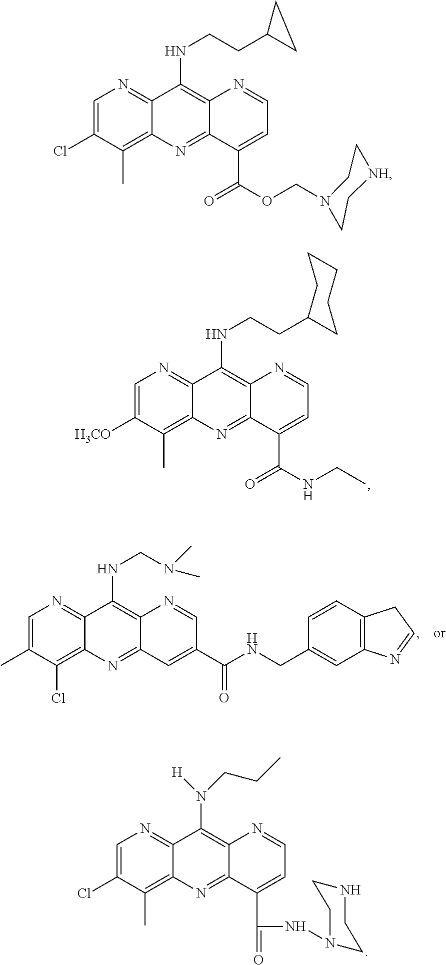

Embodiment 218. A compound having the formula:

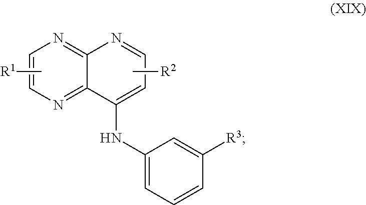

wherein,

R¹ is independently hydrogen, halogen, —CX^{a}₃, —CN, —SO₂Cl, —SOₙR⁷, —SOᵥNR⁴R⁵, —NHNH₂, —ONR⁴R⁵, —NHC═(O)NHNH₂, —NHC═(O)NR⁴R⁵, —N(O)ₘ, —NR⁴R⁵, —C(O)R⁶, —C(O)—OR⁶, —C(O)NR⁴R⁵, —OR⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R² is independently hydrogen, halogen, —CX^{b}₃, —CN, —SO₂Cl, —SOₙR^{1l}, —SOᵥNR⁸R⁹, —NHNH₂, —ONR⁸R⁹, —NHC═(O)NHNH₂, —NHC═(O)NR⁸R⁹, —N(O)ₘ, —NR⁸R⁹, —C(O)R¹⁰, —C(O)—OR¹⁰, —C(O)NR⁸R⁹, —OR¹¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R³ is independently hydrogen, halogen, —CX^{c}₃, —CN, —SO₂Cl, —SOₙR¹⁵, —SOᵥNR¹²R¹³, —NHNH₂, —ONR¹²R¹³, —NHC═(O)NHNH₂, —NHC═(O)NR¹²R¹³, —N(O)ₘ, —NR¹²R¹³, —C(O)R¹⁴, —C(O)—OR¹⁴, —C(O)NR¹²R¹³, —OR¹⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁴, R⁵, R⁶, R⁷, R⁸, R⁹, R¹⁰, R¹¹, R¹², R¹³, R¹⁴, R¹⁵ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

X^{a}, X^{b}, and X^{c} are independently —F, —Cl, —Br, or —I;

n is independently an integer from 0 to 4;

m is independently an integer from 1 to 2;

v is independently an integer from 1 to 2;

z is independently an integer from 0 to 5.

Embodiment 219. A compound having the formula:

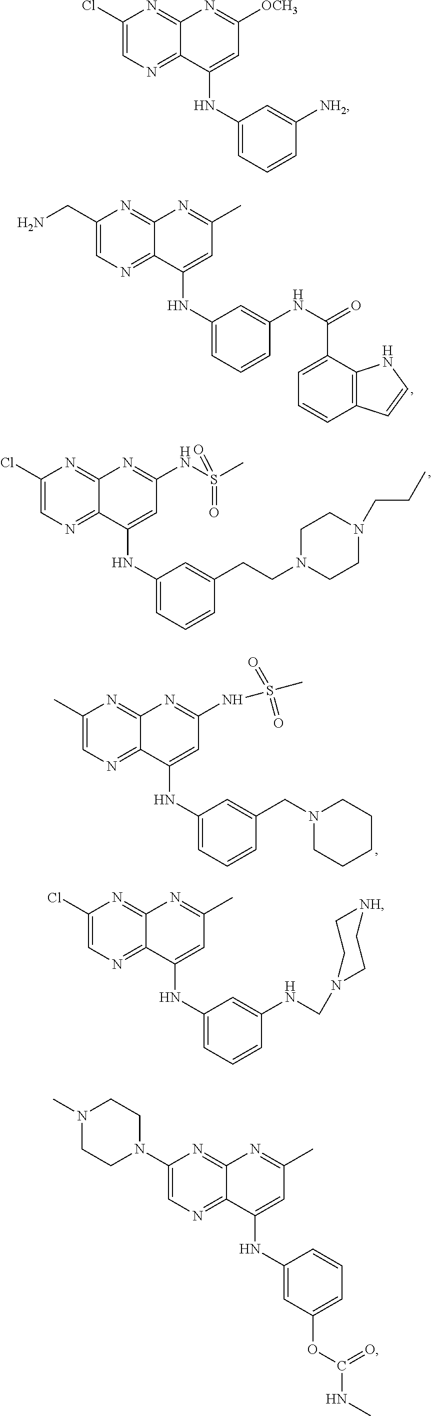

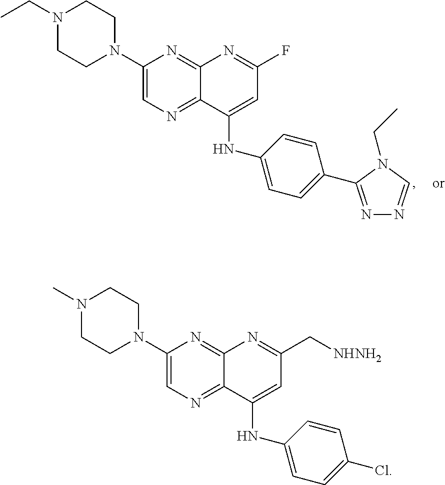 [SMILES: CCN1CCN(c2cnc3c(Nc4ccc(-c5nncn5CC)cc4)cc(F)nc3n2)CC1 ; CN1CCN(c2cnc3c(Nc4ccc(Cl)cc4)cc(CNN)nc3n2)CC1]

Embodiment 220. A pharmaceutical composition comprising a pharmaceutically acceptable excipient and a compound of one of claims 214 to 219.

Embodiment 221. A method of treating a disease in a patient in need of such treatment, said method comprising administering a therapeutically effective amount of a compound of one of claims 214 to 219.

Embodiment 222. The method of claim 57, wherein the disease is cancer.

Embodiment 223. The method of claim 222, wherein the cancer is brain cancer, glioblastoma multiforme, medulloblastoma, astrocytomas, brain stem gliomas, meningiomas, oligodendrogliomas, melanoma, lung cancer, breast cancer, or leukemia.

Embodiment 224. The method of claim 57, wherein the disease is Down's Syndrome.

Embodiment 225. A method of inhibiting the activity of Olig2 in a cell, said method comprising contacting said cell with a compound of one of claims 214 to 219.

1.-61. (canceled)

62. A method of treating cancer in a patient in need thereof, said method comprising administering a therapeutically effective amount of a compound of Formula: 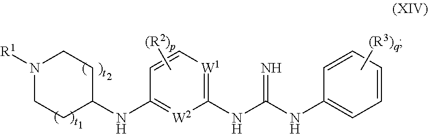
wherein:
one of W¹ and W² is —N— and the other is —CH—;
R¹ is hydrogen, —SOₙ₁R⁵, —SOᵥ₁NR⁵R⁶, —C(O)R⁵, —C(O)—OR⁵, —C(O)NR⁵R⁶, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R² is independently hydrogen, halogen, —CX^{b}₃, —CN, —SOₙ₂R⁵, —SOᵥ₂NR⁷R⁸, —NHNH₂, —ONR⁷R⁸, —NHC(O)NHNH₂, —NHC(O)NR⁷R⁸, —N(O)ₘ₂, —NR⁷R⁸, —NH—O—R⁷, —C(O)R⁷, —C(O)—OR⁷, —C(O)NR⁷R⁸, —OR⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R³ is independently hydrogen, halogen, —CX^{c}₃, —CN, —SOₙ₃R⁹, —SOₙ₃NR⁹R¹⁰, —NHNH₂, —ONR⁹R¹⁰, —NHC(O)NHNH₂, —NHC(O)NR⁹R¹⁰, —N(O)ₘ₃, —NR⁹R¹⁰, —NH—O—R⁹, —C(O)R⁹, —C(O)—OR⁹ , —C(O)NR⁹R¹⁰, —OR⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, —CF₃, —CN, —CCl₃, —COOH, —CH₂COOH, —CONH₂, —OH, —SH, —SO₃H, —SO₂NH₂, —NO₂, —NH₂, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶, R⁷, R⁸, R⁹, and R¹⁰ are independently hydrogen, —CF₃, —CN, —CCl₃, —COOH, —CH₂COOH, —CONH₂, —OH, —SH, —SO₃H, —SO₂NH₂, —NO₂, —NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
X^{b} and X^{c} are independently —F, —Cl, —Br, or —I;
n₁, n₂, and n₃ are independently an integer from 0 to 4;
m₂ and m₃ are independently an integer from 1 to 2; and
v₁, v₂, and v₃ are independently an integer from 1 to 2;
p is an integer from 1 to 2;
q is an integer from 1 to 4;
t₁ is an integer from 0 to 2; and
t₂ is an integer from 1 to 2.

63. The method of claim 62, wherein p is 1 and q is 2.

64. The method of claim 63, wherein each R³ is independently hydrogen, halogen, —CF₃, —OR⁹, or substituted or unsubstituted alkyl.

65. The method of claim 64, wherein R² is hydrogen, halogen, —CF₃, or substituted or unsubstituted alkyl.

66. The method of claim 65, wherein R¹ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

67. The method of claim 66, wherein R¹ is hydrogen, substituted or unsubstituted alkyl, substituted or unsubstituted heteroalkyl, substituted or unsubstituted cycloalkyl, or substituted or unsubstituted heterocycloalkyl.

68. The method of claim 67, wherein t₁ is 0 and t₂ is 1.

69. The method of claim 67, wherein t₁ is 1 and t₂ is 1.

70. The method of claim 67, wherein t₁ is 0 and t₂ is 2.

71. The method of claim 62, wherein said compound is of Formula:

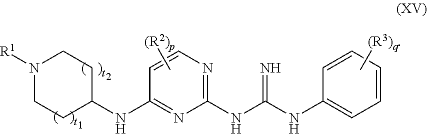

72. The method of claim 71, wherein each R³ is independently hydrogen, halogen, —CF₃, —OR⁹, or substituted or unsubstituted alkyl.

73. The method of claim 72, wherein each R² is independently hydrogen, halogen, —CF₃, or substituted or unsubstituted C₂-C₈alkyl.

74. The method of claim 73, wherein R¹ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

75. The method of claim 73, wherein R¹ is hydrogen, substituted or unsubstituted alkyl, substituted or unsubstituted heteroalkyl, substituted or unsubstituted cycloalkyl, or substituted or unsubstituted heterocycloalkyl.

76. The method of claim 71, wherein R¹ is hydrogen, —SOₙ₁R⁵, —SOᵥ₁NR⁵R⁶, —C(O)R⁵, —C(O)—OR⁵, —C(O)NR⁵R⁶, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
each R² is independently hydrogen, halogen, —CX^{b}₃, —CN, —SOₙ₂R⁵, —SOₙ₂NR⁷R⁸, —NHNH₂, —ONR⁷R⁸, —NHC(O)NHNH₂, —NHC(O)NR⁷R⁸, —N(O)ₘ₂, —NR⁷R⁸, —NH—O—R⁷, —C(O)R⁷, —C(O)—OR⁷, —C(O)NR⁷R⁸, —OR⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

77. The method of claim 62, wherein said compound is of Formula:

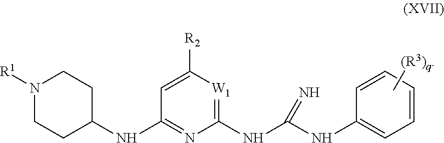

78. The method of claim 62, wherein said compound is of Formula:

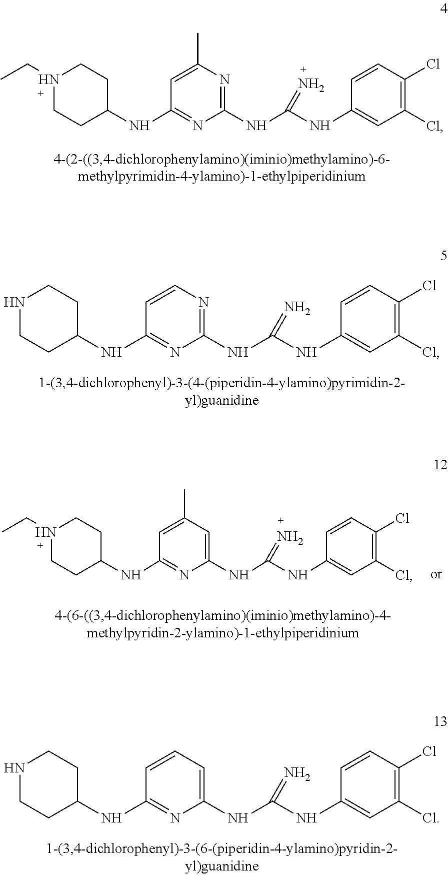 [SMILES: CCN1CCC(Nc2cc(C)nc(NC(=N)Nc3ccc(Cl)c(Cl)c3)n2)CC1 ; N=C(Nc1ccc(Cl)c(Cl)c1)Nc1nccc(NC2CCNCC2)n1 ; CCN1CCC(Nc2cc(C)cc(NC(=N)Nc3ccc(Cl)c(Cl)c3)n2)CC1 ; "or" ; N=C(Nc1ccc(Cl)c(Cl)c1)Nc1cccc(NC2CCNCC2)n1]

79. The method of one of claim 62, 71 or 77, wherein said cancer is brain cancer, glioblastoma multiforme, medulloblastoma, astrocytomas, brain stem gliomas, meningiomas, oligodendrogliomas, melanoma, lung cancer, breast cancer, or leukemia.
